(19) United States
(12) Patent Application Publication
(10) Pub. No.: US 2007/0275368 A1
(43) Pub. Date: Nov. 29, 2007

(54) Posh Associated Kinases And Related Methods

(75) Inventors: Iris Alroy, Ness-Ziona (IL); Yuval Reiss, Kiriat-Ono (IL); Daniel N. Taglicht, Lapid (IL); Shmuel Tuvia, Netanya (IL); Liora Yaar, Raanana (IL)

Correspondence Address: ROPES & GRAY LLP;PATENT DOCKETING 39/41, ONE INTERNATIONAL PLACE, BOSTON, MA, 02110-2624, US

(73) Assignees: PROTEOLOGICS, LTD., WEIZMANN SCIENCE PARK (IL); PROTEOLOGICS. INC., ORANGEBURG NEW YORK 10962 (NY)

(21) Appl. No.: 10/544,404

(22) PCT Filed: Feb. 5, 2004
(86) PCT No.: PCT/US04/03600

§ 371 Date: Jul. 13, 2006

Related U.S. Application Data
(60) Provisional application No. 60/445,534, filed on Feb. 5, 2003.
(60) Provisional application No. 60/451,437, filed on Mar. 3, 2003.
(60) Provisional application No. 60/464,285, filed on Apr. 21, 2003.
(60) Provisional application No. 60/503,931, filed on Sep. 16, 2003.

(51) Int. Cl. C12Q 1/70; C12N 9/12; C12Q 1/48

(52) U.S. Cl. 435/5; 435/15; 435/194

(57) ABSTRACT

The application provides novel complexes of POSH polypeptides and POSH asoociated kinases. The application also provides methods and compositions for treating a POSH-associated diseases such as viral disorders and cancer.

### RELATED APPLICATIONS

This application is a national stage filing under 35 U.S.C. 371 of International Application No. PCT/US2004/003600, filed Feb. 5, 2004, which claims priority from U.S. Provisional Application No. 60/445,534 filed Feb. 5, 2003; U.S. Provisional Application No. 60/451,437 filed Mar. 3, 2003; U.S. Provisional Application No. 60/464,285 filed Apr. 21, 2003; and U.S. Provisional Application No. 60/503,931 filed Sep. 16, 2003. The entire teachings of the referenced Applications are incorporated herein by reference in their entirety. International Application PCT/US2004/003600 was published under PCT Article 21(2) in English.

### BACKGROUND

Potential drug target validation involves determining whether a DNA, RNA or protein molecule is implicated in a disease process and is therefore a suitable target for development of new therapeutic drugs. Drug discovery, the process by which bioactive compounds are identified and characterized, is a critical step in the development of new treatments for human diseases. The landscape of drug discovery has changed dramatically due to the genomics revolution. DNA and protein sequences are yielding a host of new drug targets and an enormous amount of associated information.

The identification of genes and proteins involved in various disease states or key biological processes, such as inflammation and immune response, is a vital part of the drug design process. Many diseases and disorders could be treated or prevented by decreasing the expression of one or more genes involved in the molecular etiology of the condition if the appropriate molecular target could be identified and appropriate antagonists developed. For example, cancer, in which one or more cellular oncogenes become activated and result in the unchecked progression of cell cycle processes, could be treated by antagonizing appropriate cell cycle control genes. Furthermore many human genetic diseases, such as Huntington's disease, and certain prion conditions, which are influenced by both genetic and epigenetic factors, result from the inappropriate activity of a polypeptide as opposed to the complete loss of its function. Accordingly, antagonizing the aberrant function of such mutant genes would provide a means of treatment. Additionally, infectious diseases such as HIV have been successfully treated with molecular antagonists targeted to specific essential retroviral proteins such as HIV protease or reverse transcriptase. Drug therapy strategies for treating such diseases and disorders have frequently employed molecular antagonists which target the polypeptide product of the disease gene(s). However, the discovery of relevant gene or protein targets is often difficult and time consuming.

One area of particular interest is the identification of host genes and proteins that are co-opted by viruses during the viral life cycle. The serious and incurable nature of many viral diseases, coupled with the high rate of mutations found in many viruses, makes the identification of antiviral agents a high priority for the improvement of world health. Genes and proteins involved in a viral life cycle are also appealing as a subject for investigation because such genes and proteins will typically have additional activities in the host cell and may play a role in other non-viral disease states.

Other areas of interest include the identification of genes and proteins involved in cancer, apoptosis and neural disorders (particularly those associated with apoptotic neurons, such as Alzheimer's disease).

It would be beneficial to identify proteins involved in one or more of these processs for use in, among other things, drug screening methods. Additionally, once a protein involved in one or more processes of interest has been identified, it is possible to identify proteins that associate, directly or indirectly, with the initially identified protein. Knowledge of interactors will provide insight into protein assemblages and pathways that participate in disease processes, and in many cases an interacting protein will have desirable properties for the targeting of therapeutics. In some cases, an interacting protein will already be known as a drug target, but in a different biological context. Thus, by identifying a suite of proteins that interact with an initially identified protein, it is possible to identify novel drug targets and new uses for previously known therapeutics.

### SUMMARY

The disclosure provides, in part, novel interactions between protein kinases and the protein POSH (Plenty Of SH3 domains). In addition, the disclosure provides novel uses for agents that modulate POSH-associated kinases (POSH-AKs). For example, the disclosure provides methods for treating viral disorders and POSH-associated cancers by administering an agent that modulates the. activity of a POSH-associated kinase. Furthermore, the disclosure provides novel uses for agents that modulate POSH; such agents may be used to affect processes that are regulated by POSH-associated kinases. The disclosure also provides a multitude of screening assays and assays for evaluating novel effects of compounds that have already been identified as modulators of POSH or a POSH-AK. Other aspects and embodiments are presented below.

By providing novel POSH:POSH-AK interactions, the application provides, in part, methods for modulating a process that POSH participates in by targeting a POSH-AK or the POSH:POSH-AK interaction. Furthermore, by providing novel POSH:POSH-AK interactions, the application provides, in part, methods for modulating a process that a POSH-AK participates in by targeting POSH. As one of skill in the art can readily appreciate, a POSH protein may form multiple different complexes with POSH-AKs, depending on the biological context.

In certain aspects, the application provides an isolated, purified or recombinant polypeptide complex comprising a POSH polypeptide and a POSH-AK. In certain embodiments, the complex comprises a POSH-AK that interacts with a POSH polypeptide in a yeast two-hybrid assay or an immunoprecipitation. In certain embodiments, a POSH-AK is a PKA subunit polypeptide selected from the group consisting of: PRKAR1A, PRKACA, and PRKACB. In other embodiments, the POSH polypeptide is human POSH polypeptide and the POSH-AK is a kinase of a Rac-JNK signaling pathway (also referred to herein as the JNK signaling pathway), which is selected from the group consisting of MLK1, MLK2, MLK3, MKK4, MKK7, JNK1 and JNK2. In certain embodiments, the POSH polypeptide is a POSH RING domain, such as the RING domain of SEQ ID NO:26 or a polypeptide at least 90% identical to SEQ ID NO:26. In certain embodiments, the POSH polypeptide is a POSH SH3 domain, such as the SH3₄ domain of SEQ ID NO:30 or a polypeptide at least 90% identical to SEQ ID NO:30. In certain embodiments, a complex comprises a POSH polypeptide lacking a RING domain and a PKA subunit polypeptide selected from the group consisting of: PRKAR1A, PRKACA, and PRKACB. In certain embodiments, a complex comprises a portion of a naturally occurring POSH sufficient to interact with the POSH-AK.

In certain aspects the application provides methods for identifying an agent that modulates an activity of a POSH polypeptide or POSH-AK by identifying an agent that disrupts the interaction between a POSH polypeptide and a POSH-AK. In certain embodiments, the method comprises identifying an agent that disrupts a complex comprising a POSH polypeptide and a POSH-AK, wherein an agent that disrupts such a complex is an agent that modulates an activity of the POSH polypeptide or the POSH-AK. Often, an agent identified in this manner will affect both POSH and POSH-AK activities. Optionally the POSH-AK is a PKA, which may comprise a subunit such as PRKAR1A, PRKACA or PRKACB. Optionally the POSH-AK is a kinase of the JNK pathway, such as MLK1, MLK2, MLK3, MKK4, MKK7, JNK1 or JNK2.

In one embodiment, the application provides a method of identifying an antiviral agent, comprising: (a) identifying a test agent that disrupts a complex comprising a POSH polypeptide and a POSH-AK or a subunit of a POSH-AK; and (b) evaluating the effect of the test agent on a function of a virus, wherein an agent that inhibits a pro-infective or pro-replicative function of a virus is an antiviral agent. In general, the agent may inhibit any function of a virus that the virus employs in mounting and/or maintaining an infection in a host. Optionally, the virus is an envelope virus, such as a lentivirus (e.g., HIV or MMuLV), a flavivirus (e.g., West Nile virus) or a hepatitis virus (e.g., HBV, HCV). A variety of methods may be employed to evaluate the effect the test agent on a function of the virus, including in vitro (e.g. biochemical) assays, cell-based assays, animal based assays or human clinical trials. As an example, evaluating the effect of the test agent on a function of the virus may comprise evaluating the effect of the test agent on the budding or release of the virus or a virus-like particle. Optionally the POSH-AK is a PKA, which may comprise a subunit such as PRKAR1A, PRKACA or PRKACB. Optionally the POSH-AK is a kinase of the JNK pathway, such as MLK1, MLK2, MLK3, MKK4, MKK7, JNK1 or JNK2.

In one embodiment, the disclosure provides a method of identifying an anti-apoptotic agent, comprising: (a) identifying a test agent that disrupts a complex comprising a POSH polypeptide and a POSH-AK or a subunit of a POSH-AK; and (b) evaluating the effect of the test agent on apoptosis of a cell, wherein an agent that decreases apoptosis of the cell is an anti-apoptotic agent. In a preferred embodiment, the POSH polypeptide is a human POSH polypeptide (or a functional fragment thereof) and the POSH-AK is a kinase of the JNK pathway, such as MLK1, MLK2, MLK3, MKK4, MKK7, JNK1 or JNK2. A variety of methods may be employed to evaluate the effect the test agent on apoptosis of a cell, including cell-based assays using molecular markers of apoptosis or cell death, for example, animal based assays or human clinical trials.

In certain embodiments, the disclosure provides a method of identifying an anti-cancer agent, comprising: (a) identifying a test agent that disrupts a complex comprising a POSH polypeptide and a POSH-AK or a subunit of a POSH-AK; and (b) evaluating the effect of the test agent on proliferation or survival of a cancer cell, wherein an agent that decreases proliferation or survival of a cancer cell is an anti-cancer cell. In preferred embodiments, the cancer cell is derived from a POSH-associated cancer. Optionally the POSH-AK is a PKA, which may comprise a subunit such as PRKAR1A, PRKACA or PRKACB. Optionally the POSH-AK is a kinase of the JNK pathway, such as MLK1, MLK2, MLK3, MKK4, MKK7, JNK1 or JNK2.

In certain embodiments, the disclosure provides a method of identifying an agent that inhibits trafficking of a protein through the secretory pathway, comprising: (a) identifying a test agent that disrupts a complex comprising a POSH polypeptide and a POSH-AK or a subunit of a POSH-AK; and (b) evaluating the effect of the test agent on the trafficking of a protein through the secretory pathway. By trafficking is meant localization to or within the secretory pathway, processing in the secretory pathway (e.g., glycosylation, lipid modification, disulfide isomerization) or passage through the secretory pathway to a cellular or extracellular location such as the extracellular matrix, the extracellular medium, the plasma membrane or a cellular compartment such as a lysosome or endosome. Optionally, the method comprises evaluating the effect of the test agent on the trafficking of a myristoylated protein through the secretory pathway. Optionally the method comprises evaluating the effect of the test agent on the trafficking of a viral protein through the secretory pathway. Examples of proteins that may be monitored include HIV Gag, HIV Nef, Rapsyn, Src and Phospholipase D (PLD).

In certain aspects, the application provides an isolated antibody, or fragment thereof, specifically immunoreactive with an epitope of a sequence selected from the group consisting of SEQ ID NO: 2 which antibody disrupts the interaction between a polypeptide of SEQ ID NO: 2 and a POSH-AK. In a preferred embodiment, the antibody or fragment thereof disrupts the interaction between a POSH domain and a POSH-AK selected from the group consisting of: PRKAR1A, PRKACA, and PRKACB.

In certain aspects, the application provides methods of inhibiting viral infections comprising administering an agent to a subject in need thereof wherein said agent inhibits the interaction between a POSH polypeptide and a POSH-AK. Optionally, the virus is an envelope virus, such as a lentivirus (e.g., HIV or MMuLV), a flavivirus (e.g., West Nile virus) or a hepatitis virus (e.g., HBV, HCV).

In certain aspects, the application provides methods for identifying an antiviral, anti-cancer or antiapoptotic agent comprising: a) providing a POSH-AK polypeptide and a test agent; and b) identifying a test agent that binds to the POSH-AK polypeptide. In certain aspects the method comprises a) contacting a POSH-AK polypeptide with a test agent, and b) identifying a test agent that modulates an activity of the POSH-AK. Preferred POSH-AKs for use in such a method include a PKA subunit polypeptide (e.g., PRKAR1A, PRKACA, or PRKACB). In certain aspects, the application provides methods for identifying an antiviral, anti-cancer or antiapoptotic agent comprising: a) providing a POSH-AK polypeptide and a test agent; and b) identifying a test agent that modulates activity of the POSH-AK polypeptide. Preferred POSH-AKs for use in such a method include a PKA subunit polypeptide (e.g., PRKAR1A, PRKACA, or PRKACB).

In certain aspects, the application provides methods of inhibiting viral infections comprising administering an agent to a subject in need thereof wherein said agent modulates the activity of a POSH-AK. In certain preferred embodiments, the POSH-AK is a PKA subunit polypeptide (e.g., PRKAR1A, PRKACA, or PRKACB).

In certain aspects, the disclosure provides methods of treating or preventing a viral infection in a subject by inhibiting a POSH-AK. A method may comprise administering, to a subject in need thereof, an agent that inhibits a POSH-AK in an amount sufficient to inhibit the viral infection. An agent for use in such a method may be an agent that, for example, inhibits a kinase activity of the POSH-AK, inhibits expression of a POSH-AK, inhibits interaction between kinase subunits, inhibits the interaction between the POSH-AK and POSH. Optionally, the POSH-AK comprises a polypeptide selected from the group consisting of: PRKAR1A, PRKACA, and PRKACB. In certain embodiments, the subject is infected with an envelope virus, such as a lentivirus (e.g., HIV or MMuLV), a flavivirus (e.g., West Nile virus) or a hepatitis virus (e.g., HBV, HCV). The agent may be an siRNA construct comprising a nucleic acid sequence that hybridizes to an mRNA encoding the POSH-AK or a subunit of the POSH-AK. The agent may also be a small molecule inhibitor of the POSH-AK kinase activity, such as, in the case of PKA, adenosine cyclic monophosphorothioate, isoquinolinesulfonamide, piperazine, piceatannol, and ellagic acid.

In certain aspects, the disclosure provides methods for identifying an agent that modulates a POSH function, comprising: (a) identifying an agent that modulates a POSH-AK; and (b) testing the effect of the agent on a POSH function. In certain aspects the disclosure provides methods for evaluating the effect of an agent on a POSH function, comprising: (a) providing an agent that modulates a POSH-AK; and (b) testing the effect of the agent on a POSH function. Optionally, the POSH-AK is PRKAR1A, PRKACA, and PRKACB, JNK1, JNK2, MLK1, MLK2, MLK3, MKK4, and MKK7. The effect of an agent on POSH function may be assessed in any number of ways, including in vitro (e.g. biochemically), in a cell-based assay, in an animal based assay or in a human clinical trial. For example, testing the effect of the agent on a POSH function may comprise testing the effect of the agent on the production of viral particles or virus like particles in a cell (cultured or situated in a mammalian subject) infected with an envelope virus. In another embodiment, testing the effect of the agent on a POSH function comprises testing the effect of the agent on POSH-mediated phosphorylation of a JNK pathway kinase. In a further embodiment, testing the effect of the agent on a POSH function may comprise testing the effect of the agent on a POSH enzymatic activity, such as ubiquitin ligase activity (e.g., POSH autoubiquitination). In an additional embodiment, testing the effect of the agent on a POSH function comprises testing the effect of the agent on POSH-mediated localization or secretion of a protein. In an additional embodiment, testing the effect of the agent on a POSH function comprises testing the effect of the agent on the interaction of POSH with a POSH associated protein, such as a a small GTPase (e.g., Rac or Chp). The test agent may be essentially any substance, including, for example an antisense nucleic acids, siRNA constructs, small molecules, antibodies and polypeptides. Assays of this type may be used to identify agents that modulate POSH-related disorders, such as viral infections, POSH-associated cancers. Additionally, assays of this type may be used to identify agents that modulate POSH-mediated processes, such as trafficking of certain proteins (e.g., myristoylated proteins) in the secretory pathway and apoptosis. The effect of an agent on any of these POSH-related disorders and POSH-mediated processes may be evaluated.

In certain aspects, the application provides methods for identifying an antiviral agent comprising: (a) identifying a test agent that inhibits an activity of or expression of a POSH-AK or a subunit of the POSH-AK; and (b) evaluating an effect of the test agent on a function of a virus. In certain aspects, the application provides methods for evaluating an antiviral agent comprising: (a) providing a test agent that inhibits an activity of or expression of a POSH-AK or a subunit of the POSH-AK; and (b) evaluating an effect of the test agent on a function of a virus. Optionally the virus is an envelope virus, such as a lentivirus (e.g., HIV or MMuLV), a flavivirus (e.g., West Nile virus) or a hepatitis virus (e.g., HBV, HCV). A variety of methods may be used in evaluating the effect of the test agent on a function of the virus comprises. For example, one may evaluate the effect of the test agent on the budding or release of the virus or a virus-like particle. Budding or release may be measured, for example, by detecting the presence of viral particles or polypeptides thereof in the extracellular medium, which may be accomplished by Western blot, detection of a viral protein activity (e.g., reverse transcriptase activity in the case of retroviruses such as HIV), the detection of a labeled viral protein, etc. Optionally the POSH-AK is PKA. The test agent may be essentially any substance, such as an antisense nucleic acid, an siRNA construct, a small molecule, an antibody or a polypeptide.

In certain aspects, the application provides methods for identifying an agent that modulates a function of a POSH-AK Such a method may comprise (a) identifying an agent that modulates POSH; and (b) testing the effect of the agent on a POSH-AK function. In certain aspects, the application provides methods for evaluating an agent that modulates a POSH-AK function, comprising: (a) providing an agent that modulates POSH; and (b) testing the effect of the agent on a POSH-AK function. Optionally the POSH-AK is PKA. Optionally, the POSH-AK is kinase in the JNK pathway. Testing the effect of the agent on a POSH-AK function may comprise contacting a cell with the agent and measuring the effect of the agent on phosphorylation of a PKA substrate in the cell. Testing the effect of an agent on a POSH-AK may involve detecting a biological process mediated by the POSH-AK. For example, where the POSH-AK is a JNK pathway kinase, such as JNK1, JNK2, MLK1, MLK2, MLK3, MKK4, and MKK7, the method may involve detecting a JNK pathway function, such as JNK-mediated gene expression or apoptosis.

In certain aspects, the disclosure provides methods for inhibiting the Jun kinase (JNK) pathway in a human cell, comprising contacting the cell with an inhibitor of human POSH. Optionally, inhibiting the JNK pathway comprises inhibiting substrate phosphorylation by a kinase selected from among the following: JNK1, JNK2, MLK1, MLK2, MLK3, MKK4, and MKK7. In certain aspects, the disclosure provides methods for inhibiting an activity of a PKA in a cell, comprising contacting the cell with an inhibitor of POSH. Optionally, the PKA comprises a polypeptide selected from the group consisting of: PRKAR1A, PRKACA, and PRKACB. An inhibitor of POSH may be, for example, an agent that inhibits a POSH activity (e.g., ubiquitin ligase activity or interaction with a POSH-AP); or an agent that inhibits expression of a POSH.

In certain aspects the disclosuere provides methods of treating a JNK pathway-associated disease in a subject, comprising administering a POSH inhibitor to a subject in need thereof. In certain aspects, the disclosure provides methods of treating a PKA associated disease in a subject, comprising administering a POSH inhibitor to a subject in need thereof.

In certain aspects, the application provides a method of identifying an anti-viral agent, comprising: (a) forming a mixture comprising a POSH polypeptide, a PKA and a test agent; and (b) detecting phosphorylation of the POSH polypeptide, wherein an agent that inhibits phosphorylation of POSH the test agent is an anti-viral agent.

In certain aspects, the application provides a method of identifying a modulator of POSH, comprising: (a) forming a mixture comprising a POSH polypeptide, a PKA and a test agent; and (b) detecting phosphorylation of the POSH polypeptide, wherein an agent that alters phosphorylation of POSH the test agent is an agent that modulates POSH.

In certain aspects, the application provides a method of enhancing interaction of a POSH polypeptide with a second protein in a cell, comprising contacting the cell with an agent that inhibits phosphorylation of POSH by PKA. Optionally, the second protein is selected from the group consisting of: Rac, Chp, TCL, TC10, Cdc42, Wrch-1, Rac2, Rac3, and RhoG.

In further aspects, the application provides a method of inhibiting ubiquitination activity of a POSH polypeptide in a cell, comprising contacting the cell with an agent that inhibits phosphorylation of the POSH by PKA.

In an additional embodiment, the application provides a method of treating or preventing a POSH associated cancer in a subject comprising administering an agent that inhibits a POSH-AK to a subject in need thereof, wherein said agent treats or prevents cancer. Optionally the POSH-AK comprises a polypeptide selected from the group consisting of: JNK1, JNK2, MLK1, MLK2, MLK3, MKK4, and MKK7. Optionally, the POSH-AK comprises a polypeptide selected from the group consisting of: PRKAR1A, PRKACA, and PRKACB. In a preferred embodiment, the cells of the cancer, or derived therefrom, have increased POSH expression.

In certain embodiments, the application provides isolated, purified or recombinant phosphorylated POSH polypeptides. Preferably, the polypeptide is phosphorylated at a consensus PKA phosphorylation site, such as a K/R-R-X-S/T-Hy or R-X-X-S/T-Hy site (where Hy indicates a hydrophobic amino acid). A phosphorylated POSH polypeptide may be prepared, for example, by a method comprising contacting the POSH polypeptide with a PKA under conditions in which the PKA is active.

In certain aspects, the disclosure provides a portion of a POSH polypeptide consisting essentially of 15 to 100 consecutive amino acids of a mammalian POSH polypeptide which include a consensus PKA phosphorylation site. Such a portion may be phosphorylated. Optionally, the polypeptide comprises at least one modified acid amino acid or peptidomimetic moiety. In a preferred embodiment, the polypeptide inhibits PKA phosphorylation of POSH. Such a polypeptide may be formulated for delivery across a cell membrane, e.g., by mixture with lipid micelles or vesicles.

In certain aspects, a POSH-AK inhibitor may be used in the manufacture of a medicament for the treatment of a POSH-related disorder, such as a viral infection or a POSH-associated cancer. In a preferred embodiment, a protein kinase A inhibitor is used for the manufacture of a medicament for treatment of a viral infection.

Any of the various pharmaceutical agents disclosed herein may be prepared as a pharmaceutical composition and packaged with a label. A label may include, for example, instructions for use or a list of one or more recommended or approved indications. A packaged pharmaceutical for use in treating a viral infection or a POSH-associated cancer may comprise (a) a pharmaceutical composition comprising an inhibitor of a POSH-AK and a pharmaceutically acceptable carrier; and (b) instructions for use.

The practice of the present application will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Other features and advantages of the application will be apparent from the following detailed description, and from the claims.

### BRIEF DESCRIPTION OF THE DRAWINGS

FIG. 1 shows human POSH coding sequence (SEQ ID NO:1).

FIG. 2 shows human POSH amino acid sequence (SEQ ID NO:2).

FIG. 3 shows human POSH cDNA sequence (SEQ ID NO:3).

FIG. 4 shows 5′ cDNA fragment of human POSH (public gi: 10432611; SEQ ID NO:4).

FIG. 5 shows N terminus protein fragment of hPOSH (public gi: 10432612; SEQ ID NO:5).

FIG. 6 shows 3′ mRNA fragment of hPOSH (public gi:7959248; SEQ ID NO:6).

FIG. 7 shows C terminus protein fragment of hPOSH (public gi:7959249; SEQ ID NO:7).

FIG. 8 shows human POSH full mRNA, annotated sequence.

FIG. 9 shows domain analysis of human POSH.

FIG. 10 is a diagram of human POSH nucleic acids. The diagram shows the full-length POSH gene and the position of regions amplified by RT-PCR or targeted by siRNA used in FIG. 11.

FIG. 11 shows effect of knockdown of POSH mRNA by siRNA duplexes. HeLa SS-6 cells were transfected with siRNA against Lamin A/C (lanes 1, 2) or POSH (lanes 3-10). POSH siRNA was directed against the coding region (153—lanes 3, 4; 155—lanes 5, 6) or the 3′UTR (157—lanes 7, 8; 159—lanes 9, 10). Cells were harvested 24 hours post-transfection, RNA extracted, and POSH mRNA levels compared by RT-PCR of a discrete sequence in the coding region of the POSH gene (see FIG. 10). GAPDH is used an RT-PCR control in each reaction.

FIG. 12 shows that POSH affects the release of VLP from cells. A) Phosphohimages of SDS-PAGE gels of immunoprecipitations of ³⁵S pulse-chase labeled Gag proteins are presented for cell and viral lysates from transfected HeLa cells that were either untreated or treated with POSH RNAi (50 nM for 48 hours). The time during the chase period (1, 2, 3, 4, and 5 hours after the pulse) are presented from left to right for each image.

FIG. 13 shows release of VLP from cells at steady state. Hela cells were transfected with an HIV-encoding plasmid and siRNA. Lanes 1, 3 and 4 were transfected with wild-type HIV-encoding plasmid. Lane 2 was transfected with an HIV-encoding plasmids which contains a point mutation in p6 (PTAP to ATAP). Control siRNA (lamin A/C) was transfected to cells in lanes 1 and 2. siRNA to Tsg101 was transfected in lane 4 and siRNA to POSH in lane 3.

FIG. 14 shows mouse POSH mRNA sequence (public gi:10946921; SEQ ID NO: 8).

FIG. 15 shows mouse POSH Protein sequence (Public gi: 10946922; SEQ ID NO: 9).

FIG. 16 shows Drosophila melanogaster POSH mRNA sequence (public gi:17737480; SEQ ID NO:10).

FIG. 17 shows Drosophila melanogaster POSH protein sequence (public gi:17737481; SEQ IDNO:11).

FIG. 18 shows POSH domain analysis.

FIG. 19 shows that human POSH has ubiquitin ligase activity.

FIG. 20 shows that human POSH co-immunoprecipitates with RAC1.

FIG. 21 shows that POSH knockdown results in decreased secretion of phospholipase D (“PLD”).

FIG. 22 shows effect of HPOSH on Gag-EGFP intracellular distribution.

FIG. 23 shows intracellular distribution of HIV-1 Nef in hPOSH-depleted cells.

FIG. 24 shows intracellular distribution of Src in hPOSH-depleted cells.

FIG. 25 shows intracellular distribution of Rapsyn in hPOSH-depleted cells.

FIG. 26 shows that POSH reduction by siRNA abrogates West Nile virus infectivity.

FIG. 27 shows that POSH knockdown decreases the release of extracellular MMuLV particles.

FIG. 28 shows that PKA activity is required for HIV-1 virus release. Inhibition of PKA kinase activity attenuates HIV-1 virus maturation. HeLa SS6 cells were transfected with pNLenv-1PTAP or pNLenv-1ATAA (L-domain mutant). Eighteen hours post-transfection, cells were transferred to 20° C. for two hours in order to inhibit transport of viral particles from the trans-Golgi (TGN) to the plasma membrane (PM). Subsequently, the PKA inhibitor, H89 (50 μM) or DMSO were added to the cells and dishes were transferred to 37° C. to initiate transport from the TGN to the PM. Reverse transcriptase activity was assayed from virus-like-particles collected from cell supernatant twenty minutes later. H89 treatment resulted in complete inhibition of RT activity (compare H89-treated to pNLenv-1ATAA transfected cells to pNLenv-1PTAP; left and right panels with middle panel, respectively).

FIG. 29 shows that HPOSH is phosphorylated by PKA. hPOSH or c-Cbl was incubated with or without PKA as indicated. Samples were separated by SDS-PAGE and immunoblotted with PKA-substrate phospho-specific antibody followed by detection with anti-Rabbit-HRP and ECL (right). The membrane was then stripped of antibodies and re-immunoblotted with a mixture of anti-hPOSH polyclonal antibodies, followed by detection with anti-Rabbit-HRP and ECL (left panel).

FIG. 30 shows putative PKA phosphorylation sites in hPOSH. Amino acid sequence of hPOSH (70 residues per line). Motifs of the low stringency RxxS/T type are underlined. The high stringency motif R/KR/KxS/T is bordered. Putative S/T phosphorylation sites are highlighted in green. Color-coding of domains: Red—RING, Blue—SH3, Green—putative Rac-1 Binding Domain.

FIG. 31 shows that phosphorylation of hPOSH regulates binding of GTP-loaded Rac-1. Bacterially expressed HPOSH (1μg) (POSH) or GST (1 μg) (NS) were phosphorylated as in FIG. 1. Subsequently, GTPγS loaded or unloaded recombinant Rac-1 (0.2 μg) was added to hPOSH or GST. Bound rac1 was isolated as described in materials and methods and samples separated by SDS-PAGE on a 12% gel and immunobloted with anti-Rac-1. Input is 0.25 μg of Rac-1.

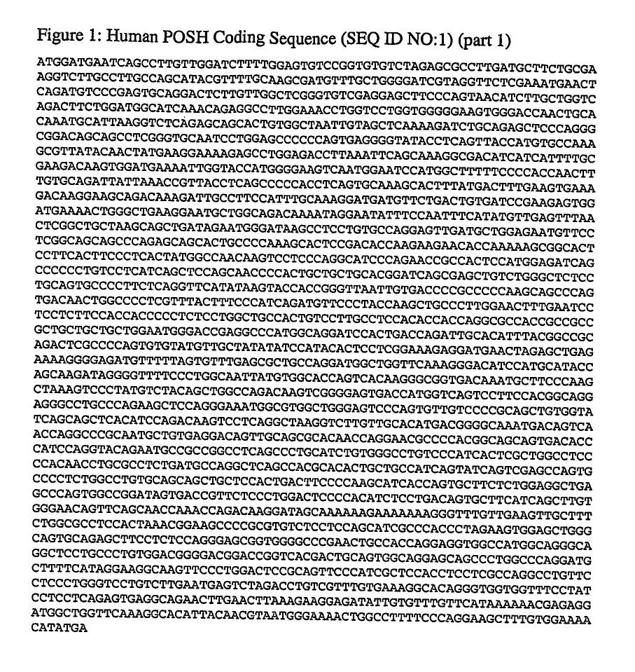

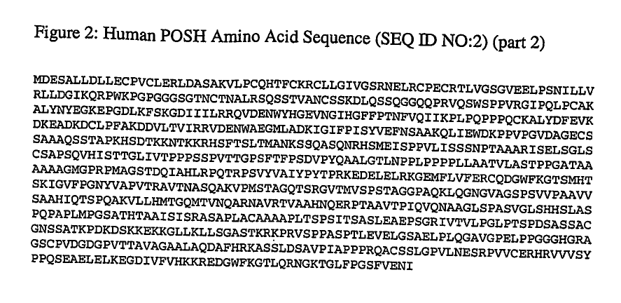

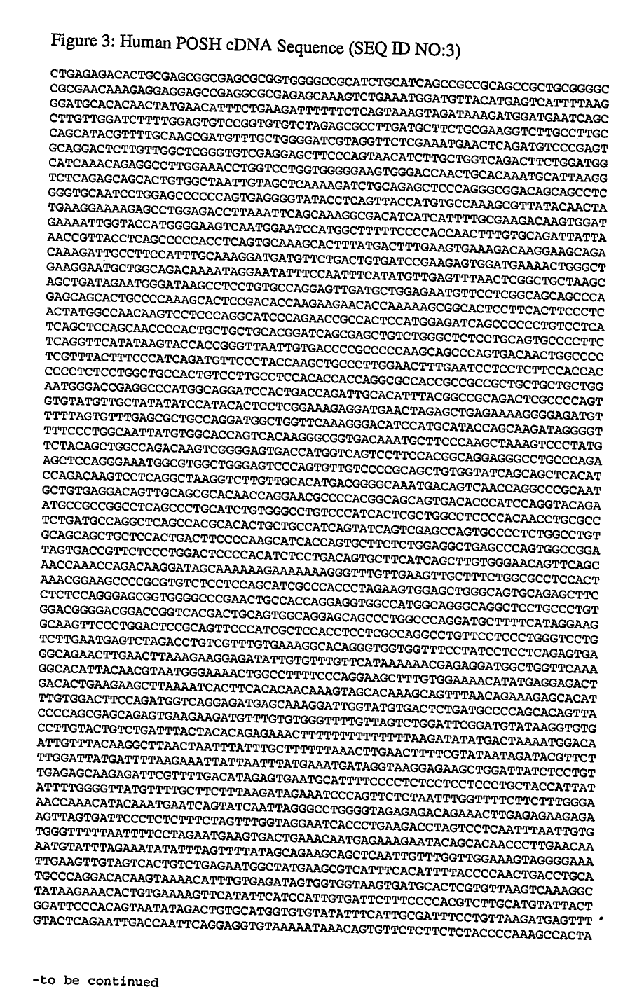

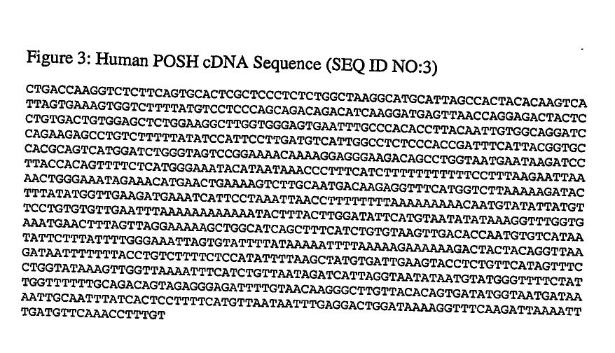

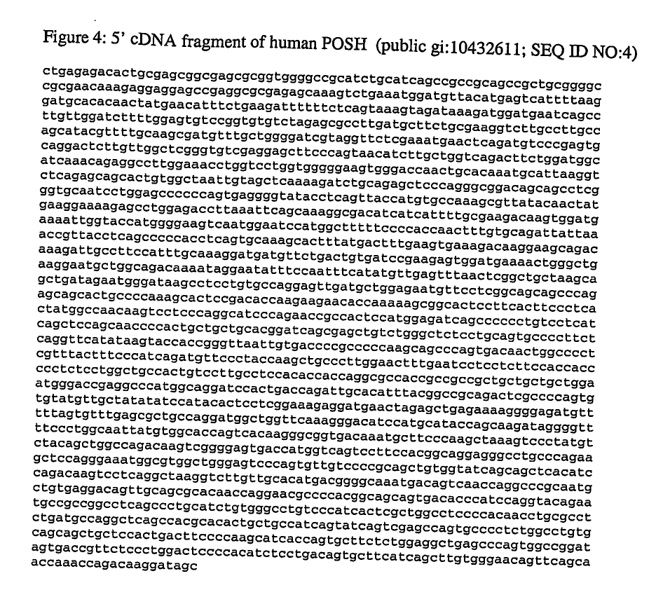

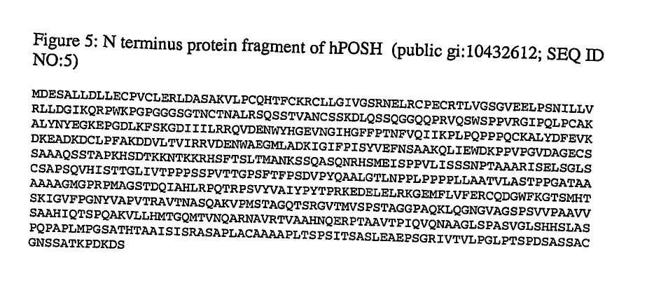

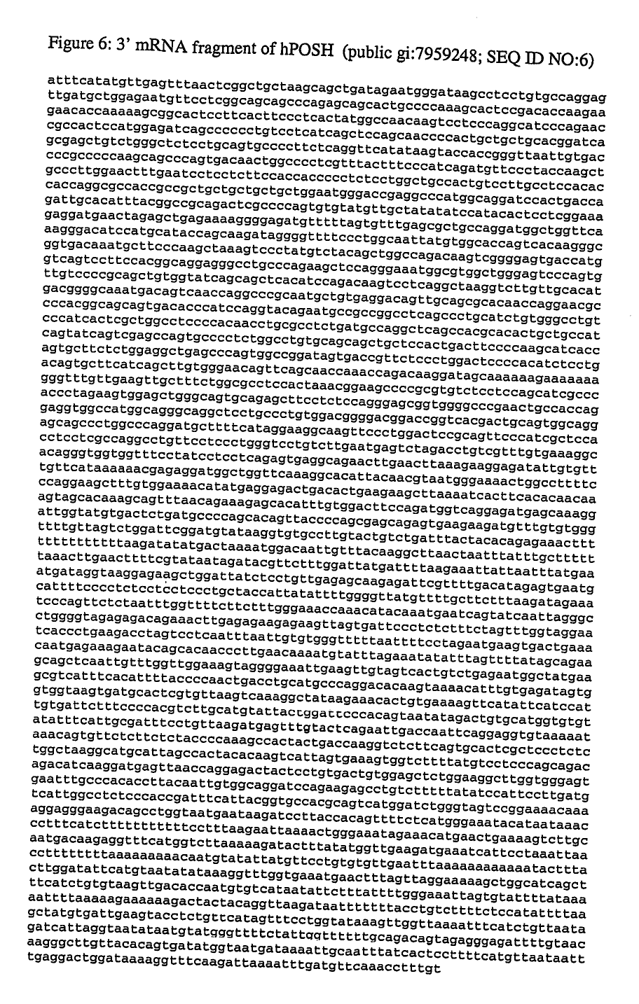

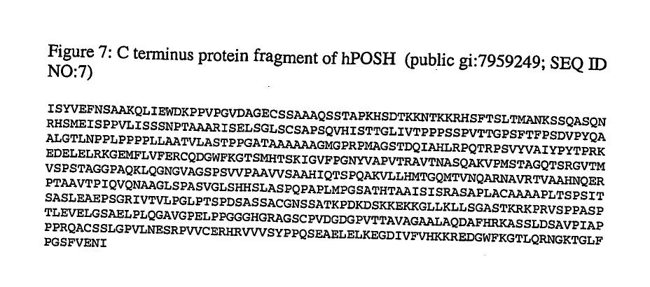

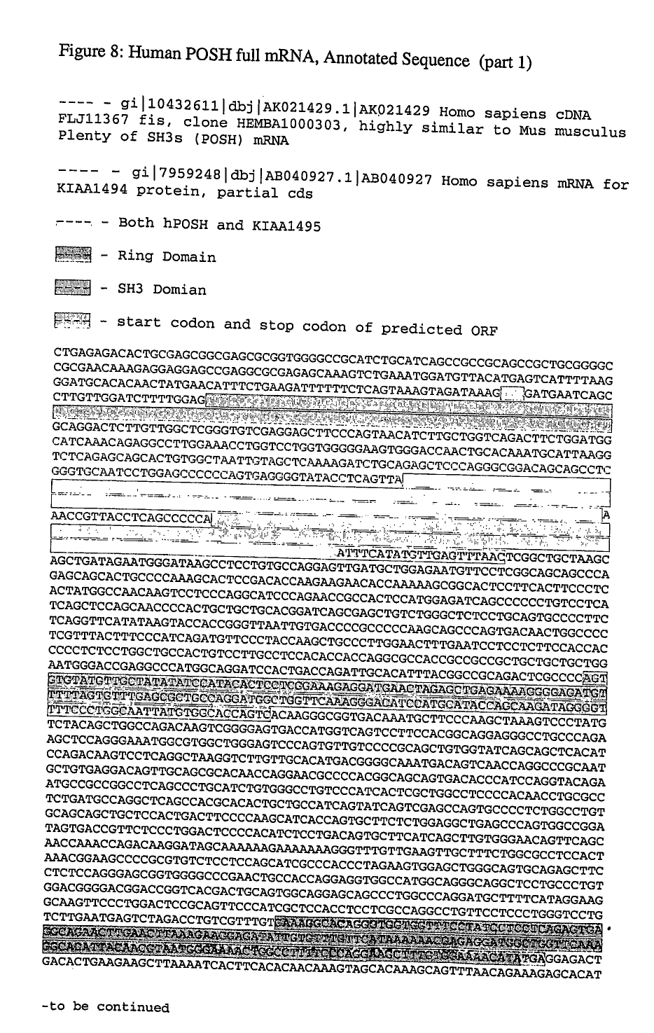

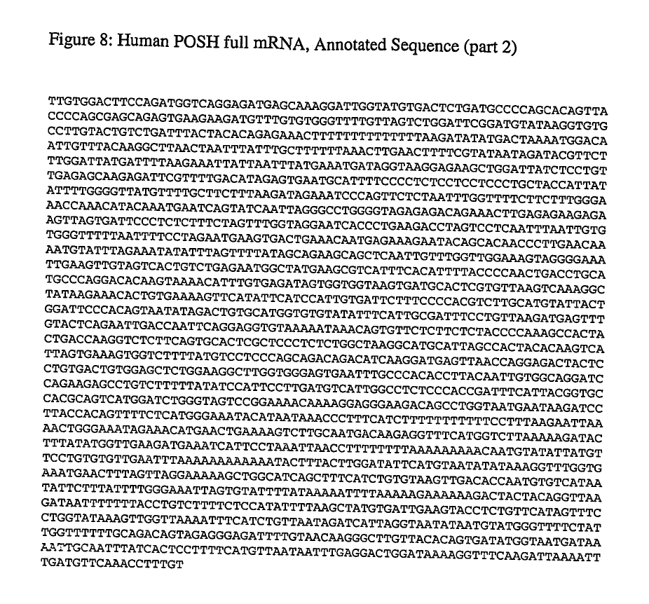

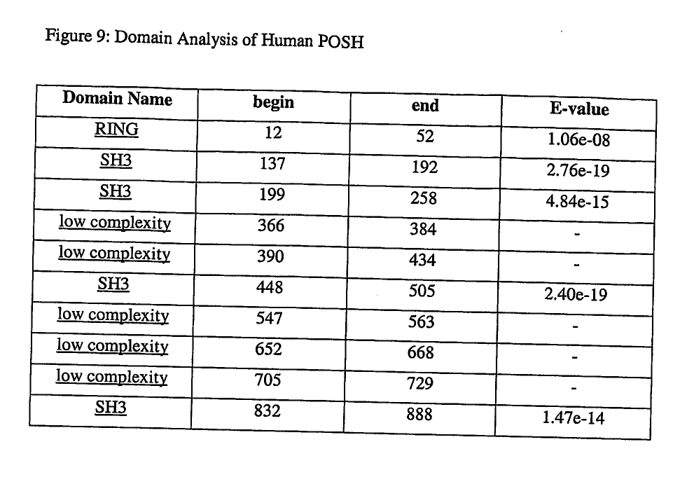

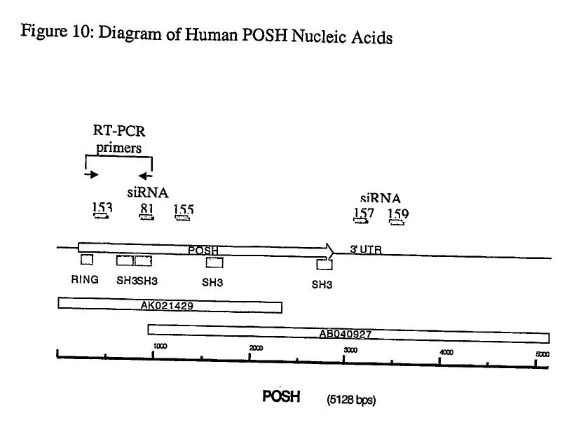

### DETAILED DESCRIPTION OF THE APPLICATION

### 1. Definitions

The term “binding” refers to a direct association between two molecules, due to, for example, covalent, electrostatic, hydrophobic, ionic and/or hydrogen-bond interactions under physiological conditions.

A “chimeric protein” or “fusion protein” is a fusion of a first amino acid sequence encoding a polypeptide with a second amino acid sequence defining a domain foreign to and not substantially homologous with any domain of the first amino acid sequence. A chimeric protein may present a foreign domain which is found (albeit in a different protein) in an organism which also expresses the first protein, or it may be an “interspecies”, “intergenic”, etc. fusion of protein structures expressed by different kinds of organisms.

The terms “compound”, “test compound” and “molecule” are used herein interchangeably and are meant to include, but are not limited to, peptides, nucleic acids, carbohydrates, small organic molecules, natural product extract libraries, and any other molecules (including, but not limited to, chemicals, metals and organometallic compounds).

The phrase “conservative amino acid substitution” refers to grouping of amino acids on the basis of certain common properties. A functional way to define common properties between individual amino acids is to analyze the normalized frequencies of amino acid changes between corresponding proteins of homologous organisms (Schulz, G. E. and R. H. Schirmer., Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer, Principles of Protein Structure, Springer-Verlag). Examples of amino acid groups defined in this manner include:
(i) a charged group, consisting of Glu and Asp, Lys, Arg and His,
(ii) a positively-charged group, consisting of Lys, Arg and His,
(iii) a negatively-charged group, consisting of Glu and Asp,
(iv) an aromatic group, consisting of Phe, Tyr and Trp,
(v) a nitrogen ring group, consisting of His and Trp,
(vi) a large aliphatic nonpolar group, consisting of Val, Leu and Ile,
(vii) a slightly-polar group, consisting of Met and Cys,
(viii) a small-residue group, consisting of Ser, Thr, Asp, Asn, Gly, Ala, Glu, Gln and Pro,
(ix) an aliphatic group consisting of Val, Leu, Ile, Met and Cys, and
(x) a small hydroxyl group consisting of Ser and Thr.

In addition to the groups presented above, each amino acid residue may form its own group, and the group formed by an individual amino acid may be referred to simply by the one and/or three letter abbreviation for that amino acid commonly used in the art.

A “conserved residue” is an amino acid that is relatively invariant across a range of similar proteins. Often conserved residues will vary only by being replaced with a similar amino acid, as described above for “conservative amino acid substitution”.

The term “domain” as used herein refers to a region of a protein that comprises a particular structure and/or performs a particular function.

The term “envelope virus” as used herein refers to any virus that uses cellular membrane and/or any organelle membrane in the viral release process.

“Homology” or “identity” or “similarity” refers to sequence similarity between two peptides or between two nucleic acid molecules. Homology and identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. A sequence which is “unrelated” or “non-homologous” shares less than 40% identity, though preferably less than 25% identity with a sequence of the present application. In comparing two sequences, the absence of residues (amino acids or nucleic acids) or presence of extra residues also decreases the identity and homology/similarity.

The term “homology” describes a mathematically based comparison of sequence similarities which is used to identify genes or proteins with similar functions or motifs. The nucleic acid and protein sequences of the present application may be used as a “query sequence” to perform a search against public databases to, for example, identify other family members, related sequences or homologs. Such searches can be performed using the NBLAST and XBLAST programs (version 2.0) of Altschul, et al. (1990) J Mol. Biol. 215:403-10. BLAST nucleotide searches can be performed with the NBLAST program, score=100, wordlength=12 to obtain nucleotide sequences homologous to nucleic acid molecules of the application. BLAST protein searches can be performed with the XBLAST program, score=50, wordlength=3 to obtain amino acid sequences homologous to protein molecules of the application. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used. See http://www.ncbi.nlm.nih.gov.

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403410 (1990). The well known Smith Waterman algorithm may also be used to determine identity.

The term “isolated”, as used herein with reference to the subject proteins and protein complexes, refers to a preparation of protein or protein complex that is essentially free from contaminating proteins that normally would be present with the protein or complex, e.g., in the cellular milieu in which the protein or complex is found endogenously. Thus, an isolated protein complex is isolated from cellular components that normally would “contaminate” or interfere with the study of the complex in isolation, for instance while screening for modulators thereof. It is to be understood, however, that such an “isolated” complex may incorporate other proteins the modulation of which, by the subject protein or protein complex, is being investigated.

The term “isolated” as also used herein with respect to nucleic acids, such as DNA or RNA, refers to molecules in a form which does not occur in nature. Moreover, an “isolated nucleic acid” is meant to include nucleic acid fragments which are not naturally occurring as fragments and would not be found in the natural state.

Lentiviruses include primate lentiviruses, e.g., human immunodeficiency virus types 1 and 2 (HIV-1/HIV-2); simian immunodeficiency virus (SIV) from Chimpanzee (SIVcpz), Sooty mangabey (SIVsmm), African Green Monkey (SIVagm), Syke's monkey (SIVsyk), Mandrill (SIVmnd) and Macaque (SIVmac). Lentiviruses also include feline lentiviruses, e.g., Feline immunodeficiency virus (FIV); Bovine lentiviruses, e.g., Bovine immunodeficiency virus (BIV); Ovine lentiviruses, e.g., Maedi/Visna virus (MVV) and Caprine arthritis encephalitis virus (CAEV); and Equine lentiviruses, e.g., Equine infectious anemia virus (EIAV). All lentiviruses express at least two additional regulatory proteins (Tat, Rev) in addition to Gag, Pol, and Env proteins. Primate lentiviruses produce other accessory proteins including Nef, Vpr, Vpu, Vpx, and Vif. Generally, lentiviruses are the causative agents of a variety of disease, including, in addition to immunodeficiency, neurological degeneration, and arthritis. Nucleotide sequences of the various lentiviruses can be found in Genbank under the following Accession Nos. (from J. M. Coffin, S. H. Hughes, and H. E. Varmus, “Retroviruses” Cold Spring Harbor Laboratory Press, 199,7 p 804): 1) HIV-1: K03455, M19921, K02013, M38431, M38429, K02007 and M17449; 2) HIV-2: M30502, J04542, M30895, J04498, M15390, M31113 and L07625; 3) SIV:M29975, M30931, M58410, M66437, L06042, M33262, M19499, M32741, M31345 and L03295; 4) FIV: M25381, M36968 and U11820; 5) BIV. M32690; 6) E1AV: M16575, M87581 and U01866; 6) Visna: M10608, M51543, L06906, M60609 and M60610; 7) CAEV: M33677; and 8) Ovine lentivirus M31646 and M34193. Lentiviral DNA can also be obtained from the American Type Culture Collection (ATCC). For example, feline immunodeficiency virus is available under ATCC Designation No. VR-2333 and VR-3112. Equine infectious anemia virus A is available under ATCC Designation No. VR-778. Caprine arthritis-encephalitis virus is available under ATCC Designation No. VR-905. Visna virus is available under ATCC Designation No. VR-779.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single-stranded (such as sense or antisense) and double-stranded polynucleotides.

The term “maturation” as used herein refers to the production, post-translational processing, assembly and/or release of proteins that form a viral particle. Accordingly, this includes the processing of viral proteins leading to the pinching off of nascent virion from the cell membrane.

A “POSH nucleic acid” is a nucleic acid comprising a sequence as represented in any of SEQ ID Nos:1, 3, 4, 6, 8, and 10 as well as any of the variants described herein.

A “POSH polypeptide” or “POSH protein” is a polypeptide comprising a sequence as represented in any of SEQ ID Nos: 2, 5, 7, 9 and 11 as well as any of the variations described herein.

A “POSH-associated protein” or “POSH-AP” refers to a protein capable of interacting with and/or binding to a POSH polypeptide. Generally, the POSH-AP may interact directly or indirectly with the POSH polypeptide. According to the application, a specific type of POSH-AP is a kinase (herein referred to as POSH-AK). POSH-AKs may comprise a single polypeptide of a complex of polypeptides, where often one or more catalytic subunits is accompanied by one or more regulatory subunits. Preferred POSH-AKs include protein kinase A (PKA) which comprises a PKA subunit polypeptide such as: PRKAR1A, PRKACA, and PRKACB. Other preferred POSH-AKs include a kinase of a Rac-JNK signaling pathway, for example, JNK1, JNK2, MLK1, MLK2, MLK3, MKK4, and MKK7. Examples of these and other POSH-AKs are provided throughout.

The terms peptides, proteins and polypeptides are used interchangeably herein.

The term “purified protein” refers to a preparation of a protein or proteins which are preferably isolated from, or otherwise substantially free of, other proteins normally associated with the protein(s) in a cell or cell lysate. The term “substantially free of other cellular proteins” (also referred to herein as “substantially free of other contaminating proteins”) is defined as encompassing individual preparations of each of the component proteins comprising less than 20% (by dry weight) contaminating protein, and preferably comprises less than 5% contaminating protein. Functional forms of each of the component proteins can be prepared as purified preparations by using a cloned gene as described in the attached examples. By “purified”, it is meant, when referring to component protein preparations used to generate a reconstituted protein mixture, that the indicated molecule is present in the substantial absence of other biological macromolecules, such as other proteins (particularly other proteins which may substantially mask, diminish, confuse or alter the characteristics of the component proteins either as purified preparations or in their function in the subject reconstituted mixture). The term “purified” as used herein preferably means at least 80% by dry weight, more preferably in the range of 85% by weight, more preferably 95-99% by weight, and most preferably at least 99.8% by weight, of biological macromolecules of the same type present (but water, buffers, and other small molecules, especially molecules having a molecular weight of less than 5000, can be present). The term “pure” as used herein preferably has the same numerical limits as “purified” immediately above.

A “recombinant nucleic acid” is any nucleic acid that has been placed adjacent to another nucleic acid by recombinant DNA techniques. A “recombined nucleic acid” also includes any nucleic acid that has been placed next to a second nucleic acid by a laboratory genetic technique such as, for example, tranformation and integration, transposon hopping or viral insertion. In general, a recombined nucleic acid is not naturally located adjacent to the second nucleic acid.

The term “recombinant protein” refers to a protein of the present application which is produced by recombinant DNA techniques, wherein generally DNA encoding the expressed protein is inserted into a suitable expression vector which is in turn used to transform a host cell to produce the heterologous protein. Moreover, the phrase “derived from”, with respect to a recombinant gene encoding the recombinant protein is meant to include within the meaning of “recombinant protein” those proteins having an amino acid sequence of a native protein, or an amino acid sequence similar thereto which is generated by mutations including substitutions and deletions of a naturally occurring protein.

A “RING domain” or “Ring Finger” is a zinc-binding domain with a defined octet of cysteine and histidine residues. Certain RING domains comprise the consensus sequences as set forth below (amino acid nomenclature is as set forth in Table 1): Cys Xaa Xaa Cys Xaa₁₀₋₂₀ Cys Xaa His Xaa₂₋₅ Cys Xaa Xaa Cys Xaa₁₃₋₅₀ Cys Xaa Xaa Cys or Cys Xaa Xaa Cys Xaa₁₀₋₂₀ Cys Xaa His Xaa₂₋₅ His Xaa Xaa Cys Xaa₁₃₋₅₀ Cys Xaa Xaa Cys. Certain RING domains are represented as amino acid sequences that are at least 80% identical to amino acids 12-52 of SEQ ID NO: 2 and is set forth in SEQ ID No: 26. Preferred RING domains are 85%, 90%, 95%, 98% and, most preferably, 100% identical to the amino acid sequence of SEQ ID NO: 26. Preferred RING domains of the application bind to various protein partners to form a complex that has ubiquitin ligase activity. RING domains preferably interact with at least one of the following protein types: F box proteins, E2 ubiquitin conjugating enzymes and cullins.

The term “RNA interference” or “RNAi” refers to any method by which expression of a gene or gene product is decreased by introducing into a target cell one or more double-stranded RNAs which are homologous to the gene of interest (particularly to the messenger RNA of the gene of interest). RNAi may also be achieved by introduction of a DNA:RNA hybrid wherein the antisense strand (relative to the target) is RNA. Either strand may include one or more modifications to the base or sugar-phosphate backbone. Any nucleic acid preparation designed to achieve an RNA interference effect is referred to herein as an siRNA construct. Phosphorothioate is a particularly common modification to the backbone of an siRNA construct.

“Small molecule” as used herein, is meant to refer to a composition, which has a molecular weight of less than about 5 kD and most preferably less than about 2.5 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures comprising arrays of small molecules, often fungal, bacterial, or algal extracts, which can be screened with any of the assays of the application.

An “SH3” or “Src Homology 3” domain is a protein domain of generally about 60 amino acid residues first identified as a conserved sequence in the non-catalytic part of several cytoplasmic protein tyrosine kinases (e.g., Src, Abl, Lck). SH3 domains mediate assembly of specific protein complexes via binding to proline-rich peptides. Exemplary SH3 domains are represented by amino acids 137-192, 199-258, 448-505 and 832-888 of SEQ ID NO:2 and are set forth in SEQ ID Nos: 27-30. In certain embodiments, an SH3 domain interacts with a consensus sequence of RXaaXaaPXaaX6P (where X6, as defined in table 1 below, is a hydrophobic amino acid). In certain embodiments, an SH3 domain interacts with one or more of the following sequences: P(T/S)AP, PFRDY, RPEPTAP, RQGPKEP, RQGPKEPFR, RPEPTAPEE and RPLPVAP.

As used herein, the term “specifically hybridizes” refers to the ability of a nucleic acid probe/primer of the application to hybridize to at least 12, 15, 20, 25, 30, 35, 40, 45, 50 or 100 consecutive nucleotides of a POSH sequence, or a sequence complementary thereto, or naturally occurring mutants thereof, such that it has less than 15%, preferably less than 10%, and more preferably less than 5% background hybridization to a cellular nucleic acid (e.g., mRNA or genomic DNA) other than the POSH gene. A variety of hybridization conditions may be used to detect specific hybridization, and the stringency is determined primarily by the wash stage of the hybridization assay. Generally high temperatures and low salt concentrations give high stringency, while low temperatures and high salt concentrations give low stringency. Low stringency hybridization is achieved by washing in, for example, about 2.0×SSC at 50° C., and high stringency is acheived with about 0.2×SSC at 50° C. Further descriptions of stringency are provided below.

As applied to polypeptides, “substantial sequence identity” means that two peptide sequences, when optimally aligned, such as by the programs GAP or BESTFIT using default gap which share at least 90 percent sequence identity, preferably at least 95 percent sequence identity, more preferably at least 99 percent sequence identity or more. Preferably, residue positions which are not identical differ by conservative amino acid substitutions. For example, the substitution of amino acids having similar chemical properties such as charge or polarity are not likely to effect the properties of a protein. Examples include glutamine for asparagine or glutamic acid for aspartic acid.

As is well known, genes for a particular polypeptide may exist in single or multiple copies within the genome of an individual. Such duplicate genes may be identical or may have certain modifications, including nucleotide substitutions, additions or deletions, which all still code for polypeptides having substantially the same activity.

A “virion” is a complete viral particle; nucleic acid and capsid (and a lipid envelope in some viruses. A “viral particle” may be incomplete, as when produced by a cell transfected with a defective virus (e.g., an HIV virus-like particle system).

TABLE 1
Abbreviations for classes of amino acids*

| | | Amino Acids |
|---|---|---|
| Symbol | Category | Represented |
| X1 | Alcohol | Ser, Thr |
| X2 | Aliphatic | Ile, Leu, Val |
| Xaa | Any | Ala, Cys, Asp, Glu, Phe, |
| | | Gly, His, Ile, Lys, Leu, |
| | | Met, Asn, Pro, Gln, Arg, |
| | | Ser, Thr, Val, Trp, Tyr |
| X4 | Aromatic | Phe, His, Trp,Tyr |
| X5 | Charged | Asp, Glu, His, Lys, Arg |
| X6 | Hydrophobic | Ala, Cys, Phe, Gly, His, |
| | | Ile, Lys, Leu, Met, Thr, |
| | | Val, Trp, Tyr |
| X7 | Negative | Asp, Glu |
| X8 | Polar | Cys, Asp, Glu, His, Lys, |
| | | Asn, Gln, Arg, Ser, Thr |
| X9 | Positive | His, Lys, Arg |
| X10 | Small | Ala, Cys, Asp, Gly, Asn, |
| | | Pro, Ser, Thr, Val |
| X11 | Tiny | Ala, Gly, Ser |
| X12 | Turnlike | Ala, Cys, Asp, Glu, Gly, |
| | | His, Lys, Asn, Gln, Arg, |
| | | Ser, Thr |
| X13 | Asparagine-Aspartate | Asn, Asp |

*Abbreviations as adopted from http://smart.embl-heidelberg.de/SMART_DATA/alignments/consensus/grouping.html.

2. Overview

In certain aspects, the application relates to the discovery of novel associations between POSH proteins and other proteins such as kinases (termed POSH-AKs), and related methods and compositions. In certain aspects, the application relates to novel associations among certain disease states, POSH nucleic acids and proteins, and POSH-AK nucleic acids and proteins.

In certain aspects, by identifying kinase proteins associated with POSH, and particularly human POSH, the present application provides a conceptual link between the POSH-AKs and cellular processes and disorders associated with POSH-AKs, and POSH itself. Accordingly, in certain embodiments of the disclosure, agents that modulate a POSH-AK may now be used to modulate POSH functions and disorders associated with POSH function, such as viral disorders and POSH-associated cancers. Additionally, test agents may be screened for an effect on a POSH-AK and then further tested for effect on a POSH function or a disorder associated with POSH function. Likewise, in certain embodiments of the disclosure, agents that modulate POSH may now be used to modulate POSH-AK functions and disorders associated with POSH-AK function, including a variety of cancers. Additionally, test agents may be screened for an effect on POSH and then further tested for effect on a POSH-AK function or a disorder associated with POSH-AK function. In further aspects, the application provides nucleic acid agents (e.g., RNAi probes, antisense nucleic acids), antibody-related agents, small molecules and other agents that affect POSH function, and the use of same in modulating POSH and/or POSH-AK activity.

POSH intersects with and regulates a wide range of key cellular functions that may be manipulated by affecting the level of and/or activity of POSH polypeptides or POSH-AK polypeptides. Many features of POSH, and particularly human POSH, are described in PCT patent publications WO03/095971A2 (application no. WO2002US0036366) and WO03/078601A2 (application no. WO2003US0008194) the teachings of which are incorporated by reference herein.

As described in the above-referenced publications, native human POSH is a large polypeptide containing a RING domain and four SH3 domains. POSH is a ubiquitin ligase (also termed an “E3” enzyme); the RING domain mediates ubiquitination of, for example, the POSH polypeptide itself. POSH interacts with a large number of proteins and participates in a host of different biological processes. As demonstrated in this disclosure, POSH associates with a number of different protein kinases in the cell. POSH co-localizes with proteins that are known to be located in the trans-Golgi network, implying that POSH participates in the trafficking of proteins in the secretory system. The term “secretory system” should be understood as referring to the membrane compartments and associated proteins and other molecules that are involved in the the movement of proteins from the site of translation to a location within a vacuole, a compartment in the secretory pathway itself, a lysosome or endosome or to a location at the plasma membrane or outside the cell. Commonly cited examples of compartments in the secretory system include the endoplasmic reticulum, the Golgi apparatus and the cis and trans Golgi networks. In addition, Applicants have demonstrated that POSH is necessary for proper secretion, localization or processing of a variety of proteins, including phospholipase D, HIV Gag, HIV Nef, Rapsyn and Src. Many of these proteins are myristoylated, indicating that POSH plays a general role in the processing and proper localization of myristoylated proteins. N-myristoylation is an acylation process, which results in covalent attachment of myristate, a 14-carbon saturated fatty acid to the N-terminal glycine of proteins (Farazi et al., J. Biol. Chem. 276:39501-04 (2001)). N-myristoylation occurs co-translationaly and promotes weak and reversible protein-membrane interaction. Myristoylated proteins are found both in the cytoplasm and associated with membrane. Membrane association is dependent on protein configuration, i.e., surface accessibility of the myristoyl group may be regulated by protein modifications, such as phosphorylation, ubiquitination etc. Modulation of intracellular transport of myristoylated proteins in the application includes effects on transport and localization of these modified proteins.

As described herein, POSH and POSH-AKs are involved in viral maturation, including the production, post-translational processing, assembly and/or release of proteins in a viral particle. Accordingly, viral infections may be ameliorated by inhibiting an activity (e.g., ubiquitin ligase activity or target protein interaction) of POSH or a POSH-AK (e.g., inhibition of kinase activity), and in preferred embodiments, the virus is a retroid virus, an RNA virus or an envelope virus, including HIV, Ebola, HBV, HCV, HTLV, West Nile Virus (WNV) or Moloney Murine Leukemia Virus (MMuLV). Additional viral species are described in greater detail below. In certain instances, a decrease of a POSH function is lethal to cells infected with a virus that employs POSH in release of viral particles.

In certain aspects, the application describes an hPOSH interaction with Rac, a small GTPase and the POSH associated kinases MLK, MKK and JNK. Rho, Rac and Cdc42 operate together to regulate organization of the actin cytoskeleton and the MLK-MKK-JNK MAP kinase pathway (referred to herein as the “JNK pathway” or “Rac-JNK pathway” (Xu et al., 2003, EMBO J. 2: 252-61). Ectopic expression of mouse POSH (“mPOSH”) activates the JNK pathway and causes nuclear localization of NF-κB. Overexpression of mPOSH in fibroblasts stimulates apoptosis. (Tapon et al. (1998) EMBO J. 17:1395-404). In Drosophila, POSH may interact with, or otherwise influence the signaling of, another GTPase, Ras. (Schnorr et al. (2001) Genetics 159: 609-22). The JNK pathway and NF-κB regulate a variety of key genes involved in, for example, immune responses, inflammation, cell proliferation and apoptosis. For example, NF-κB regulates the production of interleukin 1, interleukin 8, tumor necrosis factor and many cell adhesion molecules. NF-κB has both pro-apoptotic and anti-apoptotic roles in the cell (e.g., in FAS-induced cell death and TNF-alpha signaling, respectively). NF-κB is negatively regulated, in part, by the inhibitor proteins IκBα and IκBβ (collectively termed “IκB”). Phosphorylation of IκB permits activation and nuclear localization of NF-κB. Phosphorylation of IκB triggers its degradation by the ubiquitin system. Accordingly, in yet another embodiment, a POSH polypeptide stimulates a JNK pathway. In an additional embodiment, a POSH polypeptide promotes nuclear localization of NF-κB. In further embodiments, manipulation of POSH levels and/or activities may be used to manipulate apoptosis. By upregulating POSH or a POSH-AK, apoptosis may be stimulated in certain cells, and this will generally be desirable in conditions characterized by excessive cell proliferation (e.g., in certain cancers). By downregulating POSH or a POSH-AK, apoptosis may be diminished in certain cells, and this will generally be desirable in conditions characterized by excessive cell death, such as myocardial infarction, stroke, degenerative diseases of muscle and nerve (particularly Alzheimer's disease), and for organ preservation prior to transplant. In a further embodiment, a POSH polypeptide associates with a vesicular trafficking complex, such as a clathrin- or coatomer-containing complex, and particularly a trafficking complex that localizes to the nucleus and/or Golgi apparatus.

As described in WO03/078601A2 (application no. WO2003US0008194), POSH is overexpressed in a variety of cancers, and downregulation of POSH is associated with a decrease in proliferation in at least one cancer cell line. Accordingly, agents that modulate POSH itself or a POSH-AK may be used to treat POSH associated cancers. POSH associated cancers include those cancers in which POSH is overexpressed and/or in which downregulation of POSH leads to a decrease the proliferation or survival of cancer cells. POSH-associated cancers are described in more detail below. In addition, it is notable that many proteins shown herein to be affected by POSH downregulation are themselves involved in cancers. Phospholipase D and SRC are both aberrantly processed in a POSH-impaired cell, and therefore modulation of POSH and/or a POSH-AK may affect the wide range of cancers in which PLD and SRC play a significant role.

As described in WO03/095971A2 (application no. WO2002US0036366) and WO03/078601A2 (application no. WO2003US0008194), POSH polypeptides function as E3 enzymes in the ubiquitination system. Accordingly, downregulation or upregulation of POSH ubiquitin ligase activity can be used to manipulate biological processes that are affected by protein ubiquitination. Modulation of POSH ubiquitin ligase activity may be used to affect POSH-AKs and related biological processes, and likewise, modulation of POSH-AKs may be used to affect POSH ubiquitin ligase activity and related processes. Downregulation or upregulation may be achieved at any stage of POSH formation and regulation, including transcriptional, translational or post-translational regulation. For example, POSH transcript levels may be decreased by RNAi targeted at a POSH gene sequence. As another example, POSH ubiquitin ligase activity may be inhibited by contacting POSH with an antibody that binds to and interferes with a POSH RING domain or a domain of POSH that mediates interaction with a target protein (a protein that is ubiquitinated at least in part because of POSH activity). As a further example, small molecule inhibitors of POSH ubiquitin ligase activity are provided herein. As another example, POSH activity may be increased by causing increased expression of POSH or an active portion thereof. POSH, and POSH-AKs that modulate the POSH ubiquitin ligase activity may participate in biological processes including, for example, one or more of the various stages of a viral lifecycle, such as viral entry into a cell, production of viral proteins, assembly of viral proteins and release of viral particles from the cell. POSH may participate in diseases characterized by the accumulation of ubiquitinated proteins, such as dementias (e.g., Alzheimer's and Pick's), inclusion body myositis and myopathies, polyglucosan body myopathy, and certain forms of amyotrophic lateral sclerosis. POSH may participate in diseases characterized by the excessive or inappropriate ubiquitination and/or protein degradation.

In certain aspects, the application relates to the discovery that a POSH polypeptide interacts with one subunit of Protein Kinase A (PKA; cAMP-dependent protein kinase). Exemplary PKA subunits may include, but are not limited to, a regulatory subunit (e.g., PRKAR1A) and a catalytic subunit (e.g., PRKACA or PRKACB). PKA is an essential enzyme in the signaling pathway of the second messenger cyclic AMP (cAMP). Through phosphorylation of target proteins, PKA controls many biochemical events in the cell including regulation of metabolism, ion transport, and gene transcription. The PKA holoenzyme is composed of two regulatory and two catalytic subunits and dissociates from the regulatory subunits upon binding of cAMP. The PKA enzyme is inactive in the absence of cAMP. Activation of PKA occurs when two cAMP molecules bind to each regulatory subunit, eliciting a reversible conformational change that releases active catalytic subunits.

A number of human PKA subunits have been characterized, including a regulatory subunit (type I alpha: PRKAR1) and two catalytic subunits (C-alpha: PRKACA; and C-beta: PRKACB). Boshart et al. identified the regulatory subunit PRKAR1 of PKA as the product of the TSE1 locus (Boshart, M et al. (1991) Cell 66: 849-859). The evidence consisted of concordant expression of PRKAR1 mRNA and TSE1 genetic activity, high resolution physical mapping of the two genes on human chromosome 17, and the ability of transfected PRKAR1 cDNA to generate a phenocopy of TSE1-mediated extinction. Jones et al. independently established identity of TSE1 and the RI-alpha subunit (Jones, K W et al. (1991) Cell 66: 861-872).

Other than a role of PKA in metabolism, PKA subunits have recently been implicated in multiple diseases. For example, a specific role for localized PRKAR1 has been demonstrated in human T lymphocytes, where type I PKA localizes to the activated TCR complex and is required for attenuation of signals propagated through this complex (Skalhegg, B S et al. (1992) J Biol Chem 267:15707-15714; Skalhegg, B S et al. (1994) Science 263: 84-87). The importance of type I PKA-mediated effects in attenuation of T cell replication has led to its consideration as a therapeutic target in combined variable immunodeficiency (CVI) and acquired immune deficiency syndrome (AIDS). Furthermore, type I PKA in T cells may also serve as a potential therapeutic target in systemic lupus erythematosis (SLE). For example, a series of recently published articles has uncovered the first human disease mapping to a PKA subunit-Carney complex (Casey, M et al. (2000) J Clin Invest 106: R31-38; Kirschner, L S et al. (2000) Nat Genet 26: 89-92). Carney complex (CNC) is a multiple neoplasia syndrome characterized by spotty skin pigmentation, cardiac and skin myxomas, endocrine tumors, and psammomatous melanotic schwannomas. CNC maps to two genomic loci, 17q24 and 2p16. Familial cases mapping to the 17q24 locus reveal deletions/mutations in the PRKAR1 coding exons leading to frameshifts and premature stop codons—no mRNA and protein from the mutant alleles has been observed.

Accordingly, in certain aspects of the present disclosure, POSH participates in the formation of PKA complexes, including human PKA-containing complexes. Certain POSH polypeptides may be involved in disorders of the immune system, e.g., autoimmune disorders. Certain POSH polypeptides may be involved in the regulation of T-cell activation. In certain aspects, POSH participates in the ubiquitination of PI3K. In certain aspects, PKA subunit polypeptides participate in POSH-mediated processes.

Additionally, the disclosure relates in part to the discovery that PKA phosphorylates POSH, and further, that this phosphorylation inhibits the interaction of POSH with small GTPases, such as Rac. POSH also interacts with the small GTPase Chp, which interaction is also expected to be modulated by PKA phosphorylation. Small GTPases are important in vesicular trafficking, and therefore the findings disclosed herein demonstrate that POSH phosphorylation regulates the formation of complexes between POSH and proteins involved in the secretory system, such as Rac, Chp, TCL, TC10, Cdc42, Wrch-1, Rac2, Rac3 or RhoG. Data presented herein shows inhibition of PKA and POSH having similar effects, indicating that inhibition of PKA will achieve an effect similar to that of inhibition of POSH. However, given the effect of PKA on POSH interaction with proteins in the secretory pathway, it is expected that PKA regulates the timing of cyclical interactions that are needed to effect vesicular trafficking. Accordingly, it is expected that significant inhibition or activation of PKA will cause a disruption in POSH function.

The term “PKA subunit” is used herein to refer to a full-length human PKA subunit which includes a regulatory subunit (e.g., PRKAR1A) and a catalytic subunit (e.g., PRKACB or PRKACA), as well as an alternative PKA subunit composed of separate PKA subunit sequences (e.g., nucleic acid sequences) that may be a splice variant. The term “PKA subunit” is used herein to refer as well to various naturally occurring PKA subunit homologs, as well as functionally similar variants and fragments that retain at least 80%, 90%, 95%, or 99% sequence identity to a naturally occurring PKA subunit. The term specifically includes human PKA subunit nucleic acid and amino acid sequences and the sequences presented in the Examples.

3. Methods and Compositions for Treating POSH-Associated Diseases

In certain aspects, the application provides methods and compositions for treatment of POSH-associated diseases (disorders), including cancer and viral disorders, as well as disorders associated with unwanted apoptosis, including, for example a variety of neurodegenerative disorders, such as Alzheimer's disease.

In certain embodiments, the application relates to viral disorders (e.g., viral infections), and particularly disorders caused by retroid viruses, RNA viruses and/or envelope viruses. In view of the teachings herein, one of skill in the art will understand that the methods and compositions of the application are applicable to a wide range of viruses such as, for example, retroid viruses, RNA viruses, and envelope viruses. In a preferred embodiment, the present application is applicable to retroid viruses. In a more preferred embodiment, the present application is further applicable to retroviruses (retroviridae). In another more preferred embodiment, the present application is applicable to lentivirus, including primate lentivirus group. In a most preferred embodiment, the present application is applicable to Human Immunodeficiency virus (HIV), Human Immunodeficiency virus type-1 (HIV-1), Hepatitis B Virus (HBV) and Human T-cell Leukemia Virus (HTLV).

While not intended to be limiting, relevant retroviruses include: C-type retrovirus which causes lymphosarcoma in Northern Pike, the C-type retrovirus which infects mink, the caprine lentivirus which infects sheep, the Equine Infectious Anemia Virus (EIAV), the C-type retrovirus which infects pigs, the Avian Leukosis Sarcoma Virus (ALSV), the Feline Leukemia Virus (FeLV), the Feline Aids Virus, the Bovine Leukemia Virus (BLV), the Simian Leukemia Virus (SLV), the Simian Immuno-deficiency Virus (SIV), the Human T-cell Leukemia Virus type-I (HTLV-I), the Human T-cell Leukemia Virus type-II (HTLV-II), Human Immunodeficiency virus type-2 (HIV-2) and Human Immunodeficiency virus type-1 (HIV-1).

The method and compositions of the present application are further applicable to RNA viruses, including ssRNA negative-strand viruses and ssRNA positive-strand viruses. The ssRNA positive-strand viruses include Hepatitis C Virus (HCV). In a preferred embodiment, the present application is applicable to mononegavirales, including filoviruses. Filoviruses further include Ebola viruses and Marburg viruses.

Other RNA viruses include picornaviruses such as enterovirus, poliovirus, coxsackievirus and hepatitis A virus, the caliciviruses, including Norwalk-like viruses, the rhabdoviruses, including rabies virus, the togaviruses including alphaviruses, Semliki Forest virus, denguevirus, yellow fever virus and rubella virus, the orthomyxoviruses, including Type A, B, and C influenza viruses, the bunyaviruses, including the Rift Valley fever virus and the hantavirus, the filoviruses such as Ebola virus and Marburg virus, and the paramyxoviruses, including mumps virus and measles virus. Additional viruses that may be treated include herpes viruses.

In other embodiments, the application relates to methods of treating or preventing cancer diseases. The terms “cancer,” “tumor,” and “neoplasia” are used interchangeably herein. As used herein, a cancer (tumor or neoplasia) is characterized by one or more of the following properties: cell growth is not regulated by the normal biochemical and physical influences in the environment; anaplasia (e.g., lack of normal coordinated cell differentiation); and in some instances, metastasis. Cancer diseases include, for example, anal carcinoma, bladder carcinoma, breast carcinoma, cervix carcinoma, chronic lymphocytic leukemia, chronic myelogenous leukemia, endometrial carcinoma, hairy cell leukemia, head and neck carcinoma, lung (small cell) carcinoma, multiple myeloma, non-Hodgkin's lymphoma, follicular lymphoma, ovarian carcinoma, brain tumors, colorectal carcinoma, hepatocellular carcinoma, Kaposi's sarcoma, lung (non-small cell carcinoma), melanoma, pancreatic carcinoma, prostate carcinoma, renal cell carcinoma, and soft tissue sarcoma. Additional cancer disorders can be found in, for example, Isselbacher et al. (1994) Harrison's Principles of Internal Medicine 1814-1877, herein incorporated by reference.

In a specific embodiment, anticancer therapeutics of the application are used in treating a POSH-associated cancer. As described herein, POSH-associated cancers include, but are not limited to, the thyroid carcinoma, liver cancer (hepatocellular cancer), lung cancer, cervical cancer, ovarian cancer, renal cell carcinoma, lymphoma, osteosacoma, liposarcoma, leukemia, breast carcinoma, and breast adeno-carcinoma.

Preferred antiviral and anticancer therapeutics of the application can function by disrupting the biological activity of a POSH polypeptide or POSH complex in viral maturation. Certain therapeutics of the application function by disrupting the activity of a POSH-AK (e.g., PKA or JNK).

Exemplary therapeutics of the application include nucleic acid therapies such as, for example, RNAi constructs (small inhibitory RNAs), antisense oligonucleotides, ribozyme, and DNA enzymes. Other therapeutics include polypeptides, peptidomimetics, antibodies and small molecules. For example, therapeutics of the application include PKA inhibitors and JNK inhibitors as described above, under “Drug Screening Assays.”

Antisense therapies of the application include methods of introducing antisense nucleic acids to disrupt the expression of POSH polypeptides or proteins that are necessary for POSH function, such as certain POSH-AKs (e.g., PKA or JNK).

RNAi therapies include methods of introducing RNAi constructs to downregulate the expression of POSH polypeptides or POSH-AKs (e.g., PKA or JNK). Exemplary RNAi therapeutics include any one of SEQ ID NOs: 15, 16, 18, 19, 21, 22, 24 and 25.

Therapeutic polypeptides may be generated by designing polypeptides to mimic certain protein domains important in the formation of POSH: POSH-AK complexes, such as, for example, SH3 or RING domains. For example, a polypeptide comprising a POSH SH3 domain such as, for example, the SH3 domain as set forth in SEQ ID NO: 30 will compete for binding to a POSH SH3 domain and will therefore act to disrupt binding of a partner protein. In one embodiment, a binding partner may be a Gag polypeptide. In another embodiment, a binding partner may be Rac. In a further embodiment, a polypeptide that resembles an L domain may disrupt recruitment of Gag to the POSH complex.

In view of the specification, methods for generating antibodies directed to epitopes of POSH and POSH-AKs are known in the art. Antibodies may be introduced into cells by a variety of methods. One exemplary method comprises generating a nucleic acid encoding a single chain antibody that is capable of disrupting a POSH:POSH-AK complex. Such a nucleic acid may be conjugated to antibody that binds to receptors on the surface of target cells. It is contemplated that in certain embodiments, the antibody may target viral proteins that are present on the surface of infected cells, and in this way deliver the nucleic acid only to infected cells. Once bound to the target cell surface, the antibody is taken up by endocytosis, and the conjugated nucleic acid is transcribed and translated to produce a single chain antibody that interacts with and disrupts the targeted POSH:POSH-AK complex. Nucleic acids expressing the desired single chain antibody may also be introduced into cells using a variety of more conventional techniques, such as viral transfection (e.g., using an adenoviral system) or liposome-mediated transfection.

Small molecules of the application may be identified for their ability to modulate the formation of POSH:POSH-AK complexes.

Certain embodiments of the disclosure relate to use of a small molecule as an inhibitor of POSH. Examples of such small moclecule include the following compounds:

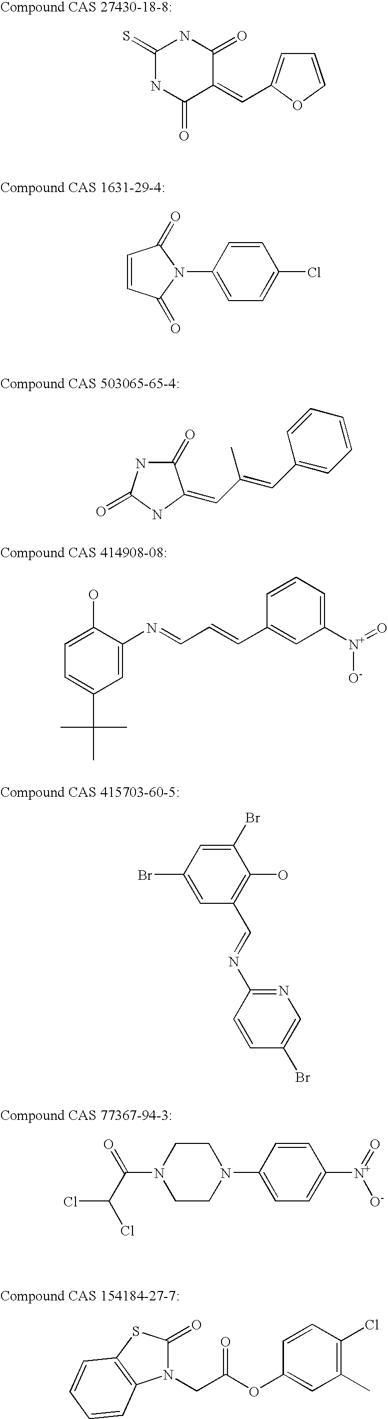

In certain embodiments, compounds useful in the instant compositions and methods include heteroarylmethylene-dihydro-2,4,6-pyrimidinetriones and their thione analogs. Preferred heteroaryl moieties include 5-membered rings such as thienyl, furyl, pyrrolyl, oxazolyl, thiazolyl, and imidazolyl moieties.

In certain embodiments, compounds useful in the instant compositions and methods include N-arylmaleimides, especially N-phenylmaleimides, in which the phenyl group may be substituted or unsubstituted.

In certain embodiments, compounds useful in the instant compositions and methods include arylallylidene-2,4-imidazolidinediones and their thione analogs. Preferred aryl groups are phenyl groups, and both the aryl and allylidene portions of the molecule may be substituted or unsubstituted.

In certain embodiments, compounds useful in the instant compositions and methods include substituted distyryl compounds and aza analogs thereof such as substituted 1,4-diphenylazabutadiene compounds.

In certain other embodiments, compounds useful in the instant compositions and methods include substituted styrenes and aza analogs thereof, such as 1,2-diphenylazaethylenes and 1-phenyl-2-pyridyl-azaethelenes.

In yet other embodiments, compounds useful in the instant compositions and methods include N-aryl-N′-acylpiperazines. In such compounds, the aryl ring, the acyl substituent, and/or the piperazine ring may be substituted or unsubstituted.

In additional embodiments, compounds useful in the instant compositions and methods include aryl esters of (2-oxo-benzooxazol-3-yl)-acetic acid, and analogs thereof in which one or more oxygen atoms are replaced by sulfur atoms.

In certain embodiments, the present application contemplates use of known PKA modulators (e.g., inhibitors or activators) in the methods of ihibiting viral infection and in the methods of treating or preventing cancer. Such PKA modulators include any compound, peptide, nucleotide derivative, nucleoside derivative, polysaccharide, sugar or other substance that can inhibit the activity of protein kinase A. Many PKA inhibitors are available and may be used. For example, many examples of PKA inhibitors including chemical structures, methods for administration and pharmacological effects are listed at the Calbiochem website at calbiochem.com. In general, inhibitors that also significantly inhibit protein kinase C activity are avoided.

In some embodiments, the PKA inhibitor is a nucleotide or nucleoside derivative. Specific examples of nucleoside or nucleotide derivatives that act as PKA inhibitors and that can be utilized in the disclosure include adenosine 3′,5′ cyclic monophosphorothioate. The H-89 inhibitor is a potent PKA inhibitor that can be used in the disclosure. The chemical name for the H-89 inhibitor is N-[2-((Pbromocinnamyl)amino)ethyl]isoquinolinesulfonamide. The KT5720 inhibitor from Calbiochem can also be used in the disclosure. Other PKA inhibitors which are available at from Calbiochem and can be used in the disclosure include ellagic acid (also named 4,4′,5,5′,6,6′-hexahydroxydiphenic acid 2,6,2′,6′-ditactone), piceatannol, 1-(5-Isoquinolinesulfonyl)methylpiperazine(H-7), N-[2-(methylamino)ethyl]isoquinolinesulfonamide(H-8), N-(2-aminoethyl)isoquinolinesulfonamide(H-9), and (5-isoquinolinesulfonyl)piperazine, 2HCI (H-100).

The PKA inhibitor can also be a peptide inhibitor (PKI). Such a peptide inhibitor can be any peptide that is recognized and bound by PKA but that PKA cannot phosphorylate. An example of a peptide inhibitor is a peptide with a “consensus sequence” for PKA recognition but with alanine in place of serine, for example, a peptide with the following sequence: Xaa-Arg-Arg-Xaa-Ala-Xaa, wherein Xaa is any amino acid, which specifically binds to the pseudoregion of the regulatory domain of PKA. Myristoylated PKA inhibitor amide (14-22, Cell-Permeable) having the sequence Myr-N-Gly-Arg-Thr-Gly-Arg-Arg-Asn-Ala-Ile-NH₂ is another example of a peptide inhibitor that can be utilized in the disclosure. A variety of other PKI peptides can be used as an inhibitor of protein kinase A in the practice of the disclosure. For example, several PKI peptides can be found in the NCBI protein database. See website at ncbi.nlm.nih.gov/Genbank/GenbankOverview. One example of a human PKI peptide can be found at Genbank Accession No. P04541 (gi: 417194). Another example of a human PKI peptide is at Genbank Accession No. NP 008997 (gi: 5902020). Another PKI that can be used as an inhibitor has the following sequence: Ile-Ala-Ser-Gly-Arg-Thr-Gly-Arg-Arg-Asn-Ala-Ile-His-Asp-11e-Leu-Val-SerSer-Ala. See published PCT application WO 03/080649.

Further examples of protein kinase A inhibitors are provided in the following references: Muniz et al., Proceedings of the National Academy of Sciences USA 1997 Dec. 23; 94(26) 14461-66; Baude et al., Journal of Biological Chemistry Vol. 269 issue 27 18128-18133 (July 1994); Scott et al.

Applicants found that POSH is phosphorylated by PKA and phosphorylation of POSH by PKA can inhibit POSH function, for example dissociating POSH from POSH interacting proteins (e.g, Rac). Therefore, in certain embodiments, the present disclosure also cotemplates use of PKA activators in treating or preventing a POSH-associated disease (e.g., viral infection or cancer). Exemplary PKA activators include, but are not limited to, forskolin, 8-Br-cAMP, and rolipram.

In certain embodiments, the present application also contemplates inhibitors of JNK pathway kinases (e.g, JNK, MLK, and MMK) as antiviral or anticancer therapeutics. Exemplary JNK inhibitors include, but are not limited to, anthrapyrazolones, e.g., anthra[1,9-cd]pyrazol-6(2H)-one (Biomol Research Laboratories Inc., Plymouth Meeting, Pa.) and those described in Bennett et al. 2001 Proc. Nat. Acad. Sci. 98(24): 13681-13686. Exemplary therapeutics of the application also include MLK inhibitors, such as pyrolocarbazoles, pyrazolones, isoindolones and those inhibitors described in U.S. Pat. No. 6,455,525 and PCT Patent Application with the following publication numbers: WO 02/095017; WO 02/17914; WO 01/85686; WO 01/32653; WO 00/47583.

The generation of nucleic acid based therapeutic agents directed to POSH and POSH-AKs is described below.

Methods for identifying and evaluating further modulators of POSH and POSH-AKs are also provided below.

4. RNA Interference, Ribozymes, Antisense and Related Constructs

In certain aspects, the application relates to RNAi, ribozyme, antisense and other nucleic acid-related methods and compositions for manipulating (typically decreasing) a POSH activity. Exemplary RNAi and ribozyme molecules may comprise a sequence as shown in any of SEQ ID Nos: 15, 16, 18, 19, 21, 22, 24 and 25.

In certain aspects, the application relates to RNAi, ribozyme, antisense and other nucleic acid-related methods and compositions for manipulating (typically decreasing) a POSH-AK activity. Specific instances of PRKAR1A, PRKACA, and PRKACB nucleic acids that may be used to design nucleic acids for RNAi, ribozyme, antisense are listed in the Examples.

Certain embodiments of the application make use of materials and methods for effecting knockdown of one or more POSH or POSH-AK genes by means of RNA interference (RNAi). RNAi is a process of sequence-specific post-transcriptional gene repression which can occur in eukaryotic cells. In general, this process involves degradation of an mRNA of a particular sequence induced by double-stranded RNA (dsRNA) that is homologous to that sequence. For example, the expression of a long dsRNA corresponding to the sequence of a particular single-stranded mRNA (ss mRNA) will labilize that message, thereby “interfering” with expression of the corresponding gene. Accordingly, any selected gene may be repressed by introducing a dsRNA which corresponds to all or a substantial part of the mRNA for that gene. It appears that when a long dsRNA is expressed, it is initially processed by a ribonuclease III into shorter dsRNA oligonucleotides of as few as 21 to 22 base pairs in length. Furthermore, Accordingly, RNAi may be effected by introduction or expression of relatively short homologous dsRNAs. Indeed the use of relatively short homologous dsRNAs may have certain advantages as discussed below.

Mammalian cells have at least two pathways that are affected by double-stranded RNA (dsRNA). In the RNAi (sequence-specific) pathway, the initiating dsRNA is first broken into short interfering (si) RNAs, as described above. The siRNAs have sense and antisense strands of about 21 nucleotides that form approximately 19 nucleotide si RNAs with overhangs of two nucleotides at each 3′ end. Short interfering RNAs are thought to provide the sequence information that allows a specific messenger RNA to be targeted for degradation. In contrast, the nonspecific pathway is triggered by dsRNA of any sequence, as long as it is at least about 30 base pairs in length. The nonspecific effects occur because dsRNA activates two enzymes: PKR, which in its active form phosphorylates the translation initiation factor eIF2 to shut down all protein synthesis, and 2′,5′ oligoadenylate synthetase (2′,5′-AS), which synthesizes a molecule that activates Rnase L, a nonspecific enzyme that targets all mRNAs. The nonspecific pathway may represent a host response to stress or viral infection, and, in general, the effects of the nonspecific pathway are preferably minimized under preferred methods of the present application. Significantly, longer dsRNAs appear to be required to induce the nonspecific pathway and, accordingly, dsRNAs shorter than about 30 bases pairs are preferred to effect gene repression by RNAi (see Hunter et al. (1975) J Biol Chem 250: 409-17; Manche et al. (1992) Mol Cell Biol 12: 5239-48; Minks et al. (1979) J Biol Chem 254: 10180-3; and Elbashir et al. (2001) Nature 411: 494-8).

RNAi has been shown to be effective in reducing or eliminating the expression of genes in a number of different organisms including Caenorhabditiis elegans (see e.g., Fire et al. (1998) Nature 391: 806-11), mouse eggs and embryos (Wianny et al. (2000) Nature Cell Biol 2: 70-5; Svoboda et al. (2000) Development 127: 4147-56), and cultured RAT-1 fibroblasts (Bahramina et al. (1999) Mol Cell Biol 19: 274-83), and appears to be an anciently evolved pathway available in eukaryotic plants and animals (Sharp (2001) Genes Dev. 15: 485-90). RNAi has proven to be an effective means of decreasing gene expression in a variety of cell types including HeLa cells, NIH/3T3 cells, COS cells, 293 cells and BHK-21 cells, and typically decreases expression of a gene to lower levels than that achieved using antisense techniques and, indeed, frequently eliminates expression entirely (see Bass (2001) Nature 411: 428-9). In mammalian cells, siRNAs are effective at concentrations that are several orders of magnitude below the concentrations typically used in antisense experiments (Elbashir et al. (2001) Nature 411: 494-8).

The double stranded oligonucleotides used to effect RNAi are preferably less than 30 base pairs in length and, more preferably, comprise about 25, 24, 23, 22, 21, 20, 19, 18 or 17 base pairs of ribonucleic acid. Optionally the dsRNA oligonucleotides of the application may include 3′ overhang ends. Exemplary 2-nucleotide 3′ overhangs may be composed of ribonucleotide residues of any type and may even be composed of 2′-deoxythymidine resides, which lowers the cost of RNA synthesis and may enhance nuclease resistance of siRNAs in the cell culture medium and within transfected cells (see Elbashir et al. (2001) Nature 411: 494-8). Longer dsRNAs of 50, 75, 100 or even 500 base pairs or more may also be utilized in certain embodiments of the application. Exemplary concentrations of dsRNAs for effecting RNAi are about 0.05 nM, 0.1 nM, 0.5 nM, 1.0 nM, 1.5 nM, 25 nM or 100 nM, although other concentrations may be utilized depending upon the nature of the cells treated, the gene target and other factors readily discernable the skilled artisan. Exemplary dsRNAs may be synthesized chemically or produced in vitro or in vivo using appropriate expression vectors. Exemplary synthetic RNAs include 21 nucleotide RNAs chemically synthesized using methods known in the art (e.g., Expedite RNA phophoramidites and thymidine phosphoramidite (Proligo, Germany). Synthetic oligonucleotides are preferably deprotected and gel-purified using methods known in the art (see e.g., Elbashir et al. (2001) Genes Dev. 15: 188-200). Longer RNAs may be transcribed from promoters, such as T7 RNA polymerase promoters, known in the art. A single RNA target, placed in both possible orientations downstream of an in vitro promoter, will transcribe both strands of the target to create a dsRNA oligonucleotide of the desired target sequence. Any of the above RNA species will be designed to include a portion of nucleic acid sequence represented in a POSH or POSH-AK nucleic acid, such as, for example, a nucleic acid that hybridizes, under stringent and/or physiological conditions, to any of SEQ ID Nos: 1, 3, 4, 6, 8 and 10 and complements thereof or any of the POSH-AK sequences presented in the Examples.

The specific sequence utilized in design of the oligonucleotides may be any contiguous sequence of nucleotides contained within the expressed gene message of the target. Programs and algorithms, known in the art, may be used to select appropriate target sequences. In addition, optimal sequences may be selected utilizing programs designed to predict the secondary structure of a specified single stranded nucleic acid sequence and allowing selection of those sequences likely to occur in exposed single stranded regions of a folded mRNA. Methods and compositions for designing appropriate oligonucleotides may be found, for example, in U.S. Pat. No. 6,251,588, the contents of which are incorporated herein by reference. Messenger RNA (mRNA) is generally thought of as a linear molecule which contains the information for directing protein synthesis within the sequence of ribonucleotides, however studies have revealed a number of secondary and tertiary structures that exist in most mRNAs. Secondary structure elements in RNA are formed largely by Watson-Crick type interactions between different regions of the same RNA molecule. Important secondary structural elements include intramolecular double stranded regions, hairpin loops, bulges in duplex RNA and internal loops. Tertiary structural elements are formed when secondary structural elements come in contact with each other or with single stranded regions to produce a more complex three dimensional structure. A number of researchers have measured the binding energies of a large number of RNA duplex structures and have derived a set of rules which can be used to predict the secondary structure of RNA (see e.g., Jaeger et al. (1989) Proc. Natl. Acad. Sci. USA 86:7706 (1989); and Turner et al. (1988) Annu. Rev. Biophys. Biophys. Chem. 17:167) . The rules are useful in identification of RNA structural elements and, in particular, for identifying single stranded RNA regions which may represent preferred segments of the mRNA to target for silencing RNAi, ribozyme or antisense technologies. Accordingly, preferred segments of the mRNA target can be identified for design of the RNAi mediating dsRNA oligonucleotides as well as for design of appropriate ribozyme and hammerheadribozyme compositions of the application.

The dsRNA oligonucleotides may be introduced into the cell by transfection with an heterologous target gene using carrier compositions such as liposomes, which are known in the art—e.g., Lipofectamine 2000 (Life Technologies) as described by the manufacturer for adherent cell lines. Transfection of dsRNA oligonucleotides for targeting endogenous genes may be carried out using Oligofectamine (Life Technologies). Transfection efficiency may be checked using fluorescence microscopy for mammalian cell lines after co-transfection of hGFP-encoding pAD3 (Kehlenback et al. (1998) J Cell Biol 141: 863-74). The effectiveness of the RNAi may be assessed by any of a number of assays following introduction of the dsRNAs. These include Western blot analysis using antibodies which recognize the POSH or POSH-AK gene product following sufficient time for turnover of the endogenous pool after new protein synthesis is repressed, reverse transcriptase polymerase chain reaction and Northern blot analysis to determine the level of existing POSH or POSH-AK target mRNA.

Further compositions, methods and applications of RNAi technology are provided in U.S. Pat. Nos. 6,278,039, 5,723,750 and 5,244,805, which are incorporated herein by reference.

Ribozyme molecules designed to catalytically cleave POSH or POSH-AK mRNA transcripts can also be used to prevent translation of suject POSH or POSH-AK mRNAs and/or expression of POSH or POSH-AKs (see, e.g., PCT International Publication WO90/11364, published Oct. 4, 1990; Sarver et al. (1990) Science 247:1222-1225 and U.S. Pat. No. 5,093,246). Ribozymes are enzymatic RNA molecules capable of catalyzing the specific cleavage of RNA. (For a review, see Rossi (1994) Current Biology 4: 469-471). The mechanism of ribozyme action involves sequence specific hybridization of the ribozyme molecule to complementary target RNA, followed by an endonucleolytic cleavage event. The composition of ribozyme molecules preferably includes one or more sequences complementary to a POSH or POSH-AK mRNA, and the well known catalytic sequence responsible for mRNA cleavage or a functionally equivalent sequence (see, e.g., U.S. Pat. No. 5,093,246, which is incorporated herein by reference in its entirety).

While ribozymes that cleave mRNA at site specific recognition sequences can be used to destroy target mRNAs, the use of hammerhead ribozymes is preferred. Hammerhead ribozymes cleave mRNAs at locations dictated by flanking regions that form complementary base pairs with the target mRNA. Preferably, the target mRNA has the following sequence of two bases: 5′-UG-3′. The construction and production of hammerhead ribozymes is well known in the art and is described more fully in Haseloff and Gerlach ((1988) Nature 334:585-591; and see PCT Appln. No. WO89/05852, the contents of which are incorporated herein by reference). Hammerhead ribozyme sequences can be embedded in a stable RNA such as a transfer RNA (tRNA) to increase cleavage efficiency in vivo (Perriman et al. (1995) Proc. Natl. Acad. Sci. USA, 92: 6175-79; de Feyter, and Gaudron, Methods in Molecular Biology, Vol. 74, Chapter 43, “Expressing Ribozymes in Plants”, Edited by Turner, P. C, Humana Press Inc., Totowa, N.J.). In particular, RNA polymerase III-mediated expression of tRNA fusion ribozymes are well known in the art (see Kawasaki et al. (1998) Nature 393: 284-9; Kuwabara et al. (1998) Nature Biotechnol. 16: 961-5; and Kuwabara et al. (1998) Mol. Cell 2: 617-27; Koseki et al. (1999) J Virol 73: 1868-77; Kuwabara et al. (1999) Proc Natl Acad Sci USA 96: 1886-91; Tanabe et al. (2000) Nature 406: 473-4). There are typically a number of potential hammerhead ribozyme cleavage sites within a given target cDNA sequence. Preferably the ribozyme is engineered so that the cleavage recognition site is located near the 5′ end of the target mRNA—to increase efficiency and minimize the intracellular accumulation of non-functional mRNA transcripts. Furthermore, the use of any cleavage recognition site located in the target sequence encoding different portions of the C-terminal amino acid domains of, for example, long and short forms of target would allow the selective targeting of one or the other form of the target, and thus, have a selective effect on one form of the target gene product.

Gene targeting ribozymes necessarily contain a hybridizing region complementary to two regions, each of at least 5 and preferably each 6, 7, 8, 9, 10, 11, 12, 13, 14, 15, 16, 17, 18, 19 or 20 contiguous nucleotides in length of a POSH or POSH-AK mRNA, such as an mRNA of a sequence represented in any of SEQ ID Nos: 1, 3, 4, 6, 8 or 10 or a POSH-AK presented in the Examples. In addition, ribozymes possess highly specific endoribonuclease activity, which autocatalytically cleaves the target sense mRNA. The present application extends to ribozymes which hybridize to a sense mRNA encoding a POSH gene such as a therapeutic drug target candidate gene, thereby hybridising to the sense mRNA and cleaving it, such that it is no longer capable of being translated to synthesize a functional polypeptide product.

The ribozymes of the present application also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al. (1984) Science 224:574-578; Zaug, et al. (1986) Science 231:470-475; Zaug, et al. (1986) Nature 324:429-433; published International patent application No. WO88/04300 by University Patents Inc.; Been, et al. (1986) Cell 47:207-216). The Cech-type ribozymes have an eight base pair active site which hybridizes to a target RNA sequence whereafter cleavage of the target RNA takes place. The application encompasses those Cech-type ribozymes which target eight base-pair active site sequences that are present in a target gene or nucleic acid sequence.

Ribozymes can be composed of modified oligonucleotides (e.g., for improved stability, targeting, etc.) and should be delivered to cells which express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target messages and inhibit translation. Because ribozymes, unlike antisense molecules, are catalytic, a lower intracellular concentration is required for efficiency.

In certain embodiments, a ribozyme may be designed by first identifying a sequence portion sufficient to cause effective knockdown by RNAi. The same sequence portion may then be incorporated into a ribozyme. In this aspect of the application, the gene-targeting portions of the ribozyme or RNAi are substantially the same sequence of at least 5 and preferably 6, 7, 8, 9, 10, 11, 12, 13, 14, 15, 16, 17, 18, 19 or 20 or more contiguous nucleotides of a POSH nucleic acid, such as a nucleic acid of any of SEQ ID Nos: 1, 3, 4, 6, 8, or 10 or POSH-AK nucleic acid, as presented in the Examples. In a long target RNA chain, significant numbers of target sites are not accessible to the ribozyme because they are hidden within secondary or tertiary structures (Birikh et al. (1997) Eur J Biochem 245: 1-16). To overcome the problem of target RNA accessibility, computer generated predictions of secondary structure are typically used to identify targets that are most likely to be single-stranded or have an “open” configuration (see Jaeger et al. (1989) Methods Enzymol 183: 281-306). Other approaches utilize a systematic approach to predicting secondary structure which involves assessing a huge number of candidate hybridizing oligonucleotides molecules (see Milner et al. (1997) Nat Biotechnol 15:537-41; and Patzel and Sczakiel (1998) Nat Biotechnol 16: 64-8). Additionally, U.S. Pat. No. 6,251,588, the contents of which are hereby incorporated herein, describes methods for evaluating oligonucleotide probe sequences so as to predict the potential for hybridization to a target nucleic acid sequence. The method of the application provides for the use of such methods to select preferred segments of a target mRNA sequence that are predicted to be single-stranded and, further, for the opportunistic utilization of the same or substantially identical target mRNA sequence, preferably comprising about 10-20 consecutive nucleotides of the target mRNA, in the design of both the RNAi oligonucleotides and ribozymes of the application.

A further aspect of the application relates to the use of the isolated “antisense” nucleic acids to inhibit expression, e.g., by inhibiting transcription and/or translation of a POSH or POSH-AK nucleic acid. The antisense nucleic acids may bind to the potential drug target by conventional base pair complementarity, or, for example, in the case of binding to DNA duplexes, through specific interactions in the major groove of the double helix. In general, these methods refer to the range of techniques generally employed in the art, and include any methods that rely on specific binding to oligonucleotide sequences.

An antisense construct of the present application can be delivered, for example, as an expression plasmid which, when transcribed in the cell, produces RNA which is complementary to at least a unique portion of the cellular mRNA which encodes a POSH or POSH-AK polypeptide. Alternatively, the antisense construct is an oligonucleotide probe, which is generated ex vivo and which, when introduced into the cell causes inhibition of expression by hybridizing with the mRNA and/or genomic sequences of a POSH or POSH-AK nucleic acid. Such oligonucleotide probes are preferably modified oligonucleotides, which are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659-2668.

With respect to antisense DNA, oligodeoxyribonucleotides derived from the translation initiation site, e.g., between the −10 and +10 regions of the target gene, are preferred. Antisense approaches involve the design of oligonucleotides (either DNA or RNA) that are complementary to mRNA encoding a POSH or POSH-AK polypeptide. The antisense oligonucleotides will bind to the mRNA transcripts and prevent translation. Absolute complementarity, although preferred, is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well. (Wagner, R. 1994. Nature 372:333). Therefore, oligonucleotides complementary to either the 5′ or 3′ untranslated, non-coding regions of a gene could be used in an antisense approach to inhibit translation of that mRNA. Oligonucleotides complementary to the 5′ untranslated region of the mRNA should include the complement of the AUG start codon. Antisense oligonucleotides complementary to mRNA coding regions are less efficient inhibitors of translation but could also be used in accordance with the application. Whether designed to hybridize to the 5′,3′ or coding region of mRNA, antisense nucleic acids should be at least six nucleotides in length, and are preferably less that about 100 and more preferably less than about 50, 25, 17 or 10 nucleotides in length.

It is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Results obtained using the antisense oligonucleotide may be compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The antisense oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. W088/09810, published Dec. 15, 1988) or the blood-brain barrier (see, e.g., PCT Publication No. W089/10134, published Apr. 25, 1988), hybridization-triggered cleavage agents. (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytiethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an alpha-anomeric oligonucleotide. An alpha-anomeric oligonucleotide forms specific double-stranded hybrids with complementary RNA in which, contrary to the usual antiparallel orientation, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

While antisense nucleotides complementary to the coding region of a POSH or POSH-AK mRNA sequence can be used, those complementary to the transcribed untranslated region may also be used.

In certain instances, it may be difficult to achieve intracellular concentrations of the antisense sufficient to suppress translation on endogenous mRNAs. Therefore a preferred approach utilizes a recombinant DNA construct in which the antisense oligonucleotide is placed under the control of a strong pol III or pol II promoter. The use of such a construct to transfect target cells will result in the transcription of sufficient amounts of single stranded RNAs that will form complementary base pairs with the endogenous potential drug target transcripts and thereby prevent translation. For example, a vector can be introduced such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al, 1982, Nature 296:3942), etc. Any type of plasmid, cosmid, YAC or viral vector can be used to prepare the recombinant DNA construct, which can be introduced directly into the tissue site.

Alternatively, POSH or POSH-AK gene expression can be reduced by targeting deoxyribonucleotide sequences complementary to the regulatory region of the gene (i.e., the promoter and/or enhancers) to form triple helical structures that prevent transcription of the gene in target cells in the body. (See generally, Helene, C. 1991, Anticancer Drug Des., 6(6):569-84; Helene, C., et al., 1992, Ann. N.Y. Acad. Sci., 660:27-36; and Maher, L. J., 1992, Bioassays 14(12):807-15).

Nucleic acid molecules to be used in triple helix formation for the inhibition of transcription are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, POSH or POSH-AK sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

A further aspect of the application relates to the use of DNA enzymes to inhibit expression of a POSH or POSH-AK gene. DNA enzymes incorporate some of the mechanistic features of both antisense and ribozyme technologies. DNA enzymes are designed so that they recognize a particular target nucleic acid sequence, much like an antisense oligonucleotide, however much like a ribozyme they are catalytic and specifically cleave the target nucleic acid.

There are currently two basic types of DNA enzymes, and both of these were identified by Santoro and Joyce (see, for example, U.S. Pat. No. 6,110,462). The 10-23 DNA enzyme comprises a loop structure which connect two arms. The two arms provide specificity by recognizing the particular target nucleic acid sequence while the loop structure provides catalytic function under physiological conditions.

Briefly, to design an ideal DNA enzyme that specifically recognizes and cleaves a target nucleic acid, one of skill in the art must first identify the unique target sequence. This can be done using the same approach as outlined for antisense oligonucleotides. Preferably, the unique or substantially sequence is a G/C rich of approximately 18 to 22 nucleotides. High G/C content helps insure a stronger interaction between the DNA enzyme and the target sequence.

When synthesizing the DNA enzyme, the specific antisense recognition sequence that will target the enzyme to the message is divided so that it comprises the two arms of the DNA enzyme, and the DNA enzyme loop is placed between the two specific arms.

Methods of making and administering DNA enzymes can be found, for example, in U.S. Pat. No. 6,110,462. Similarly, methods of delivery DNA ribozymes in vitro or in vivo include methods of delivery RNA ribozyme, as outlined in detail above. Additionally, one of skill in the art will recognize that, like antisense oligonucleotide, DNA enzymes can be optionally modified to improve stability and improve resistance to degradation.

Antisense RNA and DNA, ribozyme, RNAi and triple helix molecules of the application may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

5. Drug Screening Assays

In certain aspects, the present application provides assays for identifying therapeutic agents which either interfere with or promote POSH or POSH-AK function. In certain aspects, the present application also provides assays for identifying therapeutic agents which either interfere with or promote the complex formation between a POSH polypeptide and a POSH-AK polypeptide.

In certain embodiments, agents of the application are antiviral agents, optionally interfering with viral maturation, and preferably where the virus is an envelope virus, and optionally a retroid virus or an RNA virus. In other embodiments, agents of the application are anticancer agents. In certain embodiments, an antiviral or anticancer agent interferes with the ubiquitin ligase catalytic activity of POSH (e.g., POSH auto-ubiquitination or transfer to a target protein). In other embodiments, agents disclosed herein inhibit or promote POSH and POSH-AK mediated cellular processes such as apoptosis and protein processing in the secretory pathway.

In certain preferred embodiments, an antiviral agent interferes with the interaction between POSH and a POSH-AK polypeptide, for example an antiviral agent may disrupt or render irreversible interaction between a POSH polypeptide and POSH-AK polypeptide such as a PKA subunit polypeptide (as in the case of a POSH dimer, a heterodimer of two different POSH polypeptides, homomultimers and heteromultimers). In further embodiments, agents of the application are anti-apoptotic agents, optionally interfering with JNK and/or NF-κB signaling. In yet additional embodiments, agents of the application interfere with the signaling of a GTPase, such as Rac or Ras, optionally disrupting the interaction between a POSH polypeptide and a Rac protein. In certain embodiments, agents of the application modulate the ubiquitin ligase activity of POSH and may be used to treat certain diseases related to ubiquitin ligase activity.

In certain embodiments, the application provides assays to identify, optimize or otherwise assess agents that increase or decrease a ubiquitin-related activity of a POSH polypeptide. Ubiquitin-related activities of POSH polypeptides may include the self-ubiquitination activity of a POSH polypeptide, generally involving the transfer of ubiquitin from an E2 enzyme to the POSH polypeptide, and the ubiquitination of a target protein, generally involving the transfer of a ubiquitin from a POSH polypeptide to the target protein. In certain embodiments, a POSH activity is mediated, at least in part, by a POSH RING domain.

In certain embodiments, an assay comprises forming a mixture comprising a POSH polypeptide, an E2 polypeptide and a source of ubiquitin (which may be the E2 polypeptide pre-complexed with ubiquitin). Optionally the mixture comprises an E1 polypeptide and optionally the mixture comprises a target polypeptide. Additional components of the mixture may be selected to provide conditions consistent with the ubiquitination of the POSH polypeptide. One or more of a variety of parameters may be detected, such as POSH-ubiquitin conjugates, E2-ubiquitin thioesters, free ubiquitin and target polypeptide-ubiquitin complexes. The term “detect” is used herein to include a determination of the presence or absence of the subject of detection (e.g., POSH-ubiqutin, E2-ubiquitin, etc.), a quantitative measure of the amount of the subject of detection, or a mathematical calculation of the presence, absence or amount of the subject of detection, based on the detection of other parameters. The term “detect” includes the situation wherein the subject of detection is determined to be absent or below the level of sensitivity. Detection may comprise detection of a label (e.g., fluorescent label, radioisotope label, and other described below), resolution and identification by size (e.g., SDS-PAGE, mass spectroscopy), purification and detection, and other methods that, in view of this specification, will be available to one of skill in the art. For instance, radioisotope labeling may be measured by scintillation counting, or by densitometry after exposure to a photographic emulsion, or by using a device such as a Phosphorimager. Likewise, densitometry may be used to measure bound ubiquitin following a reaction with an enzyme label substrate that produces an opaque product when an enzyme label is used. In a preferred embodiment, an assay comprises detecting the POSH-ubiquitin conjugate.

In certain embodiments, an assay comprises forming a mixture comprising a POSH polypeptide, a target polypeptide and a source of ubiquitin (which may be the POSH polypeptide pre-complexed with ubiquitin). Optionally the mixture comprises an E1 and/or E2 polypeptide and optionally the mixture comprises an E2-ubiquitin thioester. Additional components of the mixture may be selected to provide conditions consistent with the ubiquitination of the target polypeptide. One or more of a variety of parameters may be detected, such as POSH-ubiquitin conjugates and target polypeptide-ubiquitin conjugates. In a preferred embodiment, an assay comprises detecting the target polypeptide-ubiquitin conjugate. In another preferred embodiment, an assay comprises detecting the POSH-ubiquitin conjugate.

An assay described above may be used in a screening assay to identify agents that modulate a ubiquitin-related activity of a POSH polypeptide. A screening assay will generally involve adding a test agent to one of the above assays, or any other assay designed to assess a ubiquitin-related activity of a POSH polypeptide. The parameter(s) detected in a screening assay may be compared to a suitable reference. A suitable reference may be an assay run previously, in parallel or later that omits the test agent. A suitable reference may also be an average of previous measurements in the absence of the test agent. In general the components of a screening assay mixture may be added in any order consistent with the overall activity to be assessed, but certain variations may be preferred. For example, in certain embodiments, it may be desirable to pre-incubate the test agent and the E3 (e.g., the POSH polypeptide), followed by removing the test agent and addition of other components to complete the assay. In this manner, the effects of the agent solely on the POSH polypeptide may be assessed. In certain preferred embodiments, a screening assay for an antiviral agent employs a target polypeptide comprising an L domain, and preferably an HIV L domain.

In certain embodiments, an assay is performed in a high-throughput format. For example, one of the components of a mixture may be affixed to a solid substrate and one or more of the other components is labeled. For example, the POSH polypeptide may be affixed to a surface, such as a 96-well plate, and the ubiquitin is in solution and labeled. An E2 and E1 are also in solution, and the POSH-ubiquitin conjugate formation may be measured by washing the solid surface to remove uncomplexed labeled ubiquitin and detecting the ubiquitin that remains bound. Other variations may be used. For example, the amount of ubiquitin in solution may be detected. In certain embodiments, the formation of ubiquitin complexes may be measured by an interactive technique, such as FRET, wherein a ubiquitin is labeled with a first label and the desired complex partner (e.g., POSH polypeptide or target polypeptide) is labeled with a second label, wherein the first and second label interact when they come into close proximity to produce an altered signal. In FRET, the first and second labels are fluorophores. FRET is described in greater detail below. The formation of polyubiquitin complexes may be performed by mixing two or more pools of differentially labeled ubiquitin that interact upon formation of a polyubiqutin (see, e.g., US Patent Publication 20020042083). High-throughput may be achieved by performing an interactive assay, such as FRET, in solution as well. In addition, if a polypeptide in the mixture, such as the POSH polypeptide or target polypeptide, is readily purifiable (e.g., with a specific antibody or via a tag such as biotin, FLAG, polyhistidine, etc.), the reaction may be performed in solution and the tagged polypeptide rapidly isolated, along with any polypeptides, such as ubiquitin, that are associated with the tagged polypeptide. Proteins may also be resolved by SDS-PAGE for detection.

In certain embodiments, the ubiquitin is labeled, either directly or indirectly. This typically allows for easy and rapid detection and measurement of ligated ubiquitin, making the assay useful for high-throughput screening applications. As described above, certain embodiments may employ one or more tagged or labeled proteins. A “tag” is meant to include moieties that facilitate rapid isolation of the tagged polypeptide. A tag may be used to facilitate attachment of a polypeptide to a surface. A “label” is meant to include moieties that facilitate rapid detection of the labeled polypeptide. Certain moieties may be used both as a label and a tag (e.g., epitope tags that are readily purified and detected with a well-characterized antibody). Biotinylation of polypeptides is well known, for example, a large number of biotinylation agents are known, including amine-reactive and thiol-reactive agents, for the biotinylation of proteins, nucleic acids, carbohydrates, carboxylic acids; see chapter 4, Molecular Probes Catalog, Haugland, 6th Ed. 1996, hereby incorporated by reference. A biotinylated substrate can be attached to a biotinylated component via avidin or streptavidin. Similarly, a large number of haptenylation reagents are also known.

An “E1” is a ubiquitin activating enzyme. In a preferred embodiment, E1 is capable of transferring ubiquitin to an E2. In a preferred embodiment, E1 forms a high energy thiolester bond with ubiquitin, thereby “activating” the ubiquitin. An “E2” is a ubiquitin carrier enzyme (also known as a ubiquitin conjugating enzyme). In a preferred embodiment, ubiquitin is transferred from E1 to E2. In a preferred embodiment, the transfer results in a thiolester bond formed between E2 and ubiquitin. In a preferred embodiment, E2 is capable of transferring ubiquitin to a POSH polypeptide.

In an alternative embodiment, a POSH polypeptide, E2 or target polypeptide is bound to a bead, optionally with the assistance of a tag. Following ligation, the beads may be separated from the unbound ubiquitin and the bound ubiquitin measured. In a preferred embodiment, POSH polypeptide is bound to beads and the composition used includes labeled ubiquitin. In this embodiment, the beads with bound ubiquitin may be separated using a fluorescence-activated cell sorting (FACS) machine. Methods for such use are described in U.S. patent application Ser. No. 09/047,119, which is hereby incorporated in its entirety. The amount of bound ubiquitin can then be measured.

In a screening assay, the effect of a test agent may be assessed by, for example, assessing the effect of the test agent on kinetics, steady-state and/or endpoint of the reaction.

The components of the various assay mixtures provided herein may be combined in varying amounts. In a preferred embodiment, ubiquitin (or E2 complexed ubiquitin) is combined at a final concentration of from 5 to 200 ng per 100 microliter reaction solution. Optionally E1 is used at a final concentration of from 1 to 50 ng per 100 microliter reaction solution. Optionally E2 is combined at a final concentration of 10 to 100 ng per 100 microliter reaction solution, more preferably 10-50 ng per 100 microliter reaction solution. In a preferred embodiment, POSH polypeptide is combined at a final concentration of from 1 to 500 ng per 100 microliter reaction solution.

Generally, an assay mixture is prepared so as to favor ubiquitin ligase activity and/or ubiquitination acitivty. Generally, this will be physiological conditions, such as 50-200 mM salt (e.g., NaCl, KCl), pH of between 5 and 9, and preferably between 6 and 8. Such conditions may be optimized through trial and error. Incubations may be performed at any temperature which facilitates optimal activity, typically between 4 and 40° C. Incubation periods are selected for optimum activity, but may also be optimized to facilitate rapid high through put screening. Typically between 0.5 and 1.5 hours will be sufficient. A variety of other reagents may be included in the compositions. These include reagents like salts, solvents, buffers, neutral proteins, e.g., albumin, detergents, etc. which may be used to facilitate optimal ubiquitination enzyme activity and/or reduce non-specific or background interactions. Also reagents that otherwise improve the efficiency of the assay, such as protease inhibitors, nuclease inhibitors, anti-microbial agents, etc., may be used. The compositions will also preferably include adenosine tri-phosphate (ATP). The mixture of components may be added in any order that promotes ubiquitin ligase activity or optimizes identification of candidate modulator effects. In a preferred embodiment, ubiquitin is provided in a reaction buffer solution, followed by addition of the ubiquitination enzymes. In an alternate preferred embodiment, ubiquitin is provided in a reaction buffer solution, a candidate modulator is then added, followed by addition of the ubiquitination enzymes.

In general, a test agent that decreases a POSH ubiquitin-related activity may be used to inhibit POSH function in vivo, while a test agent that increases a POSH ubiquitin-related activity may be used to stimulate POSH function in vivo. Test agent may be modified for use in vivo, e.g., by addition of a hydrophobic moiety, such as an ester.

An additional POSH-AK may be added to a POSH ubiquitination assay to assess the effect of the POSH-AK (e.g., PRKAR1A, PRKACA, or PRKACB) on POSH-mediated ubiquitination and/or to assess whether the POSH-AK is a target for POSH-mediated ubiquitination.

Certain embodiments of the application relate to assays for identifying agents that bind to a POSH or POSH-AK polypeptide, optionally a particular domain of POSH such as an SH3 or RING domain or a particular domain of a POSH-AK, particularly a kinase catalytic domain or ATP binding domain. In preferred embodiments, a POSH polypeptide is a polypeptide comprising the fourth SH3 domain of hPOSH (SEQ ID NO: 30). A wide variety of assays may be used for this purpose, including labeled in vitro protein-protein binding assays, electrophoretic mobility shift assays, immunoassays for protein binding, and the like. The purified protein may also be used for determination of three-dimensional crystal structure, which can be used for modeling intermolecular interactions and design of test agents. In one embodiment, an assay detects agents which inhibit interaction of one or more subject POSH polypeptides with a POSH-AK. In another embodiment, the assay detects agents which modulate the intrinsic biological activity of a POSH polypeptide or POSH complex, such as an enzymatic activity, binding to other cellular components, cellular compartmentalization, and the like.

Certain embodiments of the application relate to assays for identifying agents that modulate a POSH-AK polypeptide such as a PKA subunit polypeptide. Preferred PKA subunit polypeptides include PRKAR1A, PRKACA, and PRKACB. Exemplary assays used for this purpose may include detecting phosphorylation of PKA subunit, kinase activity of the PKA subunit, ability of the PKA subunit to elicit downstream signaling of the PKA pathway, and the like. For example, activity of protein kinase A can be assayed either in vitro or in vivo. PKA activity can be determined by detecting posphorylation of a PKA specific substrate. The specific PKA substrate can be any convenient peptide with a serine that is recognized as a phosphorylation site by PKA. For example, the peptide substrate can have the sequence: Leu-Arg-Arg-Ala-Ser-Leu-Gly.

In one aspect, the application provides methods and compositions for the identification of compositions that interfere with the function of POSH or POSH-AK polypeptides. Given the role of POSH polypeptides in viral production, compositions that perturb the formation or stability of the protein-protein interactions between POSH polypeptides and the proteins that they interact with, such as POSH-AKs, and particularly POSH complexes comprising a viral protein, are candidate pharmaceuticals for the treatment of viral infections.

While not wishing to be bound to mechanism, it is postulated that POSH polypeptides promote the assembly of protein complexes that are important in release of virions and other biological processes. Complexes of the application may include a combination of a POSH polypeptide and a POSH-AK. Exemplary complexes may comprise one or more of the following: a POSH polypeptide (as in the case of a POSH dimer, a heterodimer of two different POSH, homomultimers and heteromultimers); a PKA subunit polypeptide (e.g., PRKAR1A, PRKACA, or PRKACB).

In an assay for an antiviral or antiapoptotic agent, the test agent is assessed for its ability to disrupt or inhibit the formation of a complex of a POSH polypeptide and a small GTPase, such as Rac or Chp polypeptide, particularly a human Rac polypeptide, such as Rac1.

A variety of assay formats will suffice and, in light of the present disclosure, those not expressly described herein will nevertheless be comprehended by one of ordinary skill in the art. Assay formats which approximate such conditions as formation of protein complexes, enzymatic activity, and even a POSH polypeptide-mediated membrane reorganization or vesicle formation activity, may be generated in many different forms, and include assays based on cell-free systems, e.g., purified proteins or cell lysates, as well as cell-based assays which utilize intact cells. Simple binding assays can also be used to detect agents which bind to POSH. Such binding assays may also identify agents that act by disrupting the interaction between a POSH polypeptide and a POSH interacting protein, or the binding of a POSH polypeptide or complex to a substrate. Agents to be tested can be produced, for example, by bacteria, yeast or other organisms (e.g., natural products), produced chemically (e.g., small molecules, including peptidomimetics), or produced recombinantly. In a preferred embodiment, the test agent is a small organic molecule, e.g., other than a peptide or oligonucleotide, having a molecular weight of less than about 2,000 daltons.

In many drug screening programs which test libraries of compounds and natural extracts, high throughput assays are desirable in order to maximize the number of compounds surveyed in a given period of time. Assays of the present application which are performed in cell-free systems, such as may be developed with purified or semi-purified proteins or with lysates, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with other proteins or changes in enzymatic properties of the molecular target.

In preferred in vitro embodiments of the present assay, a reconstituted POSH complex comprises a reconstituted mixture of at least semi-purified proteins. By semi-purified, it is meant that the proteins utilized in the reconstituted mixture have been previously separated from other cellular or viral proteins. For instance, in contrast to cell lysates, the proteins involved in POSH complex formation are present in the mixture to at least 50% purity relative to all other proteins in the mixture, and more preferably are present at 90-95% purity. In certain embodiments of the subject method, the reconstituted protein mixture is derived by mixing highly purified proteins such that the reconstituted mixture substantially lacks other proteins (such as of cellular or viral origin) which might interfere with or otherwise alter the ability to measure POSH complex assembly and/or disassembly.

Assaying POSH complexes, in the presence and absence of a candidate inhibitor, can be accomplished in any vessel suitable for containing the reactants. Examples include microtitre plates, test tubes, and micro-centrifuge tubes.

In one embodiment of the present application, drug screening assays can be generated which detect inhibitory agents on the basis of their ability to interfere with assembly or stability of the POSH complex. In an exemplary binding assay, the compound of interest is contacted with a mixture comprising a POSH polypeptide and at least one interacting polypeptide. Detection and quantification of POSH complexes provides a means for determining the compound's efficacy at inhibiting (or potentiating) interaction between the two polypeptides. The efficacy of the compound can be assessed by generating dose response curves from data obtained using various concentrations of the test compound. Moreover, a control assay can also be performed to provide a baseline for comparison. In the control assay, the formation of complexes is quantitated in the absence of the test compound.

Complex formation between the POSH polypeptides and a substrate polypeptide may be detected by a variety of techniques, many of which are effectively described above. For instance, modulation in the formation of complexes can be quantitated using, for example, detectably labeled proteins (e.g., radiolabeled, fluorescently labeled, or enzymatically labeled), by immunoassay, or by chromatographic detection. Surface plasmon resonance systems, such as those available from Biacore International AB (Uppsala, Sweden), may also be used to detect protein-protein interaction

Often, it will be desirable to immobilize one of the polypeptides to facilitate separation of complexes from uncomplexed forms of one of the proteins, as well as to accommodate automation of the assay. In an illustrative embodiment, a fusion protein can be provided which adds a domain that permits the protein to be bound to an insoluble matrix. For example, GST-POSH fusion proteins can be adsorbed onto glutathione sepharose beads (Sigma Chemical, St. Louis, Mo.) or glutathione derivatized microtitre plates, which are then combined with a potential interacting protein, e.g., an ³⁵S-labeled polypeptide, and the test compound and incubated under conditions conducive to complex formation. Following incubation, the beads are washed to remove any unbound interacting protein, and the matrix bead-bound radiolabel determined directly (e.g., beads placed in scintillant), or in the supernatant after the complexes are dissociated, e.g., when microtitre plate is used. Alternatively, after washing away unbound protein, the complexes can be dissociated from the matrix, separated by SDS-PAGE gel, and the level of interacting polypeptide found in the matrix-bound fraction quantitated from the gel using standard electrophoretic techniques.

In a further embodiment, agents that bind to a POSH or POSH-AP may be identified by using an immobilized POSH or POSH-AP. In an illustrative embodiment, a fusion protein can be provided which adds a domain that permits the protein to be bound to an insoluble matrix. For example, GST-POSH fusion proteins can be adsorbed onto glutathione sepharose beads (Sigma Chemical, St. Louis, Mo.) or glutathione derivatized microtitre plates, which are then combined with a potential labeled binding agent and incubated under conditions conducive to binding. Following incubation, the beads are washed to remove any unbound agent, and the matrix bead-bound label determined directly, or in the supernatant after the bound agent is dissociated.

In yet another embodiment, the POSH polypeptide and potential interacting polypeptide can be used to generate an interaction trap assay (see also, U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696), for subsequently detecting agents which disrupt binding of the proteins to one and other.

In particular, the method makes use of chimeric genes which express hybrid proteins. To illustrate, a first hybrid gene comprises the coding sequence for a DNA-binding domain of a transcriptional activator can be fused in frame to the coding sequence for a “bait” protein, e.g., a POSH polypeptide of sufficient length to bind to a potential interacting protein. The second hybrid protein encodes a transcriptional activation domain fused in frame to a gene encoding a “fish” protein, e.g., a potential interacting protein of sufficient length to interact with the POSH polypeptide portion of the bait fusion protein. If the bait and fish proteins are able to interact, e.g., form a POSH complex, they bring into close proximity the two domains of the transcriptional activator. This proximity causes transcription of a reporter gene which is operably linked to a transcriptional regulatory site responsive to the transcriptional activator, and expression of the reporter gene can be detected and used to score for the interaction of the bait and fish proteins.

One aspect of the present application provides reconstituted protein preparations including a POSH polypeptide and one or more interacting polypeptides.

In still further embodiments of the present assay, the POSH complex is generated in whole cells, taking advantage of cell culture techniques to support the subject assay. For example, as described below, the POSH complex can be constituted in a eukaryotic cell culture system, including mammalian and yeast cells. Often it will be desirable to express one or more viral proteins (e.g., Gag or Env) in such a cell along with a subject POSH polypeptide. It may also be desirable to infect the cell with a virus of interest. Advantages to generating the subject assay in an intact cell include the ability to detect inhibitors which are functional in an environment more closely approximating that which therapeutic use of the inhibitor would require, including the ability of the agent to gain entry into the cell. Furthermore, certain of the in vivo embodiments of the assay, such as examples given below, are amenable to high through-put analysis of candidate agents.

The components of the POSH complex can be endogenous to the cell selected to support the assay. Alternatively, some or all of the components can be derived from exogenous sources. For instance, fusion proteins can be introduced into the cell by recombinant techniques (such as through the use of an expression vector), as well as by microinjecting the fusion protein itself or mRNA encoding the fusion protein.

In many embodiments, a cell is manipulated after incubation with a candidate agent and assayed for a POSH or POSH-AK activity. In certain embodiments a POSH or POSH-AK activity is represented by production of virus like particles. As demonstrated herein, an agent that disrupts POSH or POSH-AP activity can cause a decrease in the production of virus like particles. Other bioassays for POSH or POSH-AP activities may include apoptosis assays (e.g., cell survival assays, apoptosis reporter gene assays, etc.) and NF-κB nuclear localization assays (see e.g., Tapon et al. (1998) EMBO J. 17: 1395-1404). In certain embodiments, POSH or POSH-AK activities may include, without limitation, complex formation, ubiquitination and membrane fusion events (eg. release of viral buds or fusion of vesicles). POSH-AK activity may be assessed by the presence of phosphorylated substrate, such as, in the case of PKA, phosphorylated POSH. The interaction of POSH with a small GTPase such as Rac or Chp may also be indicative of the absence of phosphorylation of POSH by PKA. POSH complex formation may be assessed by immunoprecipitation and analysis of co-immunoprecipiated proteins or affinity purification and analysis of co-purified proteins. Fluorescence Resonance Energy Transfer (FRET)-based assays or other energy transfer assays may also be used to determine complex formation.

In a further embodiment, transcript levels may be measured in cells having higher or lower levels of POSH or POSH-AP activity in order to identify genes that are regulated by POSH or POSH-APs. Promoter regions for such genes (or larger portions of such genes) may be operatively linked to a reporter gene and used in a reporter gene-based assay to detect agents that enhance or diminish POSH-or POSH-AP-regulated gene expression. Transcript levels may be determined in any way known in the art, such as, for example, Northern blotting, RT-PCR, microarray, etc. Increased POSH activity may be achieved, for example, by introducing a strong POSH expression vector. Decreased POSH activity may be achieved, for example, by RNAi, antisense, ribozyme, gene knockout, etc.

In general, where the screening assay is a binding assay (whether protein-protein binding, agent-protein binding, etc.), one or more of the molecules may be joined to a label, where the label can directly or indirectly provide a detectable signal. Various labels include radioisotopes, fluorescers, chemiluminescers, enzymes, specific binding molecules, particles, e.g., magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

In further embodiments, the application provides methods for identifying targets for therapeutic intervention. A polypeptide that interacts with POSH or participates in a POSH-mediated process (such as viral maturation) may be used to identify candidate therapeutics. Such targets may be identified by identifying proteins that associated with POSH (POSH-APs) by, for example, immunoprecipitation with an anti-POSH antibody, in silico analysis of high-throughput binding data, two-hybrid screens, and other protein-protein interaction assays described herein or otherwise known in the art in view of this disclosure. Agents that bind to such targets or disrupt protein-protein interactions thereof, or inhibit a biochemical activity thereof may be used in such an assay. Targets that have been identified by such approaches include a PKA subunit polypeptide (e.g., PRKAR1A, PRKACA, or PRKACB).

A variety of other reagents may be included in the screening assay. These include reagents like salts, neutral proteins, e.g., albumin, detergents, etc that are used to facilitate optimal protein-protein binding and/or reduce nonspecific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4° C. and 40° C. Incubation periods are selected for optimum activity, but may also be optimized to facilitate rapid high-throughput screening.

In certain embodiments, a test agent may be assessed for antiviral or anticancer activity by assessing effects on an activity (function) of a POSH-AK. Activity (function) may be affected by an agent that acts at one or more of the transcriptional, translational or post-translational stages. For example, an siRNA directed to a POSH-AP encoding gene will decrease activity, as will a small molecule that interferes with a catalytic activity of a POSH-AK In certain embodiments, the agent inhibits the activity of one or more polypeptides selected from the group consisting of: JNK1, JNK2, MLK1, MLK2, and MLK3. JNK activity may be assessed in biochemical or cell-based assays by determining phosphorylation of a JNK substrate, such as Jun. JNK activity may also be assessed by determining expression of a nucleic acid, preferably a nucleic acid encoding a reporter gene, which is under control of a promoter that is responsive to JNK, such as a Jun regulated promoter. MLK activity may be assessed in biochemical or cell-based assays by determining phosphorylation of a MLK substrate, such as MKK4 or MKK7. MLK activity may also be assessed by determining expression of a nucleic acid, preferably a nucleic acid encoding a reporter gene, which is under control of a promoter that is responsive to MLK activity, such as a MLK-JNK pathway regulated promoter. MKK activity may be assessed in biochemical or cell-based assays by determining phosphorylation of a MKK substrate, such as a JNK. MKK activity may also be assessed by determining expression of a nucleic acid, preferably a nucleic acid encoding a reporter gene, which is under control of a promoter that is responsive to MKK activity, such as a MKK-JNK pathway regulated promoter.

6. Exemplary Nucleic Acids and Expression Vectors

In certain aspects, the application relates to nucleic acids encoding POSH polypeptides, such as, for example, SEQ ID Nos: 2, 5, 7, 9, 11, 26, 27, 28, 29 and 30. Nucleic acids of the application are further understood to include nucleic acids that comprise variants of SEQ ID Nos:1, 3, 4, 6, 8, 10, 31, 32, 33, 34, and 35. Variant nucleotide sequences include sequences that differ by one or more nucleotide substitutions, additions or deletions, such as allelic variants; and will, therefore, include coding sequences that differ from the nucleotide sequence of the coding sequence designated in SEQ ID Nos:1, 3, 4, 6, 8 10, 31, 32, 33, 34, and 35, e.g., due to the degeneracy of the genetic code. In other embodiments, variants will also include sequences that will hybridize under highly stringent conditions to a nucleotide sequence of a coding sequence designated in any of SEQ ID Nos:1, 3, 4, 6, 8 10, 31, 32, 33, 34, and 35. Preferred nucleic acids of the application are human POSH sequences, including, for example, any of SEQ ID Nos: 1, 3, 4, 6, 31, 32, 33, 34, 35 and variants thereof and nucleic acids encoding an amino acid sequence selected from among SEQ ID Nos: 2, 5, 7, 26, 27, 28, 29 and 30.

In certain aspects, the application relates to nucleic acids encoding POSH-AK polypeptides. For example, a POSH-AK of the disclosure is PKA, which may comprise one or more subunit including PRKAR1A, PRKACA, and PRKACB. Nucleic acid sequences encoding these PKA subunits are provided in Example 12. Other examples of POSH-AK of the disclosure are kinases of a Rac-JNK signaling pathway, including JNK1, JNK2, MLK1, MLK2, MLK3, MKK4, and MKK7. Nucleic acid sequences encoding these kinases (e.g., JNK, MLK and MKK) are provided in Table 7. In certain embodiments, variants will also include nucleic acid sequences that will hybridize under highly stringent conditions to a nucleotide sequence of a coding sequence of a POSH-AK. Preferred nucleic acids of the application are human POSH-AK sequences and variants thereof.

One of ordinary skill in the art will understand readily that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the application provides nucleic acids which hybridize under low stringency conditions of 6×SSC at room temperature followed by a wash at 2×SSC at room temperature.

Isolated nucleic acids which differ from the POSH nucleic acid sequences or from the POSH-AK nucleic acid sequences due to degeneracy in the genetic code are also within the scope of the application. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this application.

Optionally, a POSH or a POSH-AK nucleic acid of the application will genetically complement a partial or complete loss of function phenotype in a cell. For example, a POSH nucleic acid of the application may be expressed in a cell in which endogenous POSH has been reduced by RNAi, and the introduced POSH nucleic acid will mitigate a phenotype resulting from the RNAi. An exemplary POSH loss of function phenotype is a decrease in virus-like particle. production in a cell transfected with a viral vector, optionally an HIV vector.

Another aspect of the application relates to POSH and POSH-AK nucleic acids that are used for antisense, RNAi or ribozymes. As used herein, nucleic acid therapy refers to administration or in situ generation of a nucleic acid or a derivative thereof which specifically hybridizes (e.g., binds) under cellular conditions with the cellular mRNA and/or genomic DNA encoding one of the POSH or POSH-AK polypeptides so as to inhibit production of that protein, e.g., by inhibiting transcription and/or translation. The binding may be by conventional base pair complementarity, or, for example, in the case of binding to DNA duplexes, through specific interactions in the major groove of the double helix.

A nucleic acid therapy construct of the present application can be delivered, for example, as an expression plasmid which, when transcribed in the cell, produces RNA which is complementary to at least a unique portion of the cellular mRNA which encodes a POSH or POSH-AK polypeptide. Alternatively, the the construct is an oligonucleotide which is generated ex vivo and which, when introduced into the cell causes inhibition of expression by hybridizing with the mRNA and/or genomic sequences encoding a POSH or POSH-AK polypeptide. Such oligonucleotide probes are optionally modified oligonucleotide which are resistant to endogenous nucleases, e.g., exonucleases and/or endonucleases, and is therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in nucleic acid therapy have been reviewed, for example, by van der Krol et al., (1988) Biotechniques 6:958-976; and Stein et al., (1988) Cancer Res 48:2659-2668.

Accordingly, the modified oligomers of the application are useful in therapeutic, diagnostic, and research contexts. In therapeutic applications, the oligomers are utilized in a manner appropriate for nucleic acid therapy in general.

In another aspect of the application, the subject nucleic acid is provided in an expression vector comprising a nucleotide sequence encoding a POSH or POSH-AK polypeptide and operably linked to at least one regulatory sequence. Regulatory sequences are art-recognized and are selected to direct expression of the POSH or POSH-AK polypeptide. Accordingly, the term regulatory sequence includes promoters, enhancers and other expression control elements. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding a POSH or POSH-AK polypeptide. Such useful expression control sequences, include, for example, the early and late promoters of SV40, tet promoter, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

As will be apparent, the subject gene constructs can be used to cause expression of the POSH or POSH-AK polypeptides in cells propagated in culture, e.g., to produce proteins or polypeptides, including fusion proteins or polypeptides, for purification.

This application also pertains to a host cell transfected with a recombinant gene including a coding sequence for one or more of the POSH or POSH-AK polypeptides. The host cell may be any prokaryotic or eukaryotic cell. For example, a polypeptide of the present application may be expressed in bacterial cells such as E. coli, insect cells (e.g., using a baculovirus expression system), yeast, or mammalian cells. Other suitable host cells are known to those skilled in the art. Accordingly, the present application further pertains to methods of producing the POSH or POSH-AK polypeptides. For example, a host cell transfected with an expression vector encoding a POSH polypeptide can be cultured under appropriate conditions to allow expression of the polypeptide to occur. The polypeptide may be secreted and isolated from a mixture of cells and medium containing the polypeptide. Alternatively, the polypeptide may be retained cytoplasmically and the cells harvested, lysed and the protein isolated. A cell culture includes host cells, media and other byproducts. Suitable media for cell culture are well known in the art. The polypeptide can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the polypeptide. In a preferred embodiment, the POSH or POSH-AK polypeptide is a fusion protein containing a domain which facilitates its purification, such as a POSH-GST fusion protein, POSH-intein fusion protein, POSH-cellulose binding domain fusion protein, POSH-polyhistidine fusion protein etc.

A recombinant POSH or POSH-AK nucleic acid can be produced by ligating the cloned gene, or a portion thereof, into a vector suitable for expression in either prokaryotic cells, eukaryotic cells, or both. Expression vehicles for production of a recombinant POSH or POSH-AK polypeptides include plasmids and other vectors. For instance, suitable vectors for the expression of a POSH polypeptide include plasmids of the types: pBR322-derived plasmids, pEMBL-derived plasmids, pEX-derived plasmids, pBTac-derived plasmids and pUC-derived plasmids for expression in prokaryotic cells, such as E. coli.

The preferred mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant POSH or POSH-AK polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

Alternatively, the coding sequences for the polypeptide can be incorporated as a part of a fusion gene including a nucleotide sequence encoding a different polypeptide. This type of expression system can be useful under conditions where it is desirable, e.g., to produce an immunogenic fragment of a POSH or POSH-AK polypeptide. For example, the VP6 capsid protein of rotavirus can be used as an immunologic carrier protein for portions of polypeptide, either in the monomeric form or in the form of a viral particle. The nucleic acid sequences corresponding to the portion of the POSH or POSH-AK polypeptide to which antibodies are to be raised can be incorporated into a fusion gene construct which includes coding sequences for a late vaccinia virus structural protein to produce a set of recombinant viruses expressing fusion proteins comprising a portion of the protein as part of the virion. The Hepatitis B surface antigen can also be utilized in this role as well. Similarly, chimeric constructs coding for fusion proteins containing a portion of a POSH polypeptide and the poliovirus capsid protein can be created to enhance immunogenicity (see, for example, EP Publication NO: 0259149; and Evans et al., (1989) Nature 339:385; Huang et al., (1988) J. Virol. 62:3855; and Schlienger et al., (1992) J. Virol. 66:2).

The Multiple Antigen Peptide system for peptide-based immunization can be utilized, wherein a desired portion of a POSH or POSH-AK polypeptide is obtained directly from organo-chemical synthesis of the peptide onto an oligomeric branching lysine core (see, for example, Posnett et al., (1988) JBC 263:1719 and Nardelli et al., (1992) J. Immunol. 148:914). Antigenic determinants of a POSH or POSH-AK polypeptide can also be expressed and presented by bacterial cells.

In another embodiment, a fusion gene coding for a purification leader sequence, such as a poly-(His)/enterokinase cleavage site sequence at the N-terminus of the desired portion of the recombinant protein, can allow purification of the expressed fusion protein by affinity chromatography using a Ni²⁺ metal resin. The purification leader sequence can then be subsequently removed by treatment with enterokinase to provide the purified POSH or POSH-AK polypeptide (e.g., see Hochuli et al., (1987) J. Chromatography 411:177; and Janknecht et al., PNAS USA 88:8972).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

TABLE 2
Exemplary POSH nucleic acids

| Sequence Name | Organism | Accession Number |
|---|---|---|
| cDNA FLJ11367 fis, | Homo sapiens | AK021429 |
| clone HEMBA1000303 | | |
| Plenty of SH3 domains | Mus musculus | NM_021506 |
| (POSH) mRNA | | |
| Plenty of SH3s (POSH) | Mus musculus | AF030131 |
| mRNA | | |
| Plenty of SH3s (POSH) | Drosophila melanogaster | NM_079052 |
| mRNA | | |
| Plenty of SH3s (POSH) | Drosophila melanogaster | AF220364 |
| mRNA | | |

TABLE 3
Exemplary POSH polypeptides

| Sequence Name | Organism | Accession Number |
|---|---|---|
| SH3 domains- | Mus musculus | T09071 |
| containing protein POSH | | |
| plenty of SH3 domains | Mus musculus | NP_067481 |
| Plenty of SH3s; POSH | Mus musculus | AAC40070 |
| Plenty of SH3s | Drosophila melanogaster | AAF37265 |
| LD45365p | Drosophila melanogaster | AAK93408 |
| POSH gene product | Drosophila melanogaster | AAF57833 |
| Plenty of SH3s | Drosophila melanogaster | NP_523776 |

In addition the following Tables provide the nucleic acid sequence and related SEQ ID NOs for domains of human POSH protein and a summary of sequence identification numbers used in this application.

TABLE 4

Nucleic Acid Sequences and related
SEQ ID NOs for domains in human POSH

| Name of the | | SEQ ID |
|---|---|---|
| sequence | Sequence | NO. |
| RING domain | TGTCCGGTGTGTCTAGAGCGCCTTGATGCTTC | 31 |
| | TGCGAAGGTCTTGCCTTGCCAGCATACGTTTT | |
| | GCAAGCGATGTTTGCTGGGGATCGTAGGTTCT | |
| | CGAAATGAACTCAGATGTCCCGAGT | |
| 1^{st} SH₃ | CCATGTGCCAAAGCGTTATACAACTATGAAGG | 32 |
| domain | AAAAGAGCCTGGAGACCTTAAATTCAGCAAAG | |
| | GCGACATCATCATTTTGCGAAGACAAGTGGAT | |
| | GAAAATTGGTACCATGGGGAAGTCAATGGAAT | |
| | CCATGGCTTTTTCCCCACCAACTTTGTGCAGA | |
| | TTATT | |
| 2^{nd} SH₃ | CCTCAGTGCAAAGCACTTTATGACTTTGAAGT | 33 |
| domain | GAAAGACAAGGAAGCAGACAAAGATTGCCTTC | |
| | CATTTGCAAAGGATGATGTTCTGACTGTGATC | |
| | CGAAGAGTGGATGAAAACTGGGCTGAAGGAAT | |
| | GCTGGCAGACAAAATAGGAATATTTCCAATTT | |
| | CATATGTTGAGTTTAAC | |
| 3^{rd} SH₃ | AGTGTGTATGTTGCTATATATCCATACACTCCT | 34 |
| domain | CGGAAAGAGGATGAACTAGAGCTGAGAAAAGGG | |
| | GAGATGTTTTTAGTGTTTGAGCGCTGCCAGGAT | |
| | GGCTGGTTCAAAGGGACATCCATGCATACCAGC | |
| | AAGATAGGGGTTTTCCCTGGCAATTATGTGGCA | |
| | CCAGTC | |
| 4^{th} SH₃ | GAAAGGCACAGGGTGGTGGTTTCCTATCCTCCT | 35 |
| domain | CAGAGTGAGGCAGAACTTGAACTTAAAGAAGGA | |
| | GATATTGTGTTTGTTCATAAAAAACGAGAGGAT | |
| | GGCTGGTTCAAAGGCACATTACAACGTAATGGG | |
| | AAAACTGGCCTTTTCCCAGGAAGCTTTGTGGAA | |
| | AACA | |

TABLE 5
Summary of Sequence Identification Numbers

| | Sequence |
|---|---|
| | Identification Number |
| Sequence Information | (SEQ ID NO) |
| Human POSH Coding Sequence | SEQ ID No: 1 |
| Human POSH Amino Acid Sequence | SEQ ID No: 2 |
| Human POSH cDNA Sequence | SEQ ID No: 3 |
| 5′ cDNA Fragment of Human POSH | SEQ ID No: 4 |
| N-terminus Protein Fragment of | SEQ ID No: 5 |
| Human POSH | |
| 3′ mRNA Fragment of Human POSH | SEQ ID No: 6 |
| C-terminus Protein Fragment of | SEQ ID No: 7 |
| Human POSH | |
| Mouse POSH mRNA Sequence | SEQ ID No: 8 |
| Mouse POSH Protein Sequence | SEQ ID No: 9 |
| Drosophila melanogaster POSH | SEQ ID No: 10 |
| mRNA Sequence | |
| Drosophila melanogaster POSH | SEQ ID No: 11 |
| Protein Sequence | |
| Human POSH RING Domain Amino | SEQ ID No: 26 |
| Acid Sequence | |
| Human POSH 1^{st} SH₃ Domain Amino | SEQ ID No: 27 |
| Acid Sequence | |
| Human POSH 2^{nd} SH₃ Domain Amino | SEQ ID No: 28 |
| Acid Sequence | |
| Human POSH 3^{rd} SH₃ Domain Amino | SEQ ID No: 29 |
| Acid Sequence | |
| Human POSH 4^{th} SH₃ Domain Amino | SEQ ID No: 30 |
| Acid Sequence | |
| Human POSH RING Domain Nucleic | SEQ ID No: 31 |
| Acid Sequence | |
| Human POSH 1^{st} SH₃ Domain Nucleic | SEQ ID No: 32 |
| Acid Sequence | |
| Human POSH 2^{nd} SH₃ Domain Nucleic | SEQ ID No: 33 |
| Acid Sequence | |
| Human POSH 3^{rd} SH₃ Domain Nucleic | SEQ ID No: 34 |
| Acid Sequence | |
| Human POSH 4^{th} SH₃ Domain Nucleic | SEQ ID No: 35 |
| Acid Sequence | |

7. Exemplary Polypeptides

The present application relates to the POSH polypeptides, which are isolated from, or otherwise substantially free of, other intracellular proteins which might normally be associated with the protein or a particular complex including the protein. In certain embodiments, POSH polypeptides have an amino acid sequence that is at least 60% identical to an amino acid sequence as set forth in any of SEQ ID Nos: 2, 5, 7, 9, 11, 26, 27, 28, 29 and 30. In other embodiments, the polypeptide has an amino acid sequence at least 65%, 70%, 75%, 80%, 85%, 90%, 95%, 97%, 98%, 99% or 100% identical to an amino acid sequence as set forth in any of SEQ ID Nos: 2, 5, 7, 9, 11, 26, 27, 28, 29 and 30.

In certain aspects, the application also relates to POSH-AK polypeptides (e.g., a PKA subunit or a JNK pathway kinase). Amino acid sequences of the PKA subunits including PRKAR1A, PRKACA, and PRKACB, are provided in Example 12. Amino acid sequences of the JNK pathway kinases including JNK1, JNK2, MLK1, MLK2, MLK3, MKK4, and MKK7, are provided in Table 7. In certain embodiments, In certain embodiments, POSH-AK polypeptides have an amino acid sequence that is at least 60% identical to these amino acid sequence as set forth in Example 12 and Table 7. In other embodiments, the POSH-AK polypeptide has an amino acid sequence at least 65%, 70%, 75%, 80%, 85%, 90%, 95%, 97%, 98%, 99% or 100% identical to an amino acid sequence as set forth in Example 12 and Table 7.

Optionally, a POSH or POSH-AK polypeptide of the application will function in place of an endogenous POSH or POSH-AK polypeptide, for example by mitigating a partial or complete loss of function phenotype in a cell. For example, a POSH polypeptide of the application may be produced in a cell in which endogenous POSH has been reduced by RNAi, and the introduced POSH polypeptide will mitigate a phenotype resulting from the RNAi. An exemplary POSH loss of function phenotype is a decrease in virus-like particle production in a cell transfected with a viral vector, optionally an HIV vector. In certain embodiments, a POSH polypeptide, when produced at an effective level in a cell, induces apoptosis.

In another aspect, the application provides polypeptides that are agonists or antagonists of a POSH or POSH-AK polypeptide. Variants and fragments of a POSH or POSH-AK polypeptide may have a hyperactive or constitutive activity, or, alternatively, act to prevent POSH or POSH-AK polypeptides from performing one or more functions. For example, a truncated form lacking one or more domain may have a dominant negative effect.

Another aspect of the application relates to polypeptides derived from a full-length POSH or POSH-AK polypeptide. Isolated peptidyl portions of the subject proteins can be obtained by screening polypeptides recombinantly produced from the corresponding fragment of the nucleic acid encoding such polypeptides. In addition, fragments can be chemically synthesized using techniques known in the art such as conventional Merrifield solid phase f-Moc or t-Boc chemistry. For example, any one of the subject proteins can be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of the formation of a specific protein complex, or more generally of a POSH:POSH-AK complex, such as by microinjection assays.

It is also possible to modify the structure of the POSH or POSH-AK polypeptides for such purposes as enhancing therapeutic or prophylactic efficacy, or stability (e.g., ex vivo shelf life and resistance to proteolytic degradation in vivo). Such modified polypeptides, when designed to retain at least one activity of the naturally-occurring form of the protein, are considered functional equivalents of the POSH or POSH-AK polypeptides described in more detail herein. Such modified polypeptides can be produced, for instance, by amino acid substitution, deletion, or addition.

For instance, it is reasonable to expect, for example, that an isolated replacement of a leucine with an isoleucine or valine, an aspartate with a glutamate, a threonine with a serine, or a similar replacement of an amino acid with a structurally related amino acid (i.e,. conservative mutations) will not have a major effect on the biological activity of the resulting molecule. Conservative replacements are those that take place within a family of amino acids that are related in their side chains. Genetically encoded amino acids are can be divided into four families (see, for example, Biochemistry, 2nd ed., Ed. by L. Stryer, W.H. Freeman and Co., 1981). Whether a change in the amino acid sequence of a polypeptide results in a functional homolog can be readily determined by assessing the ability of the variant polypeptide to produce a response in cells in a fashion similar to the wild-type protein. For instance, such variant forms of a POSH polypeptide can be assessed, e.g., for their ability to bind to another polypeptide, e.g., another POSH polypeptide or another protein involved in viral maturation. Polypeptides in which more than one replacement has taken place can readily be tested in the same manner.

This application further contemplates a method of generating sets of combinatorial mutants of the POSH or POSH-AK polypeptides, as well as truncation mutants, and is especially useful for identifying potential variant sequences (e.g., homologs) that are functional in binding to a POSH or POSH-AK polypeptide. The purpose of screening such combinatorial libraries is to generate, for example, POSH homologs which can act as either agonists or antagonist, or alternatively, which possess novel activities all together. Combinatorially-derived homologs can be generated which have a selective potency relative to a naturally occurring POSH or POSH-AK polypeptide. Such proteins, when expressed from recombinant DNA constructs, can be used in gene therapy protocols.

Likewise, mutagenesis can give rise to homologs which have intracellular half-lives dramatically different than the corresponding wild-type protein. For example, the altered protein can be rendered either more stable or less stable to proteolytic degradation or other cellular process which result in destruction of, or otherwise inactivation of the POSH or POSH-AK polypeptide of interest. Such homologs, and the genes which encode them, can be utilized to alter POSH or POSH-AK levels by modulating the half-life of the protein. For instance, a short half-life can give rise to more transient biological effects and, when part of an inducible expression system, can allow tighter control of recombinant POSH or POSH-AK levels within the cell. As above, such proteins, and particularly their recombinant nucleic acid constructs, can be used in gene therapy protocols.

In similar fashion, POSH or POSH-AK homologs can be generated by the present combinatorial approach to act as antagonists, in that they are able to interfere with the ability of the corresponding wild-type protein to function.

In a representative embodiment of this method, the amino acid sequences for a population of POSH or POSH-AK homologs are aligned, preferably to promote the highest homology possible. Such a population of variants can include, for example, homologs from one or more species, or homologs from the same species but which differ due to mutation. Amino acids which appear at each position of the aligned sequences are selected to create a degenerate set of combinatorial sequences. In a preferred embodiment, the combinatorial library is produced by way of a degenerate library of genes encoding a library of polypeptides which each include at least a portion of potential POSH or POSH-AK sequences. For instance, a mixture of synthetic oligonucleotides can be enzymatically ligated into gene sequences such that the degenerate set of potential POSH or POSH-AK nucleotide sequences are expressible as individual polypeptides, or alternatively, as a set of larger fusion proteins (e.g., for phage display).

There are many ways by which the library of potential homologs can be generated from a degenerate oligonucleotide sequence. Chemical synthesis of a degenerate gene sequence can be carried out in an automatic DNA synthesizer, and the synthetic genes then be ligated into an appropriate gene for expression. The purpose of a degenerate set of genes is to provide, in one mixture, all of the sequences encoding the desired set of potential POSH or POSH-AK sequences. The synthesis of degenerate oligonucleotides is well known in the art (see for example, Narang, S A (1983) Tetrahedron 39:3; Itakura et al., (1981) Recombinant DNA, Proc. 3rd Cleveland Sympos. Macromolecules, ed. A G Walton, Amsterdam: Elsevier pp 273-289; Itakura et al., (1984) Annu. Rev. Biochem. 53:323; Itakura et al., (1984) Science 198:1056; Ike et al., (1983) Nucleic Acid Res. 11:477). Such techniques have been employed in the directed evolution of other proteins (see, for example, Scott et al., (1990) Science 249:386-390; Roberts et al., (1992) PNAS USA 89:2429-2433; Devlin et al., (1990) Science 249: 404406; Cwirla et al., (1990) PNAS USA 87: 6378-6382; as well as U.S. Pat. Nos. 5,223,409, 5,198,346, and 5,096,815).

Alternatively, other forms of mutagenesis can be utilized to generate a combinatorial library. For example, POSH or POSH-AK homologs (both agonist and antagonist forms) can be generated and isolated from a library by screening using, for example, alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J. Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell Biol. 12:2644-2652; McKnight et al., (1982) Science 232:316); by saturation mutagenesis (Meyers et al., (1986) Science 232:613); by PCR mutagenesis (Leung et al., (1989) Method Cell Mol Biol 1:11-19); or by random mutagenesis, including chemical mutagenesis, etc. (Miller et al., (1992) A Short Course in Bacterial Genetics, CSHL Press, Cold Spring Harbor, N.Y.; and Greener et al., (1994) Strategies in Mol Biol 7:32-34). Linker scanning mutagenesis, particularly in a combinatorial setting, is an attractive method for identifying truncated (bioactive) forms of POSH or POSH-AK polypeptides.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of POSH or POSH-AK homologs. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate sequences created by combinatorial mutagenesis techniques.

In an illustrative embodiment of a screening assay, candidate combinatorial gene products of one of the subject proteins are displayed on the surface of a cell or virus, and the ability of particular cells or viral particles to bind a POSH or POSH-AK polypeptide is detected in a “panning assay”. For instance, a library of POSH variants can be cloned into the gene for a surface membrane protein of a bacterial cell (Ladner et al., WO 88/06630; Fuchs et al., (1991) Bio/Technology 9:1370-1371; and Goward et al., (1992) TIBS 18:136-140), and the resulting fusion protein detected by panning, e.g., using a fluorescently labeled molecule which binds the POSH polypeptide, to score for potentially functional homologs. Cells can be visually inspected and separated under a fluorescence microscope, or, where the morphology of the cell permits, separated by a fluorescence-activated cell sorter.

In similar fashion, the gene library can be expressed as a fusion protein on the surface of a viral particle. For instance, in the filamentous phage system, foreign peptide sequences can be expressed on the surface of infectious phage, thereby conferring two significant benefits. First, since these phage can be applied to affinity matrices at very high concentrations, a large number of phage can be screened at one time. Second, since each infectious phage displays the combinatorial gene product on its surface, if a particular phage is recovered from an affinity matrix in low yield, the phage can be amplified by another round of infection. The group of almost identical E. coli filamentous phages M13, fd, and fl are most often used in phage display libraries, as either of the phage gIII or gVIII coat proteins can be used to generate fusion proteins without disrupting the ultimate packaging of the viral particle (Ladner et al., PCT publication WO 90/02909; Garrard et al., PCT publication WO 92/09690; Marks et al., (1992) J. Biol. Chem. 267:16007-16010; Griffiths et al., (1993) EMBO J. 12:725-734; Clackson et al., (1991) Nature 352:624-628; and Barbas et al., (1992) PNAS USA 89:4457-4461).

The application also provides for reduction of the POSH or POSH-AK polypeptides to generate mimetics, e.g., peptide or non-peptide agents, which are able to mimic binding of the authentic protein to another cellular partner. Such mutagenic techniques as described above, as well as the thioredoxin system, are also particularly useful for mapping the determinants of a POSH or POSH-AK polypeptide which participate in protein-protein interactions involved in, for example, binding of proteins involved in viral maturation to each other. To illustrate, the critical residues of a POSH or POSH-AK polypeptide which are involved in molecular recognition of a substrate protein can be determined and used to generate its derivative peptidomimetics which bind to the substrate protein, and by inhibiting POSH or POSH-AK binding, act to inhibit its biological activity. By employing, for example, scanning mutagenesis to map the amino acid residues of a POSH polypeptide which are involved in binding to another polypeptide, peptidomimetic compounds can be generated which mimic those residues involved in binding. For instance, non-hydrolyzable peptide analogs of such residues can be generated using benzodiazepine (e.g., see Freidinger et al., in Peptides: Chemistry and Biology, G. R. Marshall ed., ESCOM Publisher: Leiden, Netherlands, 1988), azepine (e.g., see Huffman et al., in Peptides: Chemistry and Biology, G. R. Marshall ed., ESCOM Publisher: Leiden, Netherlands, 1988), substituted gamma lactam rings (Garvey et al., in Peptides: Chemistry and Biology, G. R. Marshall ed., ESCOM Publisher: Leiden, Netherlands, 1988), keto-methylene pseudopeptides (Ewenson et al., (1986) J. Med. Chem. 29:295; and Ewenson et al., in Peptides: Structure and Function (Proceedings of the 9th American Peptide Symposium) Pierce Chemical Co. Rockland, Ill., 1985), b-turn dipeptide cores (Nagai et al., (1985) Tetrahedron Lett 26:647; and Sato et al., (1986) J Chem Soc Perkin Trans 1:1231), and b-aminoalcohols (Gordon et al., (1985) Biochem Biophys Res Commun 126:419; and Dann et al., (1986) Biochem Biophys Res Commun 134:71).

The following table provides the sequences of the RING domain and the various SH3 domains of POSH.

TABLE 6

Amino Acid Sequences and related
SEQ ID NOs for domains in human POSH

| Name of | | |
|---|---|---|
| the | | SEQ ID |
| sequence | Sequence | NO. |
| RING | CPVCLERLDASAKVLPCQHTFCKRCLLGIVGSRNEL | 26 |
| domain | RCPEC | |
| 1^{st} SH₃ | PCAKALYNYEGKEPGDLKFSKGDIIILRRQVDENWY | 27 |
| domain | HGEVNGIHGFFPTNFVQIIK | |
| 2^{nd} SH₃ | PQCKALYDFEVKDKEADKDCLPFAKDDVLTVIRRVD | 28 |
| domain | ENWAEGMLADKIGIFPISYVEFNS | |
| 3^{rd} SH₃ | SVYVAIYPYTPRKEDELELRKGEMFLVFERCQDGWF | 29 |
| domain | KGTSMHTSKIGVFPGNYVAPVT | |
| 4^{th} SH₃ | ERHRVVVSYPPQSEAELELKEGDIVFVHKKREDGWF | 30 |
| domain | KGTLQRNGKTGLFPGSFVENI | |

TABLE 7
Sequences of POSH associated kinases in a Rac-JNK signaling pathway.

| | protein sequence | mRNA sequence |
|---|---|---|
| Kinase and variant | (public gi No.) | (public gi No.) |
| Human MLK1 - var1 | 462606 | 12005723 |
| Human MLK1 - var2 | 12005724 | 27479475 |
| Human MLK1 - var3 | 14749517 | |
| Human MLK2 - var1 | 6686295 | 971419 |
| Human MLK2 - var2 | 758593 | 21735549 |
| Human MLK2 - var3 | 21735550 | 758592 |
| Human MLK3 - var1 | 1090771 | 15030036 |
| Human MLK3 - var2 | * | 488295 |
| Human MLK3 - var3 | * | 464027 |
| Human MKK4 - var1 | 1170596 | 685175 |
| Human MKK4 - var2 | * | 24497520 |
| Human MKK4 - var3 | * | 791187 |
| Human MKK7 - var1 | 2558889 | 3108200 |
| Human MKK7 - var2 | 3108199 | 21735541 |
| Human MKK7 - var3 | 23468315 | 2262234 |
| Human MKK7 - var4 | 2318119 | 2811125 |
| Human MKK7 - var5 | 2811126 | 2318118 |
| Human MKK7 - var6 | 2262235 | 23468314 |
| Human MKK7 - var7 | 21735542 | 3108198 |
| Human MKK7 - var8 | * | 21735543 |
| Human MKK7 - var9 | * | 2558888 |
| Human JNK1 - var1 | 4506095 | 20986493 |
| Human JNK1 - var2 | 1463131 | 1463130 |
| Human JNK1 - var3 | 1463137 | 1463138 |
| Human JNK1 - var4 | 1463139 | 20986522 |
| Human JNK1 - var5 | * | 1463136 |
| Human JNK2 - var1 | 21237745 | 1463128 |
| Human JNK2 - var2 | 1463135 | 607785 |
| Human JNK2 - var3 | 21237742 | 21237738 |
| Human JNK2 - var4 | 7446390 | 598182 |
| Human JNK2 - var5 | 1463133 | 21618469 |
| Human JNK2 - var6 | 21237736 | 21237735 |
| Human JNK2 - var7 | 1170598 | 1463132 |
| Human JNK2 - var8 | 21237739 | 1463134 |
| Human JNK2 - var9 | 607786 | |
| Human JNK2 - var10 | 1463129 | |

* denotes a polypeptide sequence that can be deduced from the corresponding mRNA sequence.

8. Effective Dose

Toxicity and therapeutic efficacy of such compounds can be determined by standard pharmaceutical procedures in cell cultures or experimental animals, e.g., for determining The LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic induces are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the application, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

9. Formulation and Use

Pharmaceutical compositions for use in accordance with the present application may be formulated in conventional manner using one or more physiologically acceptable carriers or excipients. Thus, the compounds and their physiologically acceptable salts and solvates may be formulated for administration by, for example, injection, inhalation or insufflation (either through the mouth or the nose) or oral, buccal, parenteral or rectal administration.

An exemplary composition of the application comprises an RNAi mixed with a delivery system, such as a liposome system, and optionally including an acceptable excipient. In a preferred embodiment, the composition is formulated for topical administration for, e.g., herpes virus infections.

For such therapy, the compounds of the application can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the compounds of the application can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present application are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives in addition, detergents may be used to facilitate permeation. Transmucosal administration may be through nasal sprays or using suppositories. For topical administration, the oligomers of the application are formulated into ointments, salves, gels, or creams as generally known in the art. A wash solution can be used locally to treat an injury or inflammation to accelerate healing.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

For therapies involving the administration of nucleic acids, the oligomers of the application can be formulated for a variety of modes of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, intranodal, and subcutaneous for injection, the oligomers of the application can be formulated in liquid solutions, preferably in physiologically compatible buffers such as Hank's solution or Ringer's solution. In addition, the oligomers may be formulated in solid form and redissolved or suspended immediately prior to use. Lyophilized forms are also included.

Systemic administration can also be by transmucosal or transdermal means, or the compounds can be administered orally. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For oral administration, the oligomers are formulated into conventional oral administration forms such as capsules, tablets, and tonics. For topical administration, the oligomers of the application are formulated into ointments, salves, gels, or creams as generally known in the art.

The application now being generally described, it will be more readily understood by reference to the following examples, which are included merely for purposes of illustration of certain aspects and embodiments of the present application, and are not intended to limit the application.

### EXAMPLES

### Example 1

### Role of POSH in Virus-Like Particle (VLP) Budding

1. Objective:

Use RNAi to inhibit POSH gene expression and compare the efficiency of viral budding and GAG expression and processing in treated and untreated cells.

2. Study Plan:

HeLa SS-6 cells are transfected with mRNA-specific RNAi in order to knockdown the target proteins. Since maximal reduction of target protein by RNAi is achieved after 48 hours, cells are transfected twice—first to reduce target mRNAs, and subsequently to express the viral Gag protein. The second transfection is performed with pNLenv (plasmid that encodes HIV) and with low amounts of RNAi to maintain the knockdown of target protein during the time of gag expression and budding of VLPs. Reduction in mRNA levels due to RNAi effect is verified by RT-PCR amplification of target mRNA.

3. Methods, Materials, Solutions

a. Methods
i. Transfections according to manufacturer's protocol and as described in procedure.
ii. Protein determined by Bradford assay.
iii. SDS-PAGE in Hoeffer miniVE electrophoresis system. Transfer in Bio-Rad mini-protean II wet transfer system. Blots visualized using Typhoon system, and ImageQuant software (ABbiotech)

b. Materials

| Material | Manufacturer | Catalog # | Batch # |
|---|---|---|---|
| Lipofectamine 2000 | Life Technologies | 11668-019 | 1112496 |
| (LF2000) | | | |
| OptiMEM | Life Technologies | 31985-047 | 3063119 |
| RNAi Lamin A/C | Self | 13 | |
| RNAi TSG101 688 | Self | 65 | |
| RNAi Posh 524 | Self | 81 | |
| plenvl1 PTAP | Self | 148 | |
| plenvl1 ATAP | Self | 149 | |
| Anti-p24 polyclonal | Seramun | | A-0236/5- |
| antibody | | | 10-01 |
| Anti-Rabbit Cy5 | Jackson | 144-175-115 | 48715 |
| conjugated antibody | | | |
| 10% acrylamide Tris- | Life Technologies | NP0321 | 1081371 |
| Glycine SDS-PAGE gel | | | |
| Nitrocellulose | Schleicher & | 401353 | BA-83 |
| membrane | Schuell | | |
| NuPAGE 20X transfer | Life Technologies | NP0006-1 | 224365 |
| buffer | | | |
| 0.45 μm filter | Schleicher & | 10462100 | CS1018-1 |
| | Schuell | | |

c. Solutions

| | Compound | Concentration |
|---|---|---|
| Lysis Buffer | Tris-HCl pH 7.6 | 50 mM |
| | MgCl₂ | 15 mM |
| | NaCl | 150 mM |
| | Glycerol | 10% |
| | EDTA | 1 mM |
| | EGTA | 1 mM |
| | ASB-14 (add immediately | 1% |
| | before use) | |
| 6X Sample | Tris-HCl, pH = 6.8 | 1M |
| Buffer | Glycerol | 30% |
| | SDS | 10% |
| | DTT | 9.3% |
| | Bromophenol Blue | 0.012% |
| TBS-T | Tris pH = 7.6 | 20 mM |
| | NaCl | 137 mM |
| | Tween-20 | 0.1% |

4. Procedure

a. Schedule

Day

| 1 | 2 | 3 | 4 | 5 |
|---|---|---|---|---|
| Plate | Transfection | Passage | Transfection II | Extract RNA |
| cells | I | cells | (RNAi and | for RT-PCR |
| | (RNAi only) | (1:3) | pNlenv) | (post |
| | | | (12:00, PM) | transfection) |
| | | | Extract RNA for | Harvest VLPs |
| | | | RT-PCR | and cells |
| | | | (pre-transfection) | |

b. Day 1

Plate HeLa SS-6 cells in 6-well plates (35 mm wells) at concentration of 5×10⁵ cells/well.

c. Day 2

2 hours before transfection replace growth medium with 2 ml growth medium without antibiotics.

Transfection I:

| | | | | | RNAi | A | B |
|---|---|---|---|---|---|---|---|
| | | | | | [20 μM] | OPtiMEM | LF2000 mix |
| Reaction | RNAi name | TAGDA# | Reactions | RNAi [nM] | μl | (μl) | (μl) |
| 1 | Lamin A/C | 13 | 2 | 50 | 12.5 | 500 | 500 |
| 2 | Lamin A/C | 13 | 1 | 50 | 6.25 | 250 | 250 |
| 3 | TSG101 688 | 65 | 2 | 20 | 5 | 500 | 500 |
| 5 | Posh 524 | 81 | 2 | 50 | 12.5 | 500 | 500 |

Transfections:
Prepare LF2000 mix: 250 μl OptiMEM+5 μl LF2000 for each reaction. Mix by inversion, 5 times. Incubate 5 minutes at room temperature.
Prepare RNA dilution in OptiMEM (Table 1, column A). Add LF2000 mix dropwise to diluted RNA (Table 1, column B). Mix by gentle vortex. Incubate at room temperature 25 minutes, covered with aluminum foil.
Add 500 μl transfection mixture to cells dropwise and mix by rocking side to side.
Incubate overnight.

d. Day 3
Split 1:3 after 24 hours. (Plate 4 wells for each reaction, except reaction 2 which is plated into 3 wells.)

e. Day 4

2 hours pre-transfection replace medium with DMEM growth medium without antibiotics.

Transfection II

| | | | | | B | | |
|---|---|---|---|---|---|---|---|
| | | | | A | RNAi | | |
| | | | | Plasmid | [20 μM] for | C | D |
| RNAi | TAG | | Reaction | for 2.4 μg | 10 nM | OPtiMEM | LF2000 mix |
| name | DA# | Plasmid | # | (μl) | (μl) | (μl) | (μl) |
| Lamin A/C | 13 | PTAP | 3 | 3.4 | 3.75 | 750 | 750 |
| Lamin A/C | 13 | ATAP | 3 | 2.5 | 3.75 | 750 | 750 |
| TSG101 688 | 65 | PTAP | 3 | 3.4 | 3.75 | 750 | 750 |
| Posh 524 | 81 | PTAP | 3 | 3.4 | 3.75 | 750 | 750 |

Prepare LF2000 mix: 250 μl OptiMEM+5 μl LF2000 for each reaction. Mix by inversion, 5 times. Incubate 5 minutes at room temperature.
Prepare RNA+DNA diluted in OptiMEM (Transfection II, A+B+C)
Add LF2000 mix (Transfection II, D) to diluted RNA+DNA dropwise, mix by gentle vortex, and incubate 1 h while protected from light with aluminum foil.
Add LF2000 and DNA+RNA to cells, 500 μl/well, mix by gentle rocking and incubate overnight.

f. Day 5
Collect samples for VLP assay (approximately 24 hours post-transfection) by the following procedure (cells from one well from each sample is taken for RNA assay, by RT-PCR).

g. Cell Extracts
i. Pellet floating cells by centrifugation (5 min, 3000 rpm at 4° C.), save supernatant (continue with supernatant immediately to step h), scrape remaining cells in the medium which remains in the well, add to the corresponding floating cell pellet and centrifuge for 5 minutes, 1800 rpm at 4° C.
ii. Wash cell pellet twice with ice-cold PBS.
iii. Resuspend cell pellet in 100 μl lysis buffer and incubate 20 minutes on ice.
iv. Centrifuge at 14,000 rpm for 15 min. Transfer supernatant to a clean tube. This is the cell extract.
v. Prepare 10 μl of cell extract samples for SDS-PAGE by adding SDS-PAGE sample buffer to 1×, and boiling for 10 minutes. Remove an aliquot of the remaining sample for protein determination to verify total initial starting material. Save remaining cell extract at −80° C.

h. Purification of VLPs from Cell Media
i. Filter the supernatant from step g through a 0.45 m filter.
ii. Centrifuge supernatant at 14,000 rpm at 4° C. for at least 2 h.
iii. Aspirate supernatant carefully.
iv. Re-suspend VLP pellet in hot (100° C. warmed for 10 min at least) 1× sample buffer.
v. Boil samples for 10 minutes, 100° C.

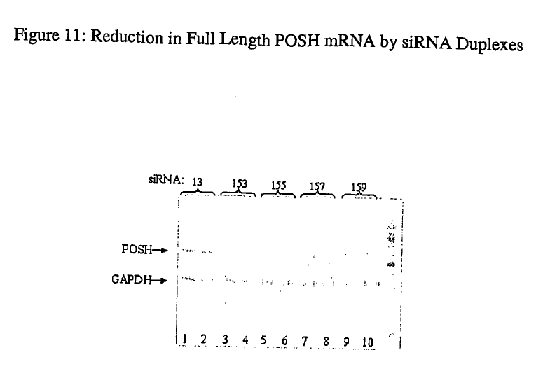

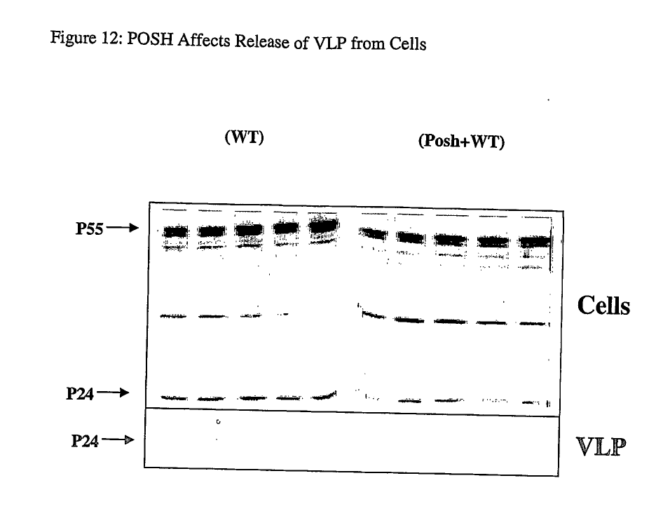

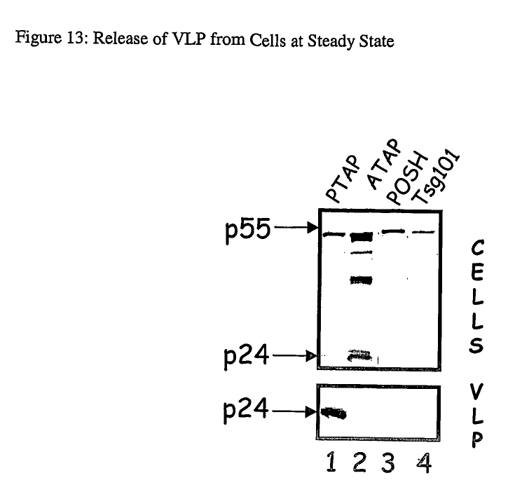

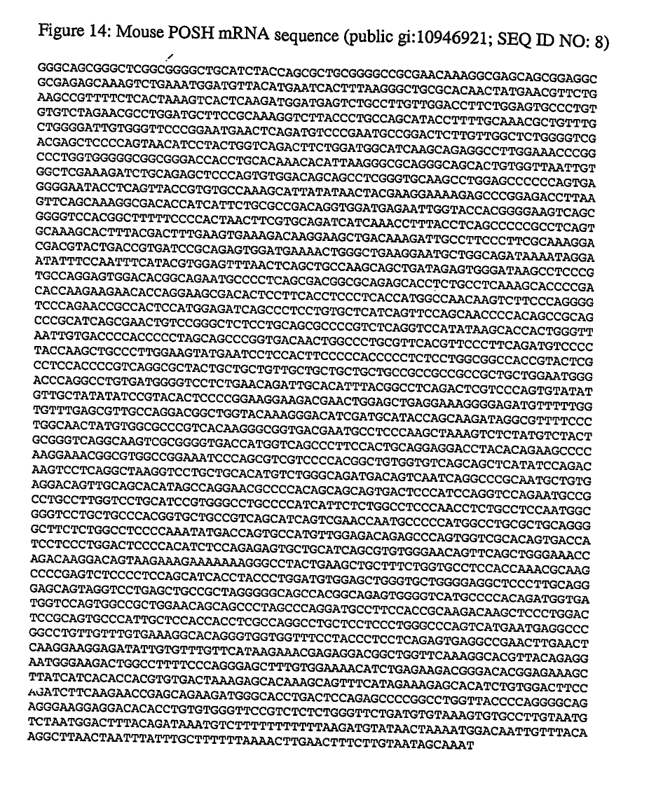

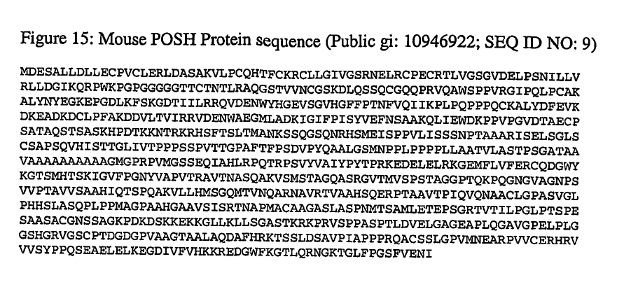

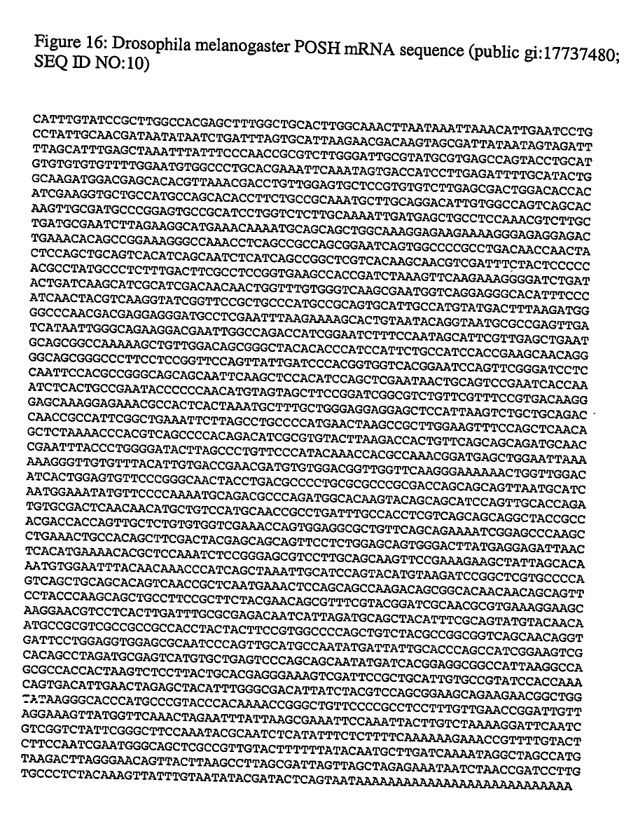

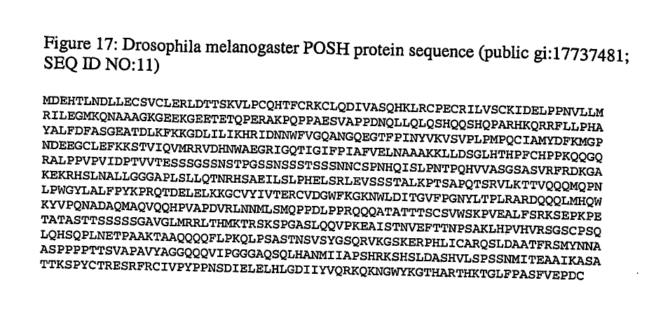

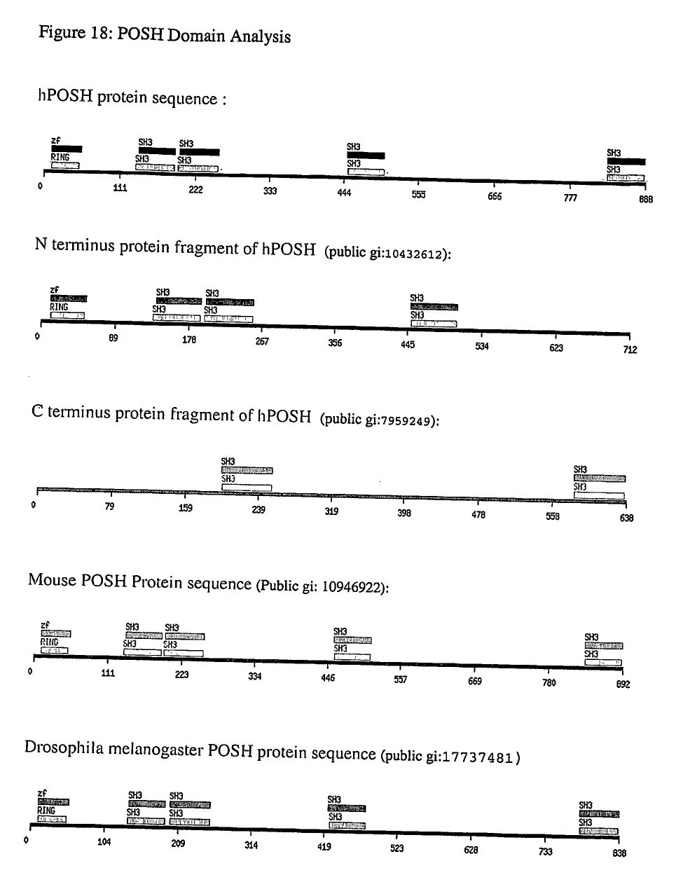

i. Western Blot Analysis
i. Run all samples from stages A and B on Tris-Glycine SDS-PAGE 10% (120V for 1.5 h).
ii. Transfer samples to nitrocellulose membrane (65V for 1.5 h).
iii. Stain membrane with ponceau S solution.
iv. Block with 10% low fat milk in TBS-T for 1 h.
v. Incubate with anti p24 rabbit 1:500 in TBS-T o/n.
vi. Wash 3 times with TBS-T for 7 min each wash.
vii. Incubate with secondary antibody anti rabbit cy5 1:500 for 30 min.
viii. Wash five times for 10 min in TBS-T.
ix. View in Typhoon gel imaging system (Molecular Dynamics/APBiotech) for fluorescence signal.
   Results are shown in FIGS. 11-13.

### Example 2

### Exemplary POSH RT-PCR Primers and siRNA Duplexes RT-PCR Primers

| | | |
|---|---|---|
| Name | Position | Sequence |

| | | | | |
|---|---|---|---|---|
| Sense primer | POSH = 271 | 271 | 5′ CTTGCCTTGCCAGCATAC 3′ | (SEQ ID NO:12) |
| Anti-sense | POSH = 926c | 926C | 5′ CTGCCAGCATTCCTTCAG 3′ | (SEQ ID NO:13) |
| primer | | | | |

siRNA Duplexes:

| | | |
|---|---|---|
| siRNA No: | 153 | |
| siRNA Name: | POSH-230 | |
| Position in mRNA | 426-446 | |
| Target sequence: | 5′ AACAGAGGCCTTGGAAACCTG 3′ | SEQ ID NO:14 |
| siRNA sense strand: | 5′ dTdTCAGAGGCCUUGGAAACCUG 3′ | SEQ ID NO:15 |
| siRNA anti-sense strand: | 5′ dTdTCAGGUUUCCAAGGCGUCUG 3′ | SEQ ID NO:16 |
| siRNA No: | 155 | |
| siRNA Name: | POSH-442 | |
| Position in mRNA | 638-658 | |
| Target sequence: | 5′ AAAGAGCCTGGAGACCTTAAA 3′ | SEQ ID NO:17 |
| siRNA sense strand: | 5′ ddTdTAGAGCCUGGAGACCUUAAA 3′ | SEQ ID NO:18 |
| siRNA anti-sense strand: | 5′ ddTdTUUUAAGGUCUCCAGGCUCU 3′ | SEQ ID NO:19 |
| siRNA No: | 157 | |
| siRNA Name: | POSH-U111 | |
| Position in mRNA | 2973-2993 | |
| Target sequence: | 5′ AAGGATTGGTATGTGACTCTG 3′ | SEQ ID NO:20 |
| siRNA sense strand: | 5′ dTdTGGAUUGGUAUGUGACUCUG 3′ | SEQ ID NO:21 |
| siRNA anti-sense strand: | 5′ dTdTCAGAGUCACAUACCAAUCC 3′ | SEQ ID NO:22 |
| siRNA No: | 159 | |
| siRNA Name: | POSH-U410 | |
| Position in mRNA | 3272-3292 | |
| Target sequence: | 5′ AAGCTGGATTATCTCCTGTTG 3′ | SEQ ID NO:23 |
| siRNA sense strand: | 5′ ddTdTGCUGGAUUAUCUCCUGUUG 3′ | SEQ ID NO:24 |
| siRNA anti-sense strand: | 5′ ddTdTCAACAGGAGAUAAUCCAGC 3′ | SEQ ID NO:25 |

### Example 3

### In-Vitro Assay of Human POSH Self-Ubiquitination

Recombinant hPOSH was incubated with ATP in the presence of E1, E2 and ubiquitin as indicated in each lane. Following incubation at 37° C. for 30 minutes, reactions were terminated by addition of SDS-PAGE sample buffer. The samples were subsequently resolved on a 10% polyacrylamide gel. The separated samples were then transferred to nitrocellulose and subjected to immunoblot analysis with an anti ubiquitin polyclonal antibody. The position of migration of molecular weight markers is indicated on the right.

Poly-Ub: Ub-hPOSHconjugates, detected as high molecular weight adducts only in reactions containing E1, E2 and ubiquitin. hPOSH-176 and hPOSH-178 are a short and a longer derivatives (respectively) of bacterially expressed hPOSH; C, control E3.

Preliminary Steps in a High-Throughput Screen

Materials

1. E1 recombinant from bacculovirus
2. E2 Ubch5c from bacteria
3. Ubiquitin
4. POSH #178 (1-361) GST fusion-purified but degraded
5. POSH # 176 (1-269) GST fusion-purified but degraded
6. hsHRD1 soluble ring containing region
5. Buffer×12 (Tris 7.6 40 mM, DTT 1 mM, MgCl₂ 5 mM, ATP 2 uM)

6. Dilution buffer (Tris 7.6 40 mM, DTT 1 mM, ovalbumin 1 ug/ul)

| | 0.1 μg/μl | 0.5 μg/μl | 5 μg/μl | 0.4 μg/μl | 2.5 μg/μ/ | 0.8 μg/μl | |
|---|---|---|---|---|---|---|---|
| | E1 | E2 | Ub | 176 | 178 | Hrd1 | Bx12 |
| −E1 (E2 + 176) | — | 0.5 | 0.5 | 1 | — | — | 10 |
| −E2 (E1 + 176) | 1 | — | 0.5 | 1 | — | — | 9.5 |
| −ub (E1 + E2 + 176) | 1 | 0.5 | — | 1 | — | — | 9.5 |
| E1 + E2 + 176 + Ub | 1 | 0.5 | 0.5 | 1 | | — | 9 |
| −E1 (E2 + 178) | — | 0.5 | 0.5 | — | 1 | — | 10 |
| −E2 (E1 + 178) | 1 | — | 0.5 | — | 1 | — | 9.5 |
| −ub (E1 + E2 + 178) | 1 | 0.5 | — | — | 1 | — | 9.5 |
| E1 + E2 + 178 + Ub | 1 | 0.5 | 0.5 | — | 1 | ------1 | 9 |
| Hrd1, E1 + E2 + Ub | 1 | 0.5 | 0.5 | — | — | 1 | 8.5 |

1. Incubate for 30 minutes at 37° C.
2. Run 12% SDS PAGE gel and transfer to nitrocellulose membrane
3. Incubate with anti-Ubiquitin antibody.

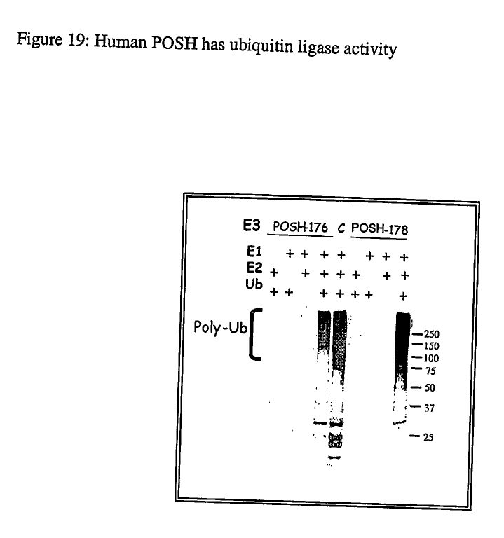

Results, shown in FIG. 19, demonstrate that human POSH has ubiquitin ligase activity.

### Example 4

### Co-Immunoprecipitation of hPOSH with myc-tagged Activated (V12) and Dominant-Negative (N17) Rac1

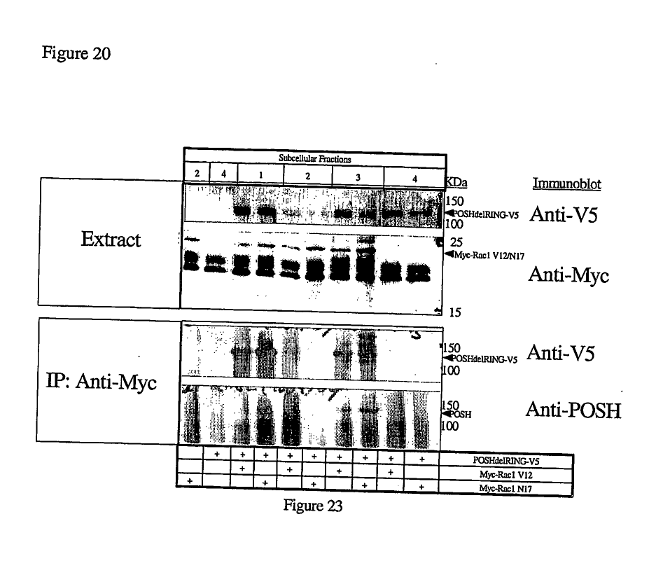

HeLa cells were transfected with combinations of myc-Rac1 V12 or N17 and hPOSHdelRING-V5. 24 hours after transfection (efficiency 80% as measured by GFP) cells were collected, washed with PBS, and swollen in hypotonic lysis buffer (10 mM HEPES pH=7.9, 15 mM KCl, 0.1 mM EDTA, 2 mM MgCl2, 1 mM DTT, and protease inhibitors). Cells were lysed by 10 strokes with dounce homogenizer and centrifuged 3000×g for 10 minutes to give supernatant (Fraction 1) and nucleii. Nucleii were washed with Fraction 2 buffer (0.2% NP-40, 10 mM HEPES pH=7.9, 40 mM KCl, 5% glycerol) to remove peripheral proteins. Nucleii were spun-down and supernatant collected (Fraction 2). Nuclear proteins were eluted in Fraction 3 buffer (20 mM HEPES pH=7.9, 0.42 M KCl, 25% glycerol, 0.1 mM EDTA, 2 mM MgCl₂, 1 mM DTT) by rotating 30 minutes in cold. Insoluble proteins were spun-down 14000×g and solubilized in Fraction 4 buffer (1% Fos-Choline 14, 50 mM HEPES pH=7.9, 150 mM NaCl, 10% glycerol, 1 mM EDTA, 1.5 mm MgCl₂, 2 mM DTT). Half of the total extract was pre-cleared against Protein A sepharose for 1.5 hours and used for IP with 1 μg anti-myc (9E10, Roche 1-667-149) and Protein A sepharose for 2 hours. Immune complexes were washed extensively, and eluted in SDS-PAGE sample buffer. Gels were run, and proteins electro-transferred to nitrocellulose for immunoblot as in FIG. 20. Endogenous POSH and transfected hPOSHdelRING-V5 are precipitated as a complex with Myc-Rac1 V12/N17. Results, shown in FIG. 20, demonstrate that POSH co-immunoprecipitates with Rac1.

### Example 5

### POSH Reduction Results in Decreased Secretion of phospholipase D (PLD)

Hela SS6 cells (two wells of 6-well plate) were transfected with POSH siRNA or control siRNA (100 nM). 24 hours later each well was split into 5 wells of a 24-well plate. The next day cells were transfected again with 100 nM of either POSH siRNA or control siRNA. The next day cells were washed three times with 1×PBS and than 0.5 ml of PLD incubation buffer (118 mM NaCl, 6 mM KCl, 1 mM CaCl₂, 1.2 mM MgSO4, 12.4 mM HEPES, pH7.5 and 1% fatty acid free bovine serum albumin) were added.

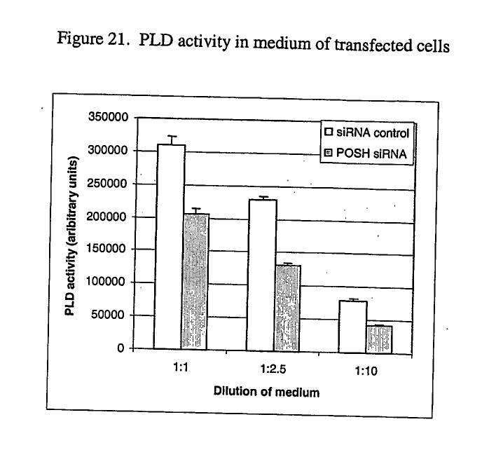

48 hours later medium was collected and centrifuged at 800×g for 15 minutes. The medium was diluted with 5×PLD reaction buffer (Amplex red PLD kit) and assayed for PLD by using the Amplex Red PLD kit (Molecular probes, A-12219). The assay results were quantified and presented below in as a bar graph. The cells were collected and lysed in 1% Triton X-100 lysis buffer (20 mM HEPES-NaOH, pH 7.4, 150 mM NaCl, 1.5 mM MgCl₂, 1 mM EDTA, 1% Triton X-100 and 1× protease inhibitors) for 15 minutes on ice. Lysates were cleared by centrifugation and protein concentration was determined. There were equal protein concentrations between the two transfectants. Equal amount of extracts were immunoprecipitated with anti-POSH antibodies, separated by SDS-PAGE and immunoblotted with anti-POSH antibodies to assess the reduction of POSH levels. There was approximately 40% reduction in POSH levels (FIG. 21).

### Example 6

### Effect of hPOSH on Gag-EGFP Intracellular Distribution

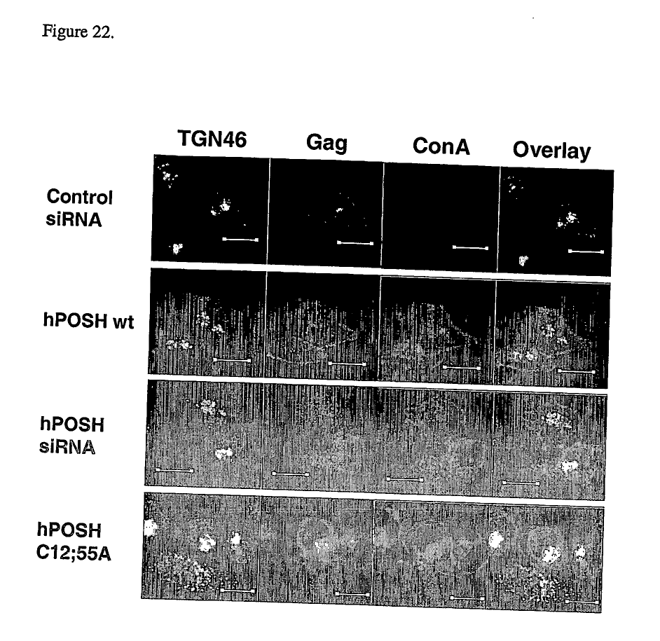

HeLa SS6 were transfected with Gag-EGFP, 24 hours after an initial transfection with either hPOSH-specific or scrambled siRNA (control) (100 nM) or with plasmids encoding either wild type hPOSH or hPOSH C(12,55)A. Fixation and staining was preformed 5 hours after Gag-EGFP transfection. Cells were fixed, stained with Alexa fluor 647-conjugated Concanavalin A (ConA) (Molecular Probes), permeabilized and then stained with sheep anti-human TGN46. After the primary antibody incubation cells were incubated with Rhodamin-conjugated goat anti-sheep. Laser scanning confocal microscopy was performed on LSM510 confocal microscope (Zeiss) equipped with Axiovert 100M inverted microscope using ×40 magnification and 1.3-numerical-aperture oil-immersion lens for imaging. For co-localization experiments, 10 optical horizontal sections with intervals of 1 μm were taken through each preparation (Z-stack). A single median section of each preparation is shown. See FIG. 22.

### Example 7

### POSH-Regulated Intracellular Transport of Myristoylated Proteins

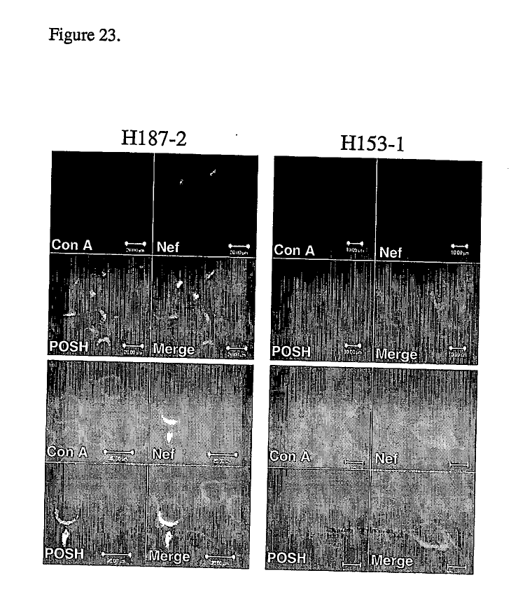

The localization of myristoylated proteins, Gag (see FIG. 22), HIV-1 Nef, Src and Rapsyn, in cells depleted of HPOSH were analyzed by immunofluorescence. In control cells, HIV-1 Nef was found in a perinuclear region co-localized with hPOSH, indicative of a TGN localization (FIG. 23). When hPOSH expression was reduced by siRNA treatment, Nef expression was weaker relative to control and nef lost its TGN, perinuclear localization. Instead it accumulated in punctated intracellular loci segregated from the TGN.

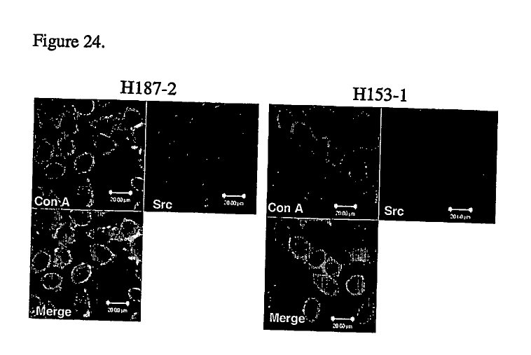

Src is expressed at the plasma membrane and in intracellular vesicles, which are found close to the plasma membrane (FIG. 24, H187 cells). However, when hPOSH levels were reduced, Src was dispersed in the cytoplasm and loses its plasma membrane proximal localization detected in control (H187) cells (FIG. 24, compare H153-1 and H187-2 panels).

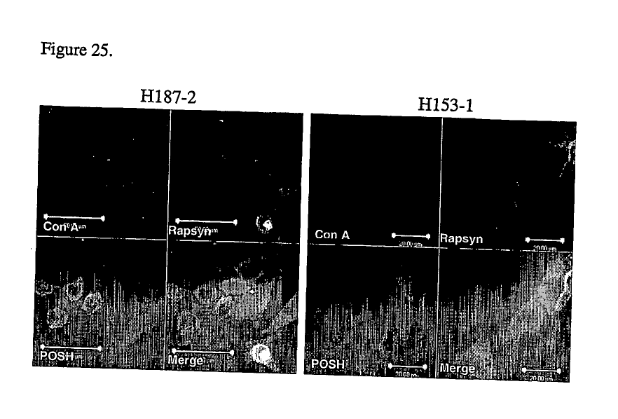

Rapsyn, a peripheral membrane protein expressed in skeletal muscle, plays a critical role in organizing the structure of the nicotinic postsynaptic membrane (Sanes and Lichtman, Annu. Rev. Neurosci. 22: 389-442 (1999)). Newly synthesized Rapsyn associates with the TGN and than transported to the plasma membrane (Marchand et al., J. Neurosci. 22: 8891-01 (2002)). In hPOSH-depleted cells (H153-1) Rapsyn was dispersed in the cytoplasm, while in control cells it had a punctuated pattern and plasma membrane localization, indicating that HPOSH influences its intracellular transport (FIG. 25).

Materials and Methods Used:

Antibodies:

Src antibody was purchased from Oncogene research products (Darmstadt, Germany). Nef antibodies were purchased from ABI (Columbia, Mass.) and Fitzgerald Industries International (Concord, Mass.). Alexa Fluor conjugated antibodies were purchased from Molecular Probes Inc. (Eugene, Oreg.).

hPOSH antibody: Glutathione S-transferase (GST) fusion plasmids were constructed by PCR amplification of HPOSH codons 285-430. The amplified PCR products was cloned into pGEX-6P-2 (Amersham Pharmacia Biotech, Buckinghamshire, UK). The truncated hPOSH protein was generated in E. coli BL21. Bacterial cultures were grown in LB media with carbenicillin (100 μg/ml) and recombinant protein production was induced with 1 mM IPTG for 4 hours at 30° C. Cells were lysed by sonication and the recombinant protein was then isolated from the cleared bacterial lysate by affinity chromatography on a glutathione-sepharose resin (Amersham Pharmacia Biotech, Buckinghamshire, UK). The hPOSH portion of the fusion protein was then released by incubation with PreScission protease (Amersham Pharmacia Biotech, Buckinghamshire, UK) according to the manufacturer's instructions and the GST portion was then removed by a second glutathione-sepharose affinity chromatography. The purified partial hPOSH polypeptide was used to immunize New Zealand white rabbits to generate antibody 15B (Washington Biotechnology, Baltimore, Md.).

Construction of siRNA Retroviral Vectors:

hPOSH scrambled oligonucleotide (5′-CACACACTGCCG TCAACT GTTCAAGAGAC AGTTGACGGCAGTGTGTGTTTTTT-3′; and 5′-AATTAAAAAACACA CACTGCCGTCAACTGTC TCTTGAACAGTTGA CGGCAGTGTGTGGGCC-3′) were annealed and cloned into the ApaI-EcoRI digested pSilencer 1.0-US (Ambion) to generate pSIL-scrambled. Subsequently, the U6-promoter and RNAi sequences were digested with BamHI, the ends filled in and the insert cloned into the Olil site in the retroviral vector, pMSVhyg (Clontech), generating pMSCVhyg-U6-scrambled. hPOSH oligonucleotide encoding RNAi against HPOSH (5′-AACAGAGGCCTTGGAAA CCTGGAAGC TTGCAGGTTT CCAAGGCCTCTGTT-3′; and 5′-GATCAACAGAG GCCTTGGAAACCTGC AAGCTTCCAGGTTTCCAA GGCCTCTGTT-3′) were annealed and cloned into the BamHI-EcoRI site of pLIT-U6, generating pLIT-U6 hPOSH-230. pLIT-U6 is an shRNA vector containing the human U6 promoter (amplified by PCR from human genomic DNA with the primers, 5′-GGCCCACTAGTCA AGGTCG GGCA GGAAGA-3′ and 5′-GCCGAATT CAAAAAGGATC CGGCGATATCCGG TGTTTCGTCCTTTCCA -3′) cloned into pLITMUS38 (New England Biolabs) digested with SpeI-EcoRI. Subsequently, the U6 promoter-hPOSH shRNA (pLIT-U6 hPOSH-230 digested with SnaBI and PvuI) was cloned into the Olil site of pMSVhyg (Clontech), generating pMSCVhyg U6-hPOSH-230.

Generation of Stable Clones:

HEK 293T cells were transfected with retroviral RNAi plasmids (pMSCVhyg-U6-Prt3-230 and pMSCVhyg-U6-scrambled and with plasmids encoding VSV-G and moloney gag-pol. Two days post transfection, medium containing retroviruses was collected and filtered and polybrene was added to a final concentration of 8 μg/ml. This was used to infect HeLa SS6 cells grown in 60 mm dishes. Forty-eight hours post-infection cells were selected for RNAi expression by the addition of hygromycin to a final concentration of 300 μg/ml. Clones expressing RNAi against hPOSH were named H153, clones expressing scrambled RNAi were named H187.

Transfection and Immunofluorescent Analysis:

Gag-EGFP experiments are described in FIG. 22.

H153 or H187 cells were transfected with Src or Rapsyn-GFP (Image clone image: 3530551 or pNLenv-1). Eighteen hours post transfection cells were washed with PBS and incubated on ice with Alexa Fluor 647 conjugated Con A to label plasma membrane glycoproteins. Subsequently cells were fixed in 3% paraformaldehyde, blocked with PBS containing 4% bovine serum albumin and 1% gelatin. Staining with rabbit anti-Src, rabbit anti-hPOSH (15B) or mouse anti-nef was followed with secondary antibodies as indicated.

Laser scanning confocal microscopy was performed on LSM510 confocal microscope (Zeiss) equipped with Axiovert 100M inverted microscope using ×40 magnification and 1.3-numerical-aperture oil-immersion lens for imaging. For co-localization experiments, 10 optical horizontal sections with intervals of 1 μm were taken through each preparation (Z-stack). A single median section of each preparation is shown.

### Example 8

### POSH Reduction by siRNA Abrogates West Nile Virus (“WNV”) Infectivity

HeLa SS6 cells were transfected with either control or POSH-specific siRNA. Cells were subsequently infected with WNV (4×10⁴ PFU/well). Viruses were harvested 24 hours and 48 hours post-infection, serially diluted, and used to infect Vero cells. As a control WNV (4×10⁴ PFU/well), that was not passed through HeLa SS6 cells, was used to infect Vero cells. Virus titer was determined by plaque assay in Vero cells.

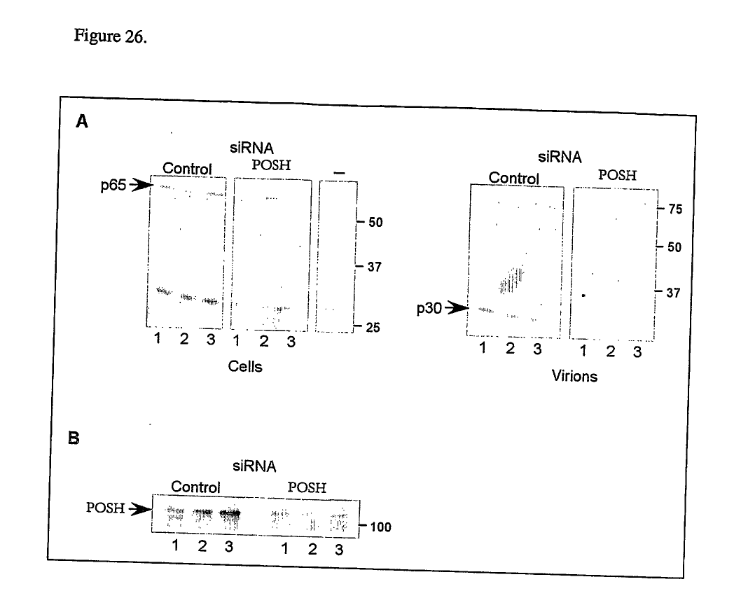

Virus titer was reduced by 2.5-log in cells treated with POSH-specific siRNA relative to cells transfected with control siRNA, thereby indicating that WNV requires POSH for virus secretion. See FIG. 26.

### Experimental Procedure

Cell Culture, Transfections and Infection:

Hela SS6 cells were grown in Dulbecco's modified Eagle's medium (DMEM) supplemented with 10% heat-inactivated fetal calf serum and 100 units/ml penicillin and 100 μg/ml streptomycin. For transfections, HeLa SS6 cells were grown to 50% confluency in DMEM containing 10% FCS without antibiotics. Cells were then transfected with the relevant double-stranded siRNA (100 nM) using lipofectamin 2000 (Invitrogen, Paisley, UK). On the day following the initial transfection, cells were split 1:3 in complete medium and transfected with a second portion of double-stranded siRNA (50 nM). Six hours post-transfection medium was replaced and cells infected with WNV (4×10⁴ PFU/well). Medium was collected from infected HeLa SS6 cells twenty-four and forty-eight post-infection (200 μl), serially diluted, and used to infect Vero cells. Virus titer was determined by plaque assay (Ben-Nathan D, Lachmi B, Lustig S, Feuerstien G (1991) Protection of dehydroepiandrosterone (DHEA) in mice infected with viral encephalitis. Arch Viro; 120, 263-271).

### Example 9

### Analysis of the Effects of POSH Knockdown on M-MuLV Expression and Budding

### Experimental Protocol

Transfections

A day before transfection, Hela SS6 cells were plated in two 6 wells plates at 5×10⁵ cells per well. 24 hours later the following transfections were performed:
4 wells were transfected with control siRNA and a plasmid encoding MMuLV.
4 wells were transfected with POSH siRNA and a plasmid encoding MMuLV.
1 well was a control without any siRNA or DNA transfected.
1 well was transfected with a plasmid encoding MMuLV.

For each well to be transfected 100 nM (12.5 μl) POSH siRNA or 100 nM (12.5 μl) control siRNA were diluted in 250 μl Opti-MEM (Invitrogen). Lipofectamin 2000 (5 μl) (Invitrogen, Cat. 11668-019) was mixed with 250 μl of OptiMEM per transfected well. The diluted siRNA was mixed with the lipofectamin 2000 mix and the solution incubated at room temperature for 30 min. The mixture was added directly to each well containing 2 ml DMEM+10% FBS (w/o antibiotics).

24 hours later, four wells of the same siRNA treatment were split to eight wells, and two wells without siRNA were split to four wells.

24 hours later all wells were transfected with 100 nM control siRNA or 100 nM POSH siRNA with or without a plasmid encoding MMuLV (see table below). 48 hours later virions and cells were harvested.

| | | Amount | Amount | The | |
|---|---|---|---|---|---|
| | | of RNAi | of DNA | volume of | |
| No of | | (μl) per | (μg) | DNA (μl) | |
| wells | RNAi | well | per well | per well | Application |
| 5 | POSH | 12.5 | MMuLV | 10 | 4 wells for |
| | 100 nM (1^{st} | | (2 μg) | | VLPs assay |
| | and 2^{nd} | | | | and 1 well for |
| | transfection) | | | | RT |
| 5 | Control | 12.5 | MMuLV | 10 | 4 wells for |
| | 100 nM (1^{st} | | (2 μg) | | VLPs assay |
| | and 2^{nd} | | | | and 1 well for |
| | transfection) | | | | RT |
| 1 | — | — | — | 10 μl H₂O | VLPs assay |
| 1 | — | — | MMuLV | 10 | VLPs assay |
| | | | (2 μg) | | |

Steady State VLP Assay
Cell Extracts:
1. Pellet floating cells by centrifugation (10 min, 500×g at 4° C.), save supernatant (continued at step 7), wash cells once, scrape cells in ice-cold 1×PBS, add to the corresponding cell pellet and centrifuge for 5 min 1800 rpm at 4° C.
2. Wash cell pellet once with ice-cold 1×PBS.
3. Resuspend cell pellet in 150 μl 1% Triton X-100 lysis buffer (20 mM HEPES-NaOH, pH 7.4, 150 mM NaCl, 1.5 mM MgCl₂, 1 mM EDTA, 1% Triton X-100 and 1× protease inhibitors) and incubate 20 minutes on ice.
4. Centrifuge at 14,000rpm for 15 min. Transfer supernatant to a clean tube.
5. Determine protein concentration by BCA.
6. Prepare samples for SDS-PAGE by adding 2 μl of 6×SB to 20 μg extract (add lysis buffer to a final volume of 12 μl), heat to 80° C. for 10 min.

Purification of Virions from Cell Media
7. Filtrate the supernatant through a 0.45 μm filter.
8. Transfer 1500 μl of virions fraction to an ultracentrifuge tube (swinging rotor).
9. Add 300 μl of fresh sucrose cushion (20% sucrose in TNE) to the bottom of the tube.
10. Centrifuge supernatant at 35000 rpm at 4° C. for 2 hr.
11. Resuspend virion pellet in 50 μl hot 1× sample buffer each (samples 153-1, 2, 3, 187-1, 2, 3). Resuspend VLPs pellet (153-4, 5 and 187 4, 5) in 25 μl hot 1× sample buffer. Vortex shortly, transfer to an eppendorf tube, unite VLPs from wells 153-4+5 and 187-4+5. Heat to 80° C. for 10 min.
12. Load equal amounts of VLPs relatively to cells extracts amounts.
   Western Blot Analysis
1. Separate all samples on 12% SDS-PAGE.
2. Transfer samples to nitrocellulose membrane (100V for 1.15 hr).
3. Dye membrane with ponceau solution.
4. Block with 10% low fat milk in TBS-T for 1 hour.
5. Incubate membranes with Goat anti p30 (81S-263) (1:5000) in 10% low fat milk in TBS-T over night at 4° C. Incubate with secondary antibody rabbit anti goat-HRP 1:8000 for 60 min at room temperature.
6. Detect signal by ECL reaction.
7. Following the ECL detection incubate memebranes with Donkey anti rabbit Cy3 (Jackson Laboratories, Cat 711-165-152) 1:500 and detect signal by Typhoon scanning and quantitate.
   Results:

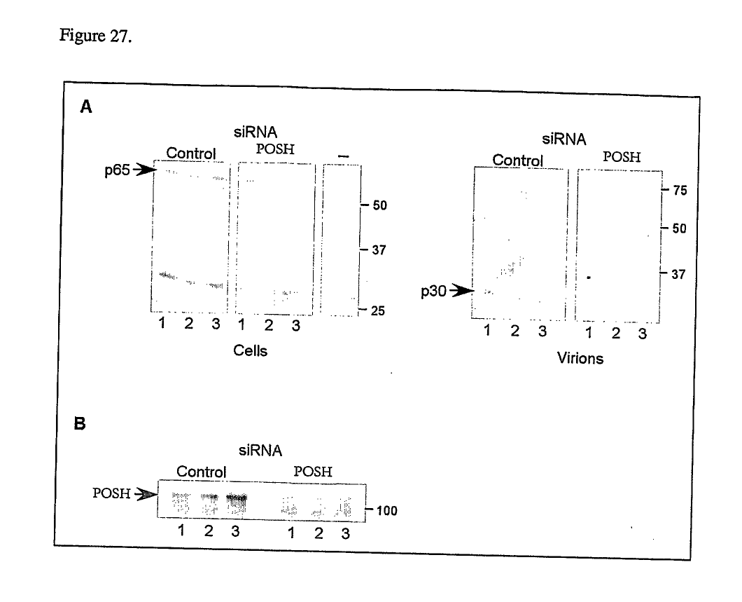

As shown in FIG. 27, POSH knockdown decreases the release of extracellular MMuLV particles.

### Example 10

### POSH Protein-Protein Interactions by Yeast Two Hybrid Assay

POSH-associated proteins were identified by using a yeast two-hybrid assay.

Procedure:

Bait plasmid (GAL4-BD) was transformed into yeast strain AH109 (Clontech) and transfromants were selected on defined media lacking tryptophan. Yeast strain Y187 containing pre-transformed Hela cDNA prey (GAL4-AD) library (Clontech) was mated according to the Clontech protocol with bait containing yeast and plated on defined media lacking tryptophan, leucine, histidine and containing 2 mM 3 amino triazol. Colonies that grew on the selective media were tested for beta-galactosidase activity and positive clones were further characterized. Prey clones were identified by amplifying cDNA insert and sequencing using vector derived primers.
Bait:
Plasmid vector: pGBK-T7 (Clontech)
Plasmid name: pPL269-pGBK-T7 GAL4 POSHdR

Protein sequence: Corresponds to aa 53-888 of POSH (RING domain deleted)

RTLVGSGVEELPSNILLVRLLDGIKQRPWKPGPGGGSGTNCTNALRSQSS
TVANCSSKDLQSSQGGQQPRVQSWSPPVRGIPQLPCAKALYNYEGKEPGD
LKFSKGDIIILRRQVDENWYHGEVNGIHGFFPTNFVQIIKPLPQPPPQCK
ALYDFEVKDKEADKDCLPFAKDDVLTVIRRVDENWAEGMLADKIGIFPIS
YVEFNSAAKQLIEWDKPPVPGVDAGECSSAAAQSSTAPKHSDTKKNTKKR
HSFTSLTMANKSSQASQNRHSMEISPPVLISSSNPTAAARISELSGLSCS
APSQVHISTTGLIVTPPPSSPVTTGPSFTFPSDVPYQAALGTLNPPLPPP
PLLAATVLASTPPGATAAAAAAGMGPRPMAGSTDQIAHLRPQTRPSVYVA
IYPYTPRKEDELELRKGEMFLVFERCQDGWFKGTSMHTSKIGVFPGNYVA
PVTRAVTNASQAKVPMSTAGQTSRGVTMVSPSTAGGPAQKLQGNGVAGSP
SVVPAAVVSAAHIQTSPQAKVLLHMTGQMTVNQARNAVRTVAAHNQERPT
AAVTPIQVQNAAGLSPASVGLSHHSLASPQPAPLMPGSATHTAAISISRA
SAPLACAAAAPLTSPSITSASLEAEPSGRIVTVLPGLPTSPDSASSACGN
SSATKPDKDSKKEKKGLLKLLSGASTKRKPRVSPPASPTLEVELGSAELP
LQGAVGPELPPGGGHGRAGSCPVDGDGPVTTAVAGAALAQDAFHRKASSL
DSAVPIAPPPRQACSSLGPVLNESRPVVCERHRVVVSYPPQSEAELELKE
GDIVFVHKKREDGWFKGTLQRNGKTGLFPGSFVENI

Library screened: Hela pretransformed library (Clontech).

One regulatory subunit (e.g., PRKAR1A) of PKA was identified as a POSH-AK by yeast two-hybrid screen. As shown below, PKA phosphorylates POSH. Since both a regulatory subunit and a catalytic subunit are required for the PKA function, a catalytic subunit of PKA such as PRKACA or PRKACB forms a complex with POSH and can be a POSH-AK.

Examples of sequences for a regulatory subunit of PKA (PRKAR1A) and two catalytic subunits of PKA (PRKACA and PRKACB) are presented below.

Human PRKAR1A mRNA sequence - var1
(public gi:23273779)
GGTGGAGCTGTCGCCTAGCCGCTATCGCAGAGTGGAGCGGGGCTGGGAGC
AAAGCGCTGAGGGAGCTCGGTACGCCGCCGCCTCGCACCCGCAGCCTCGC
GCCCGCCGCCGCCCGTCCCCAGAGAACCATGGAGTCTGGCAGTACCGCCG
CCAGTGAGGAGGCACGCAGCCTTCGAGAATGTGAGCTCTACGTCCAGAAG
CATAACATTCAAGCGCTGCTCAAAGATTCTATTGTGCAGTTGTGCACTGC
TCGACCTGAGAGACCCATGGCATTCCTCAGGGAATACTTTGAGAGGTTGG
AGAAGGAGGAGGCAAAACAGATTCAGAATCTGCAGAAAGCAGGCACTCGT
ACAGACTCAAGGGAGGATGAGATTTCTCCTCCTCCACCCAACCCAGTGGT
TAAAGGTAGGAGGCGACGAGGTGCTATCAGCGCTGAGGTCTACACGGAGG
AAGATGCGGCATCCTATGTTAGAAAGGTTATACCAAAAGATTACAAGACA
ATGGCCGCTTTAGCCAGCCAAAGCCATTGAAAAGAATGTGCTGTTTTCAC
ATCTTGATGATAATGAGAGAAGTGATATTTTTGATGCCATGTTTTCGGTC
TCCTTTATCGCAGGAGAGACTGTGATTCAGCAAGGTGATGAAGGGGATAA
CTTCTATGTGATTGATCAAGGAGAGACGGATGTCTATGTTAACAATGAAT
GGGCAACCAGTGTTGGGGAAGGAGGGAGCTTTGGAGAACTTGCTTTGATT
TATGGAACACCGAGAGCAGCCACTGTCAAAGCAAAGACAAATGTGAAATT
GTGGGGCATCGACCGAGACAGCTATAGAAGAATCCTCATGGGAAGCACAC
TGAGAAAGCGGAAGATGTATGAGGAATTCCTTAGTAAAGTCTCTATTTTA
GAGTCTCTGGACAAGTGGGAACGTCTTACGGTAGCTGATGCATTGGAACC
AGTGCAGTTTGAAGATGGGCAGAAGATTGTGGTGCAGGGAGAACCAGGGG
ATGAGTTCTTCATTATTTTAGAGGGGTCAGCTGCTGTGCTACAACGTCGG
TCAGAAAATGAAGAGTTTGTTGAAGTGGGAAGATTGGGGCCTTCTGATTA
TTTTGGTGAAATTGCACTACTGATGAATCGTCCTCGTGCTGCCACAGTTG
TTGCTCGTGGCCCCTTGAAGTGCGTTAAGCTGGACCGACCTAGATTTGAA
CGTGTTCTTGGCCCATGCTCAGACATCCTCAAACGAAACATCCAGCAGTA
CAACAGTTTTGTGTCACTGTCTGTCTGAAATCTGCCTCCTGTGCCTCCCT
TTTCTCCTCTCCCCAATCCATCCTTCACTCATGCAAACTGCTTTATTTTC
CCTACTTGCAGCGCCAAGTGGCCACTGGCATCGCAGCTTCCTGTCTGTTT
ATATATTGAAAGTTGCTTTTATTGCACCATTTTCAATTTGGAGCATTAAC
TAAATGCTCATACACAGTTAAATAAATAGAAAGAGTTCTATGGAGACTTT
GCTGTTACTGCTTCTCTTTGTGCAGTGTTAGTATTCACCCTGGGCAGTGA
GTGCCATGCTTTTTGGTGAGGGCAGATCCCAGCACCTATTGAATTACCAT
AGAGTAATGATGTAACAGTGCAAGATTTTTTTTTTAAGTGACATAATTGT
CCAGTTATAAGCGTATTTAGACTGTGGCCATATATGCTGTATTTCTTTGT
AGAATAAATGGTTTCTCATTAAACTCTAAAGATTAGGGAAAATGGATATA
GAAAATCTTAGTATAGTAGAAAGACATCTGCCTGTAATTAAACTAGTTTA
AGGGTGGAAAAATGCCCATTTTTGCTAATTATCAATGGGATATGATTGGT
TCAGTTTTTTTTTTTCCAGAGTTGTTGTTTGCCAAGCTAATCTGCCTGGT
TTTATTTATATCTTGTTATTAATGTTTCTTCTCCAATTCTGAAATACTTT
TGAGTATGGCTATCTATACCTGCCTTTTAAGTTTGAAACTAACTCATAGA
TTGCAAATATTGGTTAGTATTTAACTACATCTGCCTCGGCTCACAAATTC
CGATTAGACCTTTATCCAGCTAGTGCCAAATAATTGATCAGATGCTGAAT
TGAGAATAAGAATTTGAGGTCTACATTCTTGGTTGTTAATTTAGAGCGTT
TGGTTAAAGTATGTCCTTCAGCTGACTCCAGTATAATCTCCTCTGCTCAT
TAAACTGATTCCAGGAGATTGGATTTGCTGTGACTAGATACAGATGGAGC
AAATGTCCTAACAGAGAAATAGAGGTGATGCTGCTAAAGGGAGAAATGCC
AGGCGGACAAGTTCAGTGTCGGGAATTTTCCCCGTGACATTCACTGGGGC
ATGAGATTTTGGAAGAAGTTTTTTACTTTGGTTTAGTCTTTTTTTCCTTC
CTTTTTATTCAGCTAGAATTTCTGGTGGGTTGATGGTAGGGTATAATGTG
TCTGTGTTGCTTCAAATTGGTCTGAAAGGCTATCCTGCGGAAAGTCCTGC
TTTCCTATCTAGCATTTATTTCTCTGGCAAACTTTTCTTTCTTTTCTTTT
TTAAAGTAAACTTGTGTATTGAGTCTTAACTGTATTTCAGTATTTTCCAG
CCTTATGTGTTACATTATTCCAATGATACCCAACAGTTTATTTTTATTAT
TTTTTTAAACAAAATTTCACAGTTCTGTAATGTAGGCACTTTTATTTTCA
TTGTGATTTATATATAAGGTAATGTAGGGTTATATTTGGGAGTGACTGCA
AGCATTTTTCCATCTGTGTGCAACTAACTGACTCTGTTATTGATCCCTTC
TCCTGCCCTTTCCCAGGTAATTTAAATTGGTCATGGTAGATTTTTTTCAT
AGATTTGAAAAACTTTTAGGTTGTTACCAAGTATGAAGTATAAATCTGGG
GAAGAGGTTTTATTTACATTTTAGGGTGGGTAAGAAAGCCACCTTGTTAC
AAATTTTTTAATTTCCAAAATAATCTATATTAAATGAGGGTTTCTGATCT
GTACTTTGTGTTTAGCTACCTTTTTATATTTAAAAAATTAAAAATGAAAA
TTACGTTCTTACAAGCTTAAAGCTTGATTTGATCTTTGTTTAAATGCCAA
AATGTACTTAAATGAGTTACTTAGAATGCCATAAAATTGCAGTTTCATGT
ATGTATATAATCATGCTCATGTATATTTAGTTACGTATAATGCTTTCTGA
GTGAGTTTTACTCTTAAATCATTTGGTTAAATCATTTGGCTTGCTGTTTA
CTCCCTTCTGTAGTTTTTAATTAAAAACTTTAAAGATAAGTCTACATTAA
ACAATGATCACATCTAAAGCTTTATCTTTGTGTAATCTAAGTATATGTGA
GAAATCAGAATTGGCATAATTTGTCTTAGTTGATATTCAAGGCTTTAAAA
GTCATTATTCCTGGGCTTGGTAAGTGAATTTATGAGATTTACTGCTCTAG
AAAGTATAGATGGCGAAAGGACCGTTTTGTATTGCTTCCTGATTACCAGT
CTGATTATACCATGTGTGCTAATATACTTTTTTTGTTATAGATTGTCTTA
ATGGTAGGTCAAGTAATAAAAAGAGATGAAATAATTTAAAAAAAAAAAAA
AAA
Human PRKAR1A mRNA sequence - var2
(public gi:1658305)
AGAGGCGTCAAGGGAGGCCGGAGGGAGAGTGGGGTGGACAGAGGAGCGGA
GGGACGAGAGGGAAGCGCACGATAGCTGCGCGGAGAGAGAGCGAAGAGCA
GGAGGAGGAACAAAGGCGACCCAAGACACCCAGAGAGGGACAGAGAACCA
TGGAGTCTGGCAGTACCGCCGCCAGTGAGGAGGCACGCAGCCTTCGAGAA
TGTGAGCTCTACGTCCAGAAGCATAACATTCAAGCGCTGCTCAAAGATTC
TATTGTGCAGTTGTGCACTGCTCGACCTGAGAGACCCATGGCATTCCTCA
GGGAATACTTTGAGAGGTTGGAGAAGGAGGAGGCAAAACAGATTCAGAAT
CTGCAGAAAGCAGGCACTCGTACAGACTCAAGGGAGGATGAGATTTCTCC
TCCTCCACCCAA
Human PRKAR1A mRNA sequence - var3
(public gi:21757396)
TAATTTTCTTGTGTGTTTTTAAAAATTTTGATTATGCTAGTAGTTGGCTA
ATCAGATCCTCACTCCAGTGGTTTGCTCTGTGACGTTAGGATACTCCCAT
GGGATAGAAGTTACGTATAGGGAATGTCAGATATTCTTCATTGTGCTGAC
TTGCTTTCGCTTACAGTTGACTTTTGTGCCCTGGTAATTCTGTATCCTGT
TTACCGTTTACCTACTTCCCACGTCATCATGATTTCTTTTGAGGGAGAAC
TGAATGAAATTCCCTTAAGGGCCTGACTTCAGCACCCGTCTCTGCAGAGG
TTAGTGGCTCATACTTCCTCCCAGGAGCTGAGGTTATCGACTCTCACTGT
TGCCTACAGAGCACAGATCCTGAACTAAATGAAACATTTACTTGGAATAA
TGCTAATTCTGTACATATTTTATTCCCTAGTCCCCACTTCCCTGTTTAAA
AACAAAATCTACTTAGAAAAAAATCCCTGTGAATCAGTTGTCTAATGAAT
TTAGCAAGTTAAATGCCAGATTGACATTTTGCTTTATAGTTTATACAAGC
ATGTGTGTGTTTTTTTCTCGCAGAGAACCATGGAGTCTGGCAGTACCGCC
GCCAGTGAGGAGGCACGCAGCCTTCGAGAATGTGAGCTCTACGTCCAGAA
GCATAACATTCAAGCGCTGCTCAAAGATTCTATTGTGCAGTTGTGCACTG
CTCGACCTGAGAGACCCATGGCATTCCTCAGGGAATACTTTGAGAGGTTG
GAGAAGGAGGAGGCAAAACAGATTCAGAATCTGCAGAAAGCAGGCACTCG
TACAGACTCAAGGGAGGATGAGATTTCTCCTCCTCCACCCAACCCAGTGG
TTAAAGGTAGGAGGCGACGAGGTGCTATCAGCGCTGAGGTCTACACGGAG
GAAGATGCGGCATCCTATGTTAGAAAGGTTATACCAAAAGATTACAAGAC
AATGGCCGCTTTAGCCAAAGCCATTGAAAAGAATGTGCTGTTTTCACATC
TTGATGATAATGAGAGAAGTGATATTTTTGATGCCATGTTTTCGGTCTCC
TTTATCGCAGGAGAGACTGTGATTCAGCAAGGTGATGAAGGGGATAACTT
CTATGTGATTGATCAAGGAGAGACGGATGTCTATGTTAACAATGAATGGG
CAACCAGTGTTGGGGAAGGAGGGAGCTTTGGAGAACTTGCTTTGATTTAT
GGAACACCGAGAGCAGCCACTGTCAAAGCAAAGACAAATGTGAAATTGTG
GGGCATCGACCGAGACAGCTATAGAAGAATCCTCATGGGAAGCACACTGA
GAAAGCGGAAGATGTATGAGGAATTCCTTAGTAAAGTCTCTATTTTAGAG
TCTCTGGACAAGTGGGAACGTCTTACGGTAGCTGATGCATTGGAACCAGT
GCAGTTTGAAGATGGGCAGAAGATTGTGGTGCAGGGAGAACCAGGGGATG
AGTTCTTCATTATTTTAGAGGGGTCAGCTGCTGTGCTACAACGTCGGTCA
GAAAATGAAGAGTTTGTTGAAGTGGGAAGATTGGGGCCTTCTGATTATTT
TGGTGAAATTGCACTACTGATGAATCGTCCTCGTGCTGCCACAGTTGTTG
CTCGTGGCCCCTTGAAGTGCGTTAAGCTGGACCGACCTAGATTTGAACGT
GTTCTTGGCCCATGCTCAGACATCCTCAAACGAAACATCCAGCAGTACAA
CAGTTTTGTGTCACTGTCTGTCTGAAATCCGCCTCCTGTGCCTCCCTTTT
CTCCTCTCCCCAATCCATGCTTCACTCATGCAAACTGCTTTATTTTCCCT
ACTTGCAGCGCCAAGTGGCCACTGGCATCGCAGCTTCCTGTCTGTTTATA
TATTGAAAGTTGCTTTTATTGCACCATTTTCAATTTGGAGCATTAACTAA
ATGCTCATACACAGTTAAATAAATAGAAAGAGTTCTATGG
Human PRKAR1A mRNA sequence - var4
(public gi:1526988)
GGCAGAGTGGAGCGGGGCTGGGAGCAAAGCGCTGAGGGAGCTCGGTACGC
CGCCGCCTCGCACCCGCAGCCTCGCGCCCGCCGCCGCCCGTCCCCAGAGA
ACCATGGAGTCTGGCAGTACCGCCGCCAGTGAGGAGGCACGCAGCCTTCG
AGAATGTGAGCTCTACGTCCAGAAGCATAACATTCAAGCGCTGCTCAAAG
ATTCTATTGTGCAGTTGTGCACTGCTCGACCTGAGAGACCCATGGCATTC
CTCAGGGAATACTTTGAGAGGTTGGAGAAGGAGGAGGCAAAACAGATTCA
GAATCTGCAGAAAGCAGGCACTCGTACAGACTCAAGGGAGGATGAGATTT
CTCCTCCTCCACCCAACCCAGTGGTTAAAGGTAGGAGGCGACGAGGTGCT
ATCAGCGCTGAGGTCTACACGGAGGAAGATGCGGCATCCTATGTTAGAAA
GGTTATACCAAAAGATTACAAGACAATGGCCGCTTTAGCCAAAGCCATTG
AAAAGAATGTGCTGTTTTCACATCTTGATGATAATGAGAGAAGTGATATT
TTTGATGCCATGTTTTCGGTCTCCTTTATCGCAGGAGAGACTGTGATTCA
GCAAGGTGATGAAGGGGATAACTTCTATGTGATTGATCAAGGAGAGACGG
ATGTCTATGTTAACAATGAATGGGCAACCAGTGTTGGGGAAGGAGGGAGC
TTTGGAGAACTTGCTTTGATTTATGGAACACCGAGAGCAGCCACTGTCAA
AGCAAAGACAAATGTGAAATTGTGGGGCATCGACCGAGACAGCTATAGAA
GAATCCTCATGGGAAGCACACTGAGAAAGCGGAAGATGTATGAGGAATTC
CTTAGTAAAGTCTCTATTTTAGAGTCTCTGGACAAGTGGGAACGTCTTAC
GGTAGCTGATGCATTGGAACCAGTGCAGTTTGAAGATGGGCAGAAGATTG
TGGTGCAGGGAGAACCAGGGGATGAGTTCTTCATTATTTTAGAGGGGTCA
GCTGCTGTGCTACAACGTCGGTCAGAAAATGAAGAGTTTGTTGAAGTGGG
AAGATTGGGGCCTTCTGATTATTTTGGTGAAATTGCACTACTGATGAATC
GTCCTCGTGCTGCCACAGTTGTTGCTCGTGGCCCCTTGAAGTGCGTTAAG
CTGGACCGACCTAGATTTGAACGTGTTCTTGGCCCATGCTCAGACATCCT
CAAACGAAACATCCAGCAGTACAACAGTTTTGTGTCACTGTCTGTCTGAA
ATCTGCCTCCTGTGCCTCCCTTTTCTCCTCTCCCCAATCCATGCTTCACT
CATGCAAACTGCTTTATTTTCCCTACTTGCAGCGCCAAGTGGCCACTGGC
ATCGCAGCTTCCTGTCTGTTTATATATTAAAGTTGCTTTTATTGCACCAT
TTTCAATTTGGAGCATTAACTAAATGCTCATACACAGTTAAATAAATAGA
AAGAGTTCTATGGAAAAAAAAAAAAA
Human PRKAR1A mRNA sequence - var5
(public gi:1526989)
GCTGGGAGCAAAGCGCTGAGGGAGCTCGGTACGCCGCCGCCTCGCACCCG
CAGCCTCGCGCCCGCCGCCGCCCGTCCCCAGAGAACCATGGAGTCTGGCA
GTACCGCCGCCAGTGAGGAGGCACGCAGCCTTCGAGAATGTGAGCTCTAC
GTCCAGAAGCATAACATTCAAGCGCTGCTCAAAGATTCTATTGTGCAGTT
GTGCACTGCTCGACCTGAGAGACCCATGGCATTCCTCAGGGAATACTTTG
AGAGGTTGGAGAAGGAGGAGGCAAAACAGATTCAGAATCTGCACAAAGCA
GGCACTCGTACAGACTCAAGGGAGGATGAGATTTCTCCTCCTCCACCCAA
CCCAGTGGTTAAAGGTAGGAGGCGACGAGGTGCTATCAGCGCTGAGGTCT
ACACGGAGGAAGATGCGGCATCCTATGTTAGAAAGGTTATACCAAAAGAT
TACAAGACAATGGCCGCTTTAGCCAAAGCCATTGAAAAGAATGTGCTGTT
TTCACATCTTGATGATAATGAGAGAAGTGATATTTTTGATGCCATGTTTT
CGGTCTCCTTTATCGCAGGAGAGACTGTGATTCAGCAAGGTGATGAAGGG
GATAACTTCTATGTGATTGATCAAGGAGAGACGGATGTCTATGTTAACAA
TGAATGGGCAACCAGTGTTGGGGAAGGAGGGAGCTTTGGAGAACTTGCTT
TGATTTATGGAACACCGAGAGCAGCCACTGTCAAAGCAAAGACAAATGTG
AAATTGTGGGGCATCGACCGAGACAGCTATAGAAGAATCCTCATGGGAAG
CACACTGAGAAAGCGGAAGATGTATGAGGAATTCCTTAGTAAAGTCTCTA
TTTTAGAGTCTCTGGACAAGTGGGAACGTCTTACGGTAGCTGATGCATTG
GAACCAGTGCAGTTTGAAGATGGGCAGAAGATTGTGGTGCAGGGAGAACC
AGGGGATGAGTTCTTCATTATTTTAGAGGGGTCAGCTGCTGTGCTACAAC
GTCGGTCAGAAAATGAAGAGTTTGTTGAAGTGGGAAGATTGGGGCCTTCT
GATTATTTTGGTGAAATTGCACTACTGATGAATCGTCCTCGTGCTGCCAC
AGTTGTTGCTCGTGGCCCCTTGAAGTGCGTTAAGCTGGACCGACCTAGAT
TTGAACGTGTTCTTGGCCCATGCTCAGACATCCTCAAACGAAACATCCAG
CAGTACAACAGTTTTGTGTCACTGTCTGTCTGAAATCTGCCTCCTGTGCC
TCCCTTTTCTCCTCTCCCCAATCCATGCTTCACTCATGCAAACTGCTTTA
TTTTCCCTACTTGCAGCGCCAAGTGGCCACTGGCATCGCAGCTTCCTGTC
TGTTTATATATTGAAAGTTGCTTTTATTGCACCATTTTCAATTTGGAGCA
TTAACTAAATGCTCATACACAGTTAAATAAATAGAAAGAGTTCTATGGAG
ACTTTGCTGTTACTGCTTCTCTTTGTGCAGTGTTAGTATTCACCCTGGGC
AGTGAGTGCCATGCTTTTTGGTGAGGGCAGATCCAGCACCTATTGAATTA
CCATAGAGTAATGATGTAACAGTGCAAGATTTTTTTTTTTAAGTGACATA
ATTGTCCAGTTATAAGCGTATTTAGACTCTGGCCATATATGCTGTATTTC
TTTGTAGAATAAATGGTTTCTCATTAAACTCTAAAGATTAGGGAAATGGA
TATAGAAAATCTTAGTATAGTAGAAAGACATCTGCCTGTAATTAAACTAG
TTTAAGGGTGGAAAAATGAAAATTTTTGCTAATTATCAATGGGATATGAT
TGGTTCAGTTTTTTTTTTCCAGAGTTGTTGTTTGCCAAGCTAATCTGCCT
GGTTTATTTATATCTTGTTATTAATGTTTCTTCTCCAATTCTGAAATACT
TTTGAGTATGGCTATCTATACCTGCCTTTTAAGTTTGAAACTAACTCATA
GATGCAAATATTGGTTAGTATTTAACTACATCTGCCTCGGCTCACAAATT
CCGATTAGACCTTTATCCAGCTAGTGCCAAATAATTGATCAGATGCTGAA
TTGAGAATAAGAATTTGAGGTCTACATTCTTGGTTGTTAATTTAGAGCGT
TTGGTTAAAGTATGTCCTTCAGCTGACTCCAGTATAATCTCCTCTGCTCA
TTAAACTGATTCCAGGAGATTGGATTTGCTGTGACTAGATACAGATGGAG
CAAATGTCCTAACAGAGAAATAGAGGTGATGCTGCTAAAGGGAGAAATGC
CAGGCGGACAAAGTTCAGTGTCGGGAATTTTCCCCGTGACATTCACTGGG
GCATGAGATTTTGGAAGAAGTTTTTTACTTTGGTTTAGTCTTTTTTTCCT
CCTTTTTATTCAGCTAGAATTTCTGGTGGGTTGATGGTAGGGTATAATGT
GTCTGTGTTGCTTCAAATTGGTCTGAAAGGCTATCCTGCTGAAAGTCCTG
CTTTCCTATCTAGCATTTATTCCTCTGGCAAACTTTTCTTTCTTTTCTTT
TTTAAAGTAAACTTGTGTATTGAGTCTTAACTGTATTTCAGTATTTTCCA
GCCTTATGTGTTACATTATTCCAATGATACCCAACAGTTTATTTTTATTA
TTTTTTTAAACAAAATTTCACAGTTCTGTAATGTAGGCACTTTTATTTTC
ATTGTGATTTATATATAAGGTAATGTAGGGTTATATTTGGGAGTGACTGC
AAGCATTTTTCCATCTGTGTGCAACTAACTGACTCTGTTATTGATCCCTT
CTCCTGCCCTTTCCCAGGTAATTTAAATTGGTCATGGTAGATTTTTTTCA
TAGATTTGAAAAACTTTTAGGTTGTTACCAAGTATGAAGTATAAATCTGG
GGAAGAGGTTTTATTTACATTTTAGGGTGGGTAAGAAAGCCACCTTGTTA
CAAATTTTTTAATTTCCAAAATAATCTATATTAAATGAGGGTTTCTGATC
TGTACTTTGTGTTTAGCTACCTTTTTATATTTAAAAAATTAAAAATGAAA
ATTATGTTCTTACAAGCTTAAAGCTTGATTTGATCT
Human PRKAR1A mRNA sequence - var6
(public gi:4506062)
GCTGGGAGCAAAGCGCTGAGGGAGCTCGGTACGCCGCCGCCTCGCACCCG
CAGCCTCGCGCCCGCCGCCGCCCGTCCCCAGAGAACCATGGAGTCTGGCA
GTACCGCCGCCAGTGAGGAGGCACGCAGCCTTCGAGAATGTGAGCTCTAC
GTCCAGAAGCATAACATTCAAGCGCTGCTCAAAGATTCTATTGTGCAGTT
GTGCACTGCTCGACCTGAGAGACCCATGGCATTCCTCAGGGAATACTTTG
AGAGGTTGGAGAAGGAGGAGGCAAAACAGATTCAGAATCTGCAGAAAGCA
GGCACTCGTACAGACTCAAGGGAGGATGAGATTTCTCCTCCTCCACCCAA
CCCAGTGGTTAAAGGTAGGAGGCGACGAGGTGCTATCAGCGCTGAGGTCT
ACACGGAGGAAGATGCGGCATCCTATGTTAGAAAGGTTATACCAAAAGAT
TACAAGACAATGGCCGCTTTAGCCAAAGCCATTGAAAAGAATGTGCTGTT
TTCACATCTTGATGATAATGAGAGAAGTGATATTTTTGATGCCATGTTTT
CGGTCTCCTTTATCGCAGGAGAGACTGTGATTCAGCAAGGTGATGAAGGG
GATAACTTCTATGTGATTGATCAAGGAGAGACGGATGTCTATGTTAACAA
TGAATGGGCAACCAGTGTTGGGGAAGGAGGGAGCTTTGGAGAACTTGCTT
TGATTTATGGAACACCGAGAGCAGCCACTGTCAAAGCAAAGACAAATGTG
AAATTGTGGGGCATCGACCGAGACAGCTATAGAAGAATCCTCATGGGAAG
CACACTGAGAAAGCGGAAGATGTATGAGGAATTCCTTAGTAAAGTCTCTA
TTTTAGAGTCTCTGGACAAGTGGGAACGTCTTACGGTAGCTGATGCATTG
GAACCAGTGCAGTTTGAAGATGGGCAGAAGATTGTGGTGCAGGGAGAACC
AGGGGATGAGTTCTTCATTATTTTAGAGGGGTCAGCTGCTGTGCTACAAC
GTCGGTCAGAAAATGAAGAGTTTGTTGAAGTGGGAAGATTGGGGCCTTCT
GATTATTTTGGTGAAATTGCACTACTGATGAATCGTCCTCGTGCTGCCAC
AGTTGTTGCTCGTGGCCCCTTGAAGTGCGTTAAGCTGGACCGACCTAGAT
TTGAACGTGTTCTTGGCCCATGCTCAGACATCCTCAAACGAAACATCCAG
CAGTACAACAGTTTTGTGTCACTGTCTGTCTGAAATCTGCCTCCTGTGCC
TCCCTTTTCTCCTCTCCCCAATCCATGCTTCACTCATGCAAACTGCTTTA
TTTTCCCTACTTGCAGCGCCAAGTGGCCACTGGCATCGCAGCTTCCTGTC
TGTTTATATATTGAAAGTTGCTTTTATTGCACCATTTTCAATTTGGAGCA
TTAACTAAATGCTCATACACAGTTAAATAAATAGAAAGAGTTCTATGGAG
ACTTTGCTGTTACTGCTTCTCTTTGTGCAGTGTTAGTATTCACCCTGGGC
AGTGAGTGCCATGCTTTTTGGTGAGGGCAGATCCAGCACCTATTGAATTA
CCATAGAGTAATGATGTAACAGTGCAAGATTTTTTTTTTTAAGTGACATA
ATTGTCCAGTTATAAGCGTATTTAGACTGTGGCCATATATGCTGTATTTC
TTTGTAGAATAAATGGTTTCTCATTAAACTCTAAAGATTAGGGAAATGGA
TATAGAAAATCTTAGTATAGTAGAAAGACATCTGCCTGTAATTAAACTAG
TTTAAGGGTGGAAAAATGAAAATTTTTGCTAATTATCAATGGGATATGAT
TGGTTCAGTTTTTTTTTTCCAGAGTTGTTGTTTGCCAAGCTAATCTGCCT
GGTTTATTTATATCTTGTTATTAATGTTTCTTCTCCAATTCTGAAATACT
TTTGAGTATGGCTATCTATACCTGCCTTTTAAGTTTGAAACTAACTCATA
GATGCAAATATTGGTTAGTATTTAACTACATCTGCCTCGGCTCACAAATT
CCGATTAGACCTTTATCCAGCTAGTGCCAAATAATTGATCAGATGCTGAA
TTGAGAATAAGAATTTGAGGTCTACATTCTTGGTTGTTAATTTAGAGCGT
TTGGTTAAAGTATGTCCTTCAGCTGACTCCAGTATAATCTCCTCTGCTCA
TTAAACTGATTCCAGGAGATTGGATTTGCTGTGACTAGATACAGATGGAG
CAAATGTCCTAACAGAGAAATAGAGGTGATGCTGCTAAAGGGAGAAATGC
CAGGCGGACAAAGTTCAGTGTCGGGAATTTTCCCCGTGACATTCACTGGG
GCATGAGATTTTGGAAGAAGTTTTTTACTTTGGTTTAGTCTTTTTTTCCT
CCTTTTTATTCAGCTAGAATTTCTGGTGGGTTGATGGTAGGGTATAATGT
GTCTGTGTTGCTTCAAATTGGTCTGAAAGGCTATCCTGCTGAAAGTCCTG
CTTTCCTATCTAGCATTTATTCCTCTGGCAAACTTTTCTTTCTTTTCTTT
TTTAAAGTAAACTTGTGTATTGAGTCTTAACTGTATTTCAGTATTTTCCA
GCCTTATGTGTTACATTATTCCAATGATACCCAACAGTTTATTTTTATTA
TTTTTTTAAACAAAATTTCACAGTTCTGTAATGTAGGCACTTTTATTTTC
ATTGTGATTTATATATAAGGTAATGTAGGGTTATATTTGGGAGTGACTGC
AAGCATTTTTCCATCTGTGTGCAACTAACTGACTCTGTTATTGATCCCTT
CTCCTGCCCTTTCCCAGGTAATTTAAATTGGTCATGGTAGATTTTTTTCA
TAGATTTGAAAAACTTTTAGGTTGTTACCAAGTATGAAGTATAAATCTGG
GGAAGAGGTTTTATTTACATTTTAGGGTGGGTAAGAAAGCCACCTTGTTA
CAAATTTTTTAATTTCCAAAATAATCTATATTAAATGAGGGTTTCTGATC
TGTACTTTGTGTTTAGCTACCTTTTTATATTTAAAAAATTAAAAATGAAA
ATTATGTTCTTACAAGCTTAAAGCTTGATTTGATCT
Human PRKAR1A mRNA sequence - var7
(public gi:4884279)
TATTTTCCAGCCTTATGTGTTACATTATTCCAATGATACCCAACAGTTTA
TTTTTATTATTTTTTTAAACAAAATTTCACAGTTCTGTAATGTAGGCACT
TTTATTTTCATTGTGATTTATATATAAGGTAATGTAGGGTTATATTTGGG
AGTGACTGCAAGCATTTTTCCATCTGTGTGCAACTAACTGACTCTGTTAT
TGATCCCTTCTCCTGCCCTTTCCCAGGTAATTTAAATTGGTCATGGTAGA
TTTTTTTCATAGATTTGAAAAACTTTTAGGTTGTTACCAAGTATGAAGTA
TAAATCTGGGGAAGAGGTTTTATTTACATTTTAGGGTGGGTAAGAAAGCC
ACCTTGTTACAAATTTTTTAATTTCCAAAATAATCTATATTAAATGAGGG
TTTCTGATCTGTACTTTGTGTTTAGCTACCTTTTTATATTTAAAAAATTA
AAAATGAAAATTACGTTCTTACAAGCTTAAAGCTTGATTTGATCTTTGTT
TAAATGCCAAAATGTACTTAAATGAGTTACTTAGAATGCCATAAAATTGC
AGTTTCATGTATGTATATAATCATGCTCATGTATATTTAGTTACGTATAA
TGCTTTCTGAGTGAGTTTTACTCTTAAATCATTTGGTTAAATCATTTGGC
TTGCTGTTTACTCCCTTCTGTAGTTTTTAATTAAAAACTTTAAAGATAAG
TCTACATTAAACAATGATCACATCTAAAGCTTTATCTTTGTGTAATCTAA
GTATATGTGAGAAATCAGAATTGGCATAATTTGTCTTAGTTGATATTCAA
GGCTTTAAAAGTCATTATTCCTGGGCTTGGTAAGTGAATTTATGAGATTT
ACTGCTCTAGAAAGTATAGATGGCCAAAGGACCGTTATGTATTCCTTCCT
GATTACCAGTCTGATTATACCATGTGTGCTAATATACTTTTTTTGTTATA
GATTGTCTTAATGGTAGGTCAAGTAATAAAAAGAGATGAAATAATTTAAA
AAAAAAAAAA
Human PRKAR1A Protein sequence - var1
(public gi:1658306)
MESGSTAASEEARSLRECELYVQKHNIQALLKDSIVQLCTARPERPMAFL
REYFERLEKEEAKQIQNLQKAGTRTDSREDEISPPPP
Human PRKAR1A Protein sequence - var2
(public gi:23273780)
MESGSTAASEEARSLRECELYVQKHNIQALLKDSIVQLCTARPERPMAFL
REYFERLEKEEAKQIQNLQKAGTRTDSREDEISPPPPNPVVKGRRRRGAI
SAEVYTEEDAASYVRKVIPKDYKTMAALAKAIEKNVLFSHLDDNERSDIF
DAMFSVSFIAGETVIQQGDEGDNFYVIDQGETDVYVNNEWATSVGEGGSF
GELALIYGTPRAATVKAKTNVKLWGIDRDSYRRILMGSTLRKRKMYEEFL
SKVSILESLDKWERLTVADALEPVQFEDGQKIVVQGEPGDEFFIILEGSA
AVLQRRSENEEFVEVGRLGPSDYFGEIALLMNRPRAATVVARGPLKCVKL
DRPRFERVLGPCSDILKRNIQQYNSFVSLSV
Human PRKACA mRNA sequence - var1
(public gi:24980835)
TCGGGCTGAGGTTCCCGGGCGGGCGGGCGCGGAGAGACGCGGGAAGCAGG
GGCTGGGCGGGGGTCGCGGCGCCGCAGCTAGCGCAGCCAGCCCGAGGGCC
GCCGCCGCCGCCGCCCAGCGCGCTCCGGGGCCGCCGGCCGCAGCCAGCAC
CCGCCGCGCCGCAGCTCCGGGACCGGCCCCGGCCGCCGCCGCCGCGATGG
GCAACGCCGCCGCCGCCAAGAAGGGCAGCGAGCAGGAGAGCGTGAAAGAA
TTCTTAGCCAAAGCCAAAGAAGATTTTCTTAAAAAATGGGAAAGTCCCGC
TCAGAACACAGCCCACTTGGATCAGTTTGAACGAATCAAGACCCTCGGCA
CGGGCTCCTTCGGGCGGGTGATGCTGGTGAAACACAAGGAGACCGGGAAC
CACTATGCCATGAAGATCCTCGACAAACAGAAGGTGGTGAAACTGAAACA
GATCGAACACACCCTGAATGAAAAGCGCATCCTGCAAGCTGTCAACTTTC
CGTTCCTCGTCAAACTCGAGTTCTCCTTCAAGGACAACTCAAACTTATAC
ATGGTCATGGAGTACGTGCCCGGCGGGGAGATGTTCTCACACCTACGGCG
GATCGGAAGGTTCAGTGAGCCCCATGCCCGTTTCTACGCGGCCCAGATCG
TCCTGACCTTTGAGTATCTGCACTCGCTGGATCTCATCTACAGGGACCTG
AAGCCGGAGAATCTGCTCATTGACCAGCAGGGCTACATTCAGGTGACAGA
CTTCGGTTTCGCCAAGCGCGTGAAGGGCCGCACTTGGACCTTGTGCGGCA
CCCCTGAGTACCTGGCCCCTGAGATTATCCTGAGCAAAGGCTACAACAAG
GCCGTGGACTGGTGGGCCCTGGGGGTTCTTATCTATGAAATGGCCGCTGG
CTACCCGCCCTTCTTCGCAGACCAGCCCATCCAGATCTATGAGAAGATCG
TCTCTGGGAAGGTGCGCTTCCCTTCCCACTTCAGCTCTGACTTGAAGGAC
CTGCTGCGGAACCTCCTGCAGGTAGATCTCACCAAGCGCTTTGGGAACCT
CAAGAATGGGGTCAACGATATCAAGAACCACAAGTGGTTTGCCACAACTG
ACTGGATTGCCATCTACCAGAGGAAGGTGGAAGCTCCCTTCATACCAAAG
TTTAAAGGCCCTGGGGATACGAGTAACTTTGACGACTATGAGGAAGAAGA
AATCCGGGTCTCCATCAATGAGAAGTGTGGCAAGGAGTTTTCTGAGTTTT
AGGGGCATGCCTGTGCCCCCATGGGTTTTCTTTTTTCTTTTTTCTTTTTT
TTGGTCGGGGGGGTGGGAGGGTTGGATTGAACAGCCAGAGGGCCCCAGAG
TTCCTTGCATCTAATTTCACCCCCACCCCACCCTCCAGGGTTAGGGGGAG
CAGGAAGCCCAGATAATCAGAGGGACAGAAACACCAGCTGCTCCCCCTCA
TCCCCTTCACCCTCCTGCCCCCTCTCCCACTTTTCCCTTCCTCTTTCCCC
ACAGCCCCCCAGCCCCTCAGCCCTCCCAGCCCACTTCTGCCTGTTTTAAA
CGAGTTTCTCAACTCCAGTCAGACCAGGTCTTGCTGGTGTATCCAGGGAC
AGGGTATGGAAAGAGGGGCTCACGCTTAACTCCAGCCCCCACCCACACCC
CCATCCCACCCAACCACAGGCCCCACTTGCTAAGGGCAAATGAACGAAGC
GCCAACCTTCCTTTCGGAGTAATCCTGCCTGGGAAGGAGAGATTTTTAGT
GACATGTTCAGTGGGTTGCTTGCTAGAATTTTTTTAAAAAAACAACAATT
TAAAATCTTATTTAAGTTCCACCAGTGCCTCCCTCCCTCCTTCCTCTACT
CCCACCCCTCCCATGTCCCCCCATTCCTCAAATCCATTTTAAAGAGAAGC
AGACTGACTTTGGAAAGGGAGGCGCTGGGGTTTGAACCTCCCCGCTGCTA
ATCTCCCCTGGGCCCCTCCCCGGGGAATCCTCTCTGCCAATCCTGCGAGG
GTCTAGGCCCCTTTAGGAAGCCTCCGCTCTCTTTTTCCCCAACAGACCTG
TCTTCACCCTTGGGCTTTGAAAGCCAGACAAAGCAGCTGCCCCTCTCCCT
GCCAAAGAGGAGTCATCCCCCAAAAAGACAGAGGGGGAGCCCCAAGCCCA
AGTCTTTCCTCCCAGCAGCGTTTCCCCCCAACTCCTTAATTTTATTCTCC
GCTAGATTTTAACGTCCAGCCTTCCCTCAGCTGAGTGGGGAGGGCATCCC
TGCAAAAGGGAACAGAAGAGGCCAAGTCCCCCCAAGCCACGGCCCGGGGT
TCAAGGCTAGAGCTGCTGGGGAGGGGCTGCCTGTTTTACTCACCCACCAG
CTTCCGCCTCCCCCATCCTGGGCGCCCCTCCTCCGCTTAGCTGTCAGCTG
TCCATCACCTCTCCCCCACTTTTCTCATTTGTGCTTTTTTCTCTCGTAAT
AGAAAAGTGGGGAGCCGCTGGGGAGCCACCCCATTCATCCCCGTATTTCC
CCCTCTCATAACTTCTCCCCATCCCAGGAGGAGTTCTCAGGCCTGGGGTG
GGGCCCCGGGTGGGTGCGGGGGCGATTCAACCTGTGTGCTGCGAAGGACG
AGACTTCCTCTTGAACAGTGTGCTGTTGTAAACATATTTGAAAACTATTA
CCAATAAAGTTTTGTTTAAAAAAAAAAAAAAAAAA
Human PRKACA mRNA sequence - var2
(public gi:8489237)
GGTGCCCTGAGAACAGGACTGAGTGATGGCTTCCAACTCCAGCGATGTGA
AAGAATTCTTAGCCAAAGCCAAAGAAGATTTTCTTAAAAAATGGGAAAGT
CCCGCTCAGAACACAGCCCA
Human PRKACA mRNA sequence - var3
(public gi:4506054)
CAGTGNGCTCCGGGCCGCCGGCCGCAGCCAGCACCCGCCGCGCCGCAGCT
CCGGGACCGGCCCCGGCCGCCGCCGCCGCGATGGGCAACGCCGCCGCCGC
CAAGAAGGGCAGCGAGCAGGAGAGCGTGAAAGAATTCTTAGCCAAAGCCA
AAGAAGATTTTCTTAAAAAATGGGAAAGTCCCGCTCAGAACACAGCCCAC
TTGGATCAGTTTGAACGAATCAAGACCCTCGGCACGGGCTCCTTCGGGCG
GGTGATGCTGGTGAAACACAAGGAGACCGGGAACCACTATGCCATGAAGA
TCCTCGACAAACAGAAGGTGGTGAAACTGAAACAGATCGAACACACCCTG
AATGAAAAGCGCATCCTGCAAGCTGTCAACTTTCCGTTCCTCGTCAAACT
CGAGTTCTCCTTCAAGGACAACTCAAACTTATACATGGTCATGGAGTACG
TGCCCGGCGGGGAGATGTTCTCACACCTACGGCGGATCGGAAGGTTCAGT
GAGCCCCATGCCCGTTTCTACGCGGCCCAGATCGTCCTGACCTTTGAGTA
TCTGCACTCGCTGGATCTCATCTACAGGGACCTGAAGCCGGAGAATCTGC
TCATTGACCAGCAGGGCTACATTCAGGTGACAGACTTCGGTTTCGCCAAG
CGCGTGAAGGGCCGCACTTGGACCTTGTGCGGCACCCCTGAGTACCTGGC
CCCTGAGATTATCCTGAGCAAAGGCTACAACAAGGCCGTGGACTGGTGGG
CCCTGGGGGTTCTTATCTATGAAATGGCCGCTGGCTACCCGCCCTTCTTC
GCAGACCAGCCCATCCAGATCTATGAGAAGATCGTCTCTGGGAAGGTGCG
CTTCCCTTCCCACTTCAGCTCTGACTTGAAGGACCTGCTGCGGAACCTCC
TGCAGGTAGATCTCACCAAGCGCTTTGGGAACCTCAAGAATGGGGTCAAC
GATATCAAGAACCACAAGTGGTTTGCCACAACTGACTGGATTGCCATCTA
CCAGAGGAAGGTGGAAGCTCCCTTCATACCAAAGTTTAAAGGCCCTGGGG
ATACGAGTAACTTTGACGACTATGAGGAAGAAGAAATCCGGGTCTCCATC
AATGAGAAGTGTGGCAAGGAGTTTTCTGAGTTTTAGGGGCATGCCTGTGC
CCCCATGGGTTTTCTTTTTTCTTTTTTCTTTTTTTTGGTCGGGGGGGTGG
GAGGGTTGGATTGAACAGCCAGAGGGCCCCAGAGTTCCTTGCATCTAATT
TCACCCCCACCCCACCCTCCAGGGTTAGGGGGAGCAGGAAGCCCAGATAA
TCAGAGGGACAGAAACACCAGCTGCTCCCCCTCATCCCCTTCACCCTCCT
GCCCCCTCTCCCACTTTTCCCTTCCTCTTTCCCCACAGCCCCCCAGCCCC
TCAGCCCTCCCAGCCCACTTCTGCCTGTTTTAAACGAGTTTCTCAACTCC
AGTCAGACCAGGTCTTGCTGGTGTATCCAGGGACAGGGTATGGAAAGAGG
GGCTCACGCTTAACTCCAGCCCCCACCCACACCCCCATCCCACCCAACCA
CAGGCCCCACTTGCTAAGGGCAAATGAACGAAGCGCCAACCTTCCTTTCG
GAGTAATCCTGCCTGGGAAGGAGAGATTTTTAGTGACATGTTCAGTGGGT
TGCTTGCTAGAATTTTTTTAAAAAAACAACAATTTAAAATCTTATTTAAG
TTCCACCAGTGCCTCCCTCCCTCCTTCCTCTACTCCCACCCCTCCCATGT
CCCCCCATTCCTCAAATCCATTTTAAAGAGAAGCAGACTGACTTTGGAAA
GGGAGGCGCTGGGGTTTGAACCTCCCCGCTGCTAATCTCCCCTGGGCCCC
TCCCCGGGGAATCCTCTCTGCCAATCCTGCGAGGGTCTAGGCCCCTTTAG
GAAGCCTCCGCTCTCTTTTTCCCCAACAGACCTGTCTTCACCCTTGGGCT
TTGAAAGCCAGACAAAGCAGCTGCCCCTCTCCCTGCCAAAGAGGAGTCAT
CCCCCAAAAAGACAGAGGGGGAGCCCCAAGCCCAAGTCTTTCCTCCCAGC
AGCGTTTCCCCCCAACTCCTTAATTTTATTCTCCGCTAGATTTTAACGTC
CAGCCTTCCCTCAGCTGAGTGGGGAGGGCATCCCTGCAAAAGGGAACAGA
AGAGGCCAAGTCCCCCCAAGCCACGGCCCGGGGTTCAAGGCTAGAGCTGC
TGGGGAGGGGCTGCCTGTTTTACTCACCCACCAGCTTCCGCCTCCCCCAT
CCTGGGCGCCCCTCCTCCAGCTTAGCTGTCAGCTGTCCATCACCTCTCCC
CCACTTTCTCATTTGTGCTTTTTTCTCTCGTAATAGAAAAGTGGGGAGCC
GCTGGGGAGCCACCCCATTCATCCCCGTATTTCCCCCTCTCATAACTTCT
CCCCATCCCAGGAGGAGTTCTCACGCCTGGGGTGGGGCCCCGGGTGGGTG
CGGGGGCGATTCAACCTGTGTGCTGCGAAGGACGAGACTTCCTCTTGAAC
AGTGTGCTGTTGTAAACATATTTGAAAACTATTACCAATAAAGTTTGTT
Human PRKACA mRNA sequence - var4
(public gi: 189966)
GAATTCTTAGCCAAAGCCAAAGAAGATTTTCTTAAAAAATGGGAAAGTCC
CGCTCAGAACACAGCCCACTTGGATCAGTTTGAACGAATCAAGACCCTCG
GCACGGGCTCCTTCGGGCGGGTGATGCTGGTGAAACACAAGGAGACCGGG
AACCACTATGCCATGAAGATCCTCGACAAACAGAAGGTGGTGAAACTGAA
ACAGATCGAACACACCCTGAATGAAAAGCGCATCCTGCAAGCTGTCAACT
TTCCGTTCCTCGTCAAACTCGAGTTCTCCTTCAAGGACAACTCAAACTTA
TACATGGTCATGGAGTACGTGCCCGGCGGGGAGATGTTCTCACACCTACG
GCGGATCGGAAGGTTCAGTGAGCCCCATGCCCGTTTCTACGCGGCCCAGA
TCGTCCTGACCTTTGAGTATCTGCACTCGCTGGATCTCATCTACAGGGAC
CTGAAGCCGGAGAATCTGCTCATTGACCAGCAGGGCTACATTCAGGTGAC
AGACTTCGGTTTCGCCAAGCGCGTGAAGGGCCGCACTTGGACCTTGTGCG
GCACCCCTGAGTACCTGGCCCCTGAGATTATCCTGAGCAAAGTAGGAGCC
TCCCCAGCCCTCCCCTTCCCCTGAGGCCGGCTCTGCTCTCCTGCTCTCGC
CTCCTCCTCACCCTGTGCCCCCCCATCTTGCTCCAGGGCTACAACAAGGC
CGTGGACTGGTGGGCCCTGGGGGTTCTTATCTATGAAATGGCCGCTGGCT
ACCCGCCCTTCTTCGCAGACCAGCCCATCCAGATCTATGAGAAGATCGTC
TCTGGGAAGGTGAGGTCCGGATGTGGGACACAGCCCTGGAAGAAACAGAC
CGTTCCCTGCTCACCCATCCTATTCCCTGGGGAGCCCTGCTTGTTGTCAG
AATAATCTAGAAGTTCCTTAAAAAAAAAAAAAAAAA
Human PRKACA mRNA sequence - var5
(public gi:11493950)
TGAGAACAGGACTGAGTGATGGCTTCCAACTCCAGCGATGTGAAAGAATT
CTTAGCCAAAGCCAAAGAAGATTTTCTTAAAAAATGGGAAAGTCCCGCTC
AGAACACAGCCCACTTGGATCAGTTTGAACGAATCAAGACCCTCGGCACG
GGCTCCTTCGGGCGGGTGATGCTGGTGAAACACAAGGAGACCGCGAACCA
CTATGCCATGAAGATCCTCGACAAACAGAAGGTGGTGAAACTGAAACAGA
TCGAACACACCCTGAATGAAAAGCGCATCCTGCAAGCTGTCAACTTTCCG
TTCCTCGTCAAACTCGAGTTCTCCTTCAAGGACAACTCAAACTTATACAT
GGTCATGGAGTACGTGCCCGGCGGGGAGATGTTCTCACACCTACGGCGGA
TCGGAAGGTTCAGTGAGCCCCATGCCCGTTTCTACGCGGCCCAGATCGTC
CTGACCTTTGAGTATCTGCACTCGCTGGATCTCATCTACAGGGACCTGAA
GCCGGAGAATCTGCTCATTGACCAGCAGGGCTACATTCAGGTGACAGACT
TCGGTTTCGC
Human PRKACA mRNA sequence - var6
(public gi:8568080)
CCCAGTGGCCTCTGGGTTGGGTTTCTCTTCCTGCTCCCACCCCACGGCTC
CCTAGCTCCCCCTGCAGGCAGGGTTCTGGGGACAGACAGCCGAACAGACA
CGGCAGGTCTCATGAGCCTTCCCAGCCACCGTAGTGCCGGTGCCCTGAGA
ACAGGACTGAGTGATGGCTTCCAACTCCAGCGATGTGAAAGAATTCTTAG
CCAAAGCCAAAGAAGATTTTCTTAAAAAATGGGAAAGTCCCGCTCAGAAC
ACAGCCCACTTGGATCAGTTTGAACGAATCAAGACCCTCGGCACGGGCTC
CTTCGGGCGGGTGATGCTGGTGAAACACAAGGAGACCGGGAACCACTATG
CCATGAAGATCCTCGACAAACAGAAGGTGGTGAAACTGAAACAGATCGAA
CACACCCTGAATGAAAAGCGCATCCTGCAAGCTGTCAACTTTCCGTTCCT
CGTCAAACTCGAGTTCTCCTTCAAGGACAACTCAAACTTATACATGGTCA
TGGAGTACGTGCCCGGCGGGGAGATGTTCTCACACCTACGGCGGATCGGA
AGGTTCAGTGAGCCCCATGCCCGTTTCTACGCGGCCCAGATCGT
Human PRKACA Protein sequence - var1
(public gi:189967)
EFLAKAKEDFLKKWESPAQNTAHLDQFERIKTLGTGSFGRVMLVKHKETG
NHYAMKILDKQKVVKLKQIEHTLNEKRILQAVNFPFLVKLEFSFKDNSNL
YMVMEYVPGGEMFSHLRRIGRFSEPHARFYAAQIVLTFEYLHSLDLIYRD
LKPENLLIDQQGYIQVTDFGFAKRVKGRTWTLCGTPEYLAPEIILSKVGA
SPALPFP
Human PRKACA Protein sequence - var2
(public gi:11493951)
MASNSSDVKEFLAKAKEDFLKKWESPAQNTAHLDQFERIKTLGTGSFGRV
MLVKHKETGNHYANKILDKQKVVKLKQIEHTLNEKRILQAVNFPFLVKLE
FSFKDNSNLYMVMEYVPGGEMFSHLRRIGRFSEPHARFYAAQIVLTFEYL
HSLDLIYRDLKPENLLIDQQGYIQVTDFGFA
Human PRKACA Protein sequence - var3
(public gi:8568081)
MASNSSDVKEFLAKAKEDFLKKWESPAQNTAHLDQFERIKTLGTGSFGRV
MLVKHKETGNHYAMKILDKQKVVKLKQIEHTLNEKRILQAVNFPFLVKLE
FSFKDNSNLYMVMEYVPGGEMFSHLRRIGRFSEPHARFYAAQIV
Human PRKACA Protein sequence - var4
(public gi:8489238)
MASNSSDVKEFLAKAKEDFLKKWESPAQNTA
Human PRKACA Protein sequence - var5
(public gi:24980836)
MGNAAAAKKGSEQESVKEFLAKAKEDFLKKWESPAQNTAHLDQFERIKTL
GTGSFGRVMLVKHKETGNHYAMKILDKQKVVKLKQIEHTLNEKRILQAVN
FPFLVKLEFSFKDNSNLYMVMEYVPGGEMFSHLRRIGRFSEPHARPYAAQ
IVLTFEYLHSLDLIYRDLKPENLLIDQQGYIQVTDFGFAKRVKGRTWTLC
GTPEYLAPEIILSKGYNKAVDWWALGVLIYEMAAGYPPFFADQPIQIYEK
IVSGKVRFPSHFSSDLKDLLRNLLQVDLTKRFGNLKNGVNDIKNHKWFAT
TDWIAIYQRKVEAPFIPKFKGPGDTSNFDDYEEEEIRVSINEKCGKEFSE
F
Human PRKACB mRNA sequence - var1
(public gi:23272312)
AGCGGGTCTGCCCGCCGCCGCCACTGCTGCTCCCACCGCCGTCGCCGCCG
CCGCCGCCGCCGCCACTGCTGCTGCCGGTGCTAAGGAGTTCGCTGGAGCC
CTTTCCTCAGACCCGGCCCGGTCTTCGCGCCCGGACTCCTGGCGCCAGCG
CTAGGCGCACTCACCGCTCTGACGGGTGCAGACGCGGGAGTTGTCCCAGA
CTGTGGAGTGGCGGGCACGGCCCCAGCTCCCCTTCCGTTCCCTGACCCCT
TCTTGCCATCCGCCCAGACATGGGGAACGCGGCGACCGCCAAGAAAGGCA
GCGAGGTGGAGAGCGTGAAAGAGTTTCTAGCCAAAGCCAAAGAAGACTTT
TTGAAAAAATGGGAGAATCCAACTCAGAATAATGCCGGACTTGAAGATTT
TGAAAGGAAAAAAACCCTTGGAACAGGTTCATTTGGAAGAGTCATGTTGG
TAAAACACAAAGCCACTGAACAGTATTATGCCATGAAGATCTTAGATAAG
CAGAAGGTTGTTAAACTGAAGCAAATAGAGCATACTTTGAATGAGAAAAG
AATATTACAGGCAGTGAATTTTCCTTTCCTTGTTCGACTGGAGTATGCTT
TTAAGGATAATTCTAATTTATACATGGTTATGGAATATGTCCCTGGGGGT
GAAATGTTTTCACATCTAAGAAGAATTGGAAGGTTCAGTGAGCCCCATGC
ACGGTTCTATGCAGCTCAGATAGTGCTAACATTCGAGTACCTCAATTCAC
TAGACATCATCTACAGAGATCTAAAACCTGAAAATCTCTTAATTGACCAT
CAAGGCTATATCCAGGTCACAGACTTTGGGTTTGCCAAAAGAGTTAAAGG
CAGAACTTGGACATTATGTGGAACTCCAGAGTATTTGGCTCCAGAAATAA
TTCTCAGCAAGGGCTACAATAAGGCAGTGGATTGGTGGGCATTAGGAGTG
CTAATCTATGAAATGGCAGCTGGCTATCCCCCATTCTTTGCAGACCAACC
AATTCAGATTTATGAAAAGATTGTTTCTGGAAAGGTCCGATTCCCATCCA
ACTTCAGTTCAGATCTCAAGGACCTTCTACGGAACCTGCTGCAGGTGGAT
TTGACCAAGAGATTTGGAAATCTAAAGAATGGTGTCAGTGATATAAAAAC
TCACAAGTGGTTTGCCACGACAGATTGGATTGCTATTTACCAGAGGAAGG
TTGAAGCTCCATTCATACCAAAGTTTAGAGGCTCTGGAGATACCAGCAAC
TTTGATGACTATGAAGAAGAAGATATCCGTGTCTCTATAACAGAAAAATG
TGCAAAAGAATTTGGTGAATTTTAAAGAGGAACAAGATGACATCTGAGCT
CACACTCAGTGTTTGCACTCTGTTGAGAGATAAGGTAGAGCTGAGACCGT
CCTTGTTGAAGCAGTTACCTAGTTCCTTCATTCCAACGACTGAGTGAGGT
CTTTATTGCCATCATCCCGTGTGCGCACTCTGCATCCACCTATGTAACAA
GGCACCGCTAAGCAAGCATTGTCTGTGCCATAACACAGTACTAGACCACT
TTCTTACTTCTCTTTGGGTTGTCTTTCTCCTCTCCTATATCCATTTCTTC
CTTTTCCAATTTCATTGGTTTTCTCTAAACAGTGCTCCATTTTATTTTGT
TGGTGTTTCAGATGGGCAGTGTTATGGCTACGTGATATTTGAAGGGAAGG
ATAAGTGTTGCTTTCAGTAGTTATTGCCAATATTGTTGTTGGTCAATGGC
TTGAAGATAAACTTTCTAATAATTATTATTTCTTTGAGTAGCTCAGACTT
GGTTTTGCCAAAACTCTTGGTAATTTTTGAAGATAGACTGTCTTATCACC
AAGGAAATTTATACAAATTAAGACTAACTTTCTTGGAATTCACTATTCTG
GCAATAAATTTTGGTAGACTAATACAGTACAGCTAGACCCAGAAATTTGG
AAGGCTGTAGATCAGAGGTTCTAGTTCCCTTTCCCTCCTTTTATATCCTC
CTCTCCTTGAGTAATGAAGTGACCAGCCTGTGTAGTGTGACAAACGTGTC
TCATTCAGCAGGAAAAACTAATGATATGGATCATCACCCAGATTCTCTCA
CTTGGTACCAGCATTTCTGTAGGTATTAGAGAAGAGTTCTAAGTTTTCTA
AACCTTAACTGTTCCTTAAGGATTTTAGCCAGTATTTTAATAGAACATGA
TTAATGAAAGTGACAAATTTTAAATTTTCTCTAATAGTCCTCATCATAAA
CTTTTTAAAGGAAAATAAGCAAACTAAAAAGAACATTGGTTTAGATAAAT
ACTTATACTTTGCAAAGTCAAAAATGGCTTGATTTTTGGAAACAATATAG
AGGTATTCATATTTAAATGAGGGTTTACATTTGTTTTGTTTTGTAACCGT
TAAAAAGAAGTTGTTTCCAGCTAATTATTGTGGTGTACTATATTTGTGAG
CCTAGGGTAGGGGCACTGCTGCAACTTCTGCTTTCATCCCATGCCTCATC
AATGAGGAAAGGGAACAAAGTGTATAAAACTGCCACAATTGTATTTTAAT
TTTGAGGTATGATATTTTCAGATATTTCATAATTTCTAACCTCTGTTCTC
TCAGTAAACAGAATGTCTGATCGATCATGCAGATACAATGTTGGTATTTG
AGAGGTTAGTTTTTTTCCTACACTTTTTTTTGCCAACTGACTTAACAACA
TTGCTGTCAGGTGGAAATTTCAAGCACTTTTGCACATTTAGTTCAGTGTT
TGTTGAGAATCCATGGCTTAACCCACTTGTTTTGCTATTTTTTTCTTTGC
TTTTAATTTTCCCCATCTGATTTTATCTCTGCGTTTCAGTGACCTACCTT
AAAACAACACACGAGAAGAGTTAAACTGGGTTCATTTTAATGATCAATTT
ACCTGCATATAAAATTTATTTTTAATCAAGCTGATCTTAATGTATATAAT
CATTCTATTTGCTTTATTATCGGTGCAGGTAGGTCATTAACACCACTTCT
TTTCATCTGTACCACACCCTGGTGAAACCTTTGAAGACATAAAAAAAACC
TGTCTGAGATGTTCTTTCTACCAATCTATATGTCTTTCGGTTATCAAGTG
TTTCTGCATGGTAATGTCATGTAAATGCTGATATTGATTTCACTGGTCCA
TCTATATTTAAAACGTGCAAGAAAAAAATAAAATACTCTGCTCTAGCAAG
TTTTGTGTAACAAAGGCATATCGTCATGTTAATAAATTTAAAACATCATT
CGTATAAAATATTTTAATTTTCTTGTATTTCATTTAGACCCAAGAACATG
CTGACCAATGTGTTCTATATGTAAACTACAAATTCTATGGTAGCTTTGTT
GTATATTATTGTAAAATTATTTTAATAAGTCATGGGGATGACAATTTGAT
TATTACAATTTAGTTTTCAGTAATCAAAAAGATTTCTATGAATTCTAAAA
AATATTTTTTTCTATGAAATTACTAGTGCCCAGCTGTAGAATCTACCTTA
GGTAGATGATCCCTAGACATACGTTGGTTTTGAGGGCTATTCAGCCATTC
CATTTTACTCTCTATTTAAAGGCCGTGACCAAGCTTGTCATGAGCAAATA
TGTCAAGGGAGTCAATCTCTGACCAATCAACTACAGTAAATTAGAATATT
TTTAAAGTATGTAACATTCCCAGTTTCAGCCACAATTTAGCCAAGAATAA
GATAAAAACTTGAATAAGAAGTAAGTAGCATAAATCAGTATTTAACCTAA
AATTACATATTTGAAACAGAAGATATTATGTTATGCTCAGTAAATAATTA
AGAGATGGCATTGTGTAAGAAGGAGCCCTAGACTGAAAGTCAAGACATCT
GAATTTCAGGCTGGAAAACTATCAGTATGATCTCAGCCTCAGTTCTCTTG
TCTGTAAGATGGAAGAACTGGATTAGGCAGTTTGTAAGATTCCTCCTAAC
TTTCACAGTCGATGACAAGATTGTCTTTTTATCTGATATTTTGAAGGGTA
TATTGCTTTGAAGTAAGTCTCAATAAGGCAATATATTTTAGGGCATCTTT
CTTCTTATCTCTGACAGTGTTCTTAAAATTATTTGAATATCATAAGAGCC
TTGGTGTCTGTCCTAATTCCTTTCTCACTCACCGATGCTGAATACCCAGT
TGAATCAAACTGTCAACCTACCAAAAACGATATTGTGGCTTATGGGTATT
GCTGTCTCATTCTTGGTATATTCTTGTGTTAACTGCCCATTGGCCTGAAA
ATACTCATTGTAAGCCTGAAAAAAAAAATCTTTCCCACTGTTTTTTCTGC
TTGTTGTAAGAATCAAATGAAATAATGTATGTGAAAGCACCTTGTAAACT
GTAACCTATCAATGTAAAATGTTAAGGTGTGTTGTTATTTCATTAATTAC
TTCTTTGTTTAGAATGGAATTTCCTATGCACTACTGTAGCTAGGAAATGC
TGAAAACAACTGTGTTTTTTAATTAATCAATAACTGCAAAATTAAAGTAC
CTTCAATGGATAAGACAACAAAAAAAAAAAAAAAA
Human PRKACB mRNA sequence - var2
(public gi:4884447)
AAAAAAAATCTTTCCCACTGTTTTTTCTGCTTGTTGTAAGAATCAAATGA
AATAATGTATGTGAAAGCACCTTGTAAACTGTAACCTATCAATGTAAAAT
GTTAAGGTGTGTTGTTATTTCATTAATTACTTCTTTGTTTAGAATGGAAT
TTCCTATGCACTACTGTAGCTAGGAAATGCTGAAAACAACTGTGTTTTTT
AATTAATCAATAACTGCAAAATTAAAGTACCTTCAATGGATAAGACAAAA
AAAAAAAAAAAAAAAAAAAAAAAAAAAAAAAAAAAAAAAAAAAAAA
Human PRKACB mRNA sequence - var3
(public gi:21749785)
GTTATTTTGAGCAATATGTTTTGGAAAGGTTGGTTTTCATCATGAGTGCA
CGCAAATCATCAGATGCATCTGCTTGCTCCTCTTCAGAAATATCTGTGAA
AGAGTTTCTAGCCAAAGCCAAAGAAGACTTTTTGAAAAAATGGGAGAATC
CAACTCAGAATAATGCCCGACTTGAAGATTTTGAAAGGAAAAAAACCCTT
GGAACAGGTTCATTTGGAAGAGTCATGTTGGTAAAACACAAAGCCACTGA
ACAGTATTATGCCATGAAGATCTTAGATAAGCAGAAGGATAATTCTAATT
TATACATGGTTATGGAATATGTCCCTGGGGGTGAAATGTTTTCACATCTA
AGAAGAATTGGAAGGTTCAGTGAGCCCCATGCACGGTTCTATGCAGCTCA
GATAGTGCTAACATTCGAGTACCTCCATTCACTAGACCTCATCTACAGAG
ATCTAAAACCTGAAAATCTCTTAATTGACCATCAAGGCTATATCCAGGTC
ACAGACTTTGGGTTTGCCAAAAGAGTTAAAGGCAGAACTTGGACATTATG
TGGAACTCCAGAGTATTTGGCTCCAGAAATAATTCTCAGCAAGGGCTACA
ATAAGGCAGTGGATTGGTGGGCATTAGGAGTGCTAATCTATGAAATGGCA
GCTGGCTATCCCCCATTCTTTGCAGACCAACCAATTCAGATTTATGAAAA
GATTGTTTCTGGAAAGGTCCGATTCCCATCCCACTTCAGTTCAGATCTCA
AGGACCTTCTACGGAACCTGCTGCAGGTGGATTTGACCAAGAGATTTGGA
AATCTAAAGAATGGTGTCAGTGATATAAAAACTCACAAGTGGTTTGCCAC
GACAGATTGGATTGCTATTTACCAGAGGAAGGTTGAAGCTCCATTCATAC
CAAAGTTTAGAGGCTCTGGAGATACCAGCAACTTTCATGACTATGAAGAA
GAAGATATCCGTGTCTCTATAACAGAAAAATGTGCAAAAGAATTTGGTGA
ATTTTAAAGAGGAACAAGATGACATCTGAGCTCACACTCAGTGTTTGCAC
TCTGTTGAGAGATAAGGTAGAGCTGAGACCGTCCTTGTTGAAGCAGTTAC
CTAGTTCCTTCATTCCAACGACTGAGTGAGGTCTTTATTGCCATCATCCC
GTGTGCGCACTCTGCATCCACCTATGTAACAAGGCACCGCTAAGCAAGCA
TTGTCTGTGCCATAACACAGTACTAGACCACTTTCTTACTTCTCTTTGGG
TTGTCTTTCTCCTCTCCTACATCCATTTCTTCCTTTTCCAATTTCATTGG
TTTTCTCTAAACAGTGCTCCATTTTATTTTGTTGGTGTTTCAGATGGGCA
GTGTTATGGCTACGTGATATTTGAAGGGAAGGATAAGTGTTGCTTTCAGT
AGTTATTGCCAATATTGTTGTTGGTCAATGGCTTGAAGATAAACTTTCTA
ATAATTATTATTTCTTTGAGTAGCTCAGACTTGGTTTTGCCAAAACTCTT
GGTAATTTTTGAAGATAGACTGTCTTATCACCAAGGAAATTTATACAAAT
TAAGACTAACTTTCTTGGAATTCACTATTCTGGCAATAAATTTTGGTAGA
CTAATACAGTACAGCTAGACCCAGAAATTTGGAAGGCTGTAGATCAGAGG
TTCTAGTTCCCTTTCCCTCCTTTTATATCCTCCTCTCCTTGAGTAATGAA
GTGACCAGCCTGTGTAGTGTGACAAACGTGTCTCATTCAGCAGGAAAAAC
TAATGATATGGATCATCACCCAGATTCTCTCACTTGGTACCAGCATTTCT
GTAGGTATTAGAGAAGAGTTCTAAGTTTTCTAAACCTTAACTGTTCCTTA
AGGATTTTAGCCAGTATTTTAATAGAACATGATTAATGAAAGTGACAAAT
TTTAAATTTTCTCTAATAGTCCTCATCATAAACTTTTTAAAGGAAAATAA
GCAAACTAAAAAGAACATTGGTTTAGATAAATACTTATACTTTGCAAAGT
CAAAAATGGCTTGATTTTTGGAAACAATATAGAGGTATTCATATTTAAAT
GAGGGTTTACATTTGTTTTGTTTTGTAACCGTTAAAAAGAAGTTGTTTCC
AGCTAATTATTGTGGTGTACTATATTTGTGAGCCTAGGGTAGGGGCACTG
CTGCAACTTCTGCTTTCATCCCATGCCTCATCAATGAGGAAAGGGAACAA
AGTGTATAAAACTGCCACAATTGTATTTTAATTTTGAGGTATGATATTTT
CAGATATTTCATAATTTCTAACCTCTGTTCTCTCAGTAAACAGAATGTCT
GATCGATCATGCAGATACAATGTTGGTATTTGAGAGGTTAGTTTTTTTCC
TACACTTTTTTTTGCCAACTGACTTAACAACATTGCTGTCAGGTGGAAAT
TTCAAGCACTTTTGCACATTTAGTTCAGTGTTTGTTGAGAATCCATGGCT
TAACCCACTTGTTTTGCTATTTTTTTCTTTGCTTTTAATTTTCCCCATCT
GATTTTATCTCTGCGTTTCAGTGACCTACCTTAAAACAACACACGAGAAG
AGTTAAACTGGGTTCATTTTAATGATCAATTTACCTGCATATAAAATTTA
TTTTTAATCAAGCTGATCTTAATGTATATAATCATTCTATTTGCTTTATT
ATCGGTGCAGGTAGGTCATTAACACCACTTCTTTTCATCTGTACCACACC
CTGGTGAAACCTTTGAAGACATAAAAAAAACCTGTCTGAGATGTTCTTTC
TACCAATCTATATGTCTTTCGGTTATCAAGTGTTTCTGCATGGTAATGTC
ATGTAAATGCTGATATTGATTTCACTGGTCCATCTATATTTAAAACGTGC
Human PRKACB mRNA sequence - var4
(public gi:16740847)
GTTCGCTGGAGCCCTTTCCTCAGACCCGGCCCGGTCTTCGCGCCCGGACT
CCTGGCGCCAGCGCTAGGCGCACTCACCGCTCTGACGGGTGCAGACGCGG
GAGTTGTCCCAGACTGTGGAGTGGCGGGCACGGCCCCAGCCCCCCTTCCC
TTCCCTGACCCCTTCTTGCCATCGCCCCAGACATGGGGAACGCGGCGACC
GCCAAGAAAGGCAGCGAGGTGGAGAGCGTGAAAGAGTTTCTAGCCAAAGC
CAAAGAAGACTTTTTGAAAAAATGGGAGAATCCAACTCAGAATAATGCCG
GACTTGAAGATTTTGAAAGGAAAAAAACCCTTGGAACAGGTTCATTTGGA
AGAGTCATGTTGGTAAAACACAAAGCCACTGAACAGTATTATGCCATGAA
GATCTTAGATAAGCAGAAGGTTGTTAAACTGAAGCAAATAGAGCATACTT
TGAATGAGAAAAGAATATTACAGGCAGTGAATTTTCCTTTCCTTGTTCGA
CTGGAGTATGCTTTTAAGGATAATTCTAATTTATACATGGTTATGGAATA
TGTCCCTGGGGGTGAAATGTTTTCACATCTAAGAAGAATTGGAAGGTTCA
GTGAGCCCCATGCACGGTTCTATGCAGCTCAGATAGTGCTAACATTCGAG
TACCTCCATTCACTAGACCTCATCTACAGAGATCTAAAACCTGAAAATCT
CTTAATTGACCATCAAGGCTATATCCAGGTCACAGACTTTGGGTTTGCCA
AAAGAGTTAAAGGCAGAACTTGGACATTATGTGGAACTCCAGAGTATTTG
GCTCCAGAAATAATTCTCAGCAAGGGCTACAATAAGGCAGTGGATTGGTG
GGCATTAGGAGTGCTAATCTATGAAATGGCAGCTGGCTATCCCCCATTCT
TTGCAGACCAACCAATTCAGATTTATGAAAAGATTGTTTCTGGAAAGAAC
TTTTGATATGAACAAAACAAAACTTTGAGAAAAATTAACAGACAAGGCAG
TGATTTATTTTTGAAGAATTTGAGAAGTGTAGACTCTCAAGAGGACTAAA
GGTCATATGAAGAATGATGAGAGAACCAAAATACATTAAAATCACAAATG
GAAGAAGAATATTTTACTAATACAAAAACTAAGAATGTAAATGTTATAAT
AATTGTTTCAAATCATTTAATTGACAGTAATTATAAAGTTCTTGAATCTT
TACTATATTACTTTTATTTATACTTCATATAAGAAATCCAGTTTTCTAAC
AAGGATACTGTCATAACTAAATTTACATTTATTAAGAAAAACTGCTTTAG
TTAAAATTAATGTGTCTTCATTTTTATGCATTGGCCTCGATTTGCCAATC
ATTCTCTATTGGTTAAAATTTATATTCAGCTGTTTATGAATATATATTCA
TTTTATATCAAACTTTAAAATTTTGTATCTAATAATCAGCATATATTCTA
AAATCATAACAGTCTAAATCCTGGGCACCTTAGAAGAATGACACCAGAAA
ACCTTATTATATCACAATATTCTGTTTTCCCCTTCATTTATTTAGAAATA
TGACAGGATATTTGGTGTACTTTTGTTTTTTAACTAAAAGTACCAGATTC
TCTCTCCCCATGTGGGATATAAAATTATCCCCATCTCTTACTCCCTTTAC
TCATCTAAAGTAGAAGTCATGAAAGTGGAATTTTTGCCATTAAAAGGCTC
TGTATTATGTGAAGTTAGATTGTATTAACCATTTCCCAATAAATCATCTG
TTTCAAAACTCAAATTCAAACTAGAATGTGTCTCTATTCACATTGCAAAA
ATATTATTGTCTCTCTGGTTAGTGGCTAAAAGCCAAATTGGAAACTAACT
AGTTTTTTAAATTTTTTAAATTGTGCAAATTATTAAAAATCCAATTTGGT
CTTATAAAAAAAAAAAAAAAAAAAAAAAAAAAAAAAAAAAAAAAAAAAAA
A
Human PRKACB mRNA sequence - var5
(public gi:189982)
CCAGCCCCCCTTCCCTTCCCTGACCCCTTCTTGCCATCGCCCCAGACATG
GGGAACGCGGCGACCGCCAAGAAAGGCAGCGAGGTGGAGAGCGTGAAAGA
GTTTCTAGCCAAAGCCAAAGAAGACTTTTTGAAAAAATGGGAGAATCCAA
CTCAGAATAATGCCGGACTTGAAGATTTTGAAAGGAAAAAAACCCTTGGA
ACAGGTTCATTTGGAAGAGTCATGTTGGTAAAACACAAAGCCACTGAACA
GTATTATGCCATGAAGATCTTAGATAAGCAGAAGGTTGTTAAACTGAAGC
AAATAGAGCATACTTTGAATGAGAAAAGAATATTACAGGCAGTGAATTTT
CCTTTCCTTGTTCGACTGGAGTATGCTTTTAAGGATAATTCTAATTTATA
CATGGTTATGGAATATGTCCCTGGGGGTGAAATGTTTTCACATCTAAGAA
GAATTGGAAGGTTCAGTGAGCCCCATGCACGGTTCTATGCAGCTCAGATA
GTGCTAACATTCGAGTACCTCCATTCACTAGACCTCATCTACAGAGATCT
AAAACCTGAAAATCTCTTAATTGACCATCAAGGCTATATCCACGTCACAG
ACTTTGGGTTTGCCAAAAGAGTTAAAGGCAGAACTTGGACATTATGTGGA
ACTCCAGAGTATTTGGCTCCAGAAATAATTCTCAGCAAGGGCTACAATAA
GGCAGTGGATTGGTGGGCATTAGGAGTGCTAATCTATGAAATGGCAGCTG
GCTATCCCCCATTCTTTGCAGACCAACCAATTCAGATTTATGAAAAGATT
GTTTCTGGAAAGGTCCGATTCCCATCCCACTTCAGTTCAGATCTCAAGGA
CCTTCTACGGAACCTGCTGCAGGTGGATTTGACCAAGAGATTTGGAAATC
TAAAGAATGGTGTCAGTGATATAAAAACTCACAAGTGGTTTGCCACGACA
GATTGGATTGCTATTTACCAGAGGAAGGTTGAAGCTCCATTCATACCAAA
GTTTAGAGGCTCTGGAGATACCAGCAACTTTGATGACTATGAAGAAGAAG
ATATCCGTGTCTCTATAACAGAAAAATGTGCAAAAGAATTTGGTGAATTT
TAAAGAGGAACAAGATGACATCTGAGCTCACACTCAGTGTTTGCACTCTG
TTGAGAGATAAGGTAGAGCTGAGACCGTCCTTGTTGAAGCAGTTACCTAG
TTCCTTCATTCCAACGACTGAGTGAGGTCTTTATTGCCATCATCCGTGTG
CGCACTCTGCATCCACCTATGTAACAACGCACCGCTAAGCAAGCATTGTC
TGTGCCATAACACAGTACTAGACCACTTTCTTACTTCTCTTTGGGTTGTC
TTTCTCCTCTCCTACATCCATTTCTTCCTTTTCAATTTCATTGGTTTTCT
CTAAACAGTGCTCCATTTTATTTTGTTGGTGTTTCAGATGGGCAGTGTTA
TGGCTACGTGATATTTGAAGGGAAGGATAAGTGTTGCTTTCAGTAGTTAT
TGCCAATATTGTTGTTGGTCAATGGCTTGAAGATAAACTTTCTAATAATT
ATTATTTCTTTGAGTAGCTCAGACTTGGTTTTGCCAAAACTCTTGGTAAT
TTTTGAAGATAGACTGTCTTATCACCAAGGAAATTTATACAAATTAAGAC
TAACTTTCTTGGAATTCACTATTCTGGCAATAAATTTTGGTAGACTAATA
CAGTACAGCTAGACCCAGAAATTTGGAAGGCTGTAGATCAGAGGTTCTAG
TTCCCTTTCCCTCCTTTTATATCCTCCTCTCCTTGAGTAATGAAGTGACC
AGCCTGTGTAGTGTGACAAACGTGTCTCATTCAGCAGGAAAAACTAATGA
TATGGATCATCACCCAGATTCTCTCACTTGGTACCAGCATTTCTGTAGGT
ATTAGAGAAGAGTTCTAAGTTTTCTAAACCTTAACTGTTCCTTAAGGATT
TTAGCCAGTATTTTAATAGAACATGATTAATGAAAGTGACAAATTTTAAA
TTTTCTCTAATAGTCCTCATCATAAACTTTTTAAAGGAAAATAAGCAAAC
TAAAAAGAACATTGGTTTAGATAAATACTTATACTTTGCAAAGTCAAAAA
TGGCTTGATTTTTGGAAACAATATAGAGGTATTCATATTTAAATGAGGGT
TTACATTTGTTTTGTTTTGTAACCGTTAAAAAGAAGTTGTTTCCAGCTAA
TTATTGTGGTGTACTATATTTGTGAGCCTAGGGTAGGGGCACTGCTGCAA
CTTCTGCTTTCATCCCATGCCTCATCAATGAGGAAAGGGAACAAAGTGTA
TAAAACCTGCCACAATTGTATTTTAATTTTGAGGTATGATATTTTCAGAT
ATTTCATAATTTCTAACCTCTGTTCTCTCAGTAAACAGAATGTCTGATCG
ATCATGCAGATACAATGTTGGTATTTGAGAGGTTAGTTTTTTTCCTACAC
TTTTTTTTGCCAACTGACTTAACAACATTGCTGTCAGGTGGAAATTTCAA
GCACTTTTGCACATTTAGTTCAGTGTTTGTTGAGAATCCATGGCTTAACC
CACTTGTTTTGCTATTTTTTTCTTTGCTTTTAATTTTCCCCATCTGATTT
TATCTCTGCGTTTCAGTGACCTACCTTAAAACAACACACGAGAAGAGTTA
AACTGGGTTCATTTTAATGATCAATTTACCTGCATATAAAATTTATTTTT
AATCAAGCTGATCTTAATGTATATAATCATTCTATTTGCTTTATTATCGG
TGCAGGTAGGTCATTAACACCACTTCTTTTCATCTGTACCACACCCTGGT
GAAACCTTTGAAGACATAAAAAAAACCTGTCTGAGATGTTCTTTCTACCA
ATCTATATGTCTTTCGGTTATCAAGTGTTTCTGCATGGTAATGTCATGTA
AATGCTGATATTGATTTCACTGGTCCATCTATATTTAAAACGTGC
Human PRKACB Protein sequence - var1
(public gi:189983)
MGNAATAKKGSEVESVKEFLAKAKEDFLKKWENPTQNNAGLEDFERKKTL
GTGSFGRVMLVKHKATEQYYAMKILDKQKVVKLKQIEHTLNEKRILQAVN
FPFLVRLEYAFKDNSNLYMVMEYVPGGEMFSHLRRIGRFSEPHARFYAAQ
IVLTFEYLHSLDLIYRDLKPENLLIDHQGYIQVTDFGFAKRVKGRTWTLC
GTPEYLAPEIILSKGYNKAVDNWALGVLIYEMAAGYPPFFADQPIQIYEK
IVSGKVRFPSHFSSDLKDLLRNLLQVDLTKRFGNLKNGVSDIKTHKWFAT
TDWIAIYQRKVEAPFIPKFRGSGDTSNFDDYEEEDIRVSITEKCAKEFGE
F
Human PRKACB Protein sequence - var2
(public gi:16740848)
MGNAATAKKGSEVESVKEFLAKAKEDFLKKWENPTQNNAGLEDGERKKTL
GTGSFGRVMLVKHKATEQYYAMKILDKQKVVKLKQIEHTLNEKRILQAVN
FPFLVRLEYAFKDNSNLYMVMEYVPGGEMFSHLRRIGRFSEPHARFYAAQ
IVLTFEYLHSLDLIYRDLKPENLLIDHQGYIQVTDPGFAKRVKGRTWTLC
GTPEYLAPEIILSKGYNKAVDNWALGVLIYEMAAGYPPFFADQPIQIYEK
IVSGKNF
Human PRKACB Protein sequence - var3
(public gi:23272313)
MGNAATAKKGSEVESVKEFLAKAKEDFLKKWENPTQNNAGLEDFERKKTL
GTGSFGRVMLVKMKATEQYYAMKILDKQKVVKLKQIEHTLNEKRILQAVN
FPFLVRLEYAFKDNSNLYMVMEYVPGGEMFSHLRRIGRFSEPHARFYAAQ
IVLTPEYLNSLDIIYRDLKPENLLIDHQGYIQVTDFGFAKRVKGRTWTLC
GTPEYLAPEIILSKGYNKAVDWWALGVLIYEMAAGYPPFFADQPIQIYEK
IVSGKVRFPSNFSSDLKDLLRNLLQVDLTKRFGNLKNGVSDIKTHKWFAT
TDWIAIYQRKVEAPFIPKFRGSGDTSNFDDYEEEDIRVSITEKCAKEFGE
F

### Example 11

### Inhibition of PKA Kinase Activity Attenuates HIV-1 Virus Maturation

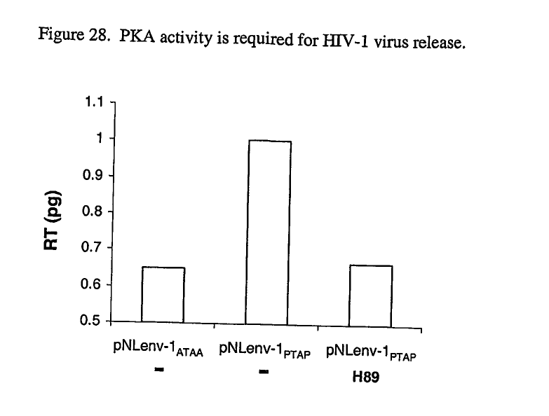

HeLa SS6 cells were transfected with pNLenv-1_{PTAP} or pNLenv-1_{ATAA} (L-domain mutant). Eighteen hours post-transfection, cells were transferred to 20° C. for two hours in order to inhibit transport of viral particles from the trans-Golgi (TGN) to the plasma membrane (PM). Subsequently, the PKA inhibitor, H89 (50 μM) (Biosource, Cat. No. PHZ1114) or DMSO were added to the cells and dishes were transferred to 37° C. to initiate transport from the TGN to the PM. Reverse transcriptase activity was assayed from virus-like-particles collected from cell supernatant twenty minutes later. H89 treatment resulted in complete inhibition of RT activity (FIG. 28: compare H89-treated to pNLenv-1_{ATAA} transfected cells to pNLenv-1_{PTAP}; left and right panels with middle panel, respectively). Thus, demonstrating that PKA activity is required for HIV-1 viral maturation.

Materials and Methods:

Cell Culture and Transfections

Hela SS6 cells were grown in Dulbecco's modified Eagle's medium (DMEM) supplemented with 10% heat-inactivated fetal calf serum and 100 units/ml penicillin and 100 μg/ml streptomycin. For transfections, HeLa SS6 cells were grown to 100% confluency in DMEM containing 10% FCS without antibiotics. Cells were then transfected with HIV-1_{NLenv1} (2 μg per 6-well) (Schubert et al., 1995).

Assays for Virus Release by RT Activity

Virus and virus-like particle (VLP) release by reverse transcriptase activity was determined one day after transfection with the pro-viral DNA as previously described (Adachi et al., 1986; Fukumori et al., 2000; Lenardo et al., 2002). The culture medium of virus-expressing cells was collected and centrifuged at 500×g for 10 minutes. The resulting supernatant was passed through a 0.45 μm-pore filter and the filtrate was centrifuged at 14,000×g for 2 hours at 4° C. The resulting supernatant was removed and the viral-pellet was re-suspended in cell solubilization buffer (50 mM Tris-HCl, pH7.8, 80 mM potassium chloride, 0.75 mM EDTA and 0.5% Triton X-100, 2.5 mM DTT and protease inhibitors). The corresponding cells were washed three times with phosphate-buffered saline (PBS) and then solubilized by incubation on ice for 15 minutes in cell solubilization buffer. The cell detergent extract was then centrifuged for 15 minutes at 14,000×g at 4° C. The sample of the cleared extract (normally 1:10 of the initial sample) were resolved on a 12.5% SDS-polyacrylamide gel, then transferred onto nitrocellulose paper and subjected to immunoblot analysis with rabbit anti-CA antibodies. The CA was detected after incubation with a secondary anti-rabbit antibody conjugated to Cy5 (Jackson Laboratories, West Grove, Pa.) and detected by fluorescence imaging (Typhoon instrument, Molecular Dynamics, Sunnyvale, Calif.). The Pr55 and CA were then quantified by densitometry. A colorimetric reverse transcriptase assay (Roche Diagnostics GmbH, Mannenheim, Germany) was used to measure reverse transcriptase activity in VLP extracts. RT activity was normalized to amount of Pr55 and CA produced in the cells.

### Example 12

### hPOSH is Phosphorylated by Protein Kinase A (PKA)

PKA is a cAMP-dependent kinase. The holoenzyme is a tetramer of two catalytic subunits (cPKA) bound to two regulatory subunits PRKR1 or PRKR2. Activation proceeds by the cooperative binding of two cAMP molecules to each R subunit, which causes the dissociation of each active C subunit from the R subunit dimer. The consensus sequence for phosphorylation by the C subunit is, stringently, K/R-R-X-S/TY and less stringently, R-X-X-S/TY, where Y tends to be a hydrophobic residue. The intracellular localization of PKA is controlled thorough association with A-kinase-anchoring proteins (AKAPs). The regulatory subunit of protein kinase A (PRKR1A) was identified as a POSH interactor by yeast-two-hybrid screen, thereby implicating POSH as an AKAP.

Protein kinase A was demonstrated to be required for the budding of transport vesicles from the TGN (Muniz et al., 1997, Proc Natl Acad Sci USA, 94:14461-6). Furthermore, it was demonstrated that an inhibitor of PKA, H89, is able to block HIV-1 release from cells (Cartier et al., 2003, J Biol Chem., 278:35211-9). Since POSH is localized at the TGN and is implicated as an AKAP, PRT3 may regulate PKA-mediated budding at the TGN of vesicles and HIV-1.

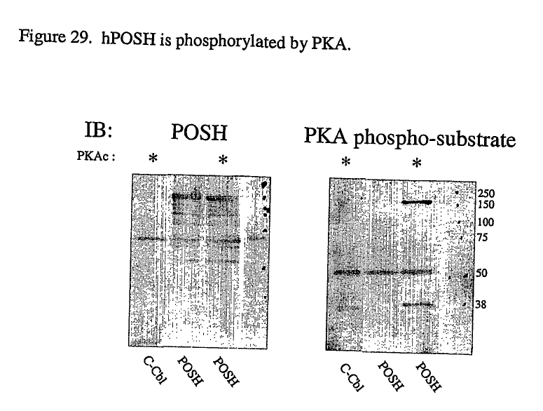

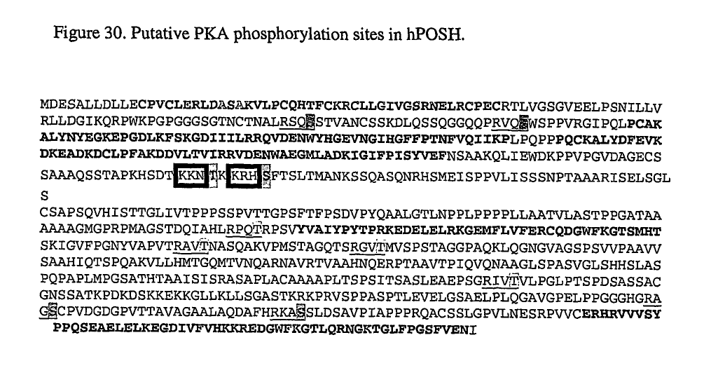

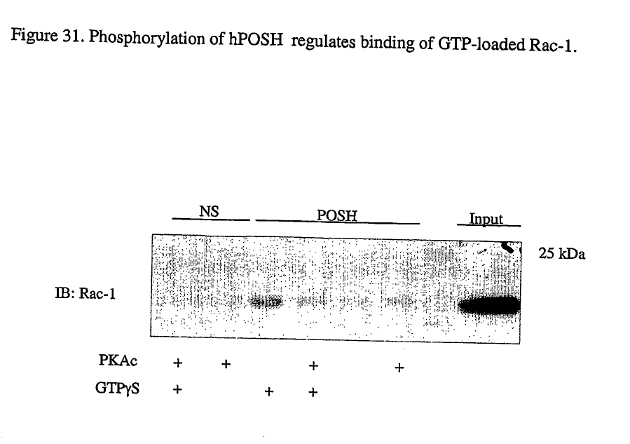

Applicants have demonstrated that POSH is phosphorylated by PKA (FIG. 29). Several putative PKA phosphorylation sites are found within hPOSH coding sequence (FIG. 30). Phosphorylation of gravin, an AKAP, by PKA modulates its binding to the b2-adrenergic receptor. This serves to regulate the mobilization of gravin and PKA to the cell membrane and regulation of b2-AR activity by PKA. Two putative PKA sites are located in the putative-rac-binding region in POSH. Toward this end, POSH was subjected to in-vitro phosphorylation and binding to the small GTPase Rac1 (FIG. 31). Indeed, only unphosphorylated POSH was able to bind activated, GTP-loaded, Rac1, demonstrating that phosphorylation regulates the binding of POSH to small GTPases, such as Rac1 . In the yeast-two hybrid screen a Rac1-releated protein, Chp, was identified as a POSH-interactor. GTPases of this sort family further include TCL, TC10, Cdc42, Wrch-1, Rac2, Rac3 or RhoG (Aspenstrom et al., 2003, Biochem J., 377(Pt 2):327-37). Small GTPases of this sort are involved in protein trafficking in the secretory system, including the trafficking of viral proteins, such as those of HIV.

Materials and Methods

PKA-Dependent Phosphorylation of hPOSH.

Bacterially expressed recombinant maltose-binding-protein (MBP)-hPOSH (3 μg) or GST-c-Cbl were incubated at 30° C. for 30 minutes with (*) or without 10 ng PKA catalytic subunit (PKAc) in a buffer containing 40 mM Tris-HCl pH 7.4, 10 mM MgCl₂, 4 mM ATP, 0.1 mg/ml BSA, 1 μM cAMP, 23 mM K₃PO₄, 7 nM DTT, and PKA peptide protection solution (Promega, Cat. No. V5340). The reaction was stopped by the addition of SDS-sample buffer, and boiling for 3 minutes. Samples were separated by SDS-PAGE on a 10% gel, and transferred to nitrocellulose and immunoblotted as detailed in the figure.

Binding of Rac1 to hPOSH

Bacterially expressed hPOSH (1 μg) or GST (1 μg) were phosphorylated as above. The reaction was terminated by the addition 0.5 ml of ice-cold 200 mM Tris-HCl pH 7.4, 5 mM EDTA. hPOSH and GST were then immobilized on NiNTA or reduced glutathione beads, respectively, by gentle mixing for 30 minutes. The immobilized proteins were washed three times with wash buffer (50 mM Tris-HCl pH 7.4, 100 mM NaCl, 5 mM MgCl2, 0.1 mM DTT). Recombinant Rac-1 (0.2 μg) (Sigma catalog # R3012) was incubated with or without 0.3 mM GTPγS (Sigma Cat. No. G8638) on ice for 15 minutes. The GTP/mock-loaded Rac-1 was then added to wash buffer (25 μl, final) and incubated for 30 minutes at 30° C. The beads were then washed three times with wash buffer containing 0.1% Tween 20. Sample buffer was added to the bead pellet and boiled for 3 minutes. Immobilized and associating proteins were then separated by SDS-PAGE on a 12% gel and immunobloted with anti-Rac-1 (Santa Cruz Biotechnology, Cat. No. sc-217). Input is 0.25 μg of Rac-1.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### Equivalents

While specific embodiments of the subject applications have been discussed, the above specification is illustrative and not restrictive. Many variations of the applications will become apparent to those skilled in the art upon review of this specification and the claims below. The full scope of the applications should be determined by reference to the claims, along with their full scope of equivalents, and the specification, along with such variations.

What is claimed:

1. An isolated, purified or recombinant complex comprising a POSH polypeptide and a POSH-associated kinase (POSH-AK) or a subunit of a POSH-AK.

2. The complex of claim 1, wherein the POSH-AK comprises a polypeptide selected from the group consisting of: JNK1, JNK2, MLK1, MLK2, MLK3, -MKK4, and MKK7, and wherein the POSH polypeptide is a human POSH polypeptide.

3. The complex of claim 1, wherein the POSH-AK comprises a PKA subunit polypeptide selected from the group consisting of: PRKAR1A, PRKACA, and PRKACB.

4. A method for identifying an agent that modulates an activity of a POSH polypeptide or POSH-AK, the method comprising identifying an agent that disrupts a complex of claim 1, wherein an agent that disrupts a complex of claim 1 is an agent that modulates an activity of the POSH polypeptide or the POSH-AK.

5. A method for identifying an agent that modulates an activity of a POSH polypeptide or POSH-AK, the method comprising identifying an agent that disrupts a complex of claim 2, wherein an agent that disrupts a complex of claim 2 is an agent that modulates an activity of the POSH polypeptide or the POSH-AK.

6. A method for identifying an agent that modulates an activity of a POSH polypeptide or POSH-AK, the method comprising identifying an agent that disrupts a complex of claim 3, wherein an agent that disrupts a complex of claim 3 is an agent that modulates an activity of the POSH polypeptide or the POSH-AK.

7. A method of identifying an antiviral agent, comprising:
(a) identifying a test agent that disrupts a complex of claim 1; and
(b) evaluating the effect of the test agent on a function of a virus,
wherein an agent that inhibits a pro-infective or pro-replicative function of a virus is an antiviral agent.

8. The method of claim 7, wherein the virus is an envelope virus.

9. The method of claim 7, wherein the virus is a Human Immunodeficiency Virus.

10. The method of claim 7, wherein evaluating the effect of the test agent on a function of the virus comprises evaluating the effect of the test agent on the budding or release of the virus or a virus-like particle.

11. A method for identifying an antiviral agent comprising:
(a) identifying a test agent that inhibits an activity of or expression of a POSH-AK or a subunit of the POSH-AK; and
(b) evaluating an effect of the test agent on a function of a virus.

12. The method of claim 11, wherein the virus is an envelope virus.

13. The method of claim 11, wherein the virus is a Human Immunodeficiency Virus.

14. The method of claim 11, wherein evaluating the effect of the test agent on a function of the virus comprises evaluating the effect of the test agent on the budding or release of the virus or a virus-like particle.

15. The method of claim 11, wherein the POSH-AK is PKA.

16. The method of claim 11, wherein the test agent is selected from among: an antisense nucleic acid, an siRNA construct, a small molecule, an antibody and a polypeptide.

17. A method of identifying an anti-viral agent, comprising:
a) forming a mixture comprising a POSH polypeptide, a PKA and a test agent; and
b) detecting phosphorylation of the POSH polypeptide,
wherein an agent that inhibits phosphorylation of the POSH polypeptide is an anti-viral agent.

18. The method of claim 17, wherein phosphorylation of the POSH polypeptide at a consensus PKA phosphorylation site is detected.

19. The method of claim 17, wherein phosphorylation of the POSH polypeptide at a site of sequence K/R-R-X-S/T-Hydrophobic is detected.

20. The method of claim 17, wherein phosphorylation of the POSH polypeptide at a site of sequence R-X-X-S/T-Hydrophobic is detected.


SEQUENCE LISTING (88 sequences)

SEQ ID NO: 1 - DNA, 2667 bp, Homo sapiens
atggatgaatcagccttgttggatcttttggagtgtccggtgtgtctagagcgccttgatgcttctgcgaaggtcttgccttgccagcatacgttttgcaagcgatgtttgctggggatcgtaggttctcgaaatgaactcagatgtcccgagtgcaggactcttgttggctcgggtgtcgaggagcttcccagtaacatcttgctggtcagacttctggatggcatcaaacagaggccttggaaacctggtcctggtgggggaagtgggaccaactgcacaaatgcattaaggtctcagagcagcactgtggctaattgtagctcaaaagatctgcagagctcccagggcggacagcagcctcgggtgcaatcctggagccccccagtgaggggtatacctcagttaccatgtgccaaagcgttatacaactatgaaggaaaagagcctggagaccttaaattcagcaaaggcgacatcatcattttgcgaagacaagtggatgaaaattggtaccatggggaagtcaatggaatccatggctttttccccaccaactttgtgcagattattaaaccgttacctcagcccccacctcagtgcaaagcactttatgactttgaagtgaaagacaaggaagcagacaaagattgccttccatttgcaaaggatgatgttctgactgtgatccgaagagtggatgaaaactgggctgaaggaatgctggcagacaaaataggaatatttccaatttcatatgttgagtttaactcggctgctaagcagctgatagaatgggataagcctcctgtgccaggagttgatgctggagaatgttcctcggcagcagcccagagcagcactgccccaaagcactccgacaccaagaagaacaccaaaaagcggcactccttcacttccctcactatggccaacaagtcctcccaggcatcccagaaccgccactccatggagatcagcccccctgtcctcatcagctccagcaaccccactgctgctgcacggatcagcgagctgtctgggctctcctgcagtgccccttctcaggttcatataagtaccaccgggttaattgtgaccccgcccccaagcagcccagtgacaactggcccctcgtttactttcccatcagatgttccctaccaagctgcccttggaactttgaatcctcctcttccaccaccccctctcctggctgccactgtccttgcctccacaccaccaggcgccaccgccgccgctgctgctgctggaatgggaccgaggcccatggcaggatccactgaccagattgcacatttacggccgcagactcgccccagtgtgtatgttgctatatatccatacactcctcggaaagaggatgaactagagctgagaaaaggggagatgtttttagtgtttgagcgctgccaggatggctggttcaaagggacatccatgcataccagcaagataggggttttccctggcaattatgtggcaccagtcacaagggcggtgacaaatgcttcccaagctaaagtccctatgtctacagctggccagacaagtcggggagtgaccatggtcagtccttccacggcaggagggcctgcccagaagctccagggaaatggcgtggctgggagtcccagtgttgtccccgcagctgtggtatcagcagctcacatccagacaagtcctcaggctaaggtcttgttgcacatgacggggcaaatgacagtcaaccaggcccgcaatgctgtgaggacagttgcagcgcacaaccaggaacgccccacggcagcagtgacacccatccaggtacagaatgccgccggcctcagccctgcatctgtgggcctgtcccatcactcgctggcctccccacaacctgcgcctctgatgccaggctcagccacgcacactgctgccatcagtatcagtcgagccagtgcccctctggcctgtgcagcagctgctccactgacttccccaagcatcaccagtgcttctctggaggctgagcccagtggccggatagtgaccgttctccctggactccccacatctcctgacagtgcttcatcagcttgtgggaacagttcagcaaccaaaccagacaaggatagcaaaaaagaaaaaaagggtttgttgaagttgctttctggcgcctccactaaacggaagccccgcgtgtctcctccagcatcgcccaccctagaagtggagctgggcagtgcagagcttcctctccagggagcggtggggcccgaactgccaccaggaggtggccatggcagggcaggctcctgccctgtggacggggacggaccggtcacgactgcagtggcaggagcagccctggcccaggatgcttttcataggaaggcaagttccctggactccgcagttcccatcgctccacctcctcgccaggcctgttcctccctgggtcctgtcttgaatgagtctagacctgtcgtttgtgaaaggcacagggtggtggtttcctatcctcctcagagtgaggcagaacttgaacttaaagaaggagatattgtgtttgttcataaaaaacgagaggatggctggttcaaaggcacattacaacgtaatgggaaaactggccttttcccaggaagctttgtggaaaacatatga

SEQ ID NO: 2 - protein, 888 aa, Homo sapiens
MDESALLDLLECPVCLERLDASAKVLPCQHTFCKRCLLGIVGSRNELRCPECRTLVGSGVEELPSNILLVRLLDGIKQRPWKPGPGGGSGTNCTNALRSQSSTVANCSSKDLQSSQGGQQPRVQSWSPPVRGIPQLPCAKALYNYEGKEPGDLKFSKGDIIILRRQVDENWYHGEVNGIHGFFPTNFVQIIKPLPQPPPQCKALYDFEVKDKEADKDCLPFAKDDVLTVIRRVDENWAEGMLADKIGIFPISYVEFNSAAKQLIEWDKPPVPGVDAGECSSAAAQSSTAPKHSDTKKNTKKRHSFTSLTMANKSSQASQNRHSMEISPPVLISSSNPTAAARISELSGLSCSAPSQVHISTTGLIVTPPPSSPVTTGPSFTFPSDVPYQAALGTLNPPLPPPPLLAATVLASTPPGATAAAAAAGMGPRPMAGSTDQIAHLRPQTRPSVYVAIYPYTPRKEDELELRKGEMFLVFERCQDGWFKGTSMHTSKIGVFPGNYVAPVTRAVTNASQAKVPMSTAGQTSRGVTMVSPSTAGGPAQKLQGNGVAGSPSVVPAAVVSAAHIQTSPQAKVLLHMTGQMTVNQARNAVRTVAAHNQERPTAAVTPIQVQNAAGLSPASVGLSHHSLASPQPAPLMPGSATHTAAISISRASAPLACAAAAPLTSPSITSASLEAEPSGRIVTVLPGLPTSPDSASSACGNSSATKPDKDSKKEKKGLLKLLSGASTKRKPRVSPPASPTLEVELGSAELPLQGAVGPELPPGGGHGRAGSCPVDGDGPVTTAVAGAALAQDAFHRKASSLDSAVPIAPPPRQACSSLGPVLNESRPVVCERHRVVVSYPPQSEAELELKEGDIVFVHKKREDGWFKGTLQRNGKTGLFPGSFVENI

SEQ ID NO: 3 - DNA, 5128 bp, Homo sapiens
ctgagagacactgcgagcggcgagcgcggtggggccgcatctgcatcagccgccgcagccgctgcggggccgcgaacaaagaggaggagccgaggcgcgagagcaaagtctgaaatggatgttacatgagtcattttaagggatgcacacaactatgaacatttctgaagattttttctcagtaaagtagataaagatggatgaatcagccttgttggatcttttggagtgtccggtgtgtctagagcgccttgatgcttctgcgaaggtcttgccttgccagcatacgttttgcaagcgatgtttgctggggatcgtaggttctcgaaatgaactcagatgtcccgagtgcaggactcttgttggctcgggtgtcgaggagcttcccagtaacatcttgctggtcagacttctggatggcatcaaacagaggccttggaaacctggtcctggtgggggaagtgggaccaactgcacaaatgcattaaggtctcagagcagcactgtggctaattgtagctcaaaagatctgcagagctcccagggcggacagcagcctcgggtgcaatcctggagccccccagtgaggggtatacctcagttaccatgtgccaaagcgttatacaactatgaaggaaaagagcctggagaccttaaattcagcaaaggcgacatcatcattttgcgaagacaagtggatgaaaattggtaccatggggaagtcaatggaatccatggctttttccccaccaactttgtgcagattattaaaccgttacctcagcccccacctcagtgcaaagcactttatgactttgaagtgaaagacaaggaagcagacaaagattgccttccatttgcaaaggatgatgttctgactgtgatccgaagagtggatgaaaactgggctgaaggaatgctggcagacaaaataggaatatttccaatttcatatgttgagtttaactcggctgctaagcagctgatagaatgggataagcctcctgtgccaggagttgatgctggagaatgttcctcggcagcagcccagagcagcactgccccaaagcactccgacaccaagaagaacaccaaaaagcggcactccttcacttccctcactatggccaacaagtcctcccaggcatcccagaaccgccactccatggagatcagcccccctgtcctcatcagctccagcaaccccactgctgctgcacggatcagcgagctgtctgggctctcctgcagtgccccttctcaggttcatataagtaccaccgggttaattgtgaccccgcccccaagcagcccagtgacaactggcccctcgtttactttcccatcagatgttccctaccaagctgcccttggaactttgaatcctcctcttccaccaccccctctcctggctgccactgtccttgcctccacaccaccaggcgccaccgccgccgctgctgctgctggaatgggaccgaggcccatggcaggatccactgaccagattgcacatttacggccgcagactcgccccagtgtgtatgttgctatatatccatacactcctcggaaagaggatgaactagagctgagaaaaggggagatgtttttagtgtttgagcgctgccaggatggctggttcaaagggacatccatgcataccagcaagataggggttttccctggcaattatgtggcaccagtcacaagggcggtgacaaatgcttcccaagctaaagtccctatgtctacagctggccagacaagtcggggagtgaccatggtcagtccttccacggcaggagggcctgcccagaagctccagggaaatggcgtggctgggagtcccagtgttgtccccgcagctgtggtatcagcagctcacatccagacaagtcctcaggctaaggtcttgttgcacatgacggggcaaatgacagtcaaccaggcccgcaatgctgtgaggacagttgcagcgcacaaccaggaacgccccacggcagcagtgacacccatccaggtacagaatgccgccggcctcagccctgcatctgtgggcctgtcccatcactcgctggcctccccacaacctgcgcctctgatgccaggctcagccacgcacactgctgccatcagtatcagtcgagccagtgcccctctggcctgtgcagcagctgctccactgacttccccaagcatcaccagtgcttctctggaggctgagcccagtggccggatagtgaccgttctccctggactccccacatctcctgacagtgcttcatcagcttgtgggaacagttcagcaaccaaaccagacaaggatagcaaaaaagaaaaaaagggtttgttgaagttgctttctggcgcctccactaaacggaagccccgcgtgtctcctccagcatcgcccaccctagaagtggagctgggcagtgcagagcttcctctccagggagcggtggggcccgaactgccaccaggaggtggccatggcagggcaggctcctgccctgtggacggggacggaccggtcacgactgcagtggcaggagcagccctggcccaggatgcttttcataggaaggcaagttccctggactccgcagttcccatcgctccacctcctcgccaggcctgttcctccctgggtcctgtcttgaatgagtctagacctgtcgtttgtgaaaggcacagggtggtggtttcctatcctcctcagagtgaggcagaacttgaacttaaagaaggagatattgtgtttgttcataaaaaacgagaggatggctggttcaaaggcacattacaacgtaatgggaaaactggccttttcccaggaagctttgtggaaaacatatgaggagactgacactgaagaagcttaaaatcacttcacacaacaaagtagcacaaagcagtttaacagaaagagcacatttgtggacttccagatggtcaggagatgagcaaaggattggtatgtgactctgatgccccagcacagttaccccagcgagcagagtgaagaagatgtttgtgtgggttttgttagtctggattcggatgtataaggtgtgccttgtactgtctgatttactacacagagaaacttttttttttttttaagatatatgactaaaatggacaattgtttacaaggcttaactaatttatttgcttttttaaacttgaacttttcgtataatagatacgttctttggattatgattttaagaaattattaatttatgaaatgataggtaaggagaagctggattatctcctgttgagagcaagagattcgttttgacatagagtgaatgcattttcccctctcctcctccctgctaccattatattttggggttatgttttgcttctttaagatagaaatcccagttctctaatttggttttcttctttgggaaaccaaacatacaaatgaatcagtatcaattagggcctggggtagagagacagaaacttgagagaagagaagttagtgattccctctctttctagtttggtaggaatcaccctgaagacctagtcctcaatttaattgtgtgggtttttaattttcctagaatgaagtgactgaaacaatgagaaagaatacagcacaacccttgaacaaaatgtatttagaaatatatttagttttatagcagaagcagctcaattgtttggttggaaagtaggggaaattgaagttgtagtcactgtctgagaatggctatgaagcgtcatttcacattttaccccaactgacctgcatgcccaggacacaagtaaaacatttgtgagatagtggtggtaagtgatgcactcgtgttaagtcaaaggctataagaaacactgtgaaaagttcatattcatccattgtgattctttccccacgtcttgcatgtattactggattcccacagtaatatagactgtgcatggtgtgtatatttcattgcgatttcctgttaagatgagtttgtactcagaattgaccaattcaggaggtgtaaaaataaacagtgttctcttctctaccccaaagccactactgaccaaggtctcttcagtgcactcgctccctctctggctaaggcatgcattagccactacacaagtcattagtgaaagtggtcttttatgtcctcccagcagacagacatcaaggatgagttaaccaggagactactcctgtgactgtggagctctggaaggcttggtgggagtgaatttgcccacaccttacaattgtggcaggatccagaagagcctgtctttttatatccattccttgatgtcattggcctctcccaccgatttcattacggtgccacgcagtcatggatctgggtagtccggaaaacaaaaggagggaagacagcctggtaatgaataagatccttaccacagttttctcatgggaaatacataataaaccctttcatctttttttttttcctttaagaattaaaactgggaaatagaaacatgaactgaaaagtcttgcaatgacaagaggtttcatggtcttaaaaagatactttatatggttgaagatgaaatcattcctaaattaaccttttttttaaaaaaaaacaatgtatattatgttcctgtgtgttgaatttaaaaaaaaaaaatactttacttggatattcatgtaatatataaaggtttggtgaaatgaactttagttaggaaaaagctggcatcagctttcatctgtgtaagttgacaccaatgtgtcataatattctttattttgggaaattagtgtattttataaaaattttaaaaagaaaaaagactactacaggttaagataatttttttacctgtcttttctccatattttaagctatgtgattgaagtacctctgttcatagtttcctggtataaagttggttaaaatttcatctgttaatagatcattaggtaatataatgtatgggttttctattggttttttgcagacagtagagggagattttgtaacaagggcttgttacacagtgatatggtaatgataaaattgcaatttatcactccttttcatgttaataatttgaggactggataaaaggtttcaagattaaaatttgatgttcaaacctttgt

SEQ ID NO: 4 - DNA, 2331 bp, Homo sapiens
ctgagagacactgcgagcggcgagcgcggtggggccgcatctgcatcagccgccgcagccgctgcggggccgcgaacaaagaggaggagccgaggcgcgagagcaaagtctgaaatggatgttacatgagtcattttaaggatgcacacaactatgaacatttctgaagattttttctcagtaaagtagataaagatggatgaatcagccttgttggatcttttggagtgtccggtgtgtctagagcgccttgatgcttctgcgaaggtcttgccttgccagcatacgttttgcaagcgatgtttgctggggatcgtaggttctcgaaatgaactcagatgtcccgagtgcaggactcttgttggctcgggtgtcgaggagcttcccagtaacatcttgctggtcagacttctggatggcatcaaacagaggccttggaaacctggtcctggtgggggaagtgggaccaactgcacaaatgcattaaggtctcagagcagcactgtggctaattgtagctcaaaagatctgcagagctcccagggcggacagcagcctcgggtgcaatcctggagccccccagtgaggggtatacctcagttaccatgtgccaaagcgttatacaactatgaaggaaaagagcctggagaccttaaattcagcaaaggcgacatcatcattttgcgaagacaagtggatgaaaattggtaccatggggaagtcaatggaatccatggctttttccccaccaactttgtgcagattattaaaccgttacctcagcccccacctcagtgcaaagcactttatgactttgaagtgaaagacaaggaagcagacaaagattgccttccatttgcaaaggatgatgttctgactgtgatccgaagagtggatgaaaactgggctgaaggaatgctggcagacaaaataggaatatttccaatttcatatgttgagtttaactcggctgctaagcagctgatagaatgggataagcctcctgtgccaggagttgatgctggagaatgttcctcggcagcagcccagagcagcactgccccaaagcactccgacaccaagaagaacaccaaaaagcggcactccttcacttccctcactatggccaacaagtcctcccaggcatcccagaaccgccactccatggagatcagcccccctgtcctcatcagctccagcaaccccactgctgctgcacggatcagcgagctgtctgggctctcctgcagtgccccttctcaggttcatataagtaccaccgggttaattgtgaccccgcccccaagcagcccagtgacaactggcccctcgtttactttcccatcagatgttccctaccaagctgcccttggaactttgaatcctcctcttccaccaccccctctcctggctgccactgtccttgcctccacaccaccaggcgccaccgccgccgctgctgctgctggaatgggaccgaggcccatggcaggatccactgaccagattgcacatttacggccgcagactcgccccagtgtgtatgttgctatatatccatacactcctcggaaagaggatgaactagagctgagaaaaggggagatgtttttagtgtttgagcgctgccaggatggctggttcaaagggacatccatgcataccagcaagataggggttttccctggcaattatgtggcaccagtcacaagggcggtgacaaatgcttcccaagctaaagtccctatgtctacagctggccagacaagtcggggagtgaccatggtcagtccttccacggcaggagggcctgcccagaagctccagggaaatggcgtggctgggagtcccagtgttgtccccgcagctgtggtatcagcagctcacatccagacaagtcctcaggctaaggtcttgttgcacatgacggggcaaatgacagtcaaccaggcccgcaatgctgtgaggacagttgcagcgcacaaccaggaacgccccacggcagcagtgacacccatccaggtacagaatgccgccggcctcagccctgcatctgtgggcctgtcccatcactcgctggcctccccacaacctgcgcctctgatgccaggctcagccacgcacactgctgccatcagtatcagtcgagccagtgcccctctggcctgtgcagcagctgctccactgacttccccaagcatcaccagtgcttctctggaggctgagcccagtggccggatagtgaccgttctccctggactccccacatctcctgacagtgcttcatcagcttgtgggaacagttcagcaaccaaaccagacaaggatagc

SEQ ID NO: 5 - protein, 712 aa, Homo sapiens
MDESALLDLLECPVCLERLDASAKVLPCQHTFCKRCLLGIVGSRNELRCPECRTLVGSGVEELPSNILLVRLLDGIKQRPWKPGPGGGSGTNCTNALRSQSSTVANCSSKDLQSSQGGQQPRVQSWSPPVRGIPQLPCAKALYNYEGKEPGDLKFSKGDIIILRRQVDENWYHGEVNGIHGFFPTNFVQIIKPLPQPPPQCKALYDFEVKDKEADKDCLPFAKDDVLTVIRRVDENWAEGMLADKIGIFPISYVEFNSAAKQLIEWDKPPVPGVDAGECSSAAAQSSTAPKHSDTKKNTKKRHSFTSLTMANKSSQASQNRHSMEISPPVLISSSNPTAAARISELSGLSCSAPSQVHISTTGLIVTPPPSSPVTTGPSFTFPSDVPYQAALGTLNPPLPPPPLLAATVLASTPPGATAAAAAAGMGPRPMAGSTDQIAHLRPQTRPSVYVAIYPYTPRKEDELELRKGEMFLVFERCQDGWFKGTSMHTSKIGVFPGNYVAPVTRAVTNASQAKVPMSTAGQTSRGVTMVSPSTAGGPAQKLQGNGVAGSPSVVPAAVVSAAHIQTSPQAKVLLHMTGQMTVNQARNAVRTVAAHNQERPTAAVTPIQVQNAAGLSPASVGLSHHSLASPQPAPLMPGSATHTAAISISRASAPLACAAAAPLTSPSITSASLEAEPSGRIVTVLPGLPTSPDSASSACGNSSATKPDKDS

SEQ ID NO: 6 - DNA, 4182 bp, Homo sapiens
atttcatatgttgagtttaactcggctgctaagcagctgatagaatgggataagcctcctgtgccaggagttgatgctggagaatgttcctcggcagcagcccagagcagcactgccccaaagcactccgacaccaagaagaacaccaaaaagcggcactccttcacttccctcactatggccaacaagtcctcccaggcatcccagaaccgccactccatggagatcagcccccctgtcctcatcagctccagcaaccccactgctgctgcacggatcagcgagctgtctgggctctcctgcagtgccccttctcaggttcatataagtaccaccgggttaattgtgaccccgcccccaagcagcccagtgacaactggcccctcgtttactttcccatcagatgttccctaccaagctgcccttggaactttgaatcctcctcttccaccaccccctctcctggctgccactgtccttgcctccacaccaccaggcgccaccgccgctgctgctgctgctggaatgggaccgaggcccatggcaggatccactgaccagattgcacatttacggccgcagactcgccccagtgtgtatgttgctatatatccatacactcctcggaaagaggatgaactagagctgagaaaaggggagatgtttttagtgtttgagcgctgccaggatggctggttcaaagggacatccatgcataccagcaagataggggttttccctggcaattatgtggcaccagtcacaagggcggtgacaaatgcttcccaagctaaagtccctatgtctacagctggccagacaagtcggggagtgaccatggtcagtccttccacggcaggagggcctgcccagaagctccagggaaatggcgtggctgggagtcccagtgttgtccccgcagctgtggtatcagcagctcacatccagacaagtcctcaggctaaggtcttgttgcacatgacggggcaaatgacagtcaaccaggcccgcaatgctgtgaggacagttgcagcgcacaaccaggaacgccccacggcagcagtgacacccatccaggtacagaatgccgccggcctcagccctgcatctgtgggcctgtcccatcactcgctggcctccccacaacctgcgcctctgatgccaggctcagccacgcacactgctgccatcagtatcagtcgagccagtgcccctctggcctgtgcagcagctgctccactgacttccccaagcatcaccagtgcttctctggaggctgagcccagtggccggatagtgaccgttctccctggactccccacatctcctgacagtgcttcatcagcttgtgggaacagttcagcaaccaaaccagacaaggatagcaaaaaagaaaaaaagggtttgttgaagttgctttctggcgcctccactaaacggaagccccgcgtgtctcctccagcatcgcccaccctagaagtggagctgggcagtgcagagcttcctctccagggagcggtggggcccgaactgccaccaggaggtggccatggcagggcaggctcctgccctgtggacggggacggaccggtcacgactgcagtggcaggagcagccctggcccaggatgcttttcataggaaggcaagttccctggactccgcagttcccatcgctccacctcctcgccaggcctgttcctccctgggtcctgtcttgaatgagtctagacctgtcgtttgtgaaaggcacagggtggtggtttcctatcctcctcagagtgaggcagaacttgaacttaaagaaggagatattgtgtttgttcataaaaaacgagaggatggctggttcaaaggcacattacaacgtaatgggaaaactggccttttcccaggaagctttgtggaaaacatatgaggagactgacactgaagaagcttaaaatcacttcacacaacaaagtagcacaaagcagtttaacagaaagagcacatttgtggacttccagatggtcaggagatgagcaaaggattggtatgtgactctgatgccccagcacagttaccccagcgagcagagtgaagaagatgtttgtgtgggttttgttagtctggattcggatgtataaggtgtgccttgtactgtctgatttactacacagagaaacttttttttttttttaagatatatgactaaaatggacaattgtttacaaggcttaactaatttatttgcttttttaaacttgaacttttcgtataatagatacgttctttggattatgattttaagaaattattaatttatgaaatgataggtaaggagaagctggattatctcctgttgagagcaagagattcgttttgacatagagtgaatgcattttcccctctcctcctccctgctaccattatattttggggttatgttttgcttctttaagatagaaatcccagttctctaatttggttttcttctttgggaaaccaaacatacaaatgaatcagtatcaattagggcctggggtagagagacagaaacttgagagaagagaagttagtgattccctctctttctagtttggtaggaatcaccctgaagacctagtcctcaatttaattgtgtgggtttttaattttcctagaatgaagtgactgaaacaatgagaaagaatacagcacaacccttgaacaaaatgtatttagaaatatatttagttttatagcagaagcagctcaattgtttggttggaaagtaggggaaattgaagttgtagtcactgtctgagaatggctatgaagcgtcatttcacattttaccccaactgacctgcatgcccaggacacaagtaaaacatttgtgagatagtggtggtaagtgatgcactcgtgttaagtcaaaggctataagaaacactgtgaaaagttcatattcatccattgtgattctttccccacgtcttgcatgtattactggattcccacagtaatatagactgtgcatggtgtgtatatttcattgcgatttcctgttaagatgagtttgtactcagaattgaccaattcaggaggtgtaaaaataaacagtgttctcttctctaccccaaagccactactgaccaaggtctcttcagtgcactcgctccctctctggctaaggcatgcattagccactacacaagtcattagtgaaagtggtcttttatgtcctcccagcagacagacatcaaggatgagttaaccaggagactactcctgtgactgtggagctctggaaggcttggtgggagtgaatttgcccacaccttacaattgtggcaggatccagaagagcctgtctttttatatccattccttgatgtcattggcctctcccaccgatttcattacggtgccacgcagtcatggatctgggtagtccggaaaacaaaaggagggaagacagcctggtaatgaataagatccttaccacagttttctcatgggaaatacataataaaccctttcatctttttttttttcctttaagaattaaaactgggaaatagaaacatgaactgaaaagtcttgcaatgacaagaggtttcatggtcttaaaaagatactttatatggttgaagatgaaatcattcctaaattaaccttttttttaaaaaaaaacaatgtatattatgttcctgtgtgttgaatttaaaaaaaaaaaatactttacttggatattcatgtaatatataaaggtttggtgaaatgaactttagttaggaaaaagctggcatcagctttcatctgtgtaagttgacaccaatgtgtcataatattctttattttgggaaattagtgtattttataaaaattttaaaaagaaaaaagactactacaggttaagataatttttttacctgtcttttctccatattttaagctatgtgattgaagtacctctgttcatagtttcctggtataaagttggttaaaatttcatctgttaatagatcattaggtaatataatgtatgggttttctattggttttttgcagacagtagagggagattttgtaacaagggcttgttacacagtgatatggtaatgataaaattgcaatttatcactccttttcatgttaataatttgaggactggataaaaggtttcaagattaaaatttgatgttcaaacctttgt

SEQ ID NO: 7 - protein, 638 aa, Homo sapiens
ISYVEFNSAAKQLIEWDKPPVPGVDAGECSSAAAQSSTAPKHSDTKKNTKKRHSFTSLTMANKSSQASQNRHSMEISPPVLISSSNPTAAARISELSGLSCSAPSQVHISTTGLIVTPPPSSPVTTGPSFTFPSDVPYQAALGTLNPPLPPPPLLAATVLASTPPGATAAAAAAGMGPRPMAGSTDQIAHLRPQTRPSVYVAIYPYTPRKEDELELRKGEMFLVFERCQDGWFKGTSMHTSKIGVFPGNYVAPVTRAVTNASQAKVPMSTAGQTSRGVTMVSPSTAGGPAQKLQGNGVAGSPSVVPAAVVSAAHIQTSPQAKVLLHMTGQMTVNQARNAVRTVAAHNQERPTAAVTPIQVQNAAGLSPASVGLSHHSLASPQPAPLMPGSATHTAAISISRASAPLACAAAAPLTSPSITSASLEAEPSGRIVTVLPGLPTSPDSASSACGNSSATKPDKDSKKEKKGLLKLLSGASTKRKPRVSPPASPTLEVELGSAELPLQGAVGPELPPGGGHGRAGSCPVDGDGPVTTAVAGAALAQDAFHRKASSLDSAVPIAPPPRQACSSLGPVLNESRPVVCERHRVVVSYPPQSEAELELKEGDIVFVHKKREDGWFKGTLQRNGKTGLFPGSFVENI

SEQ ID NO: 8 - DNA, 3206 bp, Homo sapiens
gggcagcgggctcggcggggctgcatctaccagcgctgcggggccgcgaacaaaggcgagcagcggaggcgcgagagcaaagtctgaaatggatgttacatgaatcactttaagggctgcgcacaactatgaacgttctgaagccgttttctcactaaagtcactcaagatggatgagtctgccttgttggaccttctggagtgccctgtgtgtctagaacgcctggatgcttccgcaaaggtcttaccctgccagcataccttttgcaaacgctgtttgctggggattgtgggttcccggaatgaactcagatgtcccgaatgccggactcttgttggctctggggtcgacgagctccccagtaacatcctactggtcagacttctggatggcatcaagcagaggccttggaaacccggccctggtgggggcggcgggaccacctgcacaaacacattaagggcgcagggcagcactgtggttaattgtggctcgaaagatctgcagagctcccagtgtggacagcagcctcgggtgcaagcctggagccccccagtgaggggaatacctcagttaccgtgtgccaaagcattatataactacgaaggaaaagagcccggagaccttaagttcagcaaaggcgacaccatcattctgcgccgacaggtggatgagaattggtaccacggggaagtcagcggggtccacggctttttccccactaacttcgtgcagatcatcaaacctttacctcagcccccgcctcagtgcaaagcactttacgactttgaagtgaaagacaaggaagctgacaaagattgccttcccttcgcaaaggacgacgtactgaccgtgatccgcagagtggatgaaaactgggctgaaggaatgctggcagataaaataggaatatttccaatttcatacgtggagtttaactcagctgccaagcagctgatagagtgggataagcctcccgtgccaggagtggacacggcagaatgcccctcagcgacggcgcagagcacctctgcctcaaagcaccccgacaccaagaagaacaccaggaagcgacactccttcacctccctcaccatggccaacaagtcttcccaggggtcccagaaccgccactccatggagatcagccctcctgtgctcatcagttccagcaaccccacagccgcagcccgcatcagcgaactgtccgggctctcctgcagcgccccgtctcaggtccatataagcaccactgggttaattgtgaccccaccccctagcagcccggtgacaactggccctgcgttcacgttcccttcagatgtcccctaccaagctgcccttggaagtatgaatcctccacttcccccaccccctctcctggcggccaccgtactcgcctccaccccgtcaggcgctactgctgctgttgctgctgctgctgccgccgccgccgctgctggaatgggacccaggcctgtgatggggtcctctgaacagattgcacatttacggcctcagactcgtcccagtgtatatgttgctatatatccgtacactccccggaaggaagacgaactggagctgaggaaaggggagatgtttttggtgtttgagcgttgccaggacggctggtacaaagggacatcgatgcataccagcaagataggcgttttccctggcaactatgtggcgcccgtcacaagggcggtgacgaatgcctcccaagctaaagtctctatgtctactgcgggtcaggcaagtcgcggggtgaccatggtcagcccttccactgcaggaggacctacacagaagccccaaggaaacggcgtggccggaaatcccagcgtcgtccccacggctgtggtgtcagcagctcatatccagacaagtcctcaggctaaggtcctgctgcacatgtctgggcagatgacagtcaatcaggcccgcaatgctgtgaggacagttgcagcacatagccaggaacgccccacagcagcagtgactcccatccaggtccagaatgccgcctgccttggtcctgcatccgtgggcctgccccatcattctctggcctcccaacctctgcctccaatggcgggtcctgctgcccacggtgctgccgtcagcatcagtcgaaccaatgcccccatggcctgcgctgcaggggcttctctggcctccccaaatatgaccagtgccatgttggagacagagcccagtggtcgcacagtgaccatcctccctggactccccacatctccagagagtgctgcatcagcgtgtgggaacagttcagctgggaaaccagacaaggacagtaagaaagaaaaaaagggcctactgaagctgctttctggtgcctccaccaaacgcaagccccgagtctcccctccagcatcacctaccctggatgtggagctgggtgctggggaggctcccttgcagggagcagtaggtcctgagctgccgctagggggcagccacggcagagtggggtcatgccccacagatggtgatggtccagtggccgctggaacagcagccctagcccaggatgccttccaccgcaagacaagctccctggactccgcagtgcccattgctccaccacctcgccaggcctgctcctccctgggcccagtcatgaatgaggcccggcctgttgtttgtgaaaggcacagggtggtggtttcctaccctcctcagagtgaggccgaacttgaactcaaggaaggagatattgtgtttgttcataagaaacgagaggacggctggttcaaaggcacgttacagaggaatgggaagactggccttttcccagggagctttgtggaaaacatctgagaagacgggacacggagaaagcttatcatcacaccacgtgtgactaaagagcacaaagcagtttcatagaaagagcacatctgtggacttccagatcttcaagaaccgagcagaagatgggcacctgactccagagccccggcctggttaccccaggggcagagggaaggaggacacacctgtgtgggttccgtctctctgggttctgatgtgtaaagtgtgccttgtaatgtctaatggactttacagataaatgtctttttttttttaagatgtataactaaaatggacaattgtttacaaggcttaactaatttatttgcttttttaaaacttgaactttcttgtaatagcaaat

SEQ ID NO: 9 - protein, 892 aa, Mouse
MDESALLDLLECPVCLERLDASAKVLPCQHTFCKRCLLGIVGSRNELRCPECRTLVGSGVDELPSNILLVRLLDGIKQRPWKPGPGGGGGTTCTNTLRAQGSTVVNCGSKDLQSSQCGQQPRVQAWSPPVRGIPQLPCAKALYNYEGKEPGDLKFSKGDTIILRRQVDENWYHGEVSGVHGFFPTNFVQIIKPLPQPPPQCKALYDFEVKDKEADKDCLPFAKDDVLTVIRRVDENWAEGMLADKIGIFPISYVEFNSAAKQLIEWDKPPVPGVDTAECPSATAQSTSASKHPDTKKNTRKRHSFTSLTMANKSSQGSQNRHSMEISPPVLISSSNPTAAARISELSGLSCSAPSQVHISTTGLIVTPPPSSPVTTGPAFTFPSDVPYQAALGSMNPPLPPPPLLAATVLASTPSGATAAVAAAAAAAAAAGMGPRPVMGSSEQIAHLRPQTRPSVYVAIYPYTPRKEDELELRKGEMFLVFERCQDGWYKGTSMHTSKIGVFPGNYVAPVTRAVTNASQAKVSMSTAGQASRGVTMVSPSTAGGPTQKPQGNGVAGNPSVVPTAVVSAAHIQTSPQAKVLLHMSGQMTVNQARNAVRTVAAHSQERPTAAVTPIQVQNAACLGPASVGLPHHSLASQPLPPMAGPAAHGAAVSISRTNAPMACAAGASLASPNMTSAMLETEPSGRTVTILPGLPTSPESAASACGNSSAGKPDKDSKKEKKGLLKLLSGASTKRKPRVSPPASPTLDVELGAGEAPLQGAVGPELPLGGSHGRVGSCPTDGDGPVAAGTAALAQDAFHRKTSSLDSAVPIAPPPRQACSSLGPVMNEARPVVCERHRVVVSYPPQSEAELELKEGDIVFVHKKREDGWFKGTLQRNGKTGLFPGSFVENI

SEQ ID NO: 10 - DNA, 3149 bp, Drosophila melanogaster
catttgtatccgcttggccacgagctttggctgcacttggcaaacttaataaattaaacattgaatcctgcctattgcaacgataatataatctgatttagtgcattaagaacgacaagtagcgattataatagtagattttagcatttgagctaaatttatttcccaaccgcgtcttgggattgcgtatgcgtgagccagtacctgcatgtgtgtgtgttttggaatgtggccctgcacgaaattcaaatagtgaccatccttgagattttgcatactggcaagatggacgagcacacgttaaacgacctgttggagtgctccgtgtgtcttgagcgactggacaccacatcgaaggtgctgccatgccagcacaccttctgccgcaaatgcttgcaggacattgtggccagtcagcacaagttgcgatgcccggagtgccgcatcctggtctcttgcaaaattgatgagctgcctccaaacgtcttgctgatgcgaatcttagaaggcatgaaacaaaatgcagcagctggcaaaggagaagaaaagggagaggagactgaaacacagccggaaagggccaaacctcagccgccagcggaatcagtggccccgcctgacaaccaactactccagctgcagtcacatcagcaatctcatcagccggctcgtcacaagcaacgtcgatttctactcccccacgcctatgccctctttgacttcgcctccggtgaagccaccgatctaaagttcaagaaaggggatctgatactgatcaagcatcgcatcgacaacaactggtttgtgggtcaagcgaatggtcaggagggcacatttcccatcaactacgtcaaggtatcggttccgctgcccatgccgcagtgcattgccatgtatgactttaagatggggcccaacgacgaggagggatgcctcgaatttaagaaaagcactgtaatacaggtaatgcgccgagttgatcataattgggcagaaggacgaattggccagaccatcggaatctttccaatagcattcgttgagctgaatgcagcggccaaaaagctgttggacagcgggctacacacccatccattctgccatccaccgaagcaacaggggcagcgggcccttcctccggttccagttattgatcccacggtggtcacggaatccagttcgggatcctccaattccacgccgggcagcagcaattcaagctccacatccagctcgaataactgcagtccgaatcaccaaatctcactgccgaataccccccaacatgtagtagcttccggatcggcgtctgttcgtttccgtgacaagggagcaaaggagaaacgccactcactaaatgctttgctgggaggaggagctccattaagtctgctgcagaccaaccgccattcggctgaaattcttagcctgccccatgaactaagccgcttggaagtttccagctcaacagctctaaaacccacgtcagccccacagacatcgcgtgtacttaagaccactgttcagcagcagatgcaaccgaatttaccctggggatacttagccctgttcccatacaaaccacgccaaacggatgagctggaattaaaaaagggttgtgtttacattgtgaccgaacgatgtgtggacggttggttcaagggaaaaaactggttggacatcactggagtgttcccgggcaactacctgacgcccctgcgcgcccgcgaccagcagcagttaatgcatcaatggaaatatgttccccaaaatgcagacgcccagatggcacaagtacagcagcatccagttgcaccagatgtgcgactcaacaacatgctgtccatgcaaccgcctgatttgccacctcgtcagcagcaggctaccgccacgaccaccagttgctctgtgtggtcgaaaccagtggaggcgctgttcagcagaaaatcggagcccaagcctgaaactgccacagcttcgactacgagcagcagttcctctggagcagtgggacttatgaggagattaactcacatgaaaacacgctccaaatctccgggagcgtccttgcagcaagttccgaaagaagctattagcacaaatgtggaatttacaacaaacccatcagctaaattgcatccagtacatgtaagatccggctcgtgccccagtcagctgcagcacagtcaaccgctcaatgaaactccagcagccaagacagcggcacaacaacagcagttcctacccaagcagctgccttccgcttctacgaacagcgtttcgtacggatcgcaacgcgtgaaaggaagcaaggaacgtcctcacttgatttgcgcgagacaatcattagatgcagctacatttcgcagtatgtacaacaatgccgcgtcgccgccgccacctactacttccgtggccccagctgtctacgccggcggtcagcaacaggtgattcctggaggtggagcgcaatcccagttgcatgccaatatgattattgcacccagccatcggaagtcgcacagcctagatgcgagtcatgtgctgagtcccagcagcaatatgatcacggaggcggccattaaggccagcgccaccactaagtctccttactgcacgagggaaagtcgattccgctgcattgtgccgtatccaccaaacagtgacattgaactagagctacatttgggcgacattatctacgtccagcggaagcagaagaacggctggtataagggcacccatgcccgtacccacaaaaccgggctgttccccgcctcctttgttgaaccggattgttaggaaagttatggttcaaactagaatttattaagcgaaattccaaattacttgtctaaaaggattcaatcgtcggtctattcgggcttccaaatacgcaatctcatatttctcttttcaaaaaagaaaccgttttgtactcttccaatcgaatgggcagctcgccgttgtacttttttatacaatgcttgatcaaaataggctagccatgtaagacttagggaacagttacttaagccttagcgattagttagctagagaaataatctaaccgatccttgtgccctctacaaagttatttgtaatatacgatactcagtaataaaaaaaaaaaaaaaaaaaaaaaaaaa

SEQ ID NO: 11 - protein, 838 aa, Drosophila melanogaster
MDEHTLNDLLECSVCLERLDTTSKVLPCQHTFCRKCLQDIVASQHKLRCPECRILVSCKIDELPPNVLLMRILEGMKQNAAAGKGEEKGEETETQPERAKPQPPAESVAPPDNQLLQLQSHQQSHQPARHKQRRFLLPHAYALFDFASGEATDLKFKKGDLILIKHRIDNNWFVGQANGQEGTFPINYVKVSVPLPMPQCIAMYDFKMGPNDEEGCLEFKKSTVIQVMRRVDHNWAEGRIGQTIGIFPIAFVELNAAAKKLLDSGLHTHPFCHPPKQQGQRALPPVPVIDPTVVTESSSGSSNSTPGSSNSSSTSSSNNCSPNHQISLPNTPQHVVASGSASVRFRDKGAKEKRHSLNALLGGGAPLSLLQTNRHSAEILSLPHELSRLEVSSSTALKPTSAPQTSRVLKTTVQQQMQPNLPWGYLALFPYKPRQTDELELKKGCVYIVTERCVDGWFKGKNWLDITGVFPGNYLTPLRARDQQQLMHQWKYVPQNADAQMAQVQQHPVAPDVRLNNMLSMQPPDLPPRQQQATATTTSCSVWSKPVEALFSRKSEPKPETATASTTSSSSSGAVGLMRRLTHMKTRSKSPGASLQQVPKEAISTNVEFTTNPSAKLHPVHVRSGSCPSQLQHSQPLNETPAAKTAAQQQQFLPKQLPSASTNSVSYGSQRVKGSKERPHLICARQSLDAATFRSMYNNAASPPPPTTSVAPAVYAGGQQQVIPGGGAQSQLHANMIIAPSHRKSHSLDASHVLSPSSNMITEAAIKASATTKSPYCTRESRFRCIVPYPPNSDIELELHLGDIIYVQRKQKNGWYKGTHARTHKTGLFPASFVEPDC

SEQ ID NO: 12 - DNA, 18 bp, Unknown
  Oligonucleotide primer
cttgccttgccagcatac

SEQ ID NO: 13 - DNA, 18 bp, Unknown
  Oligonucleotide primer
ctgccagcattccttcag

SEQ ID NO: 14 - DNA, 21 bp, Unknown
  Oligonucleotide sequence
aacagaggccttggaaacctg

SEQ ID NO: 15 - RNA, 19 nt, Unknown
  Oligonucleotide SiRNA
cagaggccuuggaaaccug

SEQ ID NO: 16 - RNA, 19 nt, Unknown
  Oligonucleotide SiRNA
cagguuuccaaggccucug

SEQ ID NO: 17 - DNA, 21 bp, Unknown
  Oligonucleotide sequence
aaagagcctggagaccttaaa

SEQ ID NO: 18 - RNA, 19 nt, Unknown
  Oligonucleotide SiRNA
agagccuggagaccuuaaa

SEQ ID NO: 19 - RNA, 19 nt, Unknown
  Oligonucleotide SiRNA
uuuaaggucuccaggcucu

SEQ ID NO: 20 - DNA, 21 bp, Unknown
  Oligonucleotide sequence
aaggattggtatgtgactctg

SEQ ID NO: 21 - RNA, 19 nt, Unknown
  Oligonucleotide SiRNA
ggauugguaugugacucug

SEQ ID NO: 22 - RNA, 19 nt, Unknown
  Oligonucleotide SiRNA
cagagucacauaccaaucc

SEQ ID NO: 23 - DNA, 21 bp, Unknown
  Oligonucleotide sequence
aagctggattatctcctgttg

SEQ ID NO: 24 - RNA, 19 nt, Unknown
  Oligonucleotide SiRNA
gcuggauuaucuccuguug

SEQ ID NO: 25 - RNA, 19 nt, Unknown
  Oligonucleotide SiRNA
caacaggagauaauccagc

SEQ ID NO: 26 - protein, 41 aa, Homo sapiens
CPVCLERLDASAKVLPCQHTFCKRCLLGIVGSRNELRCPEC

SEQ ID NO: 27 - protein, 56 aa, Homo sapiens
PCAKALYNYEGKEPGDLKFSKGDIIILRRQVDENWYHGEVNGIHGFFPTNFVQIIK

SEQ ID NO: 28 - protein, 60 aa, Homo sapiens
PQCKALYDFEVKDKEADKDCLPFAKDDVLTVIRRVDENWAEGMLADKIGIFPISYVEFNS

SEQ ID NO: 29 - protein, 58 aa, Homo sapiens
SVYVAIYPYTPRKEDELELRKGEMFLVFERCQDGWFKGTSMHTSKIGVFPGNYVAPVT

SEQ ID NO: 30 - protein, 57 aa, Homo sapiens
ERHRVVVSYPPQSEAELELKEGDIVFVHKKREDGWFKGTLQRNGKTGLFPGSFVENI

SEQ ID NO: 31 - DNA, 121 bp, Homo sapiens
tgtccggtgtgtctagagcgccttgatgcttctgcgaaggtcttgccttgccagcatacgttttgcaagcgatgtttgctggggatcgtaggttctcgaaatgaactcagatgtcccgagt

SEQ ID NO: 32 - DNA, 165 bp, Homo sapiens
ccatgtgccaaagcgttatacaactatgaaggaaaagagcctggagaccttaaattcagcaaaggcgacatcatcattttgcgaagacaagtggatgaaaattggtaccatggggaagtcaatggaatccatggctttttccccaccaactttgtgcagattatt

SEQ ID NO: 33 - DNA, 177 bp, Homo sapiens
cctcagtgcaaagcactttatgactttgaagtgaaagacaaggaagcagacaaagattgccttccatttgcaaaggatgatgttctgactgtgatccgaagagtggatgaaaactgggctgaaggaatgctggcagacaaaataggaatatttccaatttcatatgttgagtttaac

SEQ ID NO: 34 - DNA, 171 bp, Homo sapiens
agtgtgtatgttgctatatatccatacactcctcggaaagaggatgaactagagctgagaaaaggggagatgtttttagtgtttgagcgctgccaggatggctggttcaaagggacatccatgcataccagcaagataggggttttccctggcaattatgtggcaccagtc

SEQ ID NO: 35 - DNA, 169 bp, Homo sapiens
gaaaggcacagggtggtggtttcctatcctcctcagagtgaggcagaacttgaacttaaagaaggagatattgtgtttgttcataaaaaacgagaggatggctggttcaaaggcacattacaacgtaatgggaaaactggccttttcccaggaagctttgtggaaaaca

SEQ ID NO: 36 - DNA, 5128 bp, Homo sapiens; identical to SEQ ID NO: 3

SEQ ID NO: 37 - protein, 888 aa, Homo sapiens; identical to SEQ ID NO: 2

SEQ ID NO: 38 - protein, 5 aa, Unknown
  Concensus pKA phosphorylation site
  VARIANT 1: Xaa = K or R
  VARIANT 3: Xaa = Any amino acid
  VARIANT 4: Xaa = S or T
  VARIANT 5: Xaa = Hydrophobic amino acid
XRXXX

SEQ ID NO: 39 - protein, 5 aa, Unknown
  Concensus pKA phosphorylation site
  VARIANT 2, 3: Xaa = Any Amino Acid
  VARIANT 4: Xaa = S or T
  VARIANT 5: Xaa = Hydrophobic amino acid
RXXXX

SEQ ID NO: 40 - protein, 18 aa, Unknown
  RING/domain consensus sequence
  VARIANT 2, 3, 5, 7, 9, 11, 12, 14, 16, 17: Xaa = Any amino acid
CXXCXCXHXCXXCXCXXC

SEQ ID NO: 41 - protein, 18 aa, Unknown
  RING/domain consensus sequence
  VARIANT 2, 3, 5, 7, 9, 11, 12, 14, 16, 17: Xaa = Any amino acid
CXXCXCXHXHXXCXCXXC

SEQ ID NO: 42 - protein, 7 aa, Unknown
  SH3-binding consensus peptide sequence
  VARIANT 2, 3, 5: Xaa = Any Amino Acid
  VARIANT 6: Xaa = Hydrophobic amino acid
RXXPXXP

SEQ ID NO: 43 - protein, 4 aa, Unknown
  SH3-binding consensus peptide sequence
  VARIANT 2: Xaa = T or S
PXAP

SEQ ID NO: 44 - protein, 5 aa, Unknown
  SH3-binding consensus peptide sequence
PFRDY

SEQ ID NO: 45 - protein, 7 aa, Unknown
  SH3-binding consensus peptide sequence
RPEPTAP

SEQ ID NO: 46 - protein, 7 aa, Unknown
  SH3-binding consensus peptide sequence
RQGPKEP

SEQ ID NO: 47 - protein, 9 aa, Unknown
  SH3-binding consensus peptide sequence
RQGPKEPFR

SEQ ID NO: 48 - protein, 9 aa, Unknown
  SH3-binding consensus peptide sequence
RPEPTAPEE

SEQ ID NO: 49 - protein, 7 aa, Unknown
  SH3-binding consensus peptide sequence
RPLPVAP

SEQ ID NO: 50 - protein, 6 aa, Unknown
  pKA peptide inhibitor
  VARIANT 1, 4, 6: Xaa = Any Amino Acid
XRRXAX

SEQ ID NO: 51 - protein, 9 aa, Unknown
  pKA peptide inhibitor
  VARIANT 1: Xaa = Myristoylated glycine
XRTGRRNAI

SEQ ID NO: 52 - protein, 20 aa, Unknown
  pKA peptide inhibitor
IASGRTGRRNAIHDELVSSA

SEQ ID NO: 53 - protein, 7 aa, Unknown
  Peptide substrate for pKA
LRRASLG

SEQ ID NO: 54 - DNA, 53 bp, Unknown
  Oligonucleotide sequence
cacacactgccgtcaactgttcaagagacagttgacggcagtgtgtgtttttt

SEQ ID NO: 55 - DNA, 61 bp, Unknown
  Oligonucleotide sequence
aattaaaaaacacacactgccgtcaactgtctcttgaacagttgacggcagtgtgtgggcc

SEQ ID NO: 56 - DNA, 50 bp, Unknown
  Oligonucleotide sequence
aacagaggccttggaaacctggaagcttgcaggtttccaaggcctctgtt

SEQ ID NO: 57 - DNA, 54 bp, Unknown
  Oligonucleotide sequence
gatcaacagaggccttggaaacctgcaagcttccaggtttccaaggcctctgtt

SEQ ID NO: 58 - DNA, 29 bp, Unknown
  Oligonucleotide sequence
ggcccactagtcaaggtcgggcaggaaga

SEQ ID NO: 59 - DNA, 48 bp, Unknown
  Oligonucleotide sequence
gccgaattcaaaaaggatccggcgatatccggtgtttcgtcctttcca

SEQ ID NO: 60 - protein, 836 aa, Homo sapiens
RTLVGSGVEELPSNILLVRLLDGIKQRPWKPGPGGGSGTNCTNALRSQSSTVANCSSKDLQSSQGGQQPRVQSWSPPVRGIPQLPCAKALYNYEGKEPGDLKFSKGDIIILRRQVDENWYHGEVNGIHGFFPTNFVQIIKPLPQPPPQCKALYDFEVKDKEADKDCLPFAKDDVLTVIRRVDENWAEGMLADKIGIFPISYVEFNSAAKQLIEWDKPPVPGVDAGECSSAAAQSSTAPKHSDTKKNTKKRHSFTSLTMANKSSQASQNRHSMEISPPVLISSSNPTAAARISELSGLSCSAPSQVHISTTGLIVTPPPSSPVTTGPSFTFPSDVPYQAALGTLNPPLPPPPLLAATVLASTPPGATAAAAAAGMGPRPMAGSTDQIAHLRPQTRPSVYVAIYPYTPRKEDELELRKGEMFLVFERCQDGWFKGTSMHTSKIGVFPGNYVAPVTRAVTNASQAKVPMSTAGQTSRGVTMVSPSTAGGPAQKLQGNGVAGSPSVVPAAVVSAAHIQTSPQAKVLLHMTGQMTVNQARNAVRTVAAHNQERPTAAVTPIQVQNAAGLSPASVGLSHHSLASPQPAPLMPGSATHTAAISISRASAPLACAAAAPLTSPSITSASLEAEPSGRIVTVLPGLPTSPDSASSACGNSSATKPDKDSKKEKKGLLKLLSGASTKRKPRVSPPASPTLEVELGSAELPLQGAVGPELPPGGGHGRAGSCPVDGDGPVTTAVAGAALAQDAFHRKASSLDSAVPIAPPPRQACSSLGPVLNESRPVVCERHRVVVSYPPQSEAELELKEGDIVFVHKKREDGWFKGTLQRNGKTGLFPGSFVENI

SEQ ID NO: 61 - DNA, 3600 bp, Homo sapiens
ggtggagctgtcgcctagccgctatcgcagagtggagcggggctgggagcaaagcgctgagggagctcggtacgccgccgcctcgcacccgcagcctcgcgcccgccgccgcccgtccccagagaaccatggagtctggcagtaccgccgccagtgaggaggcacgcagccttcgagaatgtgagctctacgtccagaagcataacattcaagcgctgctcaaagattctattgtgcagttgtgcactgctcgacctgagagacccatggcattcctcagggaatactttgagaggttggagaaggaggaggcaaaacagattcagaatctgcagaaagcaggcactcgtacagactcaagggaggatgagatttctcctcctccacccaacccagtggttaaaggtaggaggcgacgaggtgctatcagcgctgaggtctacacggaggaagatgcggcatcctatgttagaaaggttataccaaaagattacaagacaatggccgctttagccaaagccattgaaaagaatgtgctgttttcacatcttgatgataatgagagaagtgatatttttgatgccatgttttcggtctcctttatcgcaggagagactgtgattcagcaaggtgatgaaggggataacttctatgtgattgatcaaggagagacggatgtctatgttaacaatgaatgggcaaccagtgttggggaaggagggagctttggagaacttgctttgatttatggaacaccgagagcagccactgtcaaagcaaagacaaatgtgaaattgtggggcatcgaccgagacagctatagaagaatcctcatgggaagcacactgagaaagcggaagatgtatgaggaattccttagtaaagtctctattttagagtctctggacaagtgggaacgtcttacggtagctgatgcattggaaccagtgcagtttgaagatgggcagaagattgtggtgcagggagaaccaggggatgagttcttcattattttagaggggtcagctgctgtgctacaacgtcggtcagaaaatgaagagtttgttgaagtgggaagattggggccttctgattattttggtgaaattgcactactgatgaatcgtcctcgtgctgccacagttgttgctcgtggccccttgaagtgcgttaagctggaccgacctagatttgaacgtgttcttggcccatgctcagacatcctcaaacgaaacatccagcagtacaacagttttgtgtcactgtctgtctgaaatctgcctcctgtgcctcccttttctcctctccccaatccatgcttcactcatgcaaactgctttattttccctacttgcagcgccaagtggccactggcatcgcagcttcctgtctgtttatatattgaaagttgcttttattgcaccattttcaatttggagcattaactaaatgctcatacacagttaaataaatagaaagagttctatggagactttgctgttactgcttctctttgtgcagtgttagtattcaccctgggcagtgagtgccatgctttttggtgagggcagatcccagcacctattgaattaccatagagtaatgatgtaacagtgcaagattttttttttaagtgacataattgtccagttataagcgtatttagactgtggccatatatgctgtatttctttgtagaataaatggtttctcattaaactctaaagattagggaaaatggatatagaaaatcttagtatagtagaaagacatctgcctgtaattaaactagtttaagggtggaaaaatgcccatttttgctaattatcaatgggatatgattggttcagtttttttttttccagagttgttgtttgccaagctaatctgcctggttttatttatatcttgttattaatgtttcttctccaattctgaaatacttttgagtatggctatctatacctgccttttaagtttgaaactaactcatagattgcaaatattggttagtatttaactacatctgcctcggctcacaaattccgattagacctttatccagctagtgccaaataattgatcagatgctgaattgagaataagaatttgaggtctacattcttggttgttaatttagagcgtttggttaaagtatgtccttcagctgactccagtataatctcctctgctcattaaactgattccaggagattggatttgctgtgactagatacagatggagcaaatgtcctaacagagaaatagaggtgatgctgctaaagggagaaatgccaggcggacaaagttcagtgtcgggaattttccccgtgacattcactggggcatgagattttggaagaagttttttactttggtttagtctttttttccttcctttttattcagctagaatttctggtgggttgatggtagggtataatgtgtctgtgttgcttcaaattggtctgaaaggctatcctgcggaaagtcctgctttcctatctagcatttatttctctggcaaacttttctttcttttcttttttaaagtaaacttgtgtattgagtcttaactgtatttcagtattttccagccttatgtgttacattattccaatgatacccaacagtttatttttattatttttttaaacaaaatttcacagttctgtaatgtaggcacttttattttcattgtgatttatatataaggtaatgtagggttatatttgggagtgactgcaagcatttttccatctgtgtgcaactaactgactctgttattgatcccttctcctgccctttcccaggtaatttaaattggtcatggtagatttttttcatagatttgaaaaacttttaggttgttaccaagtatgaagtataaatctggggaagaggttttatttacattttagggtgggtaagaaagccaccttgttacaaattttttaatttccaaaataatctatattaaatgagggtttctgatctgtactttgtgtttagctacctttttatatttaaaaaattaaaaatgaaaattacgttcttacaagcttaaagcttgatttgatctttgtttaaatgccaaaatgtacttaaatgagttacttagaatgccataaaattgcagtttcatgtatgtatataatcatgctcatgtatatttagttacgtataatgctttctgagtgagttttactcttaaatcatttggttaaatcatttggcttgctgtttactcccttctgtagtttttaattaaaaactttaaagataagtctacattaaacaatgatcacatctaaagctttatctttgtgtaatctaagtatatgtgagaaatcagaattggcataatttgtcttagttgatattcaaggctttaaaagtcattattcctgggcttggtaagtgaatttatgagatttactgctctagaaagtatagatggcgaaaggaccgttttgtattgcttcctgattaccagtctgattataccatgtgtgctaatatactttttttgttatagattgtcttaatggtaggtcaagtaataaaaagagatgaaataatttaaaaaaaaaaaaaaaa

SEQ ID NO: 62 - DNA, 412 bp, Homo sapiens
agaggcgtcaagggaggccggagggagagtggggtggacagaggagcggagggacgagagggaagcgcacgatagctgcgcggagagagagcgaagagcaggaggaggaacaaaggcgacccaagacacccagagagggacagagaaccatggagtctggcagtaccgccgccagtgaggaggcacgcagccttcgagaatgtgagctctacgtccagaagcataacattcaagcgctgctcaaagattctattgtgcagttgtgcactgctcgacctgagagacccatggcattcctcagggaatactttgagaggttggagaaggaggaggcaaaacagattcagaatctgcagaaagcaggcactcgtacagactcaagggaggatgagatttctcctcctccacccaa

SEQ ID NO: 63 - DNA, 1940 bp, Homo sapiens
taattttcttgtgtgtttttaaaaattttgattatgctagtagttggctaatcagatcctcactccagtggtttgctctgtgacgttaggatactcccatgggatagaagttacgtatagggaatgtcagatattcttcattgtgctgacttgctttcgcttacagttgacttttgtgccctggtaattctgtatcctgtttaccgtttacctacttcccacgtcatcatgatttcttttgagggagaactgaatgaaattcccttaagggcctgacttcagcacccgtctctgcagaggttagtggctcatacttcctcccaggagctgaggttatcgactctcactgttgcctacagagcacagatcctgaactaaatgaaacatttacttggaataatgctaattctgtacatattttattccctagtccccacttccctgtttaaaaacaaaatctacttagaaaaaaatccctgtgaatcagttgtctaatgaatttagcaagttaaatgccagattgacattttgctttatagtttatacaagcatgtgtgtgtttttttctcgcagagaaccatggagtctggcagtaccgccgccagtgaggaggcacgcagccttcgagaatgtgagctctacgtccagaagcataacattcaagcgctgctcaaagattctattgtgcagttgtgcactgctcgacctgagagacccatggcattcctcagggaatactttgagaggttggagaaggaggaggcaaaacagattcagaatctgcagaaagcaggcactcgtacagactcaagggaggatgagatttctcctcctccacccaacccagtggttaaaggtaggaggcgacgaggtgctatcagcgctgaggtctacacggaggaagatgcggcatcctatgttagaaaggttataccaaaagattacaagacaatggccgctttagccaaagccattgaaaagaatgtgctgttttcacatcttgatgataatgagagaagtgatatttttgatgccatgttttcggtctcctttatcgcaggagagactgtgattcagcaaggtgatgaaggggataacttctatgtgattgatcaaggagagacggatgtctatgttaacaatgaatgggcaaccagtgttggggaaggagggagctttggagaacttgctttgatttatggaacaccgagagcagccactgtcaaagcaaagacaaatgtgaaattgtggggcatcgaccgagacagctatagaagaatcctcatgggaagcacactgagaaagcggaagatgtatgaggaattccttagtaaagtctctattttagagtctctggacaagtgggaacgtcttacggtagctgatgcattggaaccagtgcagtttgaagatgggcagaagattgtggtgcagggagaaccaggggatgagttcttcattattttagaggggtcagctgctgtgctacaacgtcggtcagaaaatgaagagtttgttgaagtgggaagattggggccttctgattattttggtgaaattgcactactgatgaatcgtcctcgtgctgccacagttgttgctcgtggccccttgaagtgcgttaagctggaccgacctagatttgaacgtgttcttggcccatgctcagacatcctcaaacgaaacatccagcagtacaacagttttgtgtcactgtctgtctgaaatccgcctcctgtgcctcccttttctcctctccccaatccatgcttcactcatgcaaactgctttattttccctacttgcagcgccaagtggccactggcatcgcagcttcctgtctgtttatatattgaaagttgcttttattgcaccattttcaatttggagcattaactaaatgctcatacacagttaaataaatagaaagagttctatgg

SEQ ID NO: 64 - DNA, 1476 bp, Homo sapiens
ggcagagtggagcggggctgggagcaaagcgctgagggagctcggtacgccgccgcctcgcacccgcagcctcgcgcccgccgccgcccgtccccagagaaccatggagtctggcagtaccgccgccagtgaggaggcacgcagccttcgagaatgtgagctctacgtccagaagcataacattcaagcgctgctcaaagattctattgtgcagttgtgcactgctcgacctgagagacccatggcattcctcagggaatactttgagaggttggagaaggaggaggcaaaacagattcagaatctgcagaaagcaggcactcgtacagactcaagggaggatgagatttctcctcctccacccaacccagtggttaaaggtaggaggcgacgaggtgctatcagcgctgaggtctacacggaggaagatgcggcatcctatgttagaaaggttataccaaaagattacaagacaatggccgctttagccaaagccattgaaaagaatgtgctgttttcacatcttgatgataatgagagaagtgatatttttgatgccatgttttcggtctcctttatcgcaggagagactgtgattcagcaaggtgatgaaggggataacttctatgtgattgatcaaggagagacggatgtctatgttaacaatgaatgggcaaccagtgttggggaaggagggagctttggagaacttgctttgatttatggaacaccgagagcagccactgtcaaagcaaagacaaatgtgaaattgtggggcatcgaccgagacagctatagaagaatcctcatgggaagcacactgagaaagcggaagatgtatgaggaattccttagtaaagtctctattttagagtctctggacaagtgggaacgtcttacggtagctgatgcattggaaccagtgcagtttgaagatgggcagaagattgtggtgcagggagaaccaggggatgagttcttcattattttagaggggtcagctgctgtgctacaacgtcggtcagaaaatgaagagtttgttgaagtgggaagattggggccttctgattattttggtgaaattgcactactgatgaatcgtcctcgtgctgccacagttgttgctcgtggccccttgaagtgcgttaagctggaccgacctagatttgaacgtgttcttggcccatgctcagacatcctcaaacgaaacatccagcagtacaacagttttgtgtcactgtctgtctgaaatctgcctcctgtgcctcccttttctcctctccccaatccatgcttcactcatgcaaactgctttattttccctacttgcagcgccaagtggccactggcatcgcagcttcctgtctgtttatatattaaagttgcttttattgcaccattttcaatttggagcattaactaaatgctcatacacagttaaataaatagaaagagttctatggaaaaaaaaaaaaa

SEQ ID NO: 65 - DNA, 3036 bp, Homo sapiens
gctgggagcaaagcgctgagggagctcggtacgccgccgcctcgcacccgcagcctcgcgcccgccgccgcccgtccccagagaaccatggagtctggcagtaccgccgccagtgaggaggcacgcagccttcgagaatgtgagctctacgtccagaagcataacattcaagcgctgctcaaagattctattgtgcagttgtgcactgctcgacctgagagacccatggcattcctcagggaatactttgagaggttggagaaggaggaggcaaaacagattcagaatctgcagaaagcaggcactcgtacagactcaagggaggatgagatttctcctcctccacccaacccagtggttaaaggtaggaggcgacgaggtgctatcagcgctgaggtctacacggaggaagatgcggcatcctatgttagaaaggttataccaaaagattacaagacaatggccgctttagccaaagccattgaaaagaatgtgctgttttcacatcttgatgataatgagagaagtgatatttttgatgccatgttttcggtctcctttatcgcaggagagactgtgattcagcaaggtgatgaaggggataacttctatgtgattgatcaaggagagacggatgtctatgttaacaatgaatgggcaaccagtgttggggaaggagggagctttggagaacttgctttgatttatggaacaccgagagcagccactgtcaaagcaaagacaaatgtgaaattgtggggcatcgaccgagacagctatagaagaatcctcatgggaagcacactgagaaagcggaagatgtatgaggaattccttagtaaagtctctattttagagtctctggacaagtgggaacgtcttacggtagctgatgcattggaaccagtgcagtttgaagatgggcagaagattgtggtgcagggagaaccaggggatgagttcttcattattttagaggggtcagctgctgtgctacaacgtcggtcagaaaatgaagagtttgttgaagtgggaagattggggccttctgattattttggtgaaattgcactactgatgaatcgtcctcgtgctgccacagttgttgctcgtggccccttgaagtgcgttaagctggaccgacctagatttgaacgtgttcttggcccatgctcagacatcctcaaacgaaacatccagcagtacaacagttttgtgtcactgtctgtctgaaatctgcctcctgtgcctcccttttctcctctccccaatccatgcttcactcatgcaaactgctttattttccctacttgcagcgccaagtggccactggcatcgcagcttcctgtctgtttatatattgaaagttgcttttattgcaccattttcaatttggagcattaactaaatgctcatacacagttaaataaatagaaagagttctatggagactttgctgttactgcttctctttgtgcagtgttagtattcaccctgggcagtgagtgccatgctttttggtgagggcagatccagcacctattgaattaccatagagtaatgatgtaacagtgcaagatttttttttttaagtgacataattgtccagttataagcgtatttagactgtggccatatatgctgtatttctttgtagaataaatggtttctcattaaactctaaagattagggaaatggatatagaaaatcttagtatagtagaaagacatctgcctgtaattaaactagtttaagggtggaaaaatgaaaatttttgctaattatcaatgggatatgattggttcagttttttttttccagagttgttgtttgccaagctaatctgcctggtttatttatatcttgttattaatgtttcttctccaattctgaaatacttttgagtatggctatctatacctgccttttaagtttgaaactaactcatagatgcaaatattggttagtatttaactacatctgcctcggctcacaaattccgattagacctttatccagctagtgccaaataattgatcagatgctgaattgagaataagaatttgaggtctacattcttggttgttaatttagagcgtttggttaaagtatgtccttcagctgactccagtataatctcctctgctcattaaactgattccaggagattggatttgctgtgactagatacagatggagcaaatgtcctaacagagaaatagaggtgatgctgctaaagggagaaatgccaggcggacaaagttcagtgtcgggaattttccccgtgacattcactggggcatgagattttggaagaagttttttactttggtttagtctttttttcctcctttttattcagctagaatttctggtgggttgatggtagggtataatgtgtctgtgttgcttcaaattggtctgaaaggctatcctgctgaaagtcctgctttcctatctagcatttattcctctggcaaacttttctttcttttcttttttaaagtaaacttgtgtattgagtcttaactgtatttcagtattttccagccttatgtgttacattattccaatgatacccaacagtttatttttattatttttttaaacaaaatttcacagttctgtaatgtaggcacttttattttcattgtgatttatatataaggtaatgtagggttatatttgggagtgactgcaagcatttttccatctgtgtgcaactaactgactctgttattgatcccttctcctgccctttcccaggtaatttaaattggtcatggtagatttttttcatagatttgaaaaacttttaggttgttaccaagtatgaagtataaatctggggaagaggttttatttacattttagggtgggtaagaaagccaccttgttacaaattttttaatttccaaaataatctatattaaatgagggtttctgatctgtactttgtgtttagctacctttttatatttaaaaaattaaaaatgaaaattatgttcttacaagcttaaagcttgatttgatct

SEQ ID NO: 66 - DNA, 3036 bp, Homo sapiens; identical to SEQ ID NO: 65

SEQ ID NO: 67 - DNA, 1010 bp, Homo sapiens
tattttccagccttatgtgttacattattccaatgatacccaacagtttatttttattatttttttaaacaaaatttcacagttctgtaatgtaggcacttttattttcattgtgatttatatataaggtaatgtagggttatatttgggagtgactgcaagcatttttccatctgtgtgcaactaactgactctgttattgatcccttctcctgccctttcccaggtaatttaaattggtcatggtagatttttttcatagatttgaaaaacttttaggttgttaccaagtatgaagtataaatctggggaagaggttttatttacattttagggtgggtaagaaagccaccttgttacaaattttttaatttccaaaataatctatattaaatgagggtttctgatctgtactttgtgtttagctacctttttatatttaaaaaattaaaaatgaaaattacgttcttacaagcttaaagcttgatttgatctttgtttaaatgccaaaatgtacttaaatgagttacttagaatgccataaaattgcagtttcatgtatgtatataatcatgctcatgtatatttagttacgtataatgctttctgagtgagttttactcttaaatcatttggttaaatcatttggcttgctgtttactcccttctgtagtttttaattaaaaactttaaagataagtctacattaaacaatgatcacatctaaagctttatctttgtgtaatctaagtatatgtgagaaatcagaattggcataatttgtcttagttgatattcaaggctttaaaagtcattattcctgggcttggtaagtgaatttatgagatttactgctctagaaagtatagatggccaaaggaccgttatgtattgcttcctgattaccagtctgattataccatgtgtgctaatatactttttttgttatagattgtcttaatggtaggtcaagtaataaaaagagatgaaataatttaaaaaaaaaaaaa

SEQ ID NO: 68 - protein, 87 aa, Homo sapiens
MESGSTAASEEARSLRECELYVQKHNIQALLKDSIVQLCTARPERPMAFLREYFERLEKEEAKQIQNLQKAGTRTDSREDEISPPPP

SEQ ID NO: 69 - protein, 381 aa, Homo sapiens
MESGSTAASEEARSLRECELYVQKHNIQALLKDSIVQLCTARPERPMAFLREYFERLEKEEAKQIQNLQKAGTRTDSREDEISPPPPNPVVKGRRRRGAISAEVYTEEDAASYVRKVIPKDYKTMAALAKAIEKNVLFSHLDDNERSDIFDAMFSVSFIAGETVIQQGDEGDNFYVIDQGETDVYVNNEWATSVGEGGSFGELALIYGTPRAATVKAKTNVKLWGIDRDSYRRILMGSTLRKRKMYEEFLSKVSILESLDKWERLTVADALEPVQFEDGQKIVVQGEPGDEFFIILEGSAAVLQRRSENEEFVEVGRLGPSDYFGEIALLMNRPRAATVVARGPLKCVKLDRPRFERVLGPCSDILKRNIQQYNSFVSLSV

SEQ ID NO: 70 - DNA, 2685 bp, Homo sapiens
tcgggctgaggttcccgggcgggcgggcgcggagagacgcgggaagcaggggctgggcgggggtcgcggcgccgcagctagcgcagccagcccgagggccgccgccgccgccgcccagcgcgctccggggccgccggccgcagccagcacccgccgcgccgcagctccgggaccggccccggccgccgccgccgcgatgggcaacgccgccgccgccaagaagggcagcgagcaggagagcgtgaaagaattcttagccaaagccaaagaagattttcttaaaaaatgggaaagtcccgctcagaacacagcccacttggatcagtttgaacgaatcaagaccctcggcacgggctccttcgggcgggtgatgctggtgaaacacaaggagaccgggaaccactatgccatgaagatcctcgacaaacagaaggtggtgaaactgaaacagatcgaacacaccctgaatgaaaagcgcatcctgcaagctgtcaactttccgttcctcgtcaaactcgagttctccttcaaggacaactcaaacttatacatggtcatggagtacgtgcccggcggggagatgttctcacacctacggcggatcggaaggttcagtgagccccatgcccgtttctacgcggcccagatcgtcctgacctttgagtatctgcactcgctggatctcatctacagggacctgaagccggagaatctgctcattgaccagcagggctacattcaggtgacagacttcggtttcgccaagcgcgtgaagggccgcacttggaccttgtgcggcacccctgagtacctggcccctgagattatcctgagcaaaggctacaacaaggccgtggactggtgggccctgggggttcttatctatgaaatggccgctggctacccgcccttcttcgcagaccagcccatccagatctatgagaagatcgtctctgggaaggtgcgcttcccttcccacttcagctctgacttgaaggacctgctgcggaacctcctgcaggtagatctcaccaagcgctttgggaacctcaagaatggggtcaacgatatcaagaaccacaagtggtttgccacaactgactggattgccatctaccagaggaaggtggaagctcccttcataccaaagtttaaaggccctggggatacgagtaactttgacgactatgaggaagaagaaatccgggtctccatcaatgagaagtgtggcaaggagttttctgagttttaggggcatgcctgtgcccccatgggttttcttttttcttttttcttttttttggtcgggggggtgggagggttggattgaacagccagagggccccagagttccttgcatctaatttcacccccaccccaccctccagggttagggggagcaggaagcccagataatcagagggacagaaacaccagctgctccccctcatccccttcaccctcctgccccctctcccacttttcccttcctctttccccacagccccccagcccctcagccctcccagcccacttctgcctgttttaaacgagtttctcaactccagtcagaccaggtcttgctggtgtatccagggacagggtatggaaagaggggctcacgcttaactccagcccccacccacacccccatcccacccaaccacaggccccacttgctaagggcaaatgaacgaagcgccaaccttcctttcggagtaatcctgcctgggaaggagagatttttagtgacatgttcagtgggttgcttgctagaatttttttaaaaaaacaacaatttaaaatcttatttaagttccaccagtgcctccctccctccttcctctactcccacccctcccatgtccccccattcctcaaatccattttaaagagaagcagactgactttggaaagggaggcgctggggtttgaacctccccgctgctaatctcccctgggcccctccccggggaatcctctctgccaatcctgcgagggtctaggcccctttaggaagcctccgctctctttttccccaacagacctgtcttcacccttgggctttgaaagccagacaaagcagctgcccctctccctgccaaagaggagtcatcccccaaaaagacagagggggagccccaagcccaagtctttcctcccagcagcgtttccccccaactccttaattttattctccgctagattttaacgtccagccttccctcagctgagtggggagggcatccctgcaaaagggaacagaagaggccaagtccccccaagccacggcccggggttcaaggctagagctgctggggaggggctgcctgttttactcacccaccagcttccgcctcccccatcctgggcgcccctcctccagcttagctgtcagctgtccatcacctctcccccactttctcatttgtgcttttttctctcgtaatagaaaagtggggagccgctggggagccaccccattcatccccgtatttccccctctcataacttctccccatcccaggaggagttctcaggcctggggtggggccccgggtgggtgcgggggcgattcaacctgtgtgctgcgaaggacgagacttcctcttgaacagtgtgctgttgtaaacatatttgaaaactattaccaataaagttttgtttaaaaaaaaaaaaaaaaaa

SEQ ID NO: 71 - DNA, 120 bp, Homo sapiens
ggtgccctgagaacaggactgagtgatggcttccaactccagcgatgtgaaagaattcttagccaaagccaaagaagattttcttaaaaaatgggaaagtcccgctcagaacacagccca

SEQ ID NO: 72 - DNA, 2549 bp, Homo sapiens
  misc_feature 6: n = A,T,C or G
cagtgngctccgggccgccggccgcagccagcacccgccgcgccgcagctccgggaccggccccggccgccgccgccgcgatgggcaacgccgccgccgccaagaagggcagcgagcaggagagcgtgaaagaattcttagccaaagccaaagaagattttcttaaaaaatgggaaagtcccgctcagaacacagcccacttggatcagtttgaacgaatcaagaccctcggcacgggctccttcgggcgggtgatgctggtgaaacacaaggagaccgggaaccactatgccatgaagatcctcgacaaacagaaggtggtgaaactgaaacagatcgaacacaccctgaatgaaaagcgcatcctgcaagctgtcaactttccgttcctcgtcaaactcgagttctccttcaaggacaactcaaacttatacatggtcatggagtacgtgcccggcggggagatgttctcacacctacggcggatcggaaggttcagtgagccccatgcccgtttctacgcggcccagatcgtcctgacctttgagtatctgcactcgctggatctcatctacagggacctgaagccggagaatctgctcattgaccagcagggctacattcaggtgacagacttcggtttcgccaagcgcgtgaagggccgcacttggaccttgtgcggcacccctgagtacctggcccctgagattatcctgagcaaaggctacaacaaggccgtggactggtgggccctgggggttcttatctatgaaatggccgctggctacccgcccttcttcgcagaccagcccatccagatctatgagaagatcgtctctgggaaggtgcgcttcccttcccacttcagctctgacttgaaggacctgctgcggaacctcctgcaggtagatctcaccaagcgctttgggaacctcaagaatggggtcaacgatatcaagaaccacaagtggtttgccacaactgactggattgccatctaccagaggaaggtggaagctcccttcataccaaagtttaaaggccctggggatacgagtaactttgacgactatgaggaagaagaaatccgggtctccatcaatgagaagtgtggcaaggagttttctgagttttaggggcatgcctgtgcccccatgggttttcttttttcttttttcttttttttggtcgggggggtgggagggttggattgaacagccagagggccccagagttccttgcatctaatttcacccccaccccaccctccagggttagggggagcaggaagcccagataatcagagggacagaaacaccagctgctccccctcatccccttcaccctcctgccccctctcccacttttcccttcctctttccccacagccccccagcccctcagccctcccagcccacttctgcctgttttaaacgagtttctcaactccagtcagaccaggtcttgctggtgtatccagggacagggtatggaaagaggggctcacgcttaactccagcccccacccacacccccatcccacccaaccacaggccccacttgctaagggcaaatgaacgaagcgccaaccttcctttcggagtaatcctgcctgggaaggagagatttttagtgacatgttcagtgggttgcttgctagaatttttttaaaaaaacaacaatttaaaatcttatttaagttccaccagtgcctccctccctccttcctctactcccacccctcccatgtccccccattcctcaaatccattttaaagagaagcagactgactttggaaagggaggcgctggggtttgaacctccccgctgctaatctcccctgggcccctccccggggaatcctctctgccaatcctgcgagggtctaggcccctttaggaagcctccgctctctttttccccaacagacctgtcttcacccttgggctttgaaagccagacaaagcagctgcccctctccctgccaaagaggagtcatcccccaaaaagacagagggggagccccaagcccaagtctttcctcccagcagcgtttccccccaactccttaattttattctccgctagattttaacgtccagccttccctcagctgagtggggagggcatccctgcaaaagggaacagaagaggccaagtccccccaagccacggcccggggttcaaggctagagctgctggggaggggctgcctgttttactcacccaccagcttccgcctcccccatcctgggcgcccctcctccagcttagctgtcagctgtccatcacctctcccccactttctcatttgtgcttttttctctcgtaatagaaaagtggggagccgctggggagccaccccattcatccccgtatttccccctctcataacttctccccatcccaggaggagttctcaggcctggggtggggccccgggtgggtgcgggggcgattcaacctgtgtgctgcgaaggacgagacttcctcttgaacagtgtgctgttgtaaacatatttgaaaactattaccaataaagtttgtt

SEQ ID NO: 73 - DNA, 936 bp, Homo sapiens
gaattcttagccaaagccaaagaagattttcttaaaaaatgggaaagtcccgctcagaacacagcccacttggatcagtttgaacgaatcaagaccctcggcacgggctccttcgggcgggtgatgctggtgaaacacaaggagaccgggaaccactatgccatgaagatcctcgacaaacagaaggtggtgaaactgaaacagatcgaacacaccctgaatgaaaagcgcatcctgcaagctgtcaactttccgttcctcgtcaaactcgagttctccttcaaggacaactcaaacttatacatggtcatggagtacgtgcccggcggggagatgttctcacacctacggcggatcggaaggttcagtgagccccatgcccgtttctacgcggcccagatcgtcctgacctttgagtatctgcactcgctggatctcatctacagggacctgaagccggagaatctgctcattgaccagcagggctacattcaggtgacagacttcggtttcgccaagcgcgtgaagggccgcacttggaccttgtgcggcacccctgagtacctggcccctgagattatcctgagcaaagtaggagcctccccagccctccccttcccctgaggccggctctgctctcctgctctcgcctcctcctcaccctgtgcccccccatcttgctccagggctacaacaaggccgtggactggtgggccctgggggttcttatctatgaaatggccgctggctacccgcccttcttcgcagaccagcccatccagatctatgagaagatcgtctctgggaaggtgaggtccggatgtgggacacagccctggaagaaacagaccgttccctgctcacccatcctattccctggggagccctgcttgttgtcagaataatctagaagttccttaaaaaaaaaaaaaaaaa

SEQ ID NO: 74 - DNA, 560 bp, Homo sapiens
tgagaacaggactgagtgatggcttccaactccagcgatgtgaaagaattcttagccaaagccaaagaagattttcttaaaaaatgggaaagtcccgctcagaacacagcccacttggatcagtttgaacgaatcaagaccctcggcacgggctccttcgggcgggtgatgctggtgaaacacaaggagaccgggaaccactatgccatgaagatcctcgacaaacagaaggtggtgaaactgaaacagatcgaacacaccctgaatgaaaagcgcatcctgcaagctgtcaactttccgttcctcgtcaaactcgagttctccttcaaggacaactcaaacttatacatggtcatggagtacgtgcccggcggggagatgttctcacacctacggcggatcggaaggttcagtgagccccatgcccgtttctacgcggcccagatcgtcctgacctttgagtatctgcactcgctggatctcatctacagggacctgaagccggagaatctgctcattgaccagcagggctacattcaggtgacagacttcggtttcgc

SEQ ID NO: 75 - DNA, 594 bp, Homo sapiens
cccagtggcctctgggttgggtttctcttcctgctcccaccccacggctccctagctccccctgcaggcagggttctggggacagacagccgaacagacacggcaggtctcatgagccttcccagccaccgtagtgccggtgccctgagaacaggactgagtgatggcttccaactccagcgatgtgaaagaattcttagccaaagccaaagaagattttcttaaaaaatgggaaagtcccgctcagaacacagcccacttggatcagtttgaacgaatcaagaccctcggcacgggctccttcgggcgggtgatgctggtgaaacacaaggagaccgggaaccactatgccatgaagatcctcgacaaacagaaggtggtgaaactgaaacagatcgaacacaccctgaatgaaaagcgcatcctgcaagctgtcaactttccgttcctcgtcaaactcgagttctccttcaaggacaactcaaacttatacatggtcatggagtacgtgcccggcggggagatgttctcacacctacggcggatcggaaggttcagtgagccccatgcccgtttctacgcggcccagatcgt

SEQ ID NO: 76 - protein, 207 aa, Homo sapiens
EFLAKAKEDFLKKWESPAQNTAHLDQFERIKTLGTGSFGRVMLVKHKETGNHYAMKILDKQKVVKLKQIEHTLNEKRILQAVNFPFLVKLEFSFKDNSNLYMVMEYVPGGEMFSHLRRIGRFSEPHARFYAAQIVLTFEYLHSLDLIYRDLKPENLLIDQQGYIQVTDFGFAKRVKGRTWTLCGTPEYLAPEIILSKVGASPALPFP

SEQ ID NO: 77 - protein, 181 aa, Homo sapiens
MASNSSDVKEFLAKAKEDFLKKWESPAQNTAHLDQFERIKTLGTGSFGRVMLVKHKETGNHYAMKILDKQKVVKLKQIEHTLNEKRILQAVNFPFLVKLEFSFKDNSNLYMVMEYVPGGEMFSHLRRIGRFSEPHARFYAAQIVLTFEYLHSLDLIYRDLKPENLLIDQQGYIQVTDFGFA

SEQ ID NO: 78 - protein, 144 aa, Homo sapiens
MASNSSDVKEFLAKAKEDFLKKWESPAQNTAHLDQFERIKTLGTGSFGRVMLVKHKETGNHYAMKILDKQKVVKLKQIEHTLNEKRILQAVNFPFLVKLEFSFKDNSNLYMVMEYVPGGEMFSHLRRIGRFSEPHARFYAAQIV

SEQ ID NO: 79 - protein, 31 aa, Homo sapiens
MASNSSDVKEFLAKAKEDFLKKWESPAQNTA

SEQ ID NO: 80 - protein, 351 aa, Homo sapiens
MGNAAAAKKGSEQESVKEFLAKAKEDFLKKWESPAQNTAHLDQFERIKTLGTGSFGRVMLVKHKETGNHYAMKILDKQKVVKLKQIEHTLNEKRILQAVNFPFLVKLEFSFKDNSNLYMVMEYVPGGEMFSHLRRIGRFSEPHARFYAAQIVLTFEYLHSLDLIYRDLKPENLLIDQQGYIQVTDFGFAKRVKGRTWTLCGTPEYLAPEIILSKGYNKAVDWWALGVLIYEMAAGYPPFFADQPIQIYEKIVSGKVRFPSHFSSDLKDLLRNLLQVDLTKRFGNLKNGVNDIKNHKWFATTDWIAIYQRKVEAPFIPKFKGPGDTSNFDDYEEEEIRVSINEKCGKEFSEF

SEQ ID NO: 81 - DNA, 4535 bp, Homo sapiens
agcgggtctgcccgccgccgccactgctgctgccaccgccgtcgccgccgccgccgccgccgccactgctgctgccggtgctaaggagttcgctggagccctttcctcagacccggcccggtcttcgcgcccggactcctggcgccagcgctaggcgcactcaccgctctgacgggtgcagacgcgggagttgtcccagactgtggagtggcgggcacggccccagctccccttccgttccctgaccccttcttgccatcgccccagacatggggaacgcggcgaccgccaagaaaggcagcgaggtggagagcgtgaaagagtttctagccaaagccaaagaagactttttgaaaaaatgggagaatccaactcagaataatgccggacttgaagattttgaaaggaaaaaaacccttggaacaggttcatttggaagagtcatgttggtaaaacacaaagccactgaacagtattatgccatgaagatcttagataagcagaaggttgttaaactgaagcaaatagagcatactttgaatgagaaaagaatattacaggcagtgaattttcctttccttgttcgactggagtatgcttttaaggataattctaatttatacatggttatggaatatgtccctgggggtgaaatgttttcacatctaagaagaattggaaggttcagtgagccccatgcacggttctatgcagctcagatagtgctaacattcgagtacctcaattcactagacatcatctacagagatctaaaacctgaaaatctcttaattgaccatcaaggctatatccaggtcacagactttgggtttgccaaaagagttaaaggcagaacttggacattatgtggaactccagagtatttggctccagaaataattctcagcaagggctacaataaggcagtggattggtgggcattaggagtgctaatctatgaaatggcagctggctatcccccattctttgcagaccaaccaattcagatttatgaaaagattgtttctggaaaggtccgattcccatccaacttcagttcagatctcaaggaccttctacggaacctgctgcaggtggatttgaccaagagatttggaaatctaaagaatggtgtcagtgatataaaaactcacaagtggtttgccacgacagattggattgctatttaccagaggaaggttgaagctccattcataccaaagtttagaggctctggagataccagcaactttgatgactatgaagaagaagatatccgtgtctctataacagaaaaatgtgcaaaagaatttggtgaattttaaagaggaacaagatgacatctgagctcacactcagtgtttgcactctgttgagagataaggtagagctgagaccgtccttgttgaagcagttacctagttccttcattccaacgactgagtgaggtctttattgccatcatcccgtgtgcgcactctgcatccacctatgtaacaaggcaccgctaagcaagcattgtctgtgccataacacagtactagaccactttcttacttctctttgggttgtctttctcctctcctatatccatttcttccttttccaatttcattggttttctctaaacagtgctccattttattttgttggtgtttcagatgggcagtgttatggctacgtgatatttgaagggaaggataagtgttgctttcagtagttattgccaatattgttgttggtcaatggcttgaagataaactttctaataattattatttctttgagtagctcagacttggttttgccaaaactcttggtaatttttgaagatagactgtcttatcaccaaggaaatttatacaaattaagactaactttcttggaattcactattctggcaataaattttggtagactaatacagtacagctagacccagaaatttggaaggctgtagatcagaggttctagttccctttccctccttttatatcctcctctccttgagtaatgaagtgaccagcctgtgtagtgtgacaaacgtgtctcattcagcaggaaaaactaatgatatggatcatcacccagattctctcacttggtaccagcatttctgtaggtattagagaagagttctaagttttctaaaccttaactgttccttaaggattttagccagtattttaatagaacatgattaatgaaagtgacaaattttaaattttctctaatagtcctcatcataaactttttaaaggaaaataagcaaactaaaaagaacattggtttagataaatacttatactttgcaaagtcaaaaatggcttgatttttggaaacaatatagaggtattcatatttaaatgagggtttacatttgttttgttttgtaaccgttaaaaagaagttgtttccagctaattattgtggtgtactatatttgtgagcctagggtaggggcactgctgcaacttctgctttcatcccatgcctcatcaatgaggaaagggaacaaagtgtataaaactgccacaattgtattttaattttgaggtatgatattttcagatatttcataatttctaacctctgttctctcagtaaacagaatgtctgatcgatcatgcagatacaatgttggtatttgagaggttagtttttttcctacacttttttttgccaactgacttaacaacattgctgtcaggtggaaatttcaagcacttttgcacatttagttcagtgtttgttgagaatccatggcttaacccacttgttttgctatttttttctttgcttttaattttccccatctgattttatctctgcgtttcagtgacctaccttaaaacaacacacgagaagagttaaactgggttcattttaatgatcaatttacctgcatataaaatttatttttaatcaagctgatcttaatgtatataatcattctatttgctttattatcggtgcaggtaggtcattaacaccacttcttttcatctgtaccacaccctggtgaaacctttgaagacataaaaaaaacctgtctgagatgttctttctaccaatctatatgtctttcggttatcaagtgtttctgcatggtaatgtcatgtaaatgctgatattgatttcactggtccatctatatttaaaacgtgcaagaaaaaaataaaatactctgctctagcaagttttgtgtaacaaaggcatatcgtcatgttaataaatttaaaacatcattcgtataaaatattttaattttcttgtatttcatttagacccaagaacatgctgaccaatgtgttctatatgtaaactacaaattctatggtagctttgttgtatattattgtaaaattattttaataagtcatggggatgacaatttgattattacaatttagttttcagtaatcaaaaagatttctatgaattctaaaaaatatttttttctatgaaattactagtgcccagctgtagaatctaccttaggtagatgatccctagacatacgttggttttgagggctattcagccattccattttactctctatttaaaggccgtgagcaagcttgtcatgagcaaatatgtcaagggagtcaatctctgaccaatcaagtacactaaattagaatatttttaaagtatgtaacattcccagtttcagccacaatttagccaagaataagataaaaacttgaataagaagtaagtagcataaatcagtatttaacctaaaattacatatttgaaacagaagatattatgttatgctcagtaaataattaagagatggcattgtgtaagaaggagccctagactgaaagtcaagacatctgaatttcaggctggaaaactatcagtatgatctcagcctcagttctcttgtctgtaagatggaagaactggattaggcagtttgtaagattcctcctaactttcacagtcgatgacaagattgtctttttatctgatattttgaagggtatattgctttgaagtaagtctcaataaggcaatatattttagggcatctttcttcttatctctgacagtgttcttaaaattatttgaatatcataagagccttggtgtctgtcctaattcctttctcactcaccgatgctgaatacccagttgaatcaaactgtcaacctaccaaaaacgatattgtggcttatgggtattgctgtctcattcttggtatattcttgtgttaactgcccattggcctgaaaatactcattgtaagcctgaaaaaaaaaatctttcccactgttttttctgcttgttgtaagaatcaaatgaaataatgtatgtgaaagcaccttgtaaactgtaacctatcaatgtaaaatgttaaggtgtgttgttatttcattaattacttctttgtttagaatggaatttcctatgcactactgtagctaggaaatgctgaaaacaactgtgttttttaattaatcaataactgcaaaattaaagtaccttcaatggataagacaacaaaaaaaaaaaaaaaa

SEQ ID NO: 82 - DNA, 296 bp, Homo sapiens
aaaaaaaatctttcccactgttttttctgcttgttgtaagaatcaaatgaaataatgtatgtgaaagcaccttgtaaactgtaacctatcaatgtaaaatgttaaggtgtgttgttatttcattaattacttctttgtttagaatggaatttcctatgcactactgtagctaggaaatgctgaaaacaactgtgttttttaattaatcaataactgcaaaattaaagtaccttcaatggataagacaaaaaaaaaaaaaaaaaaaaaaaaaaaaaaaaaaaaaaaaaaaaaaaaaa

SEQ ID NO: 83 - DNA, 2850 bp, Homo sapiens
gttattttgagcaatatgttttggaaaggttggttttcatcatgagtgcacgcaaatcatcagatgcatctgcttgctcctcttcagaaatatctgtgaaagagtttctagccaaagccaaagaagactttttgaaaaaatgggagaatccaactcagaataatgccggacttgaagattttgaaaggaaaaaaacccttggaacaggttcatttggaagagtcatgttggtaaaacacaaagccactgaacagtattatgccatgaagatcttagataagcagaaggataattctaatttatacatggttatggaatatgtccctgggggtgaaatgttttcacatctaagaagaattggaaggttcagtgagccccatgcacggttctatgcagctcagatagtgctaacattcgagtacctccattcactagacctcatctacagagatctaaaacctgaaaatctcttaattgaccatcaaggctatatccaggtcacagactttgggtttgccaaaagagttaaaggcagaacttggacattatgtggaactccagagtatttggctccagaaataattctcagcaagggctacaataaggcagtggattggtgggcattaggagtgctaatctatgaaatggcagctggctatcccccattctttgcagaccaaccaattcagatttatgaaaagattgtttctggaaaggtccgattcccatcccacttcagttcagatctcaaggaccttctacggaacctgctgcaggtggatttgaccaagagatttggaaatctaaagaatggtgtcagtgatataaaaactcacaagtggtttgccacgacagattggattgctatttaccagaggaaggttgaagctccattcataccaaagtttagaggctctggagataccagcaactttgatgactatgaagaagaagatatccgtgtctctataacagaaaaatgtgcaaaagaatttggtgaattttaaagaggaacaagatgacatctgagctcacactcagtgtttgcactctgttgagagataaggtagagctgagaccgtccttgttgaagcagttacctagttccttcattccaacgactgagtgaggtctttattgccatcatcccgtgtgcgcactctgcatccacctatgtaacaaggcaccgctaagcaagcattgtctgtgccataacacagtactagaccactttcttacttctctttgggttgtctttctcctctcctacatccatttcttccttttccaatttcattggttttctctaaacagtgctccattttattttgttggtgtttcagatgggcagtgttatggctacgtgatatttgaagggaaggataagtgttgctttcagtagttattgccaatattgttgttggtcaatggcttgaagataaactttctaataattattatttctttgagtagctcagacttggttttgccaaaactcttggtaatttttgaagatagactgtcttatcaccaaggaaatttatacaaattaagactaactttcttggaattcactattctggcaataaattttggtagactaatacagtacagctagacccagaaatttggaaggctgtagatcagaggttctagttccctttccctccttttatatcctcctctccttgagtaatgaagtgaccagcctgtgtagtgtgacaaacgtgtctcattcagcaggaaaaactaatgatatggatcatcacccagattctctcacttggtaccagcatttctgtaggtattagagaagagttctaagttttctaaaccttaactgttccttaaggattttagccagtattttaatagaacatgattaatgaaagtgacaaattttaaattttctctaatagtcctcatcataaactttttaaaggaaaataagcaaactaaaaagaacattggtttagataaatacttatactttgcaaagtcaaaaatggcttgatttttggaaacaatatagaggtattcatatttaaatgagggtttacatttgttttgttttgtaaccgttaaaaagaagttgtttccagctaattattgtggtgtactatatttgtgagcctagggtaggggcactgctgcaacttctgctttcatcccatgcctcatcaatgaggaaagggaacaaagtgtataaaactgccacaattgtattttaattttgaggtatgatattttcagatatttcataatttctaacctctgttctctcagtaaacagaatgtctgatcgatcatgcagatacaatgttggtatttgagaggttagtttttttcctacacttttttttgccaactgacttaacaacattgctgtcaggtggaaatttcaagcacttttgcacatttagttcagtgtttgttgagaatccatggcttaacccacttgttttgctatttttttctttgcttttaattttccccatctgattttatctctgcgtttcagtgacctaccttaaaacaacacacgagaagagttaaactgggttcattttaatgatcaatttacctgcatataaaatttatttttaatcaagctgatcttaatgtatataatcattctatttgctttattatcggtgcaggtaggtcattaacaccacttcttttcatctgtaccacaccctggtgaaacctttgaagacataaaaaaaacctgtctgagatgttctttctaccaatctatatgtctttcggttatcaagtgtttctgcatggtaatgtcatgtaaatgctgatattgatttcactggtccatctatatttaaaacgtgc

SEQ ID NO: 84 - DNA, 1951 bp, Homo sapiens
gttcgctggagccctttcctcagacccggcccggtcttcgcgcccggactcctggcgccagcgctaggcgcactcaccgctctgacgggtgcagacgcgggagttgtcccagactgtggagtggcgggcacggccccagccccccttcccttccctgaccccttcttgccatcgccccagacatggggaacgcggcgaccgccaagaaaggcagcgaggtggagagcgtgaaagagtttctagccaaagccaaagaagactttttgaaaaaatgggagaatccaactcagaataatgccggacttgaagattttgaaaggaaaaaaacccttggaacaggttcatttggaagagtcatgttggtaaaacacaaagccactgaacagtattatgccatgaagatcttagataagcagaaggttgttaaactgaagcaaatagagcatactttgaatgagaaaagaatattacaggcagtgaattttcctttccttgttcgactggagtatgcttttaaggataattctaatttatacatggttatggaatatgtccctgggggtgaaatgttttcacatctaagaagaattggaaggttcagtgagccccatgcacggttctatgcagctcagatagtgctaacattcgagtacctccattcactagacctcatctacagagatctaaaacctgaaaatctcttaattgaccatcaaggctatatccaggtcacagactttgggtttgccaaaagagttaaaggcagaacttggacattatgtggaactccagagtatttggctccagaaataattctcagcaagggctacaataaggcagtggattggtgggcattaggagtgctaatctatgaaatggcagctggctatcccccattctttgcagaccaaccaattcagatttatgaaaagattgtttctggaaagaacttttgatatgaacaaaacaaaactttgagaaaaattaacagacaaggcagtgatttatttttgaagaatttgagaagtgtagactctcaagaggactaaaggtcatatgaagaatgatgagagaaccaaaatacattaaaatcacaaatggaagaagaatattttactaatacaaaaactaagaatgtaaatgttataataattgtttcaaatcatttaattgacagtaattataaagttcttgaatctttactatattacttttatttatacttcatataagaaatccagttttctaacaaggatactgtcataactaaatttacatttattaagaaaaactgctttagttaaaattaatgtgtcttcatttttatgcattggcctcgatttgccaatcattctctattggttaaaatttatattcagctgtttatgaatatatattcattttatatcaaactttaaaattttgtatctaataatcagcatatattctaaaatcataacagtctaaatcctgggcaccttagaagaatgacaccagaaaaccttattatatcacaatattctgttttccccttcatttatttagaaatatgacaggatatttggtgtacttttgttttttaactaaaagtaccagattctctctccccatgtgggatataaaattatccccatctcttactccctttactcatctaaagtagaagtcatgaaagtggaatttttgccattaaaaggctctgtattatgtgaagttagattgtattaaccatttcccaataaatcatctgtttcaaaactcaaattcaaactagaatgtgtctctattcacattgcaaaaatattattgtctctctggttagtggctaaaagccaaattggaaactaactagttttttaaattttttaaattgtgcaaattattaaaaatccaatttggtcttataaaaaaaaaaaaaaaaaaaaaaaaaaaaaaaaaaaaaaaaaaaaaa

SEQ ID NO: 85 - DNA, 2945 bp, Homo sapiens
ccagccccccttcccttccctgaccccttcttgccatcgccccagacatggggaacgcggcgaccgccaagaaaggcagcgaggtggagagcgtgaaagagtttctagccaaagccaaagaagactttttgaaaaaatgggagaatccaactcagaataatgccggacttgaagattttgaaaggaaaaaaacccttggaacaggttcatttggaagagtcatgttggtaaaacacaaagccactgaacagtattatgccatgaagatcttagataagcagaaggttgttaaactgaagcaaatagagcatactttgaatgagaaaagaatattacaggcagtgaattttcctttccttgttcgactggagtatgcttttaaggataattctaatttatacatggttatggaatatgtccctgggggtgaaatgttttcacatctaagaagaattggaaggttcagtgagccccatgcacggttctatgcagctcagatagtgctaacattcgagtacctccattcactagacctcatctacagagatctaaaacctgaaaatctcttaattgaccatcaaggctatatccaggtcacagactttgggtttgccaaaagagttaaaggcagaacttggacattatgtggaactccagagtatttggctccagaaataattctcagcaagggctacaataaggcagtggattggtgggcattaggagtgctaatctatgaaatggcagctggctatcccccattctttgcagaccaaccaattcagatttatgaaaagattgtttctggaaaggtccgattcccatcccacttcagttcagatctcaaggaccttctacggaacctgctgcaggtggatttgaccaagagatttggaaatctaaagaatggtgtcagtgatataaaaactcacaagtggtttgccacgacagattggattgctatttaccagaggaaggttgaagctccattcataccaaagtttagaggctctggagataccagcaactttgatgactatgaagaagaagatatccgtgtctctataacagaaaaatgtgcaaaagaatttggtgaattttaaagaggaacaagatgacatctgagctcacactcagtgtttgcactctgttgagagataaggtagagctgagaccgtccttgttgaagcagttacctagttccttcattccaacgactgagtgaggtctttattgccatcatccgtgtgcgcactctgcatccacctatgtaacaaggcaccgctaagcaagcattgtctgtgccataacacagtactagaccactttcttacttctctttgggttgtctttctcctctcctacatccatttcttccttttcaatttcattggttttctctaaacagtgctccattttattttgttggtgtttcagatgggcagtgttatggctacgtgatatttgaagggaaggataagtgttgctttcagtagttattgccaatattgttgttggtcaatggcttgaagataaactttctaataattattatttctttgagtagctcagacttggttttgccaaaactcttggtaatttttgaagatagactgtcttatcaccaaggaaatttatacaaattaagactaactttcttggaattcactattctggcaataaattttggtagactaatacagtacagctagacccagaaatttggaaggctgtagatcagaggttctagttccctttccctccttttatatcctcctctccttgagtaatgaagtgaccagcctgtgtagtgtgacaaacgtgtctcattcagcaggaaaaactaatgatatggatcatcacccagattctctcacttggtaccagcatttctgtaggtattagagaagagttctaagttttctaaaccttaactgttccttaaggattttagccagtattttaatagaacatgattaatgaaagtgacaaattttaaattttctctaatagtcctcatcataaactttttaaaggaaaataagcaaactaaaaagaacattggtttagataaatacttatactttgcaaagtcaaaaatggcttgatttttggaaacaatatagaggtattcatatttaaatgagggtttacatttgttttgttttgtaaccgttaaaaagaagttgtttccagctaattattgtggtgtactatatttgtgagcctagggtaggggcactgctgcaacttctgctttcatcccatgcctcatcaatgaggaaagggaacaaagtgtataaaacctgccacaattgtattttaattttgaggtatgatattttcagatatttcataatttctaacctctgttctctcagtaaacagaatgtctgatcgatcatgcagatacaatgttggtatttgagaggttagtttttttcctacacttttttttgccaactgacttaacaacattgctgtcaggtggaaatttcaagcacttttgcacatttagttcagtgtttgttgagaatccatggcttaacccacttgttttgctatttttttctttgcttttaattttccccatctgattttatctctgcgtttcagtgacctaccttaaaacaacacacgagaagagttaaactgggttcattttaatgatcaatttacctgcatataaaatttatttttaatcaagctgatcttaatgtatataatcattctatttgctttattatcggtgcaggtaggtcattaacaccacttcttttcatctgtaccacaccctggtgaaacctttgaagacataaaaaaaacctgtctgagatgttctttctaccaatctatatgtctttcggttatcaagtgtttctgcatggtaatgtcatgtaaatgctgatattgatttcactggtccatctatatttaaaacgtgc

SEQ ID NO: 86 - protein, 351 aa, Homo sapiens
MGNAATAKKGSEVESVKEFLAKAKEDFLKKWENPTQNNAGLEDFERKKTLGTGSFGRVMLVKHKATEQYYAMKILDKQKVVKLKQIEHTLNEKRILQAVNFPFLVRLEYAFKDNSNLYMVMEYVPGGEMFSHLRRIGRFSEPHARFYAAQIVLTFEYLHSLDLIYRDLKPENLLIDHQGYIQVTDFGFAKRVKGRTWTLCGTPEYLAPEIILSKGYNKAVDWWALGVLIYEMAAGYPPFFADQPIQIYEKIVSGKVRFPSHFSSDLKDLLRNLLQVDLTKRFGNLKNGVSDIKTHKWFATTDWIAIYQRKVEAPFIPKFRGSGDTSNFDDYEEEDIRVSITEKCAKEFGEF

SEQ ID NO: 87 - protein, 257 aa, Homo sapiens
MGNAATAKKGSEVESVKEFLAKAKEDFLKKWENPTQNNAGLEDFERKKTLGTGSFGRVMLVKHKATEQYYAMKILDKQKVVKLKQIEHTLNEKRILQAVNFPFLVRLEYAFKDNSNLYMVMEYVPGGEMFSHLRRIGRFSEPHARFYAAQIVLTFEYLHSLDLIYRDLKPENLLIDHQGYIQVTDFGFAKRVKGRTWTLCGTPEYLAPEIILSKGYNKAVDWWALGVLIYEMAAGYPPFFADQPIQIYEKIVSGKNF

SEQ ID NO: 88 - protein, 351 aa, Homo sapiens
MGNAATAKKGSEVESVKEFLAKAKEDFLKKWENPTQNNAGLEDFERKKTLGTGSFGRVMLVKHKATEQYYAMKILDKQKVVKLKQIEHTLNEKRILQAVNFPFLVRLEYAFKDNSNLYMVMEYVPGGEMFSHLRRIGRFSEPHARFYAAQIVLTFEYLNSLDIIYRDLKPENLLIDHQGYIQVTDFGFAKRVKGRTWTLCGTPEYLAPEIILSKGYNKAVDWWALGVLIYEMAAGYPPFFADQPIQIYEKIVSGKVRFPSNFSSDLKDLLRNLLQVDLTKRFGNLKNGVSDIKTHKWFATTDWIAIYQRKVEAPFIPKFRGSGDTSNFDDYEEEDIRVSITEKCAKEFGEF